(19) United States
(12) Patent Application Publication
(10) Pub. No.: US 2026/0053171 A1
(43) Pub. Date: Feb. 26, 2026

(54) METHANOGEN INHIBITORS

(71) Applicant: AGRIZERONZ LIMITED PARTNERSHIP, Westmere Auckland (NZ)

(72) Inventors: Vincenzo CARBONE, Wellington (NZ); David RENNISON, Wellington (NZ); Ian Kenneth BODDY, Wellington (NZ); Margaret BRIMBLE, Wellington (NZ)

(21) Appl. No.: 19/104,671

(22) PCT Filed: Aug. 18, 2023
(86) PCT No.: PCT/NZ2023/050080

§ 371 Date: Feb. 18, 2025

(30) Foreign Application Priority Data
Aug. 19, 2022 (NZ) 791492

(51) Int. Cl. A23K 20/137; A23K 10/30; A23K 10/40; A23K 20/147; A23K 20/174; A23K 20/20; A23K 50/10; C07D 403/10; C07D 405/10; C07D 413/10; C07D 417/10

(52) U.S. Cl. CPC A23K 20/137; A23K 10/30; A23K 10/40; A23K 20/147; A23K 20/174; A23K 20/20; A23K 50/10; C07D 403/10; C07D 405/10; C07D 413/10; C07D 417/10

(57) ABSTRACT

The present invention relates to a new class of methanogen inhibitors for ruminants. The invention also extends to the use of such compounds in ruminants to reduce methane production in the rumen and/or to enhance productivity in the ruminant.

### FIELD OF INVENTION

The present invention relates to a new class of methanogen inhibitors for ruminants, in particular new compounds of Formula I, II, III or IV

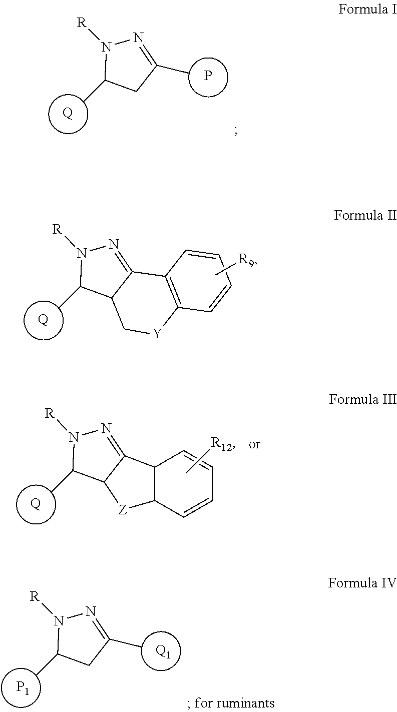

The invention also extends to the use of such compounds in ruminants to reduce methane production in the rumen and/or to enhance productivity in the ruminant.

### BACKGROUND OF INVENTION

Methane is produced as a natural consequence of digestion of feed by bacteria, fungi and protozoa in ruminant animals. This fermentation leads to the production of volatile fatty acids and peptides for the host. Fermentation also produces copious quantities of CO₂ and H₂ which are used by methanogenic archaea within the rumen to produce methane, which is ultimately released from the rumen, mostly through eructation.

The methane forming methanogens are members of the Archaea, and are quite different in a number of features to bacteria, fungi and protozoa. Methanogens have a number of unusual and archaeal-specific features, including cell wall structures, lipids, cofactors, and amino acid synthesis pathways, as well as their signature energy metabolism that is linked to methane production. They represent only about 1-4% of the rumen microbial community. It is known that, due to the unique metabolic pathways of methanogens and their low numbers, methanogen-specific inhibitors can be developed that do not adversely affect the fermentation of feed.

It has been recognised that the release of such methane is deleterious for two reasons. One is that methane is a greenhouse gas and the other is that the methane loss represents a loss of energy for the ruminant. It has been previously recognised that if one could inhibit or reduce the release of methane from ruminants that the impact of methane on the environment and atmosphere would be reduced and productivity gains might be achieved in the ruminants.

It is therefore an object of the present invention to overcome the above mentioned difficulties or to at least provide the public with a useful alternative.

### SUMMARY OF INVENTION

In one aspect the present disclosure provides a compound of Formula I

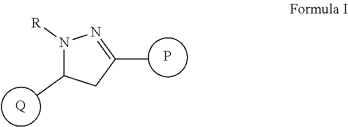

wherein R may be selected from:
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, or
—C(═O)—C₁-C₆—C(═O)—NH₂, —C(═O)—C₁-C₆—C(═O)—NH—C₁-C₆, —C(═O)—C₁-C₆—C(═O)—NH—C₃-C₈-cycloalkyl, —C(═O)—C₁-C₆—C(═O)—N(C₁-C₆)₂,

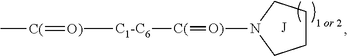

wherein J is a 5 or 6 membered heterocycle ring optionally including a second heteroatom selected from —NH—, —NC₁-C₆—, —S— or —O—; or
—C(═O)—C₁-C₆—C(═O)—NH—S(O)₂—C₁-C₆, —C(═O)—C₁-C₆—C(═O)—NH—S(O)₂—C₃-C₈-cycloalkyl, —C(═O)—C₁-C₆—C(═O)—NH—S(O)₂-aryl, C(═O)—C₁-C₆—C(═O)—NH—S(O)₂—C₁-C₆-aryl, —C(═O)—C₁-C₆—C(═O)—NH—S(O)₂-heteroaryl, —C(═O)—C₁-C₆—C(═O)—NH—S(O)₂—C₁-C₆-heteroaryl, or
—C(═O)—C₁-C₆—C(═O)—NH—S(O)₂—NH₂, —C(═O)—C₁-C₆—C(═O)—NH—S(O)₂—NH—C₁-C₆, —C(═O)—C₁-C₆—C(═O)—NH—S(O)₂—NH—C₃-C₈-cycloalkyl, —C(═O)—C₁-C₆—C(═O)—NH—S(O)₂—NH-aryl, —C(═O)—C₁-C₆—C(═O)—NH—S(O)₂—NH—C₁-C₆-aryl, —C(═O)—C₁-C₆—C(═O)—NH—S(O)₂—NH-heteroaryl, —C(═O)—C₁-C₆—C(═O)—NH—S(O)₂—NH—C₁-C₆-heteroaryl, —C(═O)—C₁-C₆—C(═O)—NH—S(O)₂—N(C₁-C₆)₂,

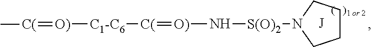

wherein J is a 5 or 6 membered heterocycle ring optionally including a second heteroatom selected from —NH—, —NC₁-C₆—, —S— or —O—; or
—C(═O)—C₁-C₆—NH—C(═O)—NH—S(O)₂—C₁-C₆, —C(═O)—C₁-C₆—NH—C(═O)—NH—S(O)₂—C₃-C₈-cycloalkyl, —C(═O)—C₁-C₆—NH—C(═O)—NH—S(O)₂-aryl, —C(═O)—C₁-C₆—NH—C(═O)—NH—S(O)₂-heteroaryl, or
—C(═O)—C₁-C₆—NH—C(═O)—NH—S(O)₂—NH₂, —C(═O)—C₁-C₆—NH—C(═O)—NH—S(O)₂—NH—C₁-C₆, —C(═O)—C₁-C₆—NH—C(═O)—NH—S(O)₂—NH—C₃-C₈-cycloalkyl, —C(═O)—C₁-C₆—NH—C(═O)—NH—S(O)₂—NH-aryl, —C(═O)—C₁-C₆—C(═O)—NH—S(O)₂—NH—C₁-C₆-aryl, —C(═O)—C₁-C₆—NH—C(═O)—NH—S(O)₂—N(C₁-C₆)₂,

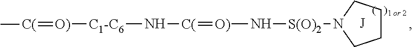

wherein J is a 5 or 6 membered heterocycle ring optionally including a second heteroatom selected from —NH—, NC₁-C₆—, —S— or —O—; or
—C₁-C₆—C(═O)—OH, —C₁-C₆—C(═O)—O—C₁-C₆, —C₁-C₆—C(═O)—O—C₁-C₆—O—(C═O)—C₁-C₆, —C₁-C₆—OH, or
—C₁-C₆—C(═O)—NH₂, —C₁-C₆—C(═O)—NH—C₁-C₆, —C₁-C₆—C(═O)—NH—C₃-C₈-cycloalkyl, —C₁-C₆—C(═O)—N(C₁-C₆)₂,

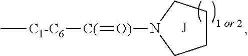

wherein J is a 5 or 6 membered heterocycle ring optionally including a second heteroatom selected from —NH—, —NC₁-C₆—, —S— or —O—; or —C₁-C₆—C(═O)—NH—S(O)₂—C₁-C₆, —C₁-C₆—C(═O)—NH—S(O)₂—C₃-C₈-cycloalkyl, —C₁-C₆—C(═O)—NH—S(O)₂-aryl, —C₁-C₆—C(═O)—NH—S(O)₂—C₁-C₆-aryl, —C₁-C₆—C(═O)—NH—S(O)₂-heteroaryl, —C₁-C₆—C(═O)—NH—S(O)₂—C₁-C₆-heteroaryl, or
—C₁-C₆—C(═O)—NH—S(O)₂—NH₂, —C₁-C₆—C(═O)—NH—S(O)₂—NH—C₁-C₆, —C₁-C₆—C(═O)—NH—S(O)₂—NH—C₃-C₈-cycloalkyl, —C₁-C₆—C(═O)—NH—S(O)₂—NH-aryl, —C₁-C₆—C(═O)—NH—S(O)₂—NH—C₁-C₆-aryl, —C₁-C₆—C(═O)—NH—S(O)₂—NH-heteroaryl, —C₁-C₆—C(═O)—NH—S(O)₂—NH—C₁-C₆-heteroaryl, —C₁-C₆—C(═O)—NH—S(O)₂—N(C₁-C₆)₂,

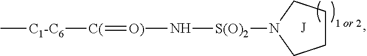

wherein J is a 5 or 6 membered heterocycle ring optionally including a second heteroatom selected from —NH—, NC₁-C₆—, —S— or —O—; or
—C₁-C₆—NH—C(═O)—NH—S(O)₂—C₁-C₆, —C₁-C₆—NH—C(═O)—NH—S(O)₂—C₃-C₈-cycloalkyl, —C₁-C₆—NH—C(═O)—NH—S(O)₂-aryl, —C₁-C₆—NH—C(═O)—NH—S(O)₂-heteroaryl, or
—C₁-C₆—NH—C(═O)—NH—S(O)₂—NH₂, —C₁-C₆—NH—C(═O)—NH—S(O)₂—NH—C₁-C₆, —C₁-C₆—NH—C(═O)—NH—S(O)₂—NH—C₃-C₈-cycloalkyl, —C₁-C₆—NH—C(═O)—NH—S(O)₂—NH-aryl, —C₁-C₆—C(═O)—NH—S(O)₂—NH—C₁-C₆-aryl, —C₁-C₆—NH—C(═O)—NH—S(O)₂—N(C₁-C₆)₂,

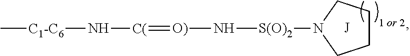

wherein J is a 5 or 6 membered heterocycle ring optionally including a second heteroatom selected from —NH—, NC₁-C₆—, —S— or —O—; or or
—C(═O)-aryl-C(═O)—OH, —C(═O)-aryl-C(═O)—O—C₁-C₆, —C(═O)-aryl-C(═O)—O—C₁-C₆—O—(C═O)—C₁-C₆, —C(═O)-aryl-OH, or
—C(═O)-aryl-C(═O)—NH₂, —C(═O)-aryl-C(═O)—NH—C₁-C₆, —C(═O)-aryl-C(═O)—NH—C₃-C₈-cycloalkyl, —C(═O)-aryl-C(═O)—N(C₁-C₆)₂,

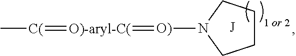

wherein J is a 5 or 6 membered heterocycle ring optionally including a second heteroatom selected from —NH—, —NC₁-C₆—, —S— or —O—; or
—C(═O)-aryl-C(═O)—NH—S(O)₂—C₁-C₆, —C(═O)-aryl-C(═O)—NH—S(O)₂—C₃-C₈-cycloalkyl, —C(═O)-aryl-C(═O)—NH—S(O)₂-aryl, —C(═O)-aryl-C(═O)—NH—S(O)₂—C₁-C₆-aryl, —C(═O)-aryl-C(═O)—NH—S(O)₂-heteroaryl, —C(═O)-aryl-C(═O)—NH—S(O)₂—C₁-C₆-heteroaryl, or
—C(═O)-aryl-C(═O)—NH—S(O)₂—NH₂, —C(═O)-aryl-C(═O)—NH—S(O)₂—NH—C₁-C₆, —C(═O)-aryl-C(═O)—NH—S(O)₂—NH—C₃-C₈-cycloalkyl, —C(═O)-aryl-C(═O)—NH—S(O)₂—NH-aryl, —C(═O)-aryl-C(═O)—NH—S(O)₂—NH—C₁-C₆-aryl, —C(═O)-aryl-C(═O)—NH—S(O)₂—NH-heteroaryl, C(═O)-aryl-C(═O)—NH—S(O)₂—NH—C₁-C₆-heteroaryl, —C(═O)-aryl-C(═O)—NH—S(O)₂—N(C₁-C₆)₂,

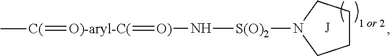

wherein J is a 5 or 6 membered heterocycle ring optionally including a second heteroatom selected from —NH—, NC₁-C₆—, —S— or —O—; or
—C(═O)-aryl-NH—C(═O)—NH—S(O)₂—C₁-C₆, —C(═O)-aryl-NH—C(═O)—NH—S(O)₂—C₃-C₈-cycloalkyl, —C(═O)-aryl-NH—C(═O)—NH—S(O)₂-aryl, —C(═O)-aryl-NH—C(═O)—NH—S(O)₂-heteroaryl, or
—C(═O)-aryl-NH—C(═O)—NH—S(O)₂—NH₂, —C(═O)-aryl-NH—C(═O)—NH—S(O)₂—NH—C₁-C₆, —C(═O)-aryl-NH—C(═O)—NH—S(O)₂—NH—C₃-C₈-cycloalkyl, —C(═O)-aryl-NH—C(═O)—NH—S(O)₂—NH-aryl, —C(═O)-aryl-C(═O)—NH—S(O)₂—NH—C₁-C₆-aryl, —C(═O)-aryl-NH—C(═O)—NH—S(O)₂—N(C₁-C₆)₂,

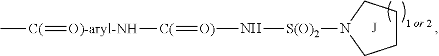

wherein J is a 5 or 6 membered heterocycle ring optionally including a second heteroatom selected from —NH—, NC₁-C₆—, —S— or —O—; or
or
-aryl-C(═O)—OH, -aryl-C(═O)—O—C₁-C₆, -aryl-C(═O)—O—C₁-C₆—O—(C═O)—C₁-C₆, -aryl-C₁-C₆—OH, or
-aryl-C(═O)—NH₂, -aryl-C(═O)—NH—C₁-C₆, -aryl-C(═O)—NH—C₃-C₈-cycloalkyl, -aryl-C(═O)—N(C₁-C₆)₂,

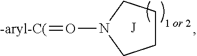

wherein J is a 5 or 6 membered heterocycle ring optionally including a second heteroatom selected from —NH—, —NC₁-C₆—, —S— or —O—; or
-aryl-C(═O)—NH—S(O)₂—C₁-C₆, -aryl-C(═O)—NH—S(O)₂—C₃-C₈-cycloalkyl, -aryl-C(═O)—NH—S(O)₂-aryl, -aryl-C(═O)—NH—S(O)₂—C₁-C₆-aryl, -aryl-C(═O)—NH—S(O)₂-heteroaryl, -aryl-C(═O)—NH—S(O)₂—C₁-C₆-heteroaryl, or
-aryl-C(═O)—NH—S(O)₂—NH₂, -aryl-C(═O)—NH—S(O)₂—NH—C₁-C₆, -aryl-C(═O)—NH—S(O)₂—NH—C₃-C₈-cycloalkyl, -aryl-C(═O)—NH—S(O)₂—NH-aryl, -aryl-C(═O)—NH—S(O)₂—NH—C₁-C₆-aryl, -aryl-C(═O)—NH—S(O)₂—NH-heteroaryl, -aryl-C(═O)—NH—S(O)₂—NH—C₁-C₆-heteroaryl, -aryl-C(═O)—NH—S(O)₂—N(C₁-C₆)₂,

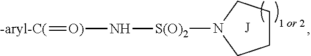

wherein J is a 5 or 6 membered heterocycle ring optionally including a second heteroatom selected from —NH—, NC₁-C₆—, —S— or —O—; or
-aryl-NH—C(═O)—NH—S(O)₂—C₁-C₆, -aryl-NH—C(═O)—NH—S(O)₂—C₃-C₈-cycloalkyl, -aryl-NH—C(═O)—NH—S(O)₂-aryl, -aryl-NH—C(═O)—NH—S(O)₂-heteroaryl, or
-aryl-NH—C(═O)—NH—S(O)₂—NH₂, -aryl-NH—C(═O)—NH—S(O)₂—NH—C₁-C₆, -aryl-NH—C(═O)—NH—S(O)₂—NH—C₃-C₈-cycloalkyl, -aryl-NH—C(═O)—NH—S(O)₂—NH-aryl, -aryl-C(═O)—NH—S(O)₂—NH—C₁-C₆-aryl, -aryl-NH—C(═O)—NH—S(O)₂—N(C₁-C₆)₂,

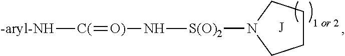

wherein J is a 5 or 6 membered heterocycle ring optionally including a second heteroatom selected from —NH—, NC₁-C₆—, —S— or —O—; or
-aryl-S(O)₂—OH, -aryl-S(O)₂—NH₂, -aryl-S(O)₂—NH—C₁-C₆, -aryl-S(O)₂—N(C₁-C₆)₂,

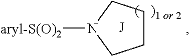

wherein J is a 5 or 6 membered heterocycle ring optionally including a second heteroatom selected from —NH—, —NC₁-C₆—, —S— or —O—; or
-aryl-S(O)₂—NH—C₃-C₈-cycloalkyl, or
—C(═O)-heteroaryl-C(═O)—OH, —C(═O)-heteroaryl-C(═O)—O—C₁-C₆, —C(═O)-heteroaryl-C(═O)—O—C₁-C₆—O—(C═O)—C₁-C₆, —C(═O)-aryl-OH, or
—C(═O)-heteroaryl-C(═O)—NH₂, —C(═O)-heteroaryl-C(═O)—NH—C₁-C₆, —C(═O)-heteroaryl-C(═O)—NH—C₃-C₈-cycloalkyl, —C(═O)-heteroaryl-C(═O)—N(C₁-C₆)₂,

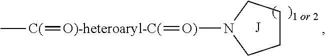

wherein J is a 5 or 6 membered heterocycle ring optionally including a second heteroatom selected from —NH—, —NC₁-C₆—, —S— or —O—; or
—C(═O)-heteroaryl-C(═O)—NH—S(O)₂—C₁-C₆, —C(═O)-heteroaryl-C(═O)—NH—S(O)₂—C₃-C₈-cycloalkyl, —C(═O)-heteroaryl-C(═O)—NH—S(O)₂-aryl, —C(═O)-heteroaryl-C(═O)—NH—S(O)₂—C₁-C₆-aryl, —C(═O)-heteroaryl-C(═O)—NH—S(O)₂-heteroaryl, —C(═O)-heteroaryl-C(═O)—NH—S(O)₂—C₁-C₆-heteroaryl, or
—C(═O)-heteroaryl-C(═O)—NH—S(O)₂—NH₂, —C(═O)-heteroaryl-C(═O)—NH—S(O)₂—NH—C₁-C₆, —C(═O)-heteroaryl-C(═O)—NH—S(O)₂—NH—C₃-C₈-cycloalkyl, —C(═O)-heteroaryl-C(═O)—NH—S(O)₂—NH-aryl, —C(═O)-heteroaryl-C(═O)—NH—S(O)₂—NH—C₁-C₆-aryl, —C(═O)-heteroaryl-C(═O)—NH—S(O)₂—NH-heteroaryl, —C(═O)-heteroaryl-C(═O)—NH—S(O)₂—NH—C₁-C₆-heteroaryl, —C(═O)-heteroaryl-C(═O)—NH—S(O)₂—N(C₁-C₆)₂,

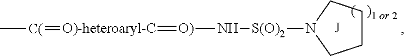

wherein J is a 5 or 6 membered heterocycle ring optionally including a second heteroatom selected from —NH—, NC₁-C₆—, —S— or —O—; or
—C(═O)-heteroaryl-NH—C(═O)—NH—S(O)₂—C₁-C₆, —C(═O)-heteroaryl-NH—C(═O)—NH—S(O)₂—C₃-C₈-cycloalkyl, —C(═O)-heteroaryl-NH—C(═O)—NH—S(O)₂-aryl, —C(═O)-heteroaryl-NH—C(═O)—NH—S(O)₂-heteroaryl, or
—C(═O)-heteroaryl-NH—C(═O)—NH—S(O)₂—NH₂, —C(═O)-heteroaryl-NH—C(═O)—NH—S(O)₂—NH—C₁-C₆, —C(═O)-heteroaryl-NH—C(═O)—NH—S(O)₂—NH—C₃-C₈-cycloalkyl, —C(═O)-heteroaryl-NH—C(═O)—NH—S(O)₂—NH-aryl, —C(═O)-heteroaryl-C(═O)—NH—S(O)₂—NH—C₁-C₆-aryl, —C(═O)-heteroaryl-NH—C(═O)—NH—S(O)₂—N(C₁-C₆)₂,

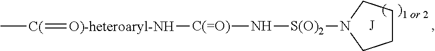

wherein J is a 5 or 6 membered heterocycle ring optionally including a second heteroatom selected from —NH—, NC₁-C₆—, —S— or —O—; or
or
-heteroaryl-C(═O)—OH, -heteroaryl-C(═O)—O—C₁-C₆, -heteroaryl-C(═O)—O—C₁-C₆—O—(C═O)—C₁-C₆, -heteroaryl-C₁-C₆—OH, or
-heteroaryl-C(═O)—NH₂, -heteroaryl-C(═O)—NH—C₁-C₆, -heteroaryl-C(═O)—NH—C₃-C₈-cycloalkyl, -heteroaryl-C(═O)—N(C₁-C₆)₂,

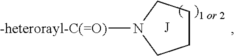

wherein J is a 5 or 6 membered heterocycle ring optionally including a second heteroatom selected from —NH—, —NC₁-C₆—, —S— or —O—; or
-heteroaryl-C(═O)—NH—S(O)₂—C₁-C₆, -heteroaryl-C(═O)—NH—S(O)₂—C₃-C₈-cycloalkyl, heteroaryl-C(═O)—NH—S(O)₂-aryl, -heteroaryl-C(═O)—NH—S(O)₂—C₁-C₆-aryl, -heteroaryl-C(═O)—NH—S(O)₂-heteroaryl, -heteroaryl-C(═O)—NH—S(O)₂—C₁-C₆-heteroaryl, or
-heteroaryl-C(═O)—NH—S(O)₂—NH₂, -heteroaryl-C(═O)—NH—S(O)₂—NH—C₁-C₆, -heteroaryl-C(═O)—NH—S(O)₂—NH—C₃-C₈-cycloalkyl, -heteroaryl-C(═O)—NH—S(O)₂—NH-aryl, -heteroaryl-C(═O)—NH—S(O)₂—NH—C₁-C₆-aryl, -heteroaryl-C(═O)—NH—S(O)₂—NH-heteroaryl, -heteroaryl-C(═O)—NH—S(O)₂—NH—C₁-C₆-heteroaryl, -heteroaryl-C(═O)—NH—S(O)₂—N(C₁-C₆)₂,

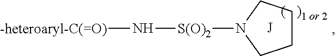

wherein J is a 5 or 6 membered heterocycle ring optionally including a second heteroatom selected from —NH—, NC₁-C₆—, —S— or —O—; or
-heteroaryl-NH—C(═O)—NH—S(O)₂—C₁-C₆, -heteroaryl-NH—C(═O)—NH—S(O)₂—C₃-C₈-cycloalkyl, -heteroaryl-NH—C(═O)—NH—S(O)₂-aryl, -heteroaryl-NH—C(═O)—NH—S(O)₂-heteroaryl, or
-heteroaryl-NH—C(═O)—NH—S(O)₂—NH₂, -heteroaryl-NH—C(═O)—NH—S(O)₂—NH—C₁-C₆, heteroaryl-NH—C(═O)—NH—S(O)₂—NH—C₃-C₈-cycloalkyl, -heteroaryl-NH—C(═O)—NH—S(O)₂—NH-aryl, -heteroaryl-C(═O)—NH—S(O)₂—NH—C₁-C₆-aryl, -heteroaryl-NH—C(═O)—NH—S(O)₂—N(C₁-C₆)₂,

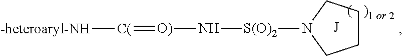

wherein J is a 5 or 6 membered heterocycle ring optionally including a second heteroatom selected from —NH—, NC₁-C₆—, —S— or —O—;
or
—C₁-C₆-aryl-C(═O)—OH, —C₁-C₆-aryl-C(═O)—O—C₁-C₆, —C₁-C₆-aryl-C(═O)—O—C₁-C₆—O—(C═O)—C₁-C₆, —C₁-C₆-aryl-C₁-C₆—OH, or
—C₁-C₆-aryl-C(═O)—NH₂, —C₁-C₆-aryl-C(═O)—NH—C₁-C₆, —C₁-C₆-aryl-C(═O)—NH—C₃-C₈-cycloalkyl, —C₁-C₆-aryl-C(═O)—N(C₁-C₆)₂,

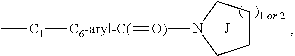

wherein J is a 5 or 6 membered heterocycle ring optionally including a second heteroatom selected from —NH—, —NC₁-C₆—, —S— or —O—; or
—C₁-C₆-aryl-C(═O)—NH—S(O)₂—C₁-C₆, —C₁-C₆-aryl-C(═O)—NH—S(O)₂—C₃-C₈-cycloalkyl, —C₁-C₆-aryl-C(═O)—NH—S(O)₂-aryl, —C₁-C₆-aryl-C(═O)—NH—S(O)₂—C₁-C₆-aryl, —C₁-C₆-aryl-C(═O)—NH—S(O)₂-heteroaryl, —C₁-C₆-aryl-C(═O)—NH—S(O)₂—C₁-C₆-heteroaryl, or
—C₁-C₆-aryl-C(═O)—NH—S(O)₂—NH₂, —C₁-C₆-aryl-C(═O)—NH—S(O)₂—NH—C₁-C₆, —C₁-C₆-aryl-C(═O)—NH—S(O)₂—NH—C₃-C₈-cycloalkyl, —C₁-C₆-aryl-C(═O)—NH—S(O)₂—NH-aryl, —C₁-C₆-aryl-C(═O)—NH—S(O)₂—NH—C₁-C₆-aryl, —C₁-C₆-aryl-C(═O)—NH—S(O)₂—NH-heteroaryl, —C₁-C₆-aryl-C(═O)—NH—S(O)₂—NH—C₁-C₆-heteroaryl, —C₁-C₆-aryl-C(═O)—NH—S(O)₂—N(C₁-C₆)₂, —C₁-C₆

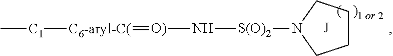

wherein J is a 5 or 6 membered heterocycle ring optionally including a second heteroatom selected from —NH—, NC₁-C₆—, —S— or —O—; or
—C₁-C₆-aryl-NH—C(═O)—NH—S(O)₂—C₁-C₆, —C₁-C₆-aryl-NH—C(═O)—NH—S(O)₂—C₃-C₈-cycloalkyl, —C₁-C₆-aryl-NH—C(═O)—NH—S(O)₂-aryl, —C₁-C₆-aryl-NH—C(═O)—NH—S(O)₂-heteroaryl, or
—C₁-C₆-aryl-NH—C(═O)—NH—S(O)₂—NH₂, —C₁-C₆-aryl-NH—C(═O)—NH—S(O)₂—NH—C₁-C₆, —C₁-C₆-aryl-NH—C(═O)—NH—S(O)₂—NH—C₃-C₈-cycloalkyl, —C₁-C₆-aryl-NH—C(═O)—NH—S(O)₂—NH-aryl, —C₁-C₆-aryl-C(═O)—NH—S(O)₂—NH—C₁-C₆-aryl, —C₁-C₆-aryl-NH—C(═O)—NH—S(O)₂—N(C₁-C₆)₂,

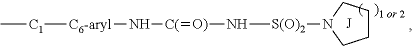

wherein J is a 5 or 6 membered heterocycle ring optionally including a second heteroatom selected from —NH—, NC₁-C₆—, —S— or —O—; or
—C₁-C₆-aryl-S(O)₂—OH, —C₁-C₆-aryl-S(O)₂—NH₂, —C₁-C₆-aryl-S(O)₂—NH—C₁-C₆, —C₁-C₆-aryl-S(O)₂—N(C₁-C₆)₂,

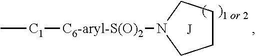

wherein J is a 5 or 6 membered heterocycle ring optionally including a second heteroatom selected from —NH—, —NC₁-C₆—, —S— or —O—; or
—C₁-C₆-aryl-S(O)₂—NH—C₃-C₈-cycloalkyl, or
—C₁-C₆-heteroaryl-C(═O)—OH, —C₁-C₆-heteroaryl-C(═O)—O—C₁-C₆, —C₁-C₆-heteroaryl-C(═O)—O—C₁-C₆—O—(C═O)—C₁-C₆, —C₁-C₆-heteroaryl-C₁-C₆—OH, or
—C₁-C₆-heteroaryl-C(═O)—NH₂, —C₁-C₆-heteroaryl-C(═O)—NH—C₁-C₆, —C₁-C₆-heteroaryl-C(═O)—NH—C₃-C₈-cycloalkyl, —C₁-C₆-heteroaryl-C(═O)—N(C₁-C₆)₂,

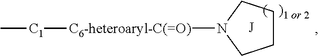

wherein J is a 5 or 6 membered heterocycle ring optionally including a second heteroatom selected from —NH—, —NC₁-C₆—, —S— or —O—; or
—C₁-C₆-heteroaryl-C(═O)—NH—S(O)₂—C₁-C₆, —C₁-C₆-heteroaryl-C(═O)—NH—S(O)₂—C₃-C₈-cycloalkyl, —C₁-C₆-heteroaryl-C(═O)—NH—S(O)₂-aryl, —C₁-C₆-heteroaryl-C(═O)—NH—S(O)₂—C₁-C₆-aryl, —C₁-C₆-heteroaryl-C(═O)—NH—S(O)₂-heteroaryl, —C₁-C₆-heteroaryl-C(═O)—NH—S(O)₂—C₁-C₆-heteroaryl, or
—C₁-C₆-heteroaryl-C(═O)—NH—S(O)₂—NH₂, —C₁-C₆-heteroaryl-C(═O)—NH—S(O)₂—NH—C₁-C₆, —C₁-C₆heteroaryl-C(═O)—NH—S(O)₂—NH—C₃-C₈-cycloalkyl, —C₁-C₆-heteroaryl-C(═O)—NH—S(O)₂—NH-aryl, —C₁-C₆-heteroaryl-C(═O)—NH—S(O)₂—NH—C₁-C₆-aryl, —C₁-C₆-heteroaryl-C(═O)—NH—S(O)₂—NH-heteroaryl, —C₁-C₆-heteroaryl-C(═O)—NH—S(O)₂—NH—C₁-C₆-heteroaryl, —C₁-C₆-heteroaryl-C(═O)—NH—S(O)₂—N(C₁-C₆)₂,

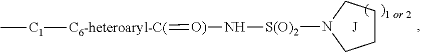

wherein J is a 5 or 6 membered heterocycle ring optionally including a second heteroatom selected from —NH—, NC₁-C₆—, —S— or —O—; or
—C₁-C₆-heteroaryl-NH—C(═O)—NH—S(O)₂—C₁-C₆, —C₁-C₆-heteroaryl-NH—C(═O)—NH—S(O)₂—C₃-C₈-cycloalkyl, —C₁-C₆-heteroaryl-NH—C(═O)—NH—S(O)₂-aryl, —C₁-C₆-heteroaryl-NH—C(═O)—NH—S(O)₂-heteroaryl, or
—C₁-C₆-heteroaryl-NH—C(═O)—NH—S(O)₂—NH₂, —C₁-C₆-heteroaryl-NH—C(═O)—NH—S(O)₂—NH—C₁-C₆, —C₁-C₆-heteroaryl-NH—C(═O)—NH—S(O)₂—NH—C₃-C₈-cycloalkyl, —C₁-C₆-heteroaryl-NH—C(═O)—NH—S(O)₂—NH-aryl, —C₁-C₆-heteroaryl-C(═O)—NH—S(O)₂—NH—C₁-C₆-aryl, —C₁-C₆-heteroaryl-NH—C(═O)—NH—S(O)₂—N(C₁-C₆)₂,

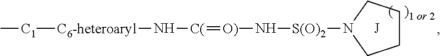

wherein J is a 5 or 6 membered heterocycle ring optionally including a second heteroatom selected from —NH—, NC₁-C₆—, —S— or —O—;
wherein P may be a 5 to 10 membered aryl ring or rings optionally containing one or more heteroatoms, and wherein P may be further substituted with one or more halo, —C₁-C₆-alkyl, —C₁-C₆-alkyl substituted with one or more halo or hydroxy; —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

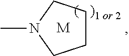

wherein M is a 5 or 6 membered heterocycle ring optionally including a second heteroatom selected from —NH—, —NC₁-C₆—, —S— or —O— or C₂-C₈ cycloheteroalkyl including one or more heteroatoms selected from —NH—, —NC₁-C₆—, —S— or —O—; or
aryl, aryl substituted with one or more, halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

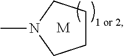

wherein M is a 5 or 6 membered heterocycle ring optionally including a second heteroatom selected from —NH—, —NC₁-C₆—, —S— or —O—; or
C₁-C₆-aryl; C₁-C₆-aryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

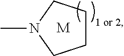

wherein M is a 5 or 6 membered heterocycle ring optionally including a second heteroatom selected from —NH—, —NC₁-C₆—, —S— or —O—; or
heteroaryl; heteroaryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

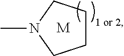

wherein M is a 5 or 6 membered heterocycle ring optionally including a second heteroatom selected from —NH—, —NC₁-C₆—, —S— or —O—; or
C₁-C₆-heteroaryl; C₁-C₆-heteroaryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

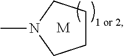

wherein M is a 5 or 6 membered heterocycle ring optionally including a second heteroatom selected from —NH—, —NC₁-C₆—, —S— or —O—; or
—O-aryl; —O-aryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

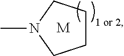

wherein M is a 5 or 6 membered heterocycle ring optionally including a second heteroatom selected from —NH—, —NC₁-C₆—, —S— or —O—; or
—O—C₁-C₆-aryl; —O—C₁-C₆-aryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

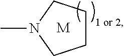

wherein M is a 5 or 6 membered heterocycle ring optionally including a second heteroatom selected from —NH—, —NC₁-C₆—, —S— or —O—; or
wherein Q may be selected from

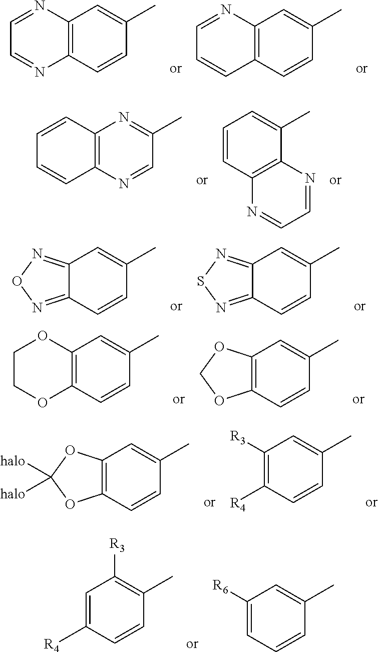

and
wherein R₃, R₄, or R₆ are independently selected from —H, halo, —C₁-C₆-alkyl, —C₁-C₆-alkyl substituted with one or more halo, —CN, hydroxy, —O—C₁-C₆-alkyl, —O—C₁-C₆-alkyl substituted with one or more halo; and
or a salt thereof;
with the proviso that the following compounds

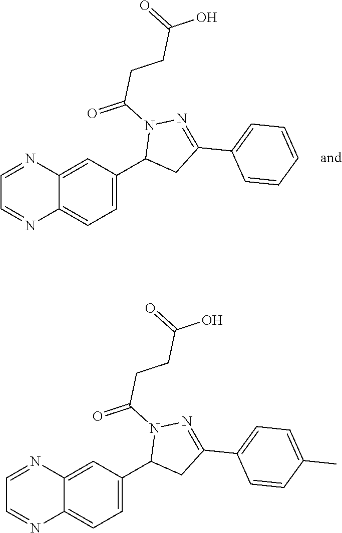 [SMILES: O=C(O)CCC(=O)N1N=C(c2ccccc2)CC1c1ccc2nccnc2c1 ; Cc1ccc(C2=NN(C(=O)CCC(=O)O)C(c3ccc4nccnc4c3)C2)cc1]

are excluded from the compounds of Formula I.

In one example R is selected from
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, or
—C(═O)—C₁-C₆—C(═O)—NH₂, —C(═O)—C₁-C₆—C(═O)—NH—C₁-C₆, —C(═O)—C₁-C₆—C(═O)—N(C₁-C₆)₂,
—C(═O)—C₁-C₆—C(═O)—NH—S(O)₂—C₁-C₆, —C(═O)—C₁-C₆—C(═O)—NH—S(O)₂-aryl, —C(═O)—C₁-C₆—C(═O)—NH—S(O)₂—N(C₁-C₆)₂,

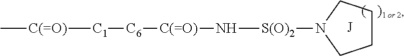

wherein J is a 5 or 6 membered heterocycle ring optionally including a second heteroatom selected from —NH—, NC₁-C₆—, —S— or —O—; or
—C₁-C₆—C(═O)—OH, or
-aryl-C(═O)—OH, or
-aryl-C(═O)—NH—S(O)₂—C₁-C₆.

In one example R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, —C₁-C₆—C(═O)—OH, -aryl-C(═O)—OH.

In one example R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, -aryl-C(═O)—OH.

In one example R is selected from —C(═O)—C₁-C₆—C(═O)—OH.

In one example R is selected from —C(═O)—C₁-C₆—C(═O)—NH—S(O)₂—C₁-C₆.

In one example R is selected from —C(═O)—C₁-C₆—C(═O)—NH—S(O)₂-aryl.

In one example R is selected from —C(═O)—C₁-C₆—C(═O)—NH—S(O)₂—N(C₁-C₆)₂.

In one example R is selected from -aryl-C(═O)—OH.

In one example R is selected from —C(═O)—CH₂—CH₂—C(═O)—OH.

In one example R is selected from —C(═O)—CH₂—CH₂—C(═O)—NH—S(O)₂-Me.

In one example R is selected from —C(═O)—CH₂—CH₂—C(═O)—NH—S(O)₂-iPr.

In one example R is selected from —C(═O)—CH₂—CH₂—C(═O)—NH—S(O)₂-Ph.

In one example R is selected from —C(═O)—CH₂—CH₂—C(═O)—NH—S(O)₂—NMe₂.

In one example R is selected from -phenyl-C(═O)—OH.

In one example P is aryl or an aryl substituted with one or more halo, —C₁-C₆-alkyl, —O—C₁-C₆-alkyl.

In one example P is aryl substituted with aryl or an aryl substituted with halo, —C₁-C₆-alkyl, —O—C₁-C₆-alkyl, —C₁-C₆-alkyl substituted with one or more halo, or —O—C₁-C₆-alkyl substituted with one or more halo.

In one example P is phenyl or a phenyl substituted with one or more halo, —C₁-C₆-alkyl, —O—C₁-C₆-alkyl, —C₁-C₆-alkyl substituted with one or more halo, or —O—C₁-C₆-alkyl substituted with one or more halo.

In one example P is phenyl substituted with phenyl or a phenyl substituted with halo, —C₁-C₆-alkyl, —O—C₁-C₆-alkyl, —C₁-C₆-alkyl substituted with one or more halo, or —O—C₁-C₆-alkyl substituted with one or more halo.

In one example P is phenyl substituted with a phenyl substituted with halo.

In one example P is phenyl substituted with a phenyl substituted with —O—C₁-C₆-alkyl.

In one example P is phenyl substituted with a phenyl substituted with —Cl.

In one example P is phenyl substituted with a phenyl substituted with —OCF₃.

In one example P is phenyl substituted with a phenyl substituted with —OCF₃ and —F.

In one example P is phenyl substituted with a phenyl substituted with —OⁱPr.

In one example P is phenyl substituted with a phenyl substituted with —O—C₃-alkyl.

In one example Q is selected from:

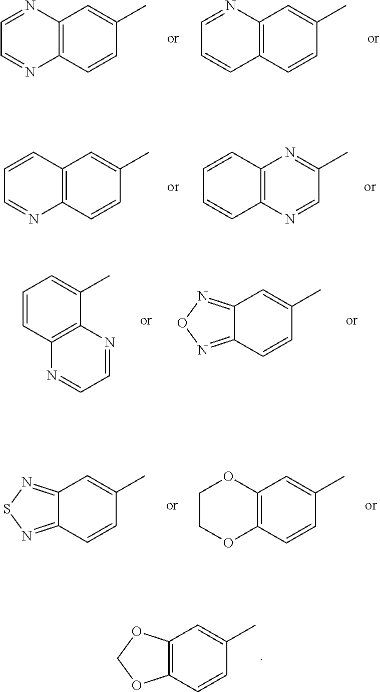

In one example Q is selected from:

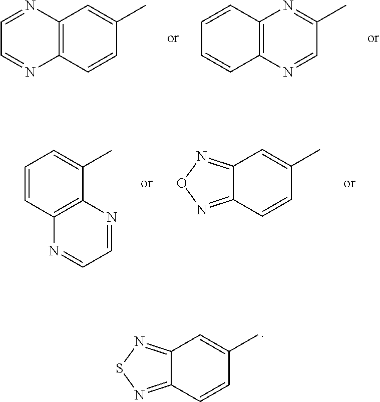

In one example Q is

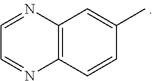

In one example the compound or salt of Formula I is selected from one or more of the following:

| Compound No | Structure |
|---|---|
| 1 | 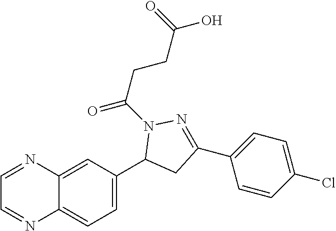 [SMILES: O=C(O)CCC(=O)N1N=C(c2ccc(Cl)cc2)CC1c1ccc2nccnc2c1] |
| 2 | 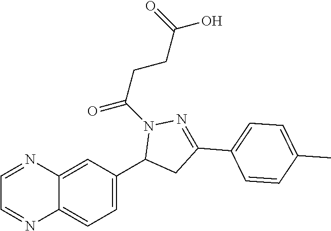 [SMILES: Cc1ccc(C2=NN(C(=O)CCC(=O)O)C(c3ccc4nccnc4c3)C2)cc1] |
| 3 | 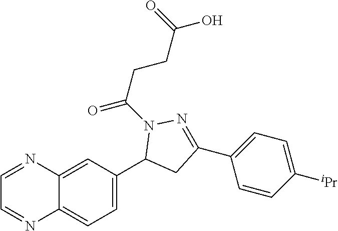 [SMILES: CC(C)c1ccc(C2=NN(C(=O)CCC(=O)O)C(c3ccc4nccnc4c3)C2)cc1] |
| 4 | 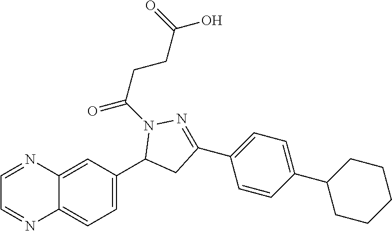 [SMILES: O=C(O)CCC(=O)N1N=C(c2ccc(C3CCCCC3)cc2)CC1c1ccc2nccnc2c1] |
| 5 | 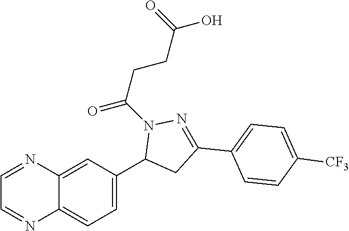 [SMILES: O=C(O)CCC(=O)N1N=C(c2ccc(C(F)(F)F)cc2)CC1c1ccc2nccnc2c1] |
| 6 | 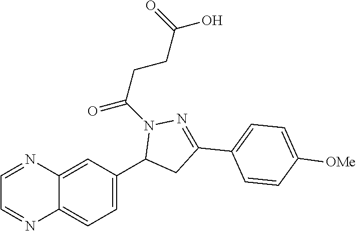 [SMILES: COc1ccc(C2=NN(C(=O)CCC(=O)O)C(c3ccc4nccnc4c3)C2)cc1] |
| 7 | 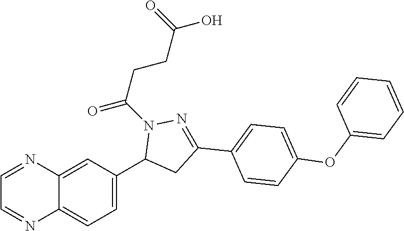 [SMILES: O=C(O)CCC(=O)N1N=C(c2ccc(Oc3ccccc3)cc2)CC1c1ccc2nccnc2c1] |
| 8 | 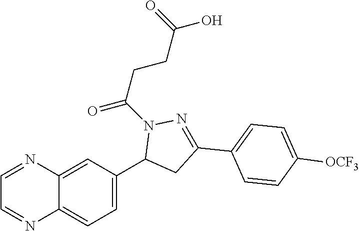 [SMILES: O=C(O)CCC(=O)N1N=C(c2ccc(OC(F)(F)F)cc2)CC1c1ccc2nccnc2c1] |
| 9 | 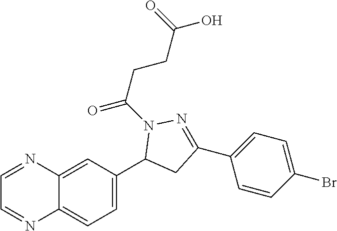 [SMILES: O=C(O)CCC(=O)N1N=C(c2ccc(Br)cc2)CC1c1ccc2nccnc2c1] |
| 10 | 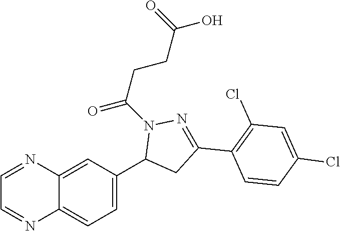 [SMILES: O=C(O)CCC(=O)N1N=C(c2ccc(Cl)cc2Cl)CC1c1ccc2nccnc2c1] |
| 11 | 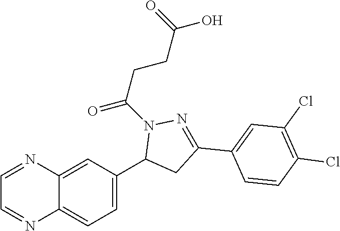 [SMILES: O=C(O)CCC(=O)N1N=C(c2ccc(Cl)c(Cl)c2)CC1c1ccc2nccnc2c1] |
| 12 | 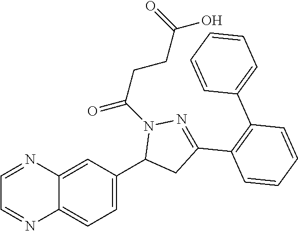 [SMILES: O=C(O)CCC(=O)N1N=C(c2ccccc2-c2ccccc2)CC1c1ccc2nccnc2c1] |
| 13 | 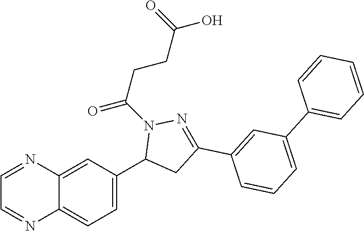 [SMILES: O=C(O)CCC(=O)N1N=C(c2cccc(-c3ccccc3)c2)CC1c1ccc2nccnc2c1] |
| 14 | 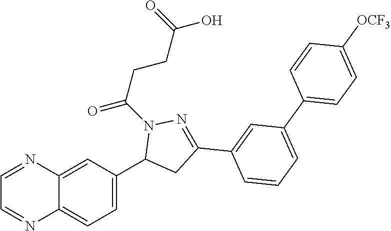 [SMILES: O=C(O)CCC(=O)N1N=C(c2cccc(-c3ccc(OC(F)(F)F)cc3)c2)CC1c1ccc2nccnc2c1] |
| 15 | 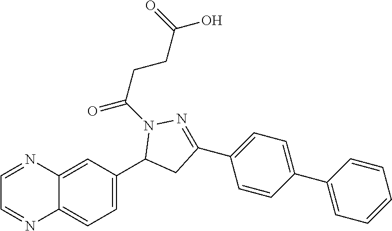 [SMILES: O=C(O)CCC(=O)N1N=C(c2ccc(-c3ccccc3)cc2)CC1c1ccc2nccnc2c1] |
| 16 | 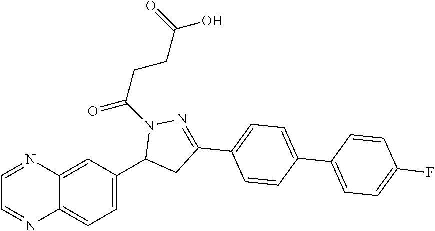 [SMILES: O=C(O)CCC(=O)N1N=C(c2ccc(-c3ccc(F)cc3)cc2)CC1c1ccc2nccnc2c1] |
| 17 | 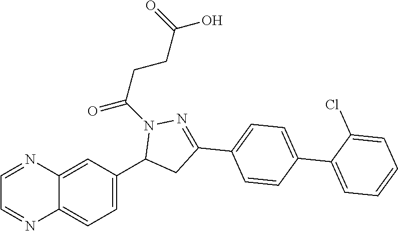 [SMILES: O=C(O)CCC(=O)N1N=C(c2ccc(-c3ccccc3Cl)cc2)CC1c1ccc2nccnc2c1] |
| 18 | 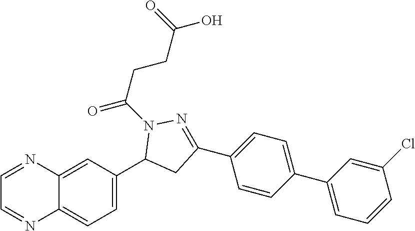 [SMILES: O=C(O)CCC(=O)N1N=C(c2ccc(-c3cccc(Cl)c3)cc2)CC1c1ccc2nccnc2c1] |
| 19 | 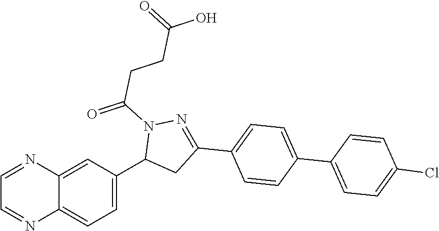 [SMILES: O=C(O)CCC(=O)N1N=C(c2ccc(-c3ccc(Cl)cc3)cc2)CC1c1ccc2nccnc2c1] |
| 20 | 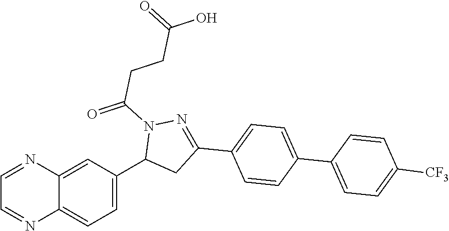 [SMILES: O=C(O)CCC(=O)N1N=C(c2ccc(-c3ccc(C(F)(F)F)cc3)cc2)CC1c1ccc2nccnc2c1] |
| 21 | 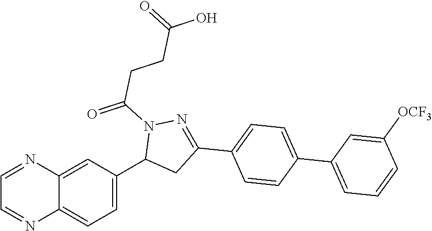 [SMILES: O=C(O)CCC(=O)N1N=C(c2ccc(-c3cccc(OC(F)(F)F)c3)cc2)CC1c1ccc2nccnc2c1] |
| 22 | 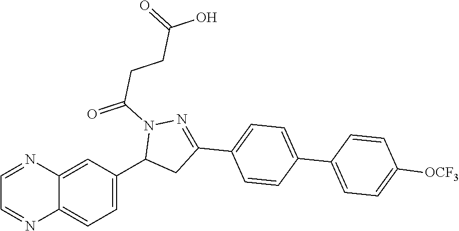 [SMILES: O=C(O)CCC(=O)N1N=C(c2ccc(-c3ccc(OC(F)(F)F)cc3)cc2)CC1c1ccc2nccnc2c1] |
| 23 | 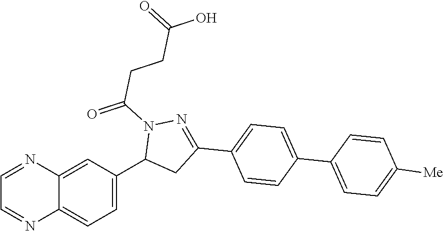 [SMILES: Cc1ccc(-c2ccc(C3=NN(C(=O)CCC(=O)O)C(c4ccc5nccnc5c4)C3)cc2)cc1] |
| 24 | 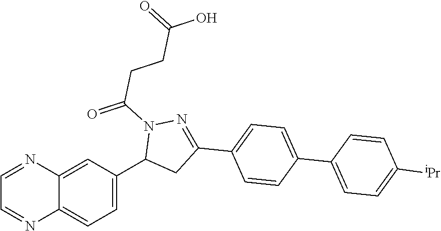 [SMILES: CC(C)c1ccc(-c2ccc(C3=NN(C(=O)CCC(=O)O)C(c4ccc5nccnc5c4)C3)cc2)cc1] |
| 25 | 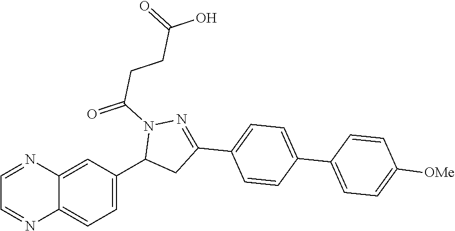 [SMILES: COc1ccc(-c2ccc(C3=NN(C(=O)CCC(=O)O)C(c4ccc5nccnc5c4)C3)cc2)cc1] |
| 26 | 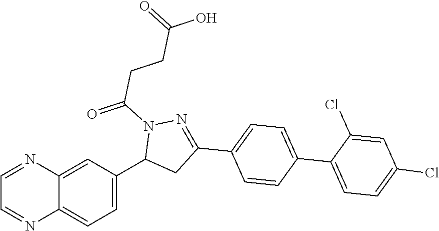 [SMILES: O=C(O)CCC(=O)N1N=C(c2ccc(-c3ccc(Cl)cc3Cl)cc2)CC1c1ccc2nccnc2c1] |
| 27 | 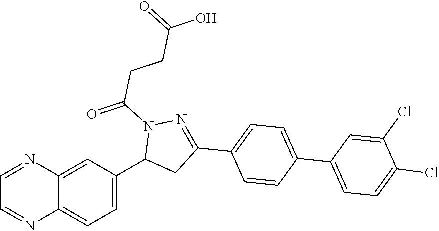 [SMILES: O=C(O)CCC(=O)N1N=C(c2ccc(-c3ccc(Cl)c(Cl)c3)cc2)CC1c1ccc2nccnc2c1] |
| 28 | 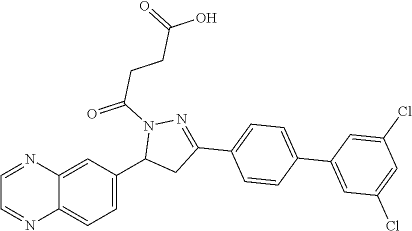 [SMILES: O=C(O)CCC(=O)N1N=C(c2ccc(-c3cc(Cl)cc(Cl)c3)cc2)CC1c1ccc2nccnc2c1] |
| 29 | 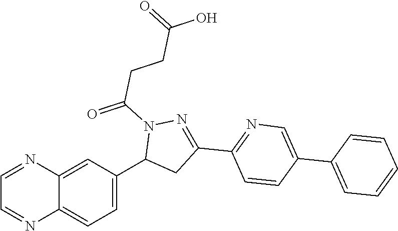 [SMILES: O=C(O)CCC(=O)N1N=C(c2ccc(-c3ccccc3)cn2)CC1c1ccc2nccnc2c1] |
| 30 | 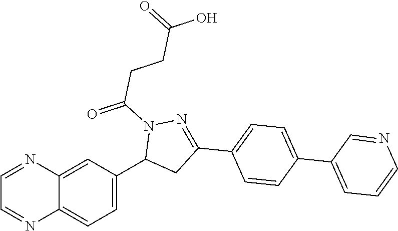 [SMILES: O=C(O)CCC(=O)N1N=C(c2ccc(-c3cccnc3)cc2)CC1c1ccc2nccnc2c1] |
| 31 | 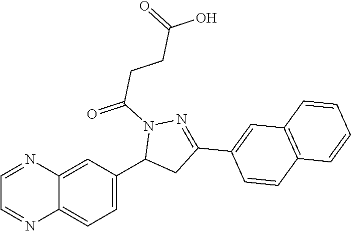 [SMILES: O=C(O)CCC(=O)N1N=C(c2ccc3ccccc3c2)CC1c1ccc2nccnc2c1] |
| 32 | 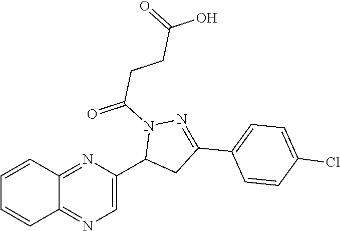 [SMILES: O=C(O)CCC(=O)N1N=C(c2ccc(Cl)cc2)CC1c1cnc2ccccc2n1] |
| 33 | 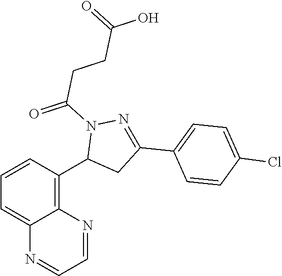 [SMILES: O=C(O)CCC(=O)N1N=C(c2ccc(Cl)cc2)CC1c1cccc2nccnc12] |
| 34 | 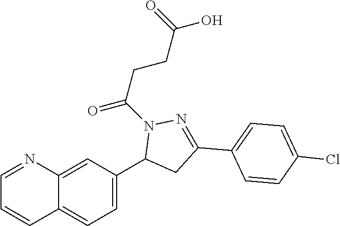 [SMILES: O=C(O)CCC(=O)N1N=C(c2ccc(Cl)cc2)CC1c1ccc2cccnc2c1] |
| 35 | 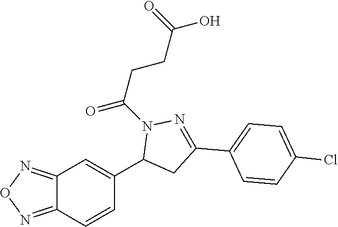 [SMILES: O=C(O)CCC(=O)N1N=C(c2ccc(Cl)cc2)CC1c1ccc2nonc2c1] |
| 36 | 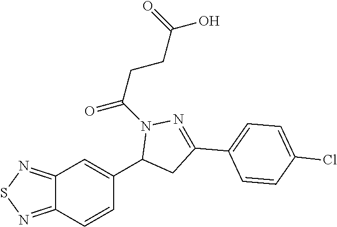 [SMILES: O=C(O)CCC(=O)N1N=C(c2ccc(Cl)cc2)CC1c1ccc2nsnc2c1] |
| 37 | 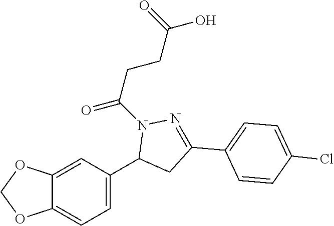 [SMILES: O=C(O)CCC(=O)N1N=C(c2ccc(Cl)cc2)CC1c1ccc2c(c1)OCO2] |
| 38 | 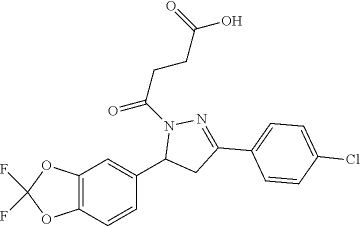 [SMILES: O=C(O)CCC(=O)N1N=C(c2ccc(Cl)cc2)CC1c1ccc2c(c1)OC(F)(F)O2] |
| 39 | 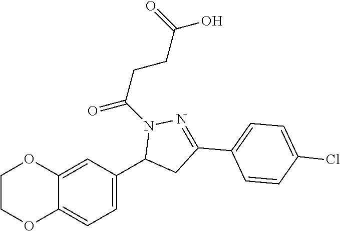 [SMILES: O=C(O)CCC(=O)N1N=C(c2ccc(Cl)cc2)CC1c1ccc2c(c1)OCCO2] |
| 40 | 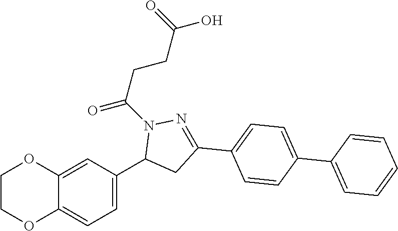 [SMILES: O=C(O)CCC(=O)N1N=C(c2ccc(-c3ccccc3)cc2)CC1c1ccc2c(c1)OCCO2] |
| 41 | 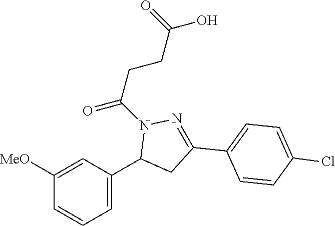 [SMILES: COc1cccc(C2CC(c3ccc(Cl)cc3)=NN2C(=O)CCC(=O)O)c1] |
| 42 | 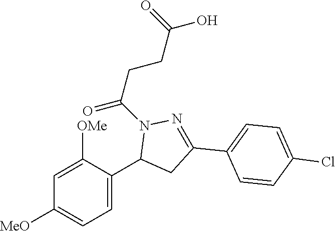 [SMILES: COc1ccc(C2CC(c3ccc(Cl)cc3)=NN2C(=O)CCC(=O)O)c(OC)c1] |
| 43 | 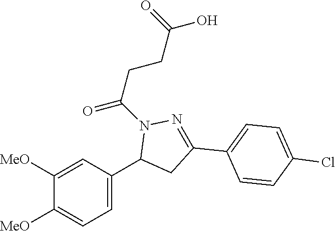 [SMILES: COc1ccc(C2CC(c3ccc(Cl)cc3)=NN2C(=O)CCC(=O)O)cc1OC] |
| 44 | 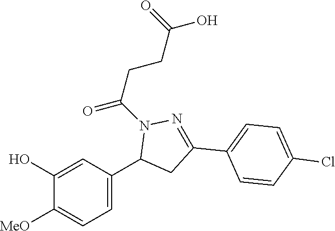 [SMILES: COc1ccc(C2CC(c3ccc(Cl)cc3)=NN2C(=O)CCC(=O)O)cc1O] |
| 45 | 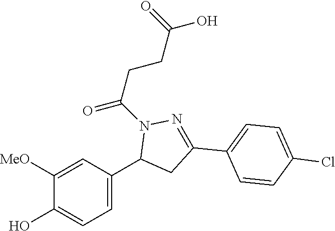 [SMILES: COc1cc(C2CC(c3ccc(Cl)cc3)=NN2C(=O)CCC(=O)O)ccc1O] |
| 46 | 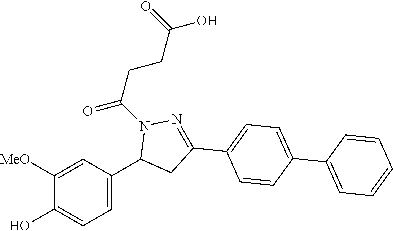 [SMILES: COc1cc(C2CC(c3ccc(-c4ccccc4)cc3)=NN2C(=O)CCC(=O)O)ccc1O] |
| 47 | 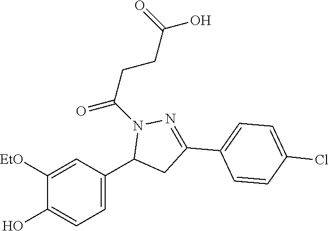 [SMILES: CCOc1cc(C2CC(c3ccc(Cl)cc3)=NN2C(=O)CCC(=O)O)ccc1O] |
| 48 | 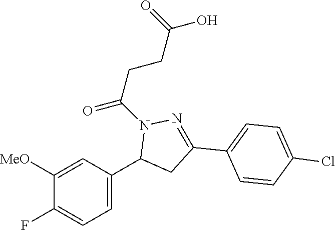 [SMILES: COc1cc(C2CC(c3ccc(Cl)cc3)=NN2C(=O)CCC(=O)O)ccc1F] |
| 49 | 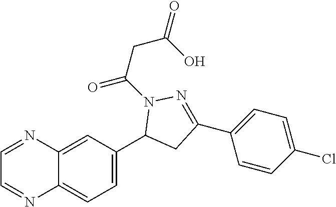 [SMILES: O=C(O)CC(=O)N1N=C(c2ccc(Cl)cc2)CC1c1ccc2nccnc2c1] |
| 50 | 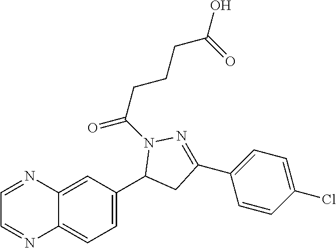 [SMILES: O=C(O)CCCC(=O)N1N=C(c2ccc(Cl)cc2)CC1c1ccc2nccnc2c1] |
| 51 | 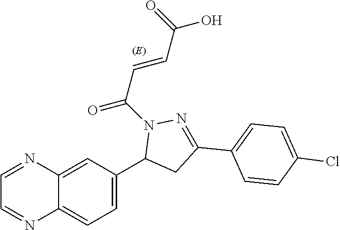 [SMILES: O=C(O)/C=C/C(=O)N1N=C(c2ccc(Cl)cc2)CC1c1ccc2nccnc2c1] |
| 52 | 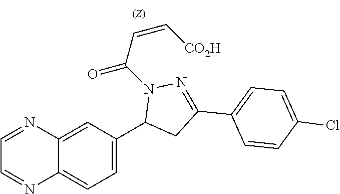 [SMILES: O=C(O)/C=C\C(=O)N1N=C(c2ccc(Cl)cc2)CC1c1ccc2nccnc2c1] |
| 53 | 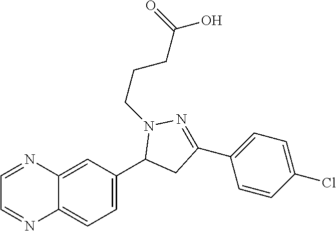 [SMILES: O=C(O)CCCN1N=C(c2ccc(Cl)cc2)CC1c1ccc2nccnc2c1] |
| 54 | 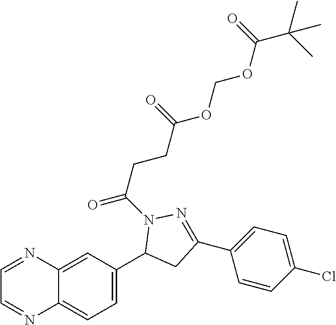 [SMILES: CC(C)(C)C(=O)OCOC(=O)CCC(=O)N1N=C(c2ccc(Cl)cc2)CC1c1ccc2nccnc2c1] |
| 55 | 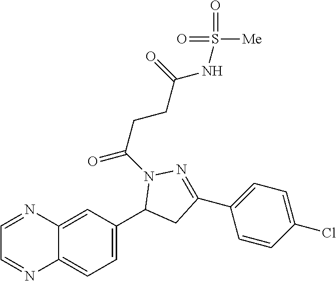 [SMILES: CS(=O)(=O)NC(=O)CCC(=O)N1N=C(c2ccc(Cl)cc2)CC1c1ccc2nccnc2c1] |
| 56 | 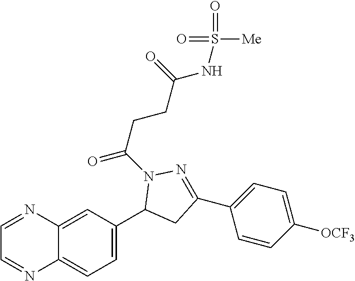 [SMILES: CS(=O)(=O)NC(=O)CCC(=O)N1N=C(c2ccc(OC(F)(F)F)cc2)CC1c1ccc2nccnc2c1] |
| 57 | 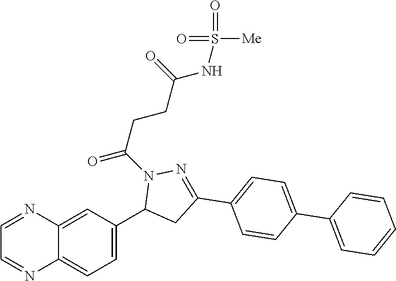 [SMILES: CS(=O)(=O)NC(=O)CCC(=O)N1N=C(c2ccc(-c3ccccc3)cc2)CC1c1ccc2nccnc2c1] |
| 58 | 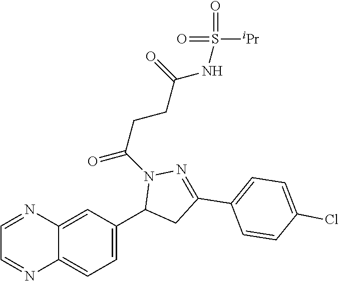 [SMILES: CC(C)S(=O)(=O)NC(=O)CCC(=O)N1N=C(c2ccc(Cl)cc2)CC1c1ccc2nccnc2c1] |
| 59 | 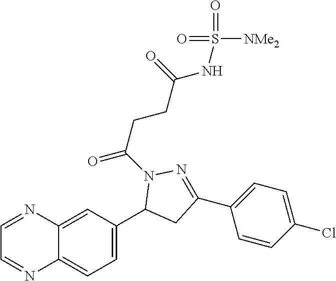 [SMILES: CN(C)S(=O)(=O)NC(=O)CCC(=O)N1N=C(c2ccc(Cl)cc2)CC1c1ccc2nccnc2c1] |
| 60 | 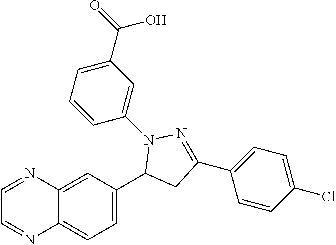 [SMILES: O=C(O)c1cccc(N2N=C(c3ccc(Cl)cc3)CC2c2ccc3nccnc3c2)c1] |
| 61 | 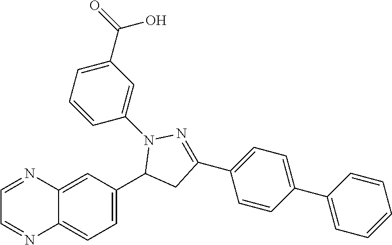 [SMILES: O=C(O)c1cccc(N2N=C(c3ccc(-c4ccccc4)cc3)CC2c2ccc3nccnc3c2)c1] |
| 62 | 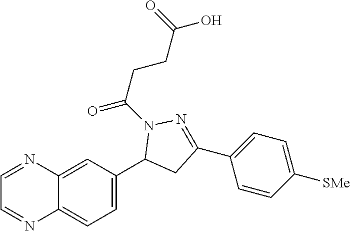 [SMILES: CSc1ccc(C2=NN(C(=O)CCC(=O)O)C(c3ccc4nccnc4c3)C2)cc1] |
| 63 | 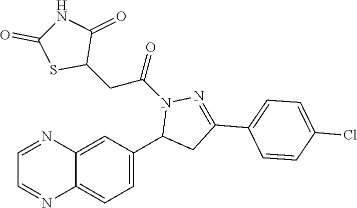 [SMILES: O=C1NC(=O)C(CC(=O)N2N=C(c3ccc(Cl)cc3)CC2c2ccc3nccnc3c2)S1] |
| 64 | 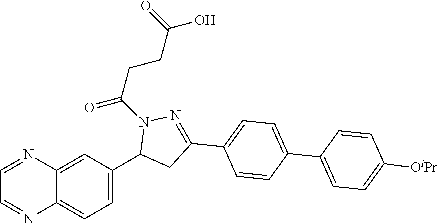 [SMILES: CC(C)Oc1ccc(-c2ccc(C3=NN(C(=O)CCC(=O)O)C(c4ccc5nccnc5c4)C3)cc2)cc1] |
| 65 | 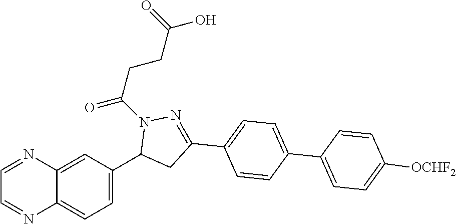 [SMILES: O=C(O)CCC(=O)N1N=C(c2ccc(-c3ccc(OC(F)F)cc3)cc2)CC1c1ccc2nccnc2c1] |
| 66 | 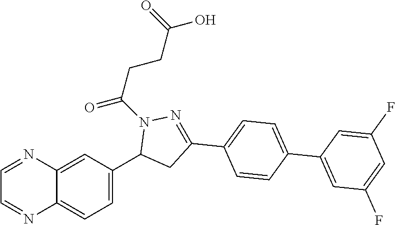 [SMILES: O=C(O)CCC(=O)N1N=C(c2ccc(-c3cc(F)cc(F)c3)cc2)CC1c1ccc2nccnc2c1] |
| 67 | 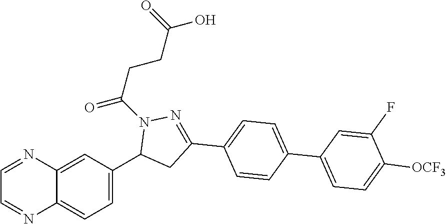 [SMILES: O=C(O)CCC(=O)N1N=C(c2ccc(-c3ccc(OC(F)(F)F)c(F)c3)cc2)CC1c1ccc2nccnc2c1] |
| 68 | 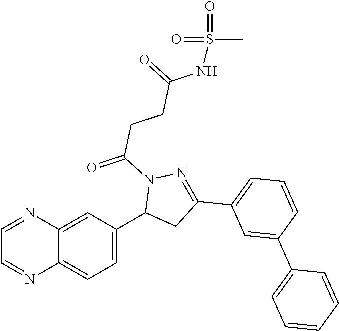 [SMILES: CS(=O)(=O)NC(=O)CCC(=O)N1N=C(c2cccc(-c3ccccc3)c2)CC1c1ccc2nccnc2c1] |
| 69 | 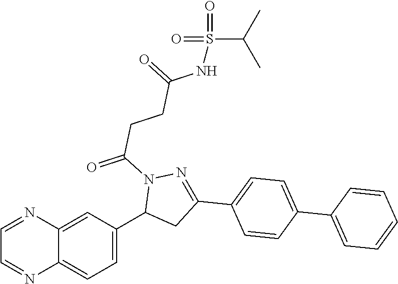 [SMILES: CC(C)S(=O)(=O)NC(=O)CCC(=O)N1N=C(c2ccc(-c3ccccc3)cc2)CC1c1ccc2nccnc2c1] |
| 70 | 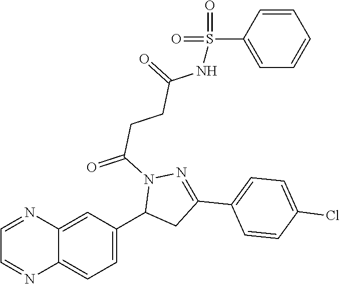 [SMILES: O=C(CCC(=O)N1N=C(c2ccc(Cl)cc2)CC1c1ccc2nccnc2c1)NS(=O)(=O)c1ccccc1] |
| 71 | 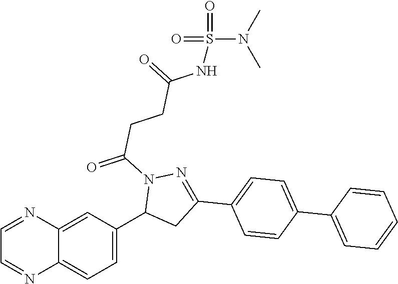 [SMILES: CN(C)S(=O)(=O)NC(=O)CCC(=O)N1N=C(c2ccc(-c3ccccc3)cc2)CC1c1ccc2nccnc2c1] |
| 72 | 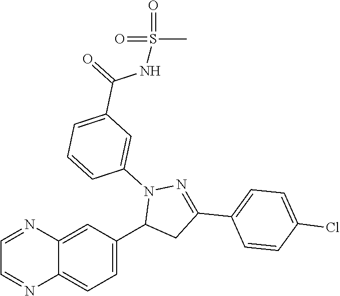 [SMILES: CS(=O)(=O)NC(=O)c1cccc(N2N=C(c3ccc(Cl)cc3)CC2c2ccc3nccnc3c2)c1] |
| 78 | 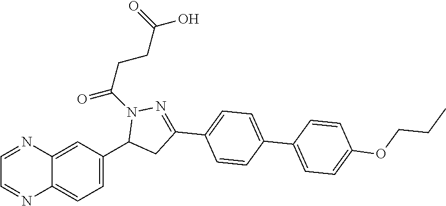 [SMILES: CCCOc1ccc(-c2ccc(C3=NN(C(=O)CCC(=O)O)C(c4ccc5nccnc5c4)C3)cc2)cc1] |
| 79 | 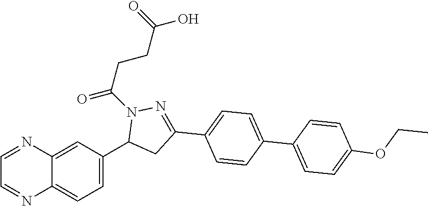 [SMILES: CCOc1ccc(-c2ccc(C3=NN(C(=O)CCC(=O)O)C(c4ccc5nccnc5c4)C3)cc2)cc1] |
| 80 | 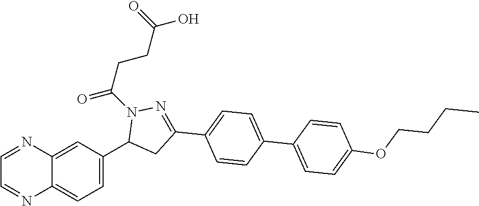 [SMILES: CCCCOc1ccc(-c2ccc(C3=NN(C(=O)CCC(=O)O)C(c4ccc5nccnc5c4)C3)cc2)cc1] |
| 81 | 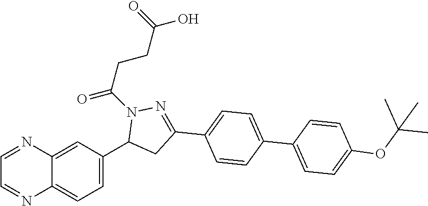 [SMILES: CC(C)(C)Oc1ccc(-c2ccc(C3=NN(C(=O)CCC(=O)O)C(c4ccc5nccnc5c4)C3)cc2)cc1] |
| 82 | 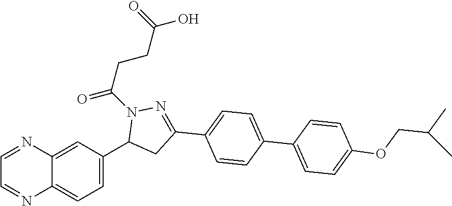 [SMILES: CC(C)COc1ccc(-c2ccc(C3=NN(C(=O)CCC(=O)O)C(c4ccc5nccnc5c4)C3)cc2)cc1] |
| 83 | 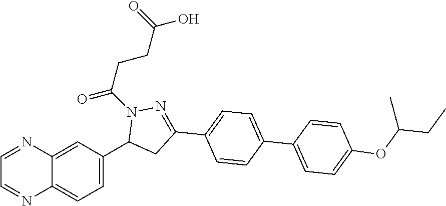 [SMILES: CCC(C)Oc1ccc(-c2ccc(C3=NN(C(=O)CCC(=O)O)C(c4ccc5nccnc5c4)C3)cc2)cc1] |
| 84 | 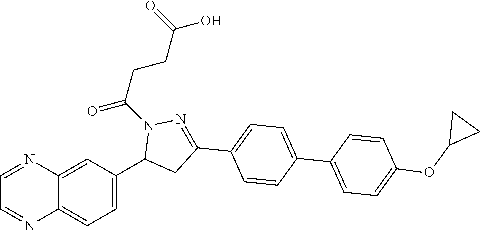 [SMILES: O=C(O)CCC(=O)N1N=C(c2ccc(-c3ccc(OC4CC4)cc3)cc2)CC1c1ccc2nccnc2c1] |
| 85 | 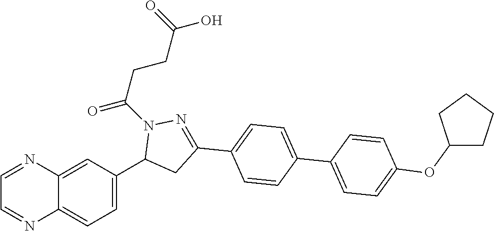 [SMILES: O=C(O)CCC(=O)N1N=C(c2ccc(-c3ccc(OC4CCCC4)cc3)cc2)CC1c1ccc2nccnc2c1] |
| 86 | 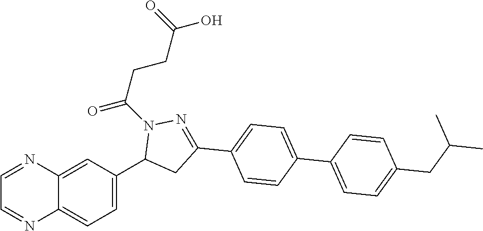 [SMILES: CC(C)Cc1ccc(-c2ccc(C3=NN(C(=O)CCC(=O)O)C(c4ccc5nccnc5c4)C3)cc2)cc1] |
| 87 | 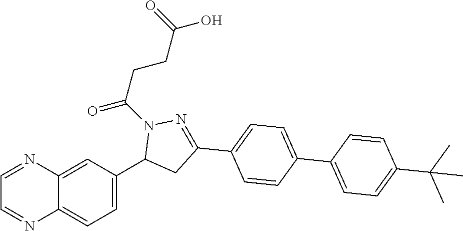 [SMILES: CC(C)(C)c1ccc(-c2ccc(C3=NN(C(=O)CCC(=O)O)C(c4ccc5nccnc5c4)C3)cc2)cc1] |
| 88 | 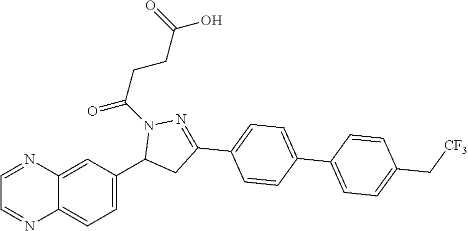 [SMILES: O=C(O)CCC(=O)N1N=C(c2ccc(-c3ccc(CC(F)(F)F)cc3)cc2)CC1c1ccc2nccnc2c1] |
| 89 | 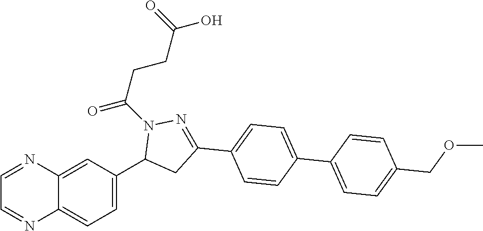 [SMILES: COCc1ccc(-c2ccc(C3=NN(C(=O)CCC(=O)O)C(c4ccc5nccnc5c4)C3)cc2)cc1] |
| 90 | 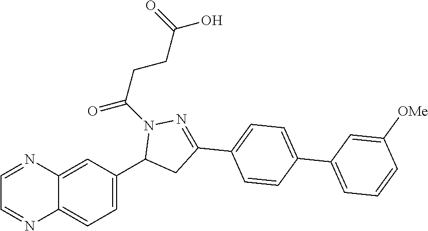 [SMILES: COc1cccc(-c2ccc(C3=NN(C(=O)CCC(=O)O)C(c4ccc5nccnc5c4)C3)cc2)c1] |
| 91 | 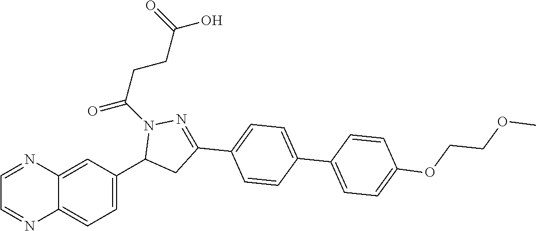 [SMILES: COCCOc1ccc(-c2ccc(C3=NN(C(=O)CCC(=O)O)C(c4ccc5nccnc5c4)C3)cc2)cc1] |
| 92 | 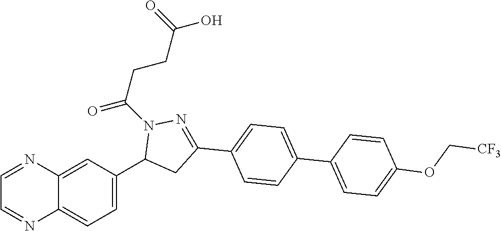 [SMILES: O=C(O)CCC(=O)N1N=C(c2ccc(-c3ccc(OCC(F)(F)F)cc3)cc2)CC1c1ccc2nccnc2c1] |
| 93 | 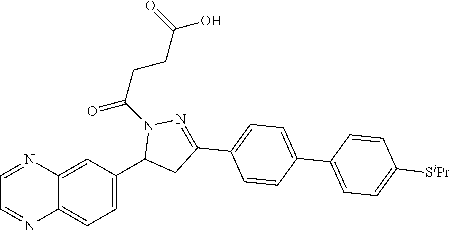 |
| 94 | 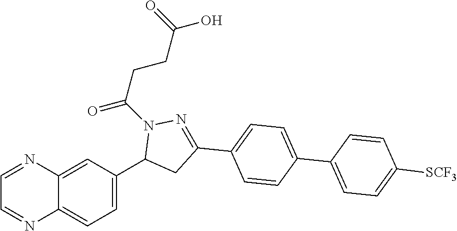 |
| 95 | 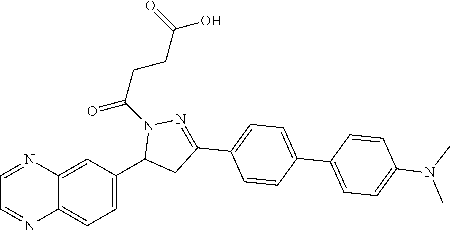 [SMILES: CN(C)c1ccc(-c2ccc(C3=NN(C(=O)CCC(=O)O)C(c4ccc5nccnc5c4)C3)cc2)cc1] |
| 96 | 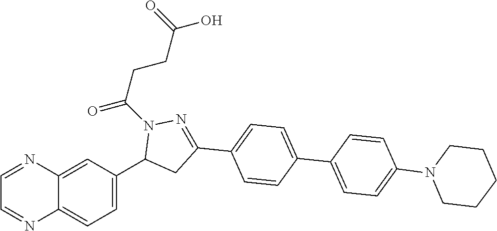 [SMILES: O=C(O)CCC(=O)N1N=C(c2ccc(-c3ccc(N4CCCCC4)cc3)cc2)CC1c1ccc2nccnc2c1] |
| 97 | 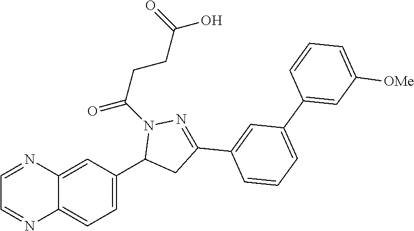 [SMILES: COc1cccc(-c2cccc(C3=NN(C(=O)CCC(=O)O)C(c4ccc5nccnc5c4)C3)c2)c1] |
| 98 | 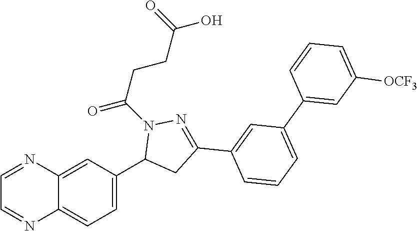 [SMILES: O=C(O)CCC(=O)N1N=C(c2cccc(-c3cccc(OC(F)(F)F)c3)c2)CC1c1ccc2nccnc2c1] |
| 99 | 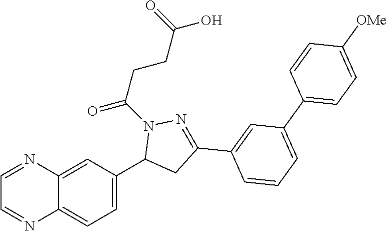 [SMILES: COc1ccc(-c2cccc(C3=NN(C(=O)CCC(=O)O)C(c4ccc5nccnc5c4)C3)c2)cc1] |
| 100 | 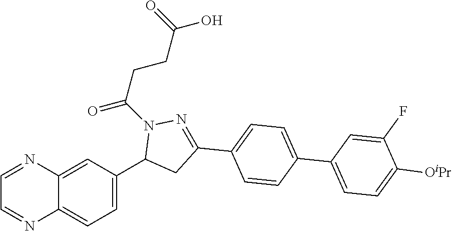 [SMILES: CC(C)Oc1ccc(-c2ccc(C3=NN(C(=O)CCC(=O)O)C(c4ccc5nccnc5c4)C3)cc2)cc1F] |
| 101 | 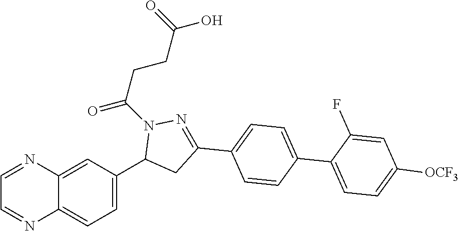 [SMILES: O=C(O)CCC(=O)N1N=C(c2ccc(-c3ccc(OC(F)(F)F)cc3F)cc2)CC1c1ccc2nccnc2c1] |
| 102 | 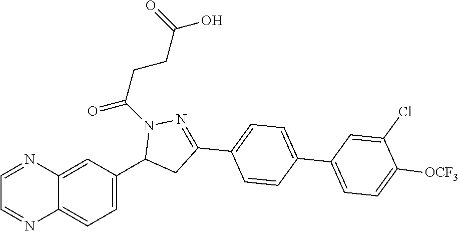 [SMILES: O=C(O)CCC(=O)N1N=C(c2ccc(-c3ccc(OC(F)(F)F)c(Cl)c3)cc2)CC1c1ccc2nccnc2c1] |
| 103 | 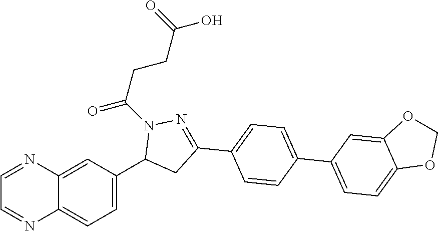 [SMILES: O=C(O)CCC(=O)N1N=C(c2ccc(-c3ccc4c(c3)OCO4)cc2)CC1c1ccc2nccnc2c1] |
| 104 | 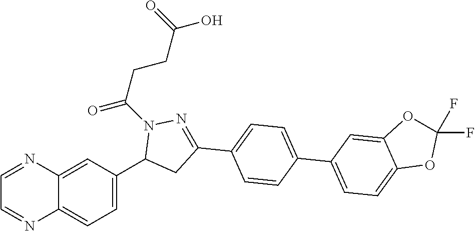 [SMILES: O=C(O)CCC(=O)N1N=C(c2ccc(-c3ccc4c(c3)OC(F)(F)O4)cc2)CC1c1ccc2nccnc2c1] |
| 105 | 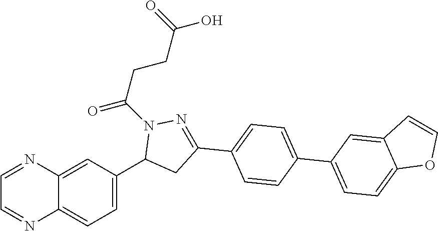 [SMILES: O=C(O)CCC(=O)N1N=C(c2ccc(-c3ccc4occc4c3)cc2)CC1c1ccc2nccnc2c1] |
| 106 | 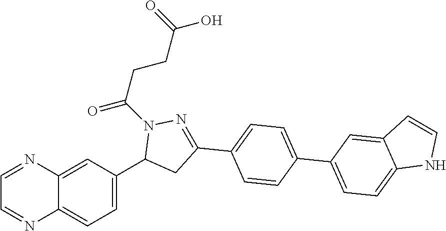 [SMILES: O=C(O)CCC(=O)N1N=C(c2ccc(-c3ccc4[nH]ccc4c3)cc2)CC1c1ccc2nccnc2c1] |
| 107 | 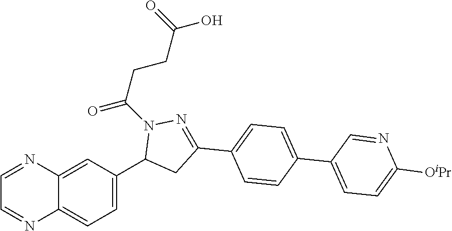 [SMILES: CC(C)Oc1ccc(-c2ccc(C3=NN(C(=O)CCC(=O)O)C(c4ccc5nccnc5c4)C3)cc2)cn1] |
| 108 | 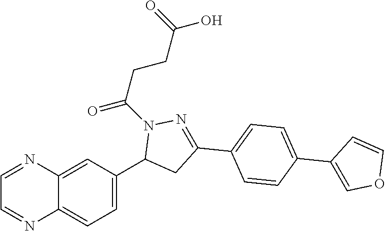 [SMILES: O=C(O)CCC(=O)N1N=C(c2ccc(-c3ccoc3)cc2)CC1c1ccc2nccnc2c1] |
| 109 | 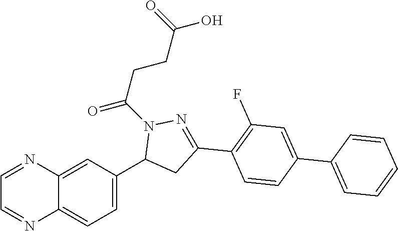 [SMILES: O=C(O)CCC(=O)N1N=C(c2ccc(-c3ccccc3)cc2F)CC1c1ccc2nccnc2c1] |
| 110 | 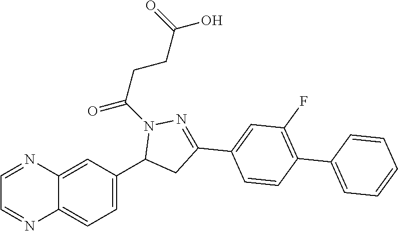 [SMILES: O=C(O)CCC(=O)N1N=C(c2ccc(-c3ccccc3)c(F)c2)CC1c1ccc2nccnc2c1] |
| 111 | 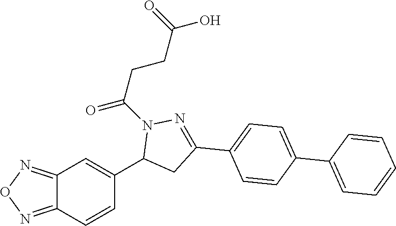 [SMILES: O=C(O)CCC(=O)N1N=C(c2ccc(-c3ccccc3)cc2)CC1c1ccc2nonc2c1] |
| 112 | 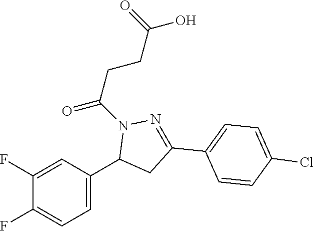 [SMILES: O=C(O)CCC(=O)N1N=C(c2ccc(Cl)cc2)CC1c1ccc(F)c(F)c1] |
| 113 | 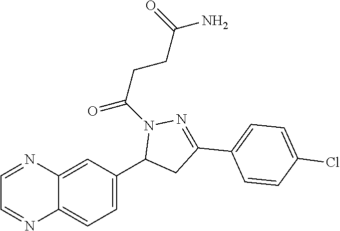 [SMILES: NC(=O)CCC(=O)N1N=C(c2ccc(Cl)cc2)CC1c1ccc2nccnc2c1] |
| 114 | 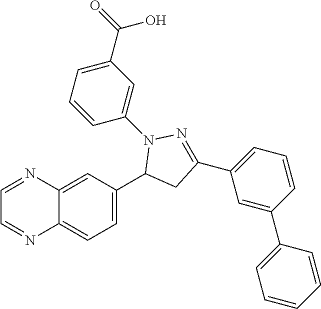 [SMILES: O=C(O)c1cccc(N2N=C(c3cccc(-c4ccccc4)c3)CC2c2ccc3nccnc3c2)c1] |
| 115 | 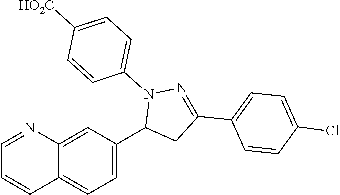 [SMILES: O=C(O)c1ccc(N2N=C(c3ccc(Cl)cc3)CC2c2ccc3cccnc3c2)cc1] |
| 116 | 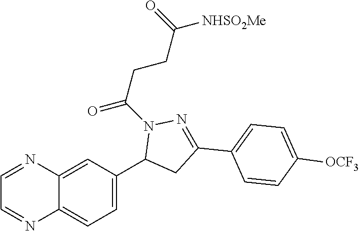 |
| 117 | 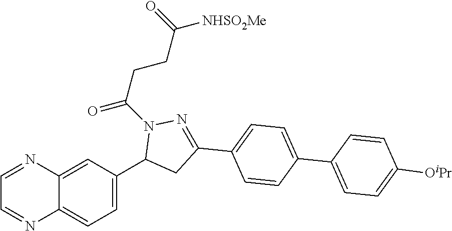 |
| 118 | 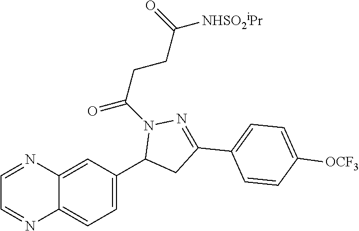 |
| 119 | 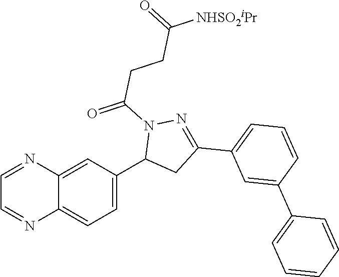 |
| 120 | 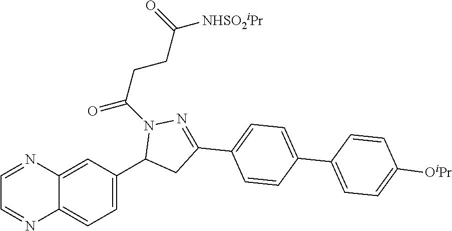 |
| 121 | 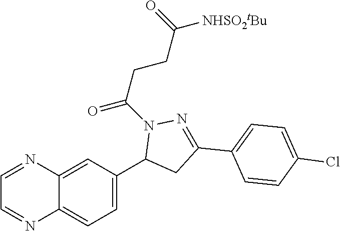 |
| 122 | 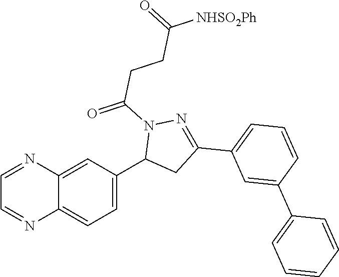 |
| 123 | 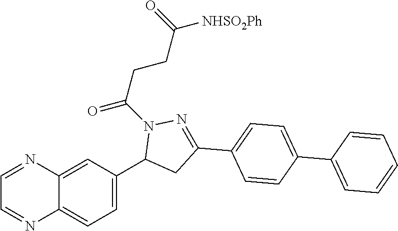 |
| 124 | 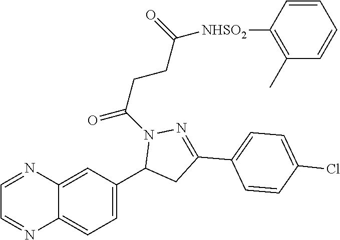 |
| 125 | 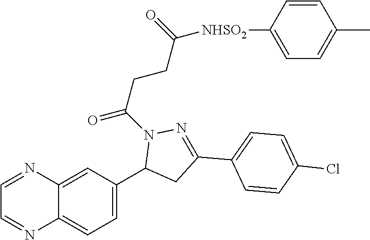 |
| 126 | 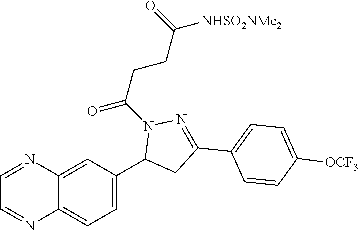 |
| 127 | 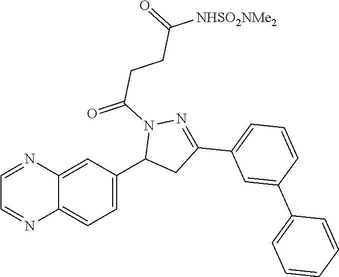 |
| 128 | 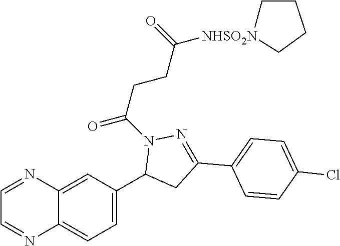 |

In one example the compound of Formula I is:

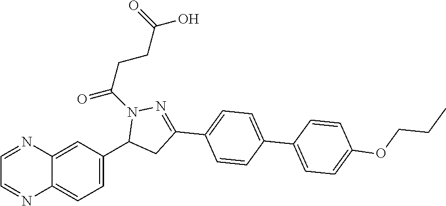 [SMILES: CCCOc1ccc(-c2ccc(C3=NN(C(=O)CCC(=O)O)C(c4ccc5nccnc5c4)C3)cc2)cc1]

or a salt thereof.

In one example the compound of Formula I is:

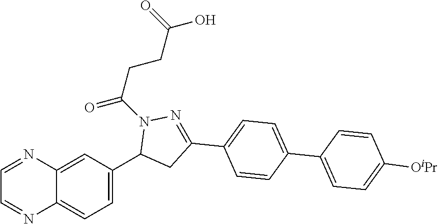 [SMILES: CC(C)Oc1ccc(-c2ccc(C3=NN(C(=O)CCC(=O)O)C(c4ccc5nccnc5c4)C3)cc2)cc1]

or a salt thereof.

In one example the compound of Formula I is:

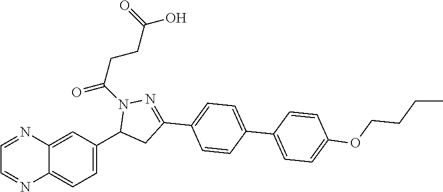 [SMILES: CCCCOc1ccc(-c2ccc(C3=NN(C(=O)CCC(=O)O)C(c4ccc5nccnc5c4)C3)cc2)cc1]

or a salt thereof.

In one example the compound of Formula I is:

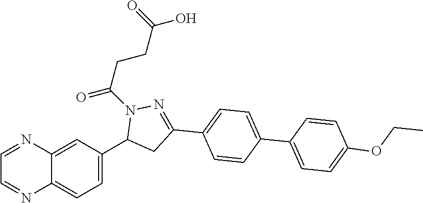 [SMILES: CCOc1ccc(-c2ccc(C3=NN(C(=O)CCC(=O)O)C(c4ccc5nccnc5c4)C3)cc2)cc1]

or a salt thereof.

In one example the compound of Formula I is:

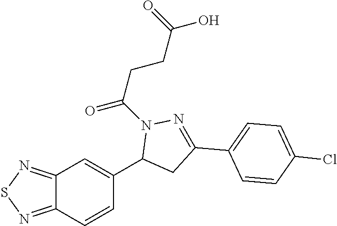 [SMILES: O=C(O)CCC(=O)N1N=C(c2ccc(Cl)cc2)CC1c1ccc2nsnc2c1]

or a salt thereof.

In one example the compound of Formula I is:

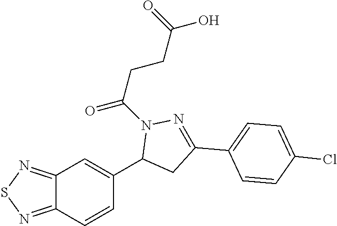 [SMILES: O=C(O)CCC(=O)N1N=C(c2ccc(Cl)cc2)CC1c1ccc2nsnc2c1]

or a salt thereof.

In one example the compound of Formula I is:

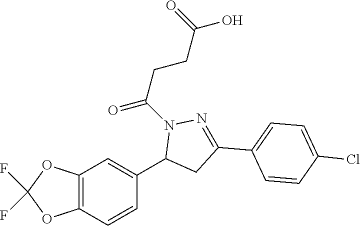 [SMILES: O=C(O)CCC(=O)N1N=C(c2ccc(Cl)cc2)CC1c1ccc2c(c1)OC(F)(F)O2]

or a salt thereof.

In one example the compound of Formula I is:

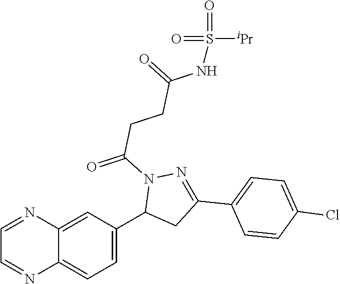 [SMILES: CC(C)S(=O)(=O)NC(=O)CCC(=O)N1N=C(c2ccc(Cl)cc2)CC1c1ccc2nccnc2c1]

or a salt thereof.

In one example the compound of Formula I is:

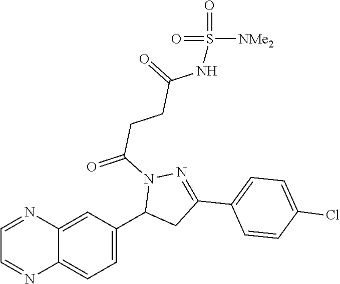 [SMILES: CN(C)S(=O)(=O)NC(=O)CCC(=O)N1N=C(c2ccc(Cl)cc2)CC1c1ccc2nccnc2c1]

or a salt thereof.

In one example the compound of Formula I is:

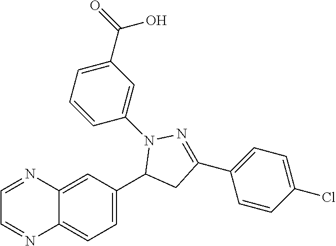 [SMILES: O=C(O)c1cccc(N2N=C(c3ccc(Cl)cc3)CC2c2ccc3nccnc3c2)c1]

or a salt thereof.

In one example the compound of Formula I is:

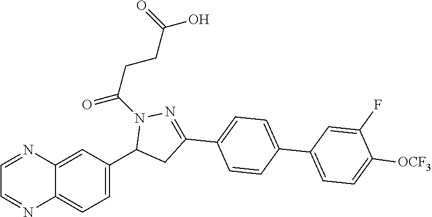 [SMILES: O=C(O)CCC(=O)N1N=C(c2ccc(-c3ccc(OC(F)(F)F)c(F)c3)cc2)CC1c1ccc2nccnc2c1]

or a salt thereof.

In one example the compound of Formula I is:

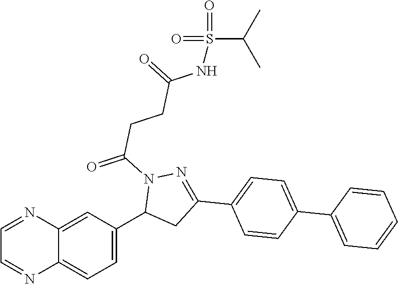 [SMILES: CC(C)S(=O)(=O)NC(=O)CCC(=O)N1N=C(c2ccc(-c3ccccc3)cc2)CC1c1ccc2nccnc2c1]

or a salt thereof.

In another aspect, there is provided a compound of Formula Ila

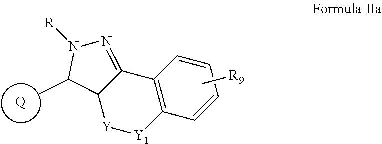

wherein R may be selected from:
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, or
—C(═O)—C₁-C₆—C(═O)—NH₂, —C(═O)—C₁-C₆—C(═O)—NH—C₁-C₆, —C(═O)—C₁-C₆—C(═O)—N(C₁-C₆)₂,
—C(═O)—C₁-C₆—C(═O)—NH—S(O)₂—C₁-C₆, —C(═O)—C₁-C₆—C(═O)—NH—S(O)₂-aryl, —C(═O)—C₁-C₆—C(═O)—NH—S(O)₂—N(C₁-C₆)₂,

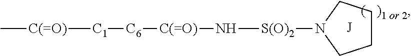

wherein J is a 5 or 6 membered heterocycle ring optionally including a second heteroatom selected from —NH—, NC₁-C₆—, —S— or —O—; or
—C₁-C₆—C(═O)—OH, or
-aryl-C(═O)—OH, or
-aryl-C(═O)—NH—S(O)₂—C₁-C₆;
wherein Q may be selected from

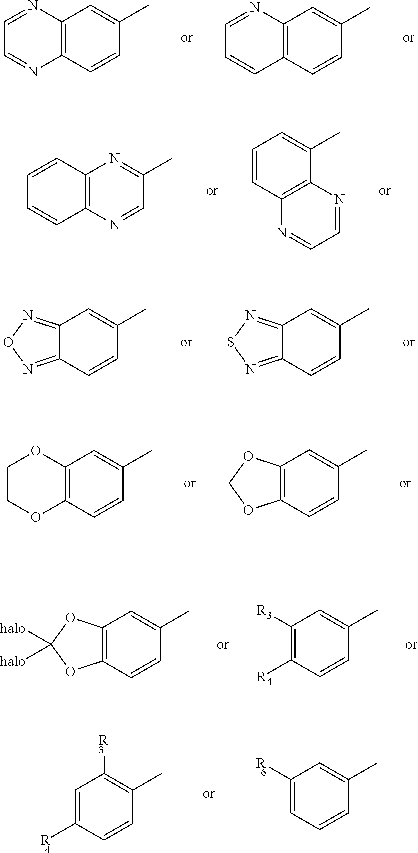

and
wherein R₃, R₄, or R₆ are independently selected from —H, halo, hydroxy, C₁-C₆ alkyl or C₁-C₆ alkoxy, alkoxy substituted with one or more halo, aryl optionally substituted with one or more halo, hydroxy, alkoxy, alkoxy substituted with one or more halo;
wherein R₉ is selected from halo, hydroxy, C₁-C₆ alkyl, C₁-C₆ alkoxy, aryl, or —O-aryl, where each C₁-C₆ alkyl, C₁-C₆ alkoxy, aryl, or —O-aryl may be further substituted with one or more halo, hydroxy, C₁-C₆ alkoxy or C₁-C₆ alkoxy further substituted with one or more halo; and
wherein Y is —CH₂—, —CHR₅, —C(R₅)₂— wherein each R₅ is independently C₁-C₆ alkyl; —O— or —S—, —S(O), —S(O)₂, —NH—, —NR₁₀—, wherein R₁₀ is C₁-C₆ alkyl;
Wherein Y₁ is optionally present and is —CH₂—, —CHR₅, —C(R₅)₂— wherein each R₅ is independently C₁-C₆ alkyl; —O— or —S—, —S(O), —S(O)₂, —NH—, —NR₁₁—, and wherein R₁₁ is C₁-C₆ alkyl;
or a salt thereof.

In one example, R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, —C₁-C₆—C(═O)—OH or -aryl-C(═O)—OH.

In another example, R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂ or -aryl-C(═O)—OH.

In another example, R is —C(═O)—C₁-C₆—C(═O)—OH.

In another example R is —C(═O)—C₁-C₆—C(═O)—NH—S(O)₂—C₁-C₆.

In another example R is selected from —C(═O)—C₁-C₆—C(═O)—NH—S(O)₂—N(C₁-C₆)₂ or —C(═O)—C₁-C₆—C(═O)—NH—S(O)₂-aryl, such as —C(═O)—C₁-C₆—C(═O)—NH—S(O)₂-Ph.

In another example R is -aryl-C(═O)—OH.

In another example R is —C(═O)—CH₂—CH₂—C(═O)—OH.

In another example R is selected from —C(═O)—CH₂—CH₂—C(═O)—NH—S(O)₂-Me or —C(═O)—CH₂—CH₂—C(═O)—NH—S(O)₂-iPr.

In another example R is —C(═O)—CH₂—CH₂—C(═O)—NH—S(O)₂—NMe₂.

In another example R is -phenyl-C(═O)—OH.

In another example Q is selected from:

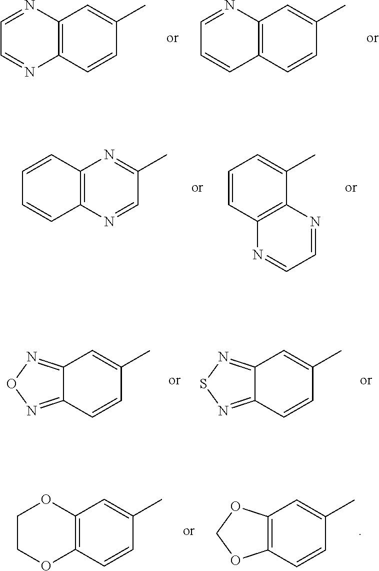

In another example Q is selected from:

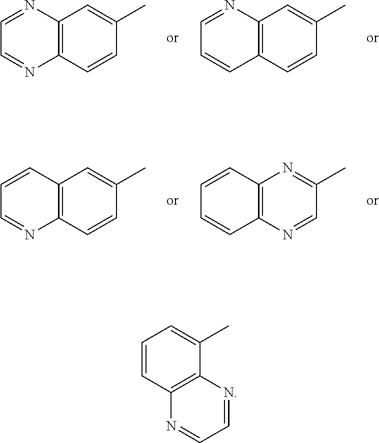

In another example Q is

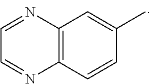

In one example, R₉ is aryl or aryl substituted with C₁-C₆ alkoxy further substituted with one or more halo.

In one example, R₉ is phenyl or phenyl substituted with C₁-C₆ alkoxy. further substituted with one or more halo

In one example Y and Y₁ are each —CH₂—.

In one example the compound of Formula II is selected from

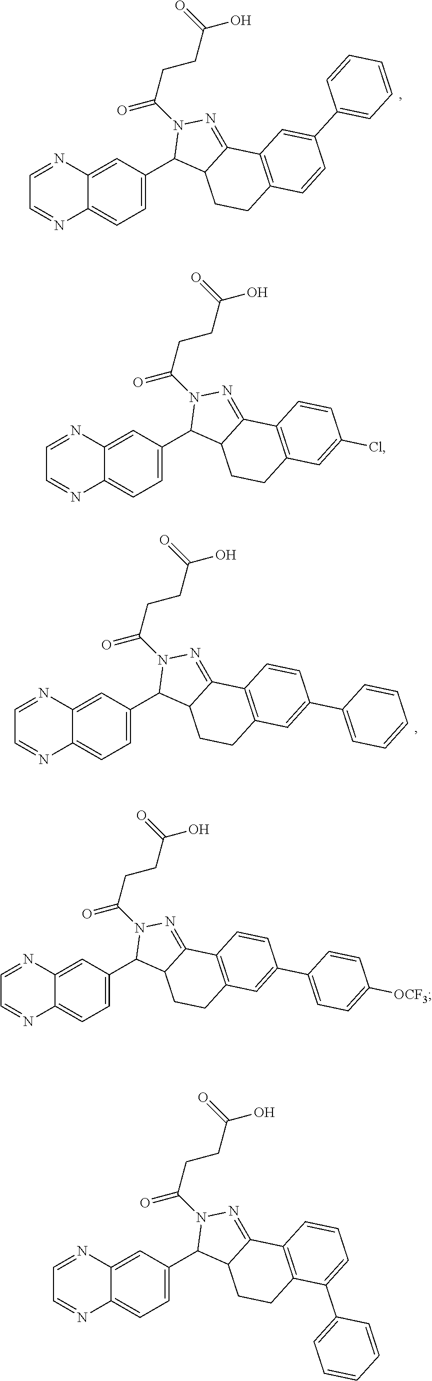 [SMILES: O=C(O)CCC(=O)N1N=C2c3cc(-c4ccccc4)ccc3CCC2C1c1ccc2nccnc2c1 ; O=C(O)CCC(=O)N1N=C2c3ccc(Cl)cc3CCC2C1c1ccc2nccnc2c1 ; O=C(O)CCC(=O)N1N=C2c3ccc(-c4ccccc4)cc3CCC2C1c1ccc2nccnc2c1 ; O=C(O)CCC(=O)N1N=C2c3ccc(-c4ccc(OC(F)(F)F)cc4)cc3CCC2C1c1ccc2nccnc2c1 ; O=C(O)CCC(=O)N1N=C2c3cccc(-c4ccccc4)c3CCC2C1c1ccc2nccnc2c1]

or a salt thereof.

In another aspect, the present invention provides the compound of Formula I or IIa as defined above for reducing the formation of methane from the digestive actions of ruminants and/or for improving ruminant performance.

In one example a compound of Formula I or IIa as defined above is for reducing the formation of methane from the digestive actions of ruminants.

In one example a compound of Formula I or IIa as defined above is for reducing the formation of methane from the digestive actions of ruminants by at least 10%.

In another example of a compound of Formula I or IIa as defined above is for reducing the formation of methane from the digestive actions of ruminants by at least 15%.

In yet another example the compound of Formula I, or IIa as defined above is for reducing the formation of methane from the digestive actions of ruminants by at least 20%.

In one aspect the present invention further provides a method for reducing the production of methane emanating from a ruminant and/or for improving ruminant animal performance, comprising administering orally to the ruminant an effective amount of at least one compound of Formula I or IIa or a salt thereof to the ruminant. Oral administration is to be understood as a route involving drenching, addition to feed, water source or pasture; or manual administration of a bolus or a capsule.

In one example the effective amount of at least one compound of Formula I or IIa or a salt thereof is administered at least once-daily to the ruminant.

In one example the effective amount of at least one compound of Formula I or IIa or a salt thereof reduces the production of methane emanating from the ruminant by at least 10% per day.

In one example the effective amount of at least one compound of Formula I or IIa or a salt thereof reduces the production of methane emanating from the ruminant by at least 15% per day.

In one example the effective amount of at least one compound of Formula I or IIa or a salt thereof reduces the production of methane emanating from the ruminant by at least 20% per day.

In a further aspect of the present invention there is provided a composition for oral administration comprising at least one compound of Formula I or IIa or a salt thereof for reducing the production of methane emanating from a ruminant, and the composition further including at least one agriculturally and orally acceptable excipient.

In one example the composition is adapted for use as a feed additive.

In one example the composition is adapted for use as a water additive.

In another example the composition is adapted for use as a ruminant lick.

In one example the composition is adapted for use as an oral drench.

In another example the composition is adapted for use as a rumen bolus or capsule.

In one example the composition is adapted to reduce the production of methane emanating from the ruminant by at least 10% per day.

In one example the composition is adapted to reduce the production of methane emanating from the ruminant by at least 15% per day.

In one example the composition is adapted to reduce the production of methane emanating from the ruminant by at least 20% per day.

In one example the excipient may include one or more minerals and/or one or more vitamins.

In one example the excipient may include one or more vitamins selected from vitamin A, vitamin D3, vitamin E, and vitamin K, e.g. vitamin K3, vitamin B12, biotin and choline, vitamin B1, vitamin B2, vitamin B6, niacin, folic acid or the like.

In one example the excipient may include one or more minerals selected from calcium, phosphorus, sodium, manganese, zinc, iron, copper, chlorine, sulphur, magnesium, iodine, selenium, and cobalt or the like.

In one example the composition may further include sunflower oil, electrolytes such as ammonium chloride, calcium carbonates, starch, proteins or the like.

The compounds of the present invention have potential for use in a ruminant to reduce the formation of methane without affecting microbial fermentation in a way that would be detrimental to the ruminant.

### DETAILED DESCRIPTION OF INVENTION

### Definitions

The term “ruminant” as used herein is a mammal that is able to acquire nutrients from plant-based food by fermenting it in a specialized foregut (the rumen) prior to digestion, principally through microbial activity. Representative examples of ruminants and other foregut fermenters include cattle, goats, sheep, giraffes, bison, moose, elk, yaks, water buffalo, deer, camels, alpacas, llamas, and antelope.

The term “effective amount” as used herein refers to an amount of at least one compound of Formula I or a salt thereof that either reduces the production of methane emanating from the ruminant or improves ruminant performance.

The term “ruminant performance” as used herein refers to improving the productivity of the ruminant, such as increased weight gain, milk yield or quality, wool growth or quality, surviving offspring per parturition, or the like.

The term —C₁-C₆-means a carbon chain having from 1 to 6 carbons and being straight chained or branched, saturated or unsaturated, including but not limited to methyl, ethyl, ethylene, propyl, isopropyl, propylene, butyl, tert-butyl, butylene, pentyl, pentene, hexyl, hexene and the like, wherein the —C₁-C₆ group is optionally substituted with one or more halo.

The term —C₁-C₆ alkyl-means an alkyl chain having from 1 to 6 carbons, being straight chained or branched including but not limited to methyl, ethyl, propyl, isopropyl, butyl, tertiary butyl, pentyl and hexyl, wherein the —C₁-C₆ alkyl group is optionally substituted with one or more halo.

The term “halo” as used herein refers to a halogen atom selected from Cl, Br, or F.

The term “aryl” as used herein refers to “aryl”, unless specifically limited, denotes a C₅₋₁₂ aryl group, suitably a C₆₋₁₀ aryl group, more suitably a C₆₋₈ aryl group. Aryl groups will contain at least one aromatic ring (e.g. one, two or three rings). Examples of a typical aryl group with one aromatic ring is phenyl. An example of a typical aryl group with two aromatic rings is naphthyl. The aryl group is optionally substituted with one or more —CN, -halo, —C(halo)₃, —NH₂, —NO₂, —NH—(C₁-C₆ alkyl), —N(C₁-C₆ alkyl)₂, —C₁-C₆ alkyl, —C₁-C₆ alkyl substituted with one or more halo, —C₁-C₆ alkenyl, —C₁-C₆ alkynyl, —O—C₁-C₆-alkyl, —O—C₁-C₆-alkyl substituted with one or more halo, aryl or substituted aryl, the substituted aryl, may include a substituted phenyl (benzyl group), a substituted thiophenol, a tosyl group, the substituted aryl group being substituted with one or more —CN, -halo, —C(halo)₃, —NH₂, —NO₂—NH—(C₁-C₆ alkyl), —N(C₁-C₆ alkyl)₂, C₁-C₆ alkyl, C₁-C₆ alkenyl and C₁-C₆ alkynyl.

The term “heteroaryl” as used herein refers to”, unless specifically limited, denotes an aryl residue, wherein one or more (e.g., 1, 2, 3, or 4, suitably 1, 2 or 3) ring atoms are replaced by heteroatoms selected from N, S and O, or else a 5-membered aromatic ring containing one or more (e.g., 1, 2, 3, or 4, suitably 1, 2 or 3) ring atoms selected from N, S and O. Exemplary monocyclic heteroaryl groups having one heteroatom include: five membered rings (e.g., pyrrole, furan, thiophene); and six membered rings (e.g., pyridine, such as pyridin-2-yl, pyridin-3-yl and pyridin-4-yl). Exemplary monocyclic heteroaryl groups having two heteroatoms include: five membered rings (e.g., pyrazole, oxazole, isoxazole, thiazole, isothiazole, imidazole, such as imidazol-1-yl, imidazol-2-yl imidazol-4-yl); six membered rings (e.g., pyridazine, pyrimidine, pyrazine). Exemplary monocyclic heteroaryl groups having three heteroatoms include: 1,2,3-triazole and 1,2,4-triazole, oxadiazole, oxadiazolone. Exemplary monocyclic heteroaryl groups having four heteroatoms include tetrazole. Exemplary bicyclic heteroaryl groups include: indole (e.g., indol-6-yl), benzofuran, benzothiophene, quinoline, isoquinoline, indazole, benzimidazole, benzthiazole, quinazoline and purine. The heteroaryl group is optionally substituted with one or more —CN, -halo, —C(halo)₃, —NH₂, —NH—(C₁-C₆ alkyl), —N(C₁-C₆ alkyl)₂, —NO₂, C₁-C₆ alkyl, C₁-C₆ alkenyl, C₁-C₆ alkynyl, aryl or substituted aryl, the substituted aryl, may include a substituted phenyl (benzyl group), a substituted thiophenol, a tosyl group, the substituted aryl group being substituted with one or more —CN, -halo, —C(halo)₃, —NH₂, —NH—(C₁-C₆ alkyl), —N(C₁-C₆ alkyl)₂, alkyl, alkenyl and alkynyl.

The term “heterocyclyl” as used herein includes a “carbocycle” as defined herein, wherein one or more (e.g, 1, 2, 3, or 4) carbon atoms have been replaced with a heteroatom (e.g. O, N, or S). The terms “heterocycle” or “heterocyclyl” includes saturated rings (i.e., heterocycloalkyls), partially unsaturated rings, and aromatic rings (i.e., heteroaromatic rings). Substituted heterocyclyls include, for example, heterocyclic rings substituted with any of the substituents disclosed herein including carbonyl groups.

Examples of heterocycles include by way of example and not limitation pyridyl, dihydroypyridyl, tetrahydropyridyl (piperidyl), thiazolyl,thiazolidine-dione; tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidirtyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

All possible stereoisomers of the claimed compounds are included in the present disclosure. Where a compound described herein has at least one chiral center, it may accordingly exist as enantiomers. Where a compound possesses two or more chiral centers it may additionally exist as diastereomers. It is to be understood that all such isomers and mixtures thereof are encompassed within the scope of the present disclosure.

Some of the crystalline forms of the compounds may exist in more than one polymorphic form and as such all forms are intended to be included in the present disclosure. In addition, some of the compounds may form solvates with water (i.e., hydrates) or common organic solvents, and such solvates are also intended to be encompassed within the scope of this disclosure. The compounds, including their salts, can also be obtained in the form of their hydrates, or include other solvents used for their crystallization.

The present disclosure further includes within its scope prodrugs of the compounds described herein. In general, such prodrugs will be functional derivatives of the compounds which are readily convertible in vivo into the desired active compound. Thus, in these cases, the methods of treatment of the present invention, the term “administering” shall encompass the treatment of the various disorders described with prodrug versions of one or more of the claimed compounds, but which converts to the above specified compound in vivo after administration to the subject.

As used herein, the term “composition” is intended to encompass a product comprising a claimed compound(s) in a therapeutically effective amount, as well as any product which results, directly or indirectly, from combinations of the claimed compounds.

The term “or a salt thereof,” as used herein, is a salt of an acid or a basic nitrogen atom. Illustrative salts include, but are not limited, to sodium salt, potassium salt, lithium salt, calcium salt, ammonium salt, sulfate, citrate, acetate, oxalate, chloride, bromide, iodide, nitrate, bisulfate, phosphate, and the like.

### Methods of Making the Compounds of Formula I, Formula II and Formula IV

### 4-(3-(4-Chlorophenyl)-5-(quinoxalin-6-yl)-4,5-dihydro-1H-pyrazol-1-yl)-4-oxobutanoic acid (Compound 1)

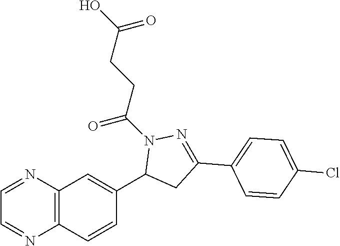 [SMILES: O=C(O)CCC(=O)N1N=C(c2ccc(Cl)cc2)CC1c1ccc2nccnc2c1]

To a solution of quinoxaline-6-carbaldehyde (2.00 g, 12.6 mmol) and 4-chloroacetophenone (1.64 mL, 12.6 mmol) in EtOH (120 mL) at 0° C. was added dropwise 2 M NaOH (32 mL, 63.2 mmol), and the reaction mixture stirred for 18 h (while gradually warming to room temperature). The resulting precipitated solids were then collected by filtration, washed with water and dried in vacuo to afford (E)-1-(4-chlorophenyl)-3-(quinoxalin-6-yl)prop-2-en-1-one as a colourless solid (2.43 g), which was used directly in the next step without further purification.

To a stirred solution of (E)-1-(4-chlorophenyl)-3-(quinoxalin-6-yl)prop-2-en-1-one (2.43 g) in EtOH (80 mL) was added hydrazine hydrate (0.77 mL, 50% w/w), and the reaction mixture heated under reflux for 2 h. The reaction mixture was allowed to cool to room temperature and the precipitated solids were then collected by filtration, washed with water and dried in vacuo to afford 6-(3-(4-chlorophenyl)-4,5-dihydro-1H-pyrazol-5-yl)quinoxaline as a yellow-orange solid (1.80 g), which was used directly in the next step without further purification.

To a stirred solution of 6-(3-(4-chlorophenyl)-4,5-dihydro-1H-pyrazol-5-yl)quinoxaline (0.50 g) in THF (20 mL) was added succinic anhydride (162 mg, 1.62 mmol), and the reaction mixture heated at 50° C. for 18 h. The resulting reaction mixture was then concentrated in vacuo, with purification by flash column chromatography (EtOAc, neat→CH₂Cl₂/MeOH, 9:1) affording Compound 1 as a brown solid (0.45 g, 31% over 3 steps). ¹H NMR (400 MHz, CDCl₃) δ 8.82 (2H, s), 8.11 (1H, d, J=8.7 Hz), 7.96 (1H, d, J=1.9 Hz), 7.69 (2H, d, J=8.6 Hz), 7.66 (1H, dd, J=8.8, 2.0 Hz), 7.41 (2H, d, J=8.6 Hz), 5.83 (1H, dd, J=11.9, 4.9 Hz), 3.88 (1H, dd, J=17.7, 12.0 Hz), 3.32-3.12 (3H, m), 2.75 (2H, t, J=6.7 Hz).

### 4-Oxo-4-(3-phenyl-5-(quinoxalin-6-yl)-4,5-dihydro-1H-pyrazol-1-yl)butanoic acid (Compound 1A)

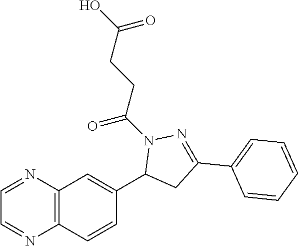 [SMILES: O=C(O)CCC(=O)N1N=C(c2ccccc2)CC1c1ccc2nccnc2c1]

To a solution of quinoxaline-6-carbaldehyde (200 mg, 1.26 mmol) and acetophenone (0.15 mL, 1.26 mmol) in EtOH (12 mL) at 0° C. was added dropwise 2 M NaOH (3.2 mL, 6.32 mmol), and the reaction mixture stirred for 18 h (while gradually warming to room temperature). The resulting precipitated solids were then collected by filtration, washed with water and dried in vacuo to afford (E)-1-phenyl-3-(quinoxalin-6-yl)prop-2-en-1-one as a colourless solid (190 g), which was used directly in the next step without further purification.

To a stirred solution of (E)-1-phenyl-3-(quinoxalin-6-yl)prop-2-en-1-one (190 g) in EtOH (8 mL) was added hydrazine hydrate (90 μL, 50% w/w), and the reaction mixture heated under reflux for 18 h. The reaction mixture was allowed to cool to room temperature and the precipitated solids were then collected by filtration, washed with water and dried in vacuo to afford 6-(3-phenyl-4,5-dihydro-1H-pyrazol-5-yl)quinoxaline as a yellow solid (160 mg), which was used directly in the next step without further purification.

To a stirred solution 6-(3-phenyl-4,5-dihydro-1H-pyrazol-5-yl)quinoxaline (160 mg) in THF (5 mL) was added succinic anhydride (58 mg, 0.58 mmol), and the reaction mixture heated at 50° C. for 18 h. The resulting reaction mixture was then concentrated in vacuo, with purification by flash column chromatography (EtOAc, neat→CH₂Cl/MeOH, 9:1) affording Compound 1A as a colourless solid (15 mg, 3% over 3 steps). ¹H NMR (400 MHz, CDCl₃) δ 8.80 (2H, s), 8.09 (1H, d, J=8.7 Hz), 7.98 (1H, s), 7.77-7.74 (2H, m), 7.66 (1H, dd. J=8.7, 1.6 Hz), 7.45-7.42 (3H, m), 5.83 (1H, dd, J=11.9, 4.9 Hz), 3.89 (1H, dd. J=17.8, 12.0 Hz), 3.32-3.13 (3H, m), 2.75 (2H, t, J=6.7 Hz).

### 4-(3-(4-Isopropylphenyl)-5-(quinoxalin-6-yl)-4,5-dihydro-1H-pyrazol-1-yl)-4-oxobutanoic acid (Compound 3)

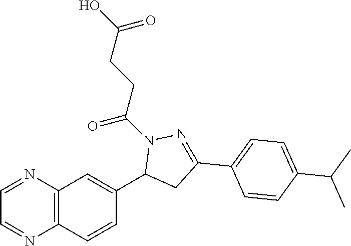 [SMILES: CC(C)c1ccc(C2=NN(C(=O)CCC(=O)O)C(c3ccc4nccnc4c3)C2)cc1]

To a solution of quinoxaline-6-carbaldehyde (200 mg, 1.26 mmol) and 4-isopropylacetophenone (204 mg, 1.26 mmol) in EtOH (12 mL) at 0° C. was added dropwise 2 M NaOH (3.2 mL, 6.32 mmol), and the reaction mixture stirred for 18 h (while gradually warming to room temperature). The resulting precipitated solids were then collected by filtration, washed with water and dried in vacuo to afford (E)-1-(4-isopropylphenyl)-3-(quinoxalin-6-yl)prop-2-en-1-one as a colourless solid (300 mg), which was used directly in the next step without further purification.

To a stirred solution of (E)-1-(4-isopropylphenyl)-3-(quinoxalin-6-yl)prop-2-en-1-one (200 mg) in EtOH (10 mL) was added hydrazine hydrate (82 μL, 50% w/w), and the reaction mixture heated under reflux for 2 h. The solvent was then removed in vacuo and the resulting residue was poured onto ice water, collected by filtration and dried in vacuo to afford 6-(3-(4-isopropylphenyl)-4,5-dihydro-1H-pyrazol-5-yl)quinoxaline as a yellow solid (150 mg), which was used in the next step without further purification.

In a sealed vial, a mixture of 6-(3-(4-isopropylphenyl)-4,5-dihydro-1H-pyrazol-5-yl)quinoxaline (100 mg) and succinic anhydride (63 mg, 0.63 mmol) in THF (4 mL) was subjected to microwave irradiation at 120° C. for 45 min. The resulting reaction mixture was concentrated in vacuo. Purification by flash column chromatography (EtOAc, neat→CH₂Cl₂/MeOH, 9:1) afforded Compound 3 as a yellow solid (40 mg, 17% over 3 steps). ¹H NMR (500 MHz, CDCl₃) δ 8.82-8.81 (2H, m), 8.09 (1H, d, J=8.7 Hz), 7.97 (1H, d, J=1.8 Hz), 7.69 (2H, d, J=8.4 Hz), 7.66 (1H, dd, J=8.7, 2.0 Hz), 7.29 (2H, d, J=8.2 Hz), 5.82 (1H, dd. J=11.8, 4.7 Hz), 3.88 (1H, dd, J=17.6, 11.8 Hz), 3.29 (1H, dt, J=17.5, 6.6 Hz), 3.24 (1H, dd, J=17.8, 4.9 Hz), 3.16 (1H, dt, J=17.5, 6.7 Hz), 2.95 (1H, septet, J=6.9 Hz), 2.74 (2H, t, J=6.7 Hz), 1.26 (6H, d, J=6.7 Hz); ¹³C NMR (125 MHz, CDCl₃) δ 176.2 (C), 170.4 (C), 154.9 (C), 152.3 (C), 145.4 (CH), 145.1 (CH), 143.8 (C), 143.1 (C), 142.6 (C), 130.6 (CH), 128.6 (C), 128.1 (CH), 127.1 (2×CH), 127.0 (2×CH), 125.9 (CH), 60.1 (CH), 42.4 (CH₂), 34.3 (CH), 29.2 (CH₂), 29.0 (CH₂), 23.9 (2×CH₃).

### 4-(3-(4-Cyclohexylphenyl)-5-(quinoxalin-6-yl)-4,5-dihydro-1H-pyrazol-1-yl)-4-oxobutanoic acid (Compound 4)

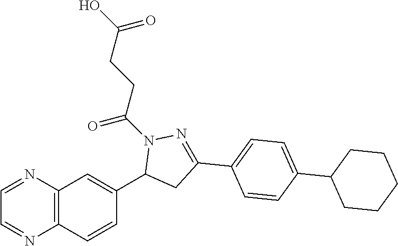 [SMILES: O=C(O)CCC(=O)N1N=C(c2ccc(C3CCCCC3)cc2)CC1c1ccc2nccnc2c1]

Compound 4 was prepared over 3 steps using a method similar to that described for the preparation of Compound 3-step 1: quinoxaline-6-carbaldehyde (200 mg, 1.26 mmol), 4-cyclohexylacetophenone (255 mg, 1.26 mmol) and 2 M NaOH (3.2 mL, 6.32 mmol) in EtOH (12 mL) to afford (E)-1-(4-cyclohexylphenyl)-3-(quinoxalin-6-yl)prop-2-en-1-one as a colourless solid (300 mg); step 2: (E)-1-(4-cyclohexylphenyl)-3-(quinoxalin-6-yl)prop-2-en-1-one (250 mg) and hydrazine hydrate (91 μL, 50% w/w) in EtOH (10 mL) to afford 6-(3-(4-cyclohexylphenyl)-4,5-dihydro-1H-pyrazol-5-yl)quinoxaline as a yellow solid (120 mg); and step 3:6-(3-(4-cyclohexylphenyl)-4,5-dihydro-1H-pyrazol-5-yl)quinoxaline (100 mg) and succinic anhydride (56 mg, 0.56 mmol) in THF (5 mL). Purification by flash column chromatography (EtOAc, neat) afforded Compound 4 as a colourless solid (70 mg, 18% over 3 steps). ¹H NMR (500 MHz, CDCl₃) δ 8.83-8.81 (2H, m), 8.10 (1H, d, J=8.7 Hz), 7.98 (1H, d, J=1.9 Hz), 7.68 (2H, d, J=8.4 Hz), 7.66 (1H, dd, J=8.8, 2.1 Hz), 7.27 (2H, d, J=8.4 Hz), 5.81 (1H, dd, J=11.8, 4.8 Hz), 3.87 (1H, dd, J=17.8, 11.8 Hz), 3.29 (1H, dt, J=17.5, 6.9 Hz), 3.23 (1H, dd, J=17.6, 4.6 Hz), 3.15 (1H, dt, J=17.5, 6.7 Hz), 2.74 (2H, t, J=6.5 Hz), 2.56-2.51 (1H, m), 1.88-1.84 (4H, m), 1.46-1.35 (4H, m), 1.30-1.23 (2H, m); ¹³C NMR (125 MHz, CDCl₃) δ 176.4 (C), 170.4 (C), 154.9 (C), 151.5 (C), 145.3 (CH), 145.0 (CH), 143.9 (C), 143.0 (C), 142.5 (C), 130.5 (CH), 128.5 (C), 128.2 (CH), 127.5 (2×CH), 127.0 (2×CH), 125.9 (CH), 60.1 (CH), 44.7 (CH), 42.4 (CH₂), 34.3 (2×CH₂), 29.1 (CH₂), 29.0 (CH₂), 26.9 (2×CH₂), 26.2 (CH₂).

### 4-Oxo-4-(5-(quinoxalin-6-yl)-3-(4-(trifluoromethyl)phenyl)-4,5-dihydro-1H-pyrazol-1-yl)butanoic acid (Compound 5)

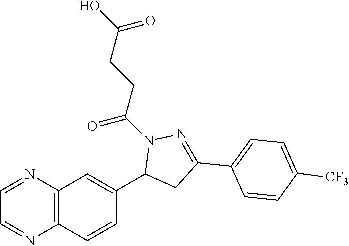 [SMILES: O=C(O)CCC(=O)N1N=C(c2ccc(C(F)(F)F)cc2)CC1c1ccc2nccnc2c1]

Compound 5 was prepared over 3 steps using a method similar to that described for the preparation of Compound 1A-step 1: quinoxaline-6-carbaldehyde (200 mg, 1.26 mmol), 4-(trifluoromethyl) acetophenone (237 mg, 1.26 mmol) and 2 M NaOH (3.2 mL, 6.32 mmol) in EtOH (12 mL) to afford (E)-3-(quinoxalin-6-yl)-1-(4-(trifluoromethyl)phenyl)prop-2-en-1-one as a beige solid (240 mg); step 2: (E)-3-(quinoxalin-6-yl)-1-(4-(trifluoromethyl)phenyl)prop-2-en-1-one (240 mg) and hydrazine hydrate (91 μL, 50% w/w) in EtOH (8 mL) to afford 6-(3-(4-(trifluoromethyl)phenyl)-4,5-dihydro-1H-pyrazol-5-yl)quinoxaline as a beige solid (190 mg); and step 3:6-(3-(4-(trifluoromethyl)phenyl)-4,5-dihydro-1H-pyrazol-5-yl)quinoxaline (190 mg) and succinic anhydride (56 mg, 0.56 mmol) in THF (5 mL). Purification by flash column chromatography (EtOAc, neat→CH₂Cl₂/MeOH, 9:1) afforded Compound 5 as a colourless solid (27 mg, 5% over 3 steps). ¹H NMR (400 MHz, CDCl₃) δ 8.81 (2H, s), 8.11 (1H, d, J=8.8 Hz), 7.97 (1H, s), 7.86 (2H, d, J=8.1 Hz), 7.69-7.65 (3H, m), 5.86 (1H, dd, J=12.0, 4.8 Hz), 3.90 (1H, dd, J=17.8, 12.1 Hz), 3.31-3.11 (3H, m), 2.75 (2H, t, J=6.6 Hz).

### 4-(3-(4-Methoxyphenyl)-5-(quinoxalin-6-yl)-4,5-dihydro-1H-pyrazol-1-yl)-4-oxobutanoic acid (Compound 6)

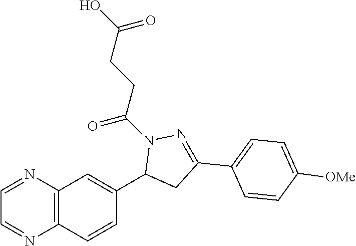 [SMILES: COc1ccc(C2=NN(C(=O)CCC(=O)O)C(c3ccc4nccnc4c3)C2)cc1]

Compound 6 was prepared over 3 steps using a method similar to that described for the preparation of Compound 3-step 1: quinoxaline-6-carbaldehyde (200 mg, 1.26 mmol), 4-methoxyacetophenone (189 mg, 1.26 mmol) and 2 M NaOH (3.2 mL, 6.32 mmol) in EtOH (12 mL) to afford (E)-1-(4-methoxyphenyl)-3-(quinoxalin-6-yl)prop-2-en-1-one as a colourless solid (316 mg); step 2: (E)-1-(4-methoxyphenyl)-3-(quinoxalin-6-yl)prop-2-en-1-one (110 mg) and hydrazine hydrate (47 μL, 50% w/w) in EtOH (10 mL) to afford 6-(3-(4-methoxyphenyl)-4,5-dihydro-1H-pyrazol-5-yl)quinoxaline as a yellow solid (107 mg); and step 3:6-(3-(4-methoxyphenyl)-4,5-dihydro-1H-pyrazol-5-yl)quinoxaline (60 mg) and succinic anhydride (39 mg, 0.39 mmol) in THF (3 mL). Purification by flash column chromatography (EtOAc, neat→CH₂Cl₂/MeOH, 9:1) afforded Compound 6 as a yellow solid (20 mg, 20% over 3 steps). ¹H NMR (500 MHz, CDCl₃) δ 8.82-8.81 (2H, m), 8.10 (1H, d, J=8.7 Hz), 7.97 (1H, d, J=2.0 Hz), 7.71-7.69 (2H, m), 7.67 (1H, dd, J=8.7, 2.1 Hz), 6.96-6.93 (2H, m), 5.81 (1H, dd, J=11.7, 4.7 Hz), 3.87 (1H, dd, J=17.9, 11.9 Hz), 3.86 (3H, s), 3.29 (1H, dt, J=17.5, 6.5 Hz), 3.23 (1H, dd, J=17.5, 4.7 Hz), 3.14 (1H, dt, J=17.5, 6.7 Hz), 2.74 (2H, t, J=6.6 Hz); 13C NMR (125 MHz, CDCl₃) δ 175.4 (C), 170.5 (C), 161.9 (C), 154.8 (C), 145.4 (CH), 145.2 (CH), 143.8 (C), 143.1 (C), 142.7 (C), 130.7 (CH), 128.6 (2×CH), 128.2 (CH), 125.9 (CH), 123.5 (C), 114.4 (2×CH), 60.1 (CH), 55.6 (CH₃), 42.5 (CH₂), 29.2 (2×CH₂); HRMS (ESI/Q-TOF) m/z: [M+Na]⁺ calcd for C₂₂H₂₀N₄NaO₄ 427.1377; found 427.1374.

### 4-Oxo-4-(3-(4-phenoxyphenyl)-5-(quinoxalin-6-yl)-4,5-dihydro-1H-pyrazol-1-yl)butanoic acid (Compound 7)

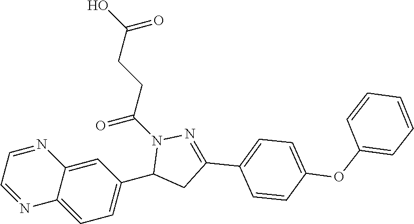 [SMILES: O=C(O)CCC(=O)N1N=C(c2ccc(Oc3ccccc3)cc2)CC1c1ccc2nccnc2c1]

To a solution of quinoxaline-6-carbaldehyde (500 mg, 3.16 mmol) and 4-phenoxyacetophenone (670 mg, 3.16 mmol) in EtOH (32 mL) at room temperature was added dropwise 2 M NaOH (7.9 mL, 15.8 mmol), and the reaction mixture stirred for 4 h. After, the mixture was cooled down to 0° C., and the resulting precipitated solids were then collected by filtration, washed with water and dried in vacuo to afford (E)-1-(4-phenoxyphenyl)-3-(quinoxalin-6-yl)prop-2-en-1-one as an off-white solid (1.00 g), which was used directly in the next step without further purification.

To a stirred solution of (E)-1-(4-phenoxyphenyl)-3-(quinoxalin-6-yl)prop-2-en-1-one (500) mg) in EtOH (8 mL) was added hydrazine hydrate (0.26 mL, 50% w/w), and the reaction mixture heated under reflux for 2 h. The solvent was then removed in vacuo and the resulting residue was poured onto ice water, collected by filtration and dried in vacuo to afford 6-(3-(4-phenoxyphenyl)-4,5-dihydro-1H-pyrazol-5-yl)quinoxaline as a brown solid (350 mg), which was used in the next step without further purification.

In a sealed vial, a mixture of 6-(3-(4-phenoxyphenyl)-4,5-dihydro-1H-pyrazol-5-yl)quinoxaline (350 mg) and succinic anhydride (191 mg, 1.91 mmol) in THF (10 mL) was subjected to microwave irradiation at 120° C. for 45 min. The resulting precipitate was then collected by filtration, washed with water (3×5 mL), CH₂Ch (3×5 mL), EtOAc (3×5 mL) then petroleum ether (3×5 mL), and dried in vacuo to afford Compound 7 as a colourless solid (147 mg, 20% over 3 steps). ¹H NMR (400 MHz, DMSO-d₆) δ 12.12 (1H, br s), 8.94-8.92 (2H, m), 8.08 (1H, d, J=8.7 Hz), 7.89 (1H, d, J=1.9 Hz), 7.85-7.81 (2H, m), 7.71 (1H, dd, J=8.7, 2.0 Hz), 7.46-7.40 (2H, m), 7.22-7.18 (1H, m), 7.09-7.06 (4H, m), 5.83 (1H, dd, J=12.0, 4.9 Hz), 3.97 (1H, dd, J=18.2, 12.0 Hz), 3.26 (1H, dd, J=18.2, 4.9 Hz), 3.11-2.91 (2H, m), 2.49 (2H, t, J=7.2 Hz); ¹³C NMR (101 MHz, DMSO-d₆) δ 173.7 (C), 169.1 (C), 158.7 (C), 155.8 (C), 153.9 (C), 146.0 (CH), 145.6 (CH), 144.2 (C), 142.1 (C), 141.6 (C), 130.2 (2×CH), 129.8 (CH), 128.8 (2×CH), 128.1 (CH), 126.0 (C), 125.3 (CH), 124.2 (CH), 119.3 (2×CH), 118.3 (2×CH), 59.5 (CH), 41.8 (CH₂), 28.7 (CH₂), 28.4 (CH₂); HRMS (ESI/Q-TOF) m/z: [M+H]⁺ calcd for C₂₇H₂₃N₄O₄, 467.1714; found, 467.1697.

### 4-Oxo-4-(5-(quinoxalin-6-yl)-3-(4-(trifluoromethoxy)phenyl)-4,5-dihydro-1H-pyrazol-1-yl)butanoic acid (Compound 8)

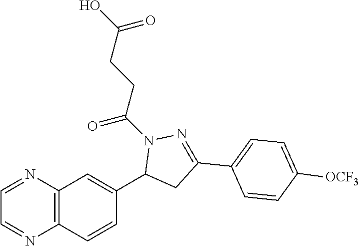 [SMILES: O=C(O)CCC(=O)N1N=C(c2ccc(OC(F)(F)F)cc2)CC1c1ccc2nccnc2c1]

Compound 8 was prepared over 3 steps using a method similar to that described for the preparation of Compound 3-step 1: quinoxaline-6-carbaldehyde (200 mg, 1.26 mmol), 4-(trifluoromethoxy) acetophenone (257 mg, 1.26 mmol) and 2 M NaOH (3.2 mL, 6.32 mmol) in EtOH (12 mL) to afford (E)-3-(quinoxalin-6-yl)-1-(4-(trifluoromethoxy)phenyl)prop-2-en-1-one as a colourless solid (200 mg); step 2: (E)-3-(quinoxalin-6-yl)-1-(4-(trifluoromethoxy)phenyl)prop-2-en-1-one (200 mg) and hydrazine hydrate (72 μL, 50% w/w) in EtOH (10 mL) to afford 6-(3-(4-(trifluoromethoxy)phenyl)-4,5-dihydro-1H-pyrazol-5-yl)quinoxaline as a yellow solid (170 mg); and step 3:6-(3-(4-(trifluoromethoxy)phenyl)-4,5-dihydro-1H-pyrazol-5-yl)quinoxaline (100 mg) and succinic anhydride (56 mg, 0.56 mmol) in THF (4 mL). Purification by flash column chromatography (EtOAc, neat→CH₂Cl₂/MeOH, 9:1) afforded Compound 8 as a yellow solid (72 mg, 21% over 3 steps). ¹H NMR (500 MHz, CDCl₃) δ 8.82 (2H, s), 8.11 (1H, d, J=8.7 Hz), 7.97 (1H, d, J=1.9 Hz), 7.80 (2H, d, J=9.0 Hz), 7.66 (1H, dd, J=8.7 and 2.1 Hz), 7.29-7.26 (2H, m), 5.84 (1H, dd, J=11.6, 4.9 Hz), 3.88 (1H, dd, J=17.8, 12.0 Hz), 3.26 (1H, dt, J=17.4, 6.6 Hz), 3.23 (1H, dd, J=17.5, 5.0 Hz), 3.14 (1H, dt, J=17.5, 6.7 Hz), 2.74 (2H, t, J=6.6 Hz).

### 4-(3-(4-Bromophenyl)-5-(quinoxalin-6-yl)-4,5-dihydro-1H-pyrazol-1-yl)-4-oxobutanoic acid (Compound 9)

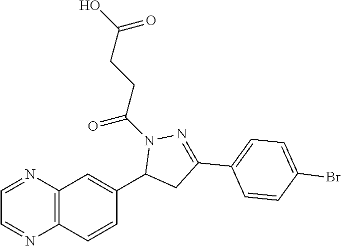 [SMILES: O=C(O)CCC(=O)N1N=C(c2ccc(Br)cc2)CC1c1ccc2nccnc2c1]

Compound 9 was prepared over 3 steps using a method similar to that described for the preparation of Compound 3-step 1: quinoxaline-6-carbaldehyde (200 mg, 1.26 mmol), 4-bromoacetophenone (251 mg, 1.26 mmol) and 2 M NaOH (3.2 mL, 6.32 mmol) in EtOH (12 mL) to afford (E)-1-(4-bromophenyl)-3-(quinoxalin-6-yl)prop-2-en-1-one as a colourless solid (360 mg); step 2: (E)-1-(4-bromophenyl)-3-(quinoxalin-6-yl)prop-2-en-1-one (360 mg) and hydrazine hydrate (0.13 mL, 50% w/w) in EtOH (10 mL) to afford 6-(3-(4-bromophenyl)-4,5-dihydro-1H-pyrazol-5-yl)quinoxaline as a yellow solid (150 mg); and step 3:6-(3-(4-bromophenyl)-4,5-dihydro-1H-pyrazol-5-yl)quinoxaline (150 mg) and succinic anhydride (85 mg, 0.85 mmol) in THF (5 mL). Purification by flash column chromatography (EtOAc, neat→CH₂Cl₂/MeOH, 9:1) afforded Compound 9 as a pale-yellow solid (91 mg, 16% over 3 steps). ¹H NMR (400 MHz, DMSO-d₆) δ 12.09 (1H, s), 8.94-8.93 (2H, m), 8.08 (1H, d, J=8.7 Hz), 7.90 (1H, d, J=1.9 Hz), 7.79-7.66 (5H, m), 5.84 (1H, dd, J=12.0, 5.1 Hz), 3.98 (1H, dd. J=18.3, 12.0 Hz), 3.35-3.28 (1H, m), 3.10 (1H, dt, J=17.2, 6.7 Hz), 2.97 (1H, dt, J=17.2, 6.6 Hz), 2.52-2.49 (2H, m); ¹³C NMR (101 MHz, DMSO-d₆) δ 173.7 (C), 169.3 (C), 153.6 (C), 146.0 (CH), 145.6 (CH), 144.0 (C), 142.1 (C), 141.6 (C), 131.8 (2×CH), 130.2 (C), 129.8 (CH), 128.7 (2×CH), 128.1 (CH), 125.3 (CH), 123.8 (C), 59.7 (CH), 41.6 (CH₂), 28.7 (CH₂) 28.3 (CH₂).

### 4-(3-(2,4-Dichlorophenyl)-5-(quinoxalin-6-yl)-4,5-dihydro-1H-pyrazol-1-yl)-4-oxobutanoic acid (Compound 10)

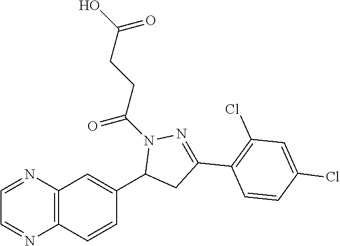 [SMILES: O=C(O)CCC(=O)N1N=C(c2ccc(Cl)cc2Cl)CC1c1ccc2nccnc2c1]

Compound 10 was prepared over 3 steps using a method similar to that described for the preparation of Compound 3-step 1: quinoxaline-6-carbaldehyde (200 mg, 1.26 mmol), 2,4-dichloroacetophenone (238 mg, 1.26 mmol) and 2 M NaOH (3.2 mL, 6.32 mmol) in EtOH (12 mL) to afford (E)-1-(2,4-dichlorophenyl)-3-(quinoxalin-6-yl)prop-2-en-1-one as an off-white solid (310 mg); step 2: (E)-1-(2,4-dichlorophenyl)-3-(quinoxalin-6-yl)prop-2-en-1-one (310 mg) and hydrazine hydrate (0.12 mL, 50% w/w) in EtOH (8 mL) to afford 6-(3-(2,4-dichlorophenyl)-4,5-dihydro-1H-pyrazol-5-yl)quinoxaline as a yellow solid (270 mg); and step 3:6-(3-(2,4-dichlorophenyl)-4,5-dihydro-1H-pyrazol-5-yl)quinoxaline (100 mg) and succinic anhydride (58 mg, 0.58 mmol) in THF (5 mL). Purification by flash column chromatography (EtOAc, neat→CH₂Cl₂/MeOH, 9:1) afforded Compound 10 as a pale-yellow solid (112 mg, 54%). ¹H NMR (400 MHz, CDCl₃) δ 8.84-8.82 (2H, m), 8.12 (1H, d, J=8.7 Hz), 7.97 (1H, d, J=2.0 Hz), 7.79 (1H, d, J=8.5 Hz), 7.68 (1H, dd, J=8.7, 2.1 Hz), 7.46 (1H, d, J=2.0 Hz), 7.32 (1H, dd, J=8.5, 2.1 Hz), 5.83 (1H, dd, J=12.0, 4.9 Hz), 4.08 (1H, dd, J=18.3, 12.0 Hz), 3.43 (1H, dd, J=18.3, 4.9 Hz), 3.30-3.22 (1H, m), 3.16-3.08 (1H, m), 2.74 (2H, t, J=6.7 Hz).

### 4-(3-(3,4-Dichlorophenyl)-5-(quinoxalin-6-yl)-4,5-dihydro-1H-pyrazol-1-yl)-4-oxobutanoic acid (Compound 11)

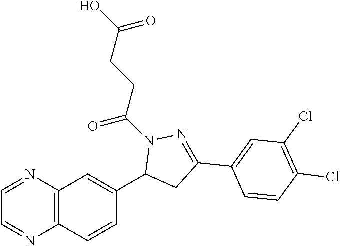 [SMILES: O=C(O)CCC(=O)N1N=C(c2ccc(Cl)c(Cl)c2)CC1c1ccc2nccnc2c1]

Compound 11 was prepared over 3 steps using a method similar to that described for the preparation of Compound 3-step 1: quinoxaline-6-carbaldehyde (200 mg, 1.26 mmol), 3,4-dichloroacetophenone (238 mg, 1.26 mmol) and 2 M NaOH (3.2 mL, 6.32 mmol) in EtOH (12 mL) to afford (E)-1-(3,4-dichlorophenyl)-3-(quinoxalin-6-yl)prop-2-en-1-one as a colourless solid (350 mg); step 2: (E)-1-(3,4-dichlorophenyl)-3-(quinoxalin-6-yl)prop-2-en-1-one (350 mg) and hydrazine hydrate (0.13 mL, 50% w/w) in EtOH (10 mL) to afford 6-(3-(3,4-dichlorophenyl)-4,5-dihydro-1H-pyrazol-5-yl)quinoxaline as a colourless solid (70 mg); and step 3:6-(3-(3,4-dichlorophenyl)-4,5-dihydro-1H-pyrazol-5-yl)quinoxaline (70 mg) and succinic anhydride (41 mg, 0.41 mmol) in THF (4 mL). Purification by flash column chromatography (EtOAc, neat→CH₂Cl/MeOH, 9:1) afforded Compound 11 as a brown solid (32 mg, 6%). ¹H NMR (500 MHz, CDCl₃) δ 8.82-8.80 (2H, m), 8.10 (1H, d, J=8.8 Hz), 7.95 (1H, d, J=1.6 Hz), 7.82 (1H, d, J=1.9 Hz), 7.64 (1H, dd, J=8.7, 1.9 Hz), 7.57 (1H, dd, J=8.3, 2.0 Hz), 7.50 (1H, d, J=8.5 Hz), 5.84 (1H, dd, J=12.0, 5.0 Hz), 3.84 (1H, dd, J=17.8, 12.1 Hz), 3.29 (1H, dt, J=17.6, 7.6 Hz), 3.19 (1H, dd, J=17.8, 5.1 Hz), 3.13 (1H, dt, J=17.6, 6.7 Hz), 2.75 (2H, t, J=6.8 Hz); ¹³C NMR (125 MHz, CDCl₃) δ 176.4 (C), 170.4 (C), 152.2 (C), 145.4 (CH), 145.1 (CH), 143.4 (C), 143.0 (C), 142.5 (C), 134.9 (C), 133.4 (C), 131.1 (C), 131.0 (CH), 130.7 (CH), 128.6 (CH), 128.0 (CH), 125.9 (2×CH), 60.5 (CH₂), 42.1 (CH₂), 29.1 (CH₂), 28.7 (CH₂); HRMS (ESI/Q-TOF) m/z: [M+Na]⁺ calcd for C₂₁H₁₆C₁₂N₄NaO₃, 465.0492; found 465.0506.

### 4-(3-([1,1′-Biphenyl]-2-yl)-5-(quinoxalin-6-yl)-4,5-dihydro-1H-pyrazol-1-yl)-4-oxobutanoic acid (Compound 12)

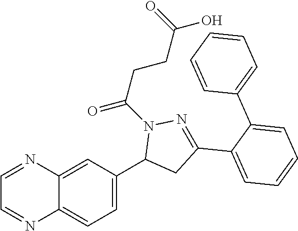 [SMILES: O=C(O)CCC(=O)N1N=C(c2ccccc2-c2ccccc2)CC1c1ccc2nccnc2c1]

To a solution of quinoxaline-6-carbaldehyde (237 mg, 1.50 mmol) and 2-acetylbiphenyl (294 mg, 1.50 mmol) in EtOH (12 mL) at room temperature was added dropwise 2 M NaOH (3.8 mL, 7.50 mmol), and the reaction mixture was stirred for 1 h. After, the solution was cooled down to 0° C., and water was added to the reaction mixture (12 mL). The resulting precipitated solids were then collected by filtration, washed with water and dried in vacuo to afford (E)-1-([1,1′-biphenyl]-2-yl)-3-(quinoxalin-6-yl)prop-2-en-1-one as a yellow solid (353 mg), which was used directly in the next step without further purification.

A mixture of (E)-1-([1,1′-biphenyl]-2-yl)-3-(quinoxalin-6-yl)prop-2-en-1-one (329 mg) and hydrazine hydrate (0.18 mL, 50% w/w) in THF (8 mL) was stirred under reflux for 2 h. After, succinic anhydride (294 mg, 2.94 mmol) was added and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. Purification by flash column chromatography (EtOAc, neat→EtOAc/MeOH, 9:1) afforded Compound 12 as a yellow solid (194 mg, 31% over 2 steps). ¹H NMR (400 MHz, CDCl₃) δ 8.84-8.81 (2H, m), 8.05 (1H, d, J=8.7 Hz), 7.82 (1H, d, J=1.9 Hz), 7.75 (1H, dd, J=7.3, 1.6 Hz), 7.51-7.41 (3H, m), 7.35 (1H, dd, J=7.1, 1.6 Hz), 7.27-7.25 (2H, m), 7.22-7.15 (3H, m), 5.59 (1H, dd, J=11.8, 4.5 Hz), 3.21 (1H, dd, J=18.1, 11.8 Hz), 3.10 (1H, dt, J=17.4, 6.6 Hz), 2.99 (1H, dt, J=17.4, 6.8 Hz), 2.69 (2H, t, J=7.0 Hz), 2.50 (1H, dd, J=18.1, 4.6 Hz); ¹³C NMR (101 MHz, CDCl₃) δ 176.4 (C), 170.4 (C), 156.8 (C), 145.3 (CH), 145.0 (CH), 143.5 (C), 142.9 (C), 142.5 (C), 142.0 (C), 141.1 (C), 130.9 (CH), 130.6 (C), 130.4 (CH), 130.1 (CH), 129.4 (CH), 129.0 (2×CH), 128.5 (2×CH), 128.2 (CH), 127.8 (CH), 127.7 (CH), 125.7 (CH), 60.0 (CH), 44.5 (CH₂), 29.1 (CH₂), 28.9 (CH₂); HRMS (ESI/Q-TOF) m/z: [M+H]⁺ calcd for C₂₇H₂₃N₄O₃ 451.1765; found 451.1757.

### 4-(3-([1,1′-Biphenyl]-3-yl)-5-(quinoxalin-6-yl)-4,5-dihydro-1H-pyrazol-1-yl)-4-oxobutanoic acid (Compound 13)

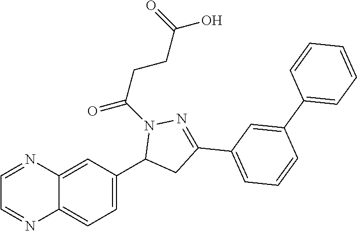 [SMILES: O=C(O)CCC(=O)N1N=C(c2cccc(-c3ccccc3)c2)CC1c1ccc2nccnc2c1]

To a solution of quinoxaline-6-carbaldehyde (237 mg, 1.50 mmol) and 3-acetylbiphenyl (294 mg, 1.50 mmol) in EtOH (12 mL) at room temperature was added dropwise 2 M NaOH (3.8 mL, 7.50 mmol), and the reaction mixture was stirred for 1 h. After, the solution was cooled down to 0° C., and the resulting precipitated solids were then collected by filtration, washed with water and dried in vacuo to afford (E)-1-([1,1′-biphenyl]-3-yl)-3-(quinoxalin-6-yl)prop-2-en-1-one as a yellow solid (405 mg), which was used directly in the next step without further purification.

A mixture of (E)-1-([1,1′-biphenyl]-3-yl)-3-(quinoxalin-6-yl)prop-2-en-1-one (405 mg) and hydrazine hydrate (0.22 mL, 50% w/w) in THF (10 mL) was stirred under reflux for 22 h. After, succinic anhydride (361 mg, 3.61 mmol) was added, and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. Purification by flash column chromatography (CH₂Cl₂, neat→CH₂Cl₂/MeOH, 9:1) afforded Compound 13 as a yellow solid (115 mg, 17% over 2 steps). ¹H NMR (400 MHz, CDCl₃) δ 8.80-8.78 (2H, m), 8.10 (1H, d, J=8.7 Hz), 7.99 (1H, d, J=1.9 Hz), 7.95 (1H, t, J=1.6 Hz), 7.73 (1H, dt, J=7.7, 1.3 Hz), 7.70-7.64 (2H, m), 7.62-7.59 (2H, m), 7.53-7.43 (3H, m), 7.40-7.34 (1H, m), 5.84 (1H, dd, J=11.9, 4.9 Hz), 3.92 (1H, dd, J=17.7, 11.9 Hz), 3.33-3.15 (3H, m), 2.75 (2H, t, J=6.8 Hz); ¹³C NMR (101 MHz, CDCl₃) δ 176.2 (C), 170.4 (C), 154.5 (C), 145.4 (CH), 145.1 (CH), 143.8 (C), 143.0 (C), 142.6 (C), 142.1 (C), 140.4 (C), 131.6 (C), 130.6 (CH), 129.6 (CH), 129.4 (CH), 129.0 (2×CH), 128.2 (CH), 127.9 (CH), 127.3 (2×CH), 125.9 (CH), 125.7 (CH), 125.6 (CH), 60.2 (CH), 42.5 (CH₂), 29.2 (CH₂), 28.9 (CH₂); HRMS (ESI/Q-TOF) m/z: [M+H]⁺ calcd for C₂₇H₂₃N₄O₃ 451.1765; found 451.1775.

### 4-Oxo-4-(5-(quinoxalin-6-yl)-3-(4′-(trifluoromethoxy)-[1,1′-biphenyl]-3-yl)-4,5-dihydro-1H-pyrazol-1-yl)butanoic acid (Compound 14)

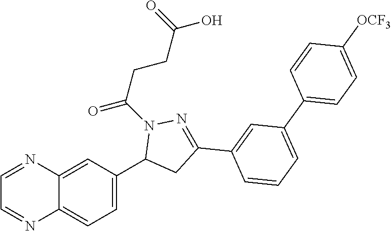 [SMILES: O=C(O)CCC(=O)N1N=C(c2cccc(-c3ccc(OC(F)(F)F)cc3)c2)CC1c1ccc2nccnc2c1]

Compound 14 was prepared over 2 steps using a method similar to that described for the preparation of Compound 20-step 1: quinoxaline-6-carbaldehyde (237 mg, 1.50 mmol), 1-(4′-(trifluoromethoxy) biphenyl-3-yl) ethanone (420 mg, 1.50 mmol) and 2 M NaOH (3.8 mL, 7.50 mmol) in EtOH (12 mL) to afford (E)-3-(quinoxalin-6-yl)-1-(4′-(trifluoromethoxy)-[1,1′-biphenyl]-3-yl)prop-2-en-1-one as an off-white solid (631 mg); step 2: (E)-3-(quinoxalin-6-yl)-1-(4′-(trifluoromethoxy)-[1,1′-biphenyl]-3-yl)prop-2-en-1-one (350 mg) and hydrazine hydrate (0.10 mL, 50% w/w) in THF (7 mL), followed by the addition of succinic anhydride (250 mg, 2.50 mmol). Purification by flash column chromatography (petroleum ether/EtOAc, 1:1→EtOAc, neat→CH₂Cl₂/MeOH, 9:1) afforded Compound 14 as an off-white solid (45 mg, 10% over 2 steps). ¹H NMR (400 MHz, DMSO-d₆) δ 12.08 (1H, s), 8.94-8.92 (2H, m), 8.09 (1H, d, J=8.7 Hz), 8.02 (1H, t, J=1.4 Hz), 7.92-7.78 (5H, m), 7.74 (1H, dd, J=8.7, 2.0 Hz), 7.60 (1H, t, J=7.8 Hz), 7.46 (2H, d, J=8.0 Hz), 5.87 (1H, dd, J=12.0, 5.0 Hz), 4.04 (1H, dd, J=18.3, 12.0 Hz), 3.46 (1H, dd, J=18.3, 5.0 Hz), 3.17-2.96 (2H, m), 2.52 (2H, t, J=6.9 Hz); ¹³C NMR (101 MHz, DMSO-d₆) δ 173.7 (C), 169.3 (C), 154.4 (C), 148.1 (C, J_{C-F}=1.8 Hz), 146.0 (CH), 145.6 (CH), 144.2 (C), 142.1 (C), 141.6 (C), 139.3 (C), 138.8 (C), 131.7 (C), 129.8 (CH), 129.5 (CH), 128.8 (2×CH), 128.8 (CH), 128.2 (CH), 125.9 (CH), 125.4 (CH), 125.3 (CH), 121.5 (2×CH), 120.1 (C, J_{C-F}=255 Hz), 59.6 (CH), 41.8 (CH₂), 28.7 (CH₂), 28.3 (CH₂); HRMS (ESI/Q-TOF) m/z: [M+Na]⁺ calcd for C₂₈H₂₁F₃N₄NaO₄, 557.1407; found, 557.1412.

### 4-(3-([1,1′-Biphenyl]-4-yl)-5-(quinoxalin-6-yl)-4,5-dihydro-1H-pyrazol-1-yl)-4-oxobutanoic acid (Compound 15)

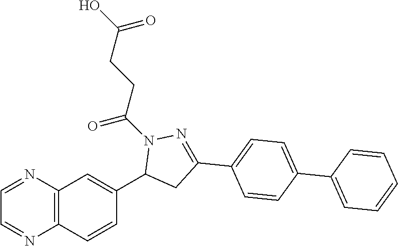 [SMILES: O=C(O)CCC(=O)N1N=C(c2ccc(-c3ccccc3)cc2)CC1c1ccc2nccnc2c1]

Compound 15 was prepared over 3 steps using a method similar to that described for the preparation of Compound 3-step 1: quinoxaline-6-carbaldehyde (200 mg, 1.26 mmol), 4-acetylbiphenyl (248 mg, 1.26 mmol) and 2 M NaOH (3.2 mL, 6.32 mmol) in EtOH (12 mL) to afford (E)-1-([1,1′-biphenyl]-4-yl)-3-(quinoxalin-6-yl)prop-2-en-1-one as a beige solid (350 mg); step 2: (E)-1-([1,1′-biphenyl]-4-yl)-3-(quinoxalin-6-yl)prop-2-en-1-one (200 mg) and hydrazine hydrate (73 μL, 50% w/w) in EtOH (10 mL) to afford 6-(3-([1,1′-biphenyl]-4-yl)-4,5-dihydro-1H-pyrazol-5-yl)quinoxaline as a yellow solid (163 mg); and step 3:6-(3-([1,1′-biphenyl]-4-yl)-4,5-dihydro-1H-pyrazol-5-yl)quinoxaline (100 mg) and succinic anhydride (59 mg, 0.59 mmol) in THF (4 mL). Purification by flash column chromatography (EtOAc, neat→CH₂Cl₂/MeOH, 9:1) afforded Compound 15 as a yellow solid (27 mg, 14% over 3 steps). ¹H NMR (500 MHz, CDCl₃) δ 8.83 (2H, s), 8.12 (1H, d, J=8.7 Hz), 7.99 (1H, d, J=1.9 Hz), 7.84 (2H, d, J=8.5 Hz), 7.70-7.66 (3H, m), 7.63 (2H, dd, J=8.5, 1.4 Hz), 7.47 (2H, t, J=8.0 Hz), 7.39 (1H, tt, J=7.2, 2.0 Hz), 5.86 (1H, dd, J=11.9, 4.7 Hz), 3.94 (1H, dd, J=17.9, 11.9 Hz), 3.34 (1H, dt, J=17.4, 6.6 Hz), 3.31 (1H, dd, J=17.5, 4.9 Hz), 3.20 (1H, dt, J=17.5, 6.8 Hz), 2.76 (2H, t, J=6.6 Hz); ¹³C NMR (125 MHz, CDCl₃) δ 174.9 (C), 170.6 (C), 154.7 (C), 145.5 (CH), 145.2 (CH), 143.7 (C), 143.6 (C), 143.1 (C), 142.7 (C), 140.1 (C), 130.8 (CH), 129.8 (C), 129.1 (2×CH), 128.2 (CH), 128.1 (CH), 127.6 (2×CH), 127.4 (2×CH), 127.2 (2×CH), 126.0 (CH), 60.3 (CH), 42.4 (CH₂), 29.2 (CH₂), 29.0 (CH₂).

### 4-(3-(4′-Fluoro-[1,1′-biphenyl]-4-yl)-5-(quinoxalin-6-yl)-4,5-dihydro-1H-pyrazol-1-yl)-4-oxobutanoic acid (Compound 16)

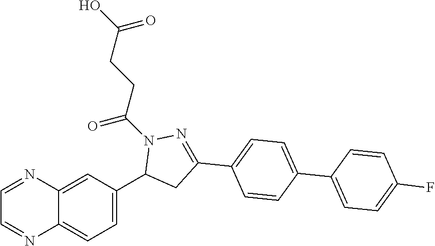 [SMILES: O=C(O)CCC(=O)N1N=C(c2ccc(-c3ccc(F)cc3)cc2)CC1c1ccc2nccnc2c1]

To a solution of quinoxaline-6-carbaldehyde (316 mg, 2.000) mmol) and 1-(4′-fluorobiphenyl-4-yl) ethanone (428 mg, 2.00 mmol) in EtOH (20 mL) at room temperature was added dropwise 2 M NaOH (5.00 mL, 10.00 mmol), and the reaction mixture stirred for 2 h. After, the mixture was cooled down to 0° C., and resulting precipitated solids were then collected by filtration, washed with water and dried in vacuo to afford (E)-1-(4′-fluoro-[1,1′-biphenyl]-4-yl)-3-(quinoxalin-6-yl)prop-2-en-1-one as an off-white solid (709 mg), which was used directly in the next step without further purification.

To a stirred solution of (E)-1-(4′-fluoro-[1,1′-biphenyl]-4-yl)-3-(quinoxalin-6-yl)prop-2-en-1-one (300 mg) in EtOH (5 mL) was added hydrazine hydrate (0.16 mL, 50% w/w), and the reaction mixture heated under reflux for 3 h. The solvent was then removed in vacuo, the residue poured onto ice water and the resulting precipitated solids were collected by filtration and dried in vacuo to afford 6-(3-(4′-fluoro-[1,1′-biphenyl]-4-yl)-4,5-dihydro-1H-pyrazol-5-yl)quinoxaline as a brown solid (310 mg), which was used in the next step without further purification.

In a sealed vial, a mixture of 6-(3-(4′-fluoro-[1,1′-biphenyl]-4-yl)-4,5-dihydro-1H-pyrazol-5-yl)quinoxaline (310 mg) and succinic anhydride (168 mg, 1.68 mmol) in THF (4 mL) was subjected to microwave irradiation at 120° C. for 45 min. The resulting reaction mixture was diluted with CH₂Cl₂ (20 mL), washed with water (20 mL) and the separated aqueous layer further extracted with CH₂Cl₂ (3×30 mL). The combined organic layers were washed with water (3×30 mL), then brine (30 mL), dried over anhydrous Na₂SO₄, and concentrated in vacuo. Purification by flash column chromatography (CH₂Ch/MeOH, 20:1) afforded Compound 16 as a beige solid (63 mg, 16% over 3 steps). ¹H NMR (400 MHz, DMSO-d₆) δ 12.08 (1H, br s), 8.94-8.92 (2H, m), 8.09 (1H, d, J=8.7 Hz), 7.92-7.89 (3H, m), 7.79-7.71 (5H, m), 7.32 (2H, t, J=8.9 Hz), 5.85 (1H, dd, J=12.0, 5.0 Hz), 4.01 (1H, dd, J=18.3, 12.0 Hz), 3.35 (1H, dd, J=18.3, 5.0 Hz), 3.15-2.95 (2H, m), 2.52 (2H, t, J=6.9 Hz); ¹³C NMR (101 MHz, DMSO-d₆) δ 173.7 (C), 169.2 (C), 162.1 (C, J_{C-F}=244 Hz), 154.1 (C), 146.0 (CH), 145.6 (CH), 144.2 (C), 142.1 (C), 141.6 (C), 140.8 (C), 135.6 (C, J_{C-F}=3.1 Hz), 130.0 (C), 129.8 (CH), 128.8 (2×CH, J_{C-F}=8.2 Hz), 128.1 (2×CH), 127.4 (CH), 126.9 (2×CH), 125.3 (CH), 115.8 (2×CH, J_{C-F}=21.2 Hz), 59.6 (CH), 41.8 (CH₂), 28.8 (CH₂), 28.4 (CH₂); HRMS (ESI/Q-TOF) m/z: [M+Na]⁺ calcd for C₂₇H₂₁FN₄NaO₃, 491.1490; found, 491.1488.

### 4-(3-(2′-Chloro-[1,1′-biphenyl]-4-yl)-5-(quinoxalin-6-yl)-4,5-dihydro-1H-pyrazol-1-yl)-4-oxobutanoic acid (Compound 17)

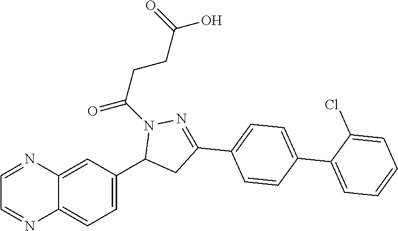 [SMILES: O=C(O)CCC(=O)N1N=C(c2ccc(-c3ccccc3Cl)cc2)CC1c1ccc2nccnc2c1]

To a solution of quinoxaline-6-carbaldehyde (200 mg, 1.26 mmol) and 1-(2′-chlorobiphenyl-4-yl) ethanone (291 mg, 1.26 mmol) in EtOH (12 mL) at 0° C. was added dropwise 2 M NaOH (3.2 mL, 6.32 mmol), and the reaction mixture stirred for 18 h (while gradually warming to room temperature). The resulting precipitated solids were then collected by filtration, washed with water and dried in vacuo to afford (E)-1-(2′-chloro-[1,1′-biphenyl]-4-yl)-3-(quinoxalin-6-yl)prop-2-en-1-one as an off-white solid (402 mg), which was used directly in the next step without further purification.

To a stirred solution of (E)-1-(2′-chloro-[1,1′-biphenyl]-4-yl)-3-(quinoxalin-6-yl)prop-2-en-1-one (200 mg) in EtOH (5 mL) was added hydrazine hydrate (67 μL, 50% w/w), and the reaction mixture heated under reflux for 2 h. The solvent was then removed in vacuo and the resulting residue was diluted in EtOAc (30 mL) and washed with water (2×20 mL), then brine (20 mL), dried over anhydrous Na₂SO₄, filtered, and concentrated in vacuo to afford 6-(3-(2′-chloro-[1,1′-biphenyl]-4-yl)-4,5-dihydro-1H-pyrazol-5-yl)quinoxaline as an orange wax (208 mg), which was used in the next step without further purification.

In a sealed vial, a mixture of 6-(3-(2′-chloro-[1,1′-biphenyl]-4-yl)-4,5-dihydro-1H-pyrazol-5-yl)quinoxaline (208 mg) and succinic anhydride (108 mg, 1.08 mmol) in THF (4 mL) was subjected to microwave irradiation at 120° C. for 45 min. The resulting reaction mixture was concentrated in vacuo. Purification by flash column chromatography (petroleum ether/EtOAc, 1:1→EtOAc, neat→CH₂Cl₂/MeOH, 9:1) afforded Compound 17 as an orange solid (21 mg, 7% over 3 steps). ¹H NMR (400 MHz, DMSO-d₆) δ 12.13 (1H, br s), 8.95-8.92 (2H, m), 8.09 (1H, d, J=8.7 Hz), 7.92-7.90 (3H, m), 7.73 (1H, dd, J=8.7, 2.0 Hz), 7.62-7.52 (3H, m), 7.48-7.40 (3H, m), 5.86 (1H, dd, J=11.9, 4.9 Hz), 4.03 (1H, dd, J=17.9, 12.2 Hz), 3.36 (1H, dd, J=18.3, 5.1 Hz), 3.11 (1H, dt, J=17.2, 6.7 Hz), 2.99 (1H, dt, J=17.2, 6.6 Hz), 2.54-2.50 (2H, m); ¹³C NMR (101 MHz, DMSO-d₆) δ 173.7 (C), 169.3 (C), 154.1 (C), 146.0 (CH), 145.6 (CH), 144.2 (C), 142.1 (C), 141.6 (C), 140.6 (C), 139.0 (C), 131.4 (CH), 131.2 (C), 130.4 (C), 129.9 (CH), 129.8 (CH), 129.7 (2×CH), 129.6 (CH), 128.1 (CH), 127.6 (CH), 126.6 (2×CH), 125.4 (CH), 59.6 (CH), 41.7 (CH₂), 28.8 (CH₂), 28.4 (CH₂); HRMS (ESI/Q-TOF) m/z: [M+Na]⁺ calcd for C₂₇H₂₁ClN₄NaO₃ 507.1194; found, 507.1187.

### 4-(3-(3′-Chloro-[1,1′-biphenyl]-4-yl)-5-(quinoxalin-6-yl)-4,5-dihydro-1H-pyrazol-1-yl)-4-oxobutanoic acid (Compound 18)

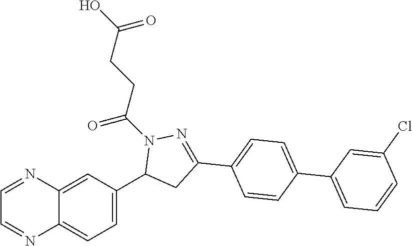 [SMILES: O=C(O)CCC(=O)N1N=C(c2ccc(-c3cccc(Cl)c3)cc2)CC1c1ccc2nccnc2c1]

To a solution of quinoxaline-6-carbaldehyde (350 mg, 2.21 mmol) and 1-(3′-chlorobiphenyl-4-yl) ethanone (510 mg, 2.21 mmol) in EtOH (22 mL) at 0° C. was added dropwise 2 M NaOH (5.5 mL, 11.0 mmol), and the reaction mixture stirred for 16 h (while gradually warming to room temperature). The resulting precipitated solids were collected by filtration, washed with water and dried in vacuo to afford (E)-1-(3′-chloro-[1,1′-biphenyl]-4-yl)-3-(quinoxalin-6-yl)prop-2-en-1-one as a pale-pink solid (820 mg), which was used directly in the next step without further purification.

To a stirred solution of (E)-1-(3′-chloro-[1,1′-biphenyl]-4-yl)-3-(quinoxalin-6-yl)prop-2-en-1-one (500 mg) in EtOH (8 mL) was added hydrazine hydrate (0.25 mL, 50% w/w), and the reaction mixture heated under reflux for 3 h. The solvent was then removed in vacuo, the residue poured onto ice water and the resulting precipitated solids were collected by filtration and dried in vacuo to afford 6-(3-(3′-chloro-[1,1′-biphenyl]-4-yl)-4,5-dihydro-1H-pyrazol-5-yl)quinoxaline as a brown solid (350 mg), which was used in the next step without further purification.

In a sealed vial, a mixture of 6-(3-(3′-chloro-[1,1′-biphenyl]-4-yl)-4,5-dihydro-1H-pyrazol-5-yl)quinoxaline (350 mg) and succinic anhydride (182 mg, 1.82 mmol) in THF (10 mL) was subjected to microwave irradiation at 120° C. for 45 min. The resulting reaction mixture was diluted with CH₂Ch (20 mL), washed with water (20 mL) and the separated aqueous layer further extracted with CH₂Cl₂ (3×30 mL). The combined organic layers were washed with water (3×30 mL), then brine (30 mL), dried over anhydrous Na₂SO₄, and concentrated in vacuo. Purification by flash column chromatography (CH₂Cl/MeOH, 100:1→CH₂Cl/MeOH, 25:1) afforded Compound 18 as a pale-brown solid (262 mg, 40% over 3 steps). ¹H NMR (400 MHz, CDCl₃) δ 8.80-8.79 (2H, m), 8.09 (1H, d, J=8.7 Hz), 7.99 (1H, d, J=1.7 Hz), 7.83-7.80 (2H, m), 7.67 (1H, dd, J=8.7, 2.0 Hz), 7.62-7.58 (3H, m), 7.49-7.46 (1H, m), 7.39-7.34 (2H, m), 5.84 (1H, dd, J=11.9, 4.9 Hz), 3.89 (1H, dd, J=17.7, 11.9 Hz), 3.33-3.13 (3H, m), 2.75 (2H, t, J=6.8 Hz); ¹³C NMR (101 MHz, CDCl₃) δ 176.9 (C), 170.3 (C), 153.9 (C), 145.3 (CH), 145.0 (CH), 143.9 (C), 142.9 (C), 142.4 (C), 142.0 (C), 141.9 (C), 135.0 (C), 130.6 (C), 130.5 (CH), 130.3 (CH), 128.2 (CH), 128.0 (CH), 127.5 (2×CH), 127.4 (2×CH), 127.3 (CH), 125.8 (CH), 125.3 (CH), 60.2 (CH), 42.3 (CH₂), 29.2 (CH₂), 28.9 (CH₂); HRMS (ESI/Q-TOF) m/z: [M+H]⁺ calcd for C₂₇H₂₂ClN₄O₃, 485.1375; found, 485.1386.

### 4-(3-(4′-Chloro-[1,1′-biphenyl]-4-yl)-5-(quinoxalin-6-yl)-4,5-dihydro-1H-pyrazol-1-yl)-4-oxobutanoic acid (Compound 19)

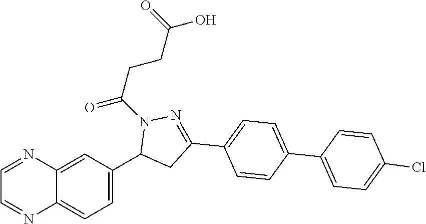 [SMILES: O=C(O)CCC(=O)N1N=C(c2ccc(-c3ccc(Cl)cc3)cc2)CC1c1ccc2nccnc2c1]

Compound 19 was prepared over 3 steps using a method similar to that described for the preparation of Compound 3-step 1: quinoxaline-6-carbaldehyde (200 mg, 1.26 mmol), 1-(4′-chlorobiphenyl-4-yl) ethanone (291 mg, 1.26 mmol) and 2 M NaOH (3.2 mL, 6.32 mmol) in EtOH (12 mL) to afford (E)-1-(4′-chloro-[1,1′-biphenyl]-4-yl)-3-(quinoxalin-6-yl)prop-2-en-1-one as an off-white solid (402 mg); step 2: (E)-1-(4′-chloro-[1,1′-biphenyl]-4-yl)-3-(quinoxalin-6-yl)prop-2-en-1-one (200 mg) and hydrazine hydrate (67 μL, 50% w/w) in EtOH (5 mL) to afford 6-(3-(4′-chloro-[1,1′-biphenyl]-4-yl)-4,5-dihydro-1H-pyrazol-5-yl)quinoxaline as an off-white solid (168 mg); and step 3:6-(3-(4′-chloro-[1,1′-biphenyl]-4-yl)-4,5-dihydro-1H-pyrazol-5-yl)quinoxaline (150 mg) and succinic anhydride (78 mg, 0.78 mmol) in THF (3 mL). Purification by flash column chromatography (EtOAc, neat EtOAc/MeOH, 9:1) afforded Compound 19 as an off-white solid (28 mg, 10% over 3 steps). ¹H NMR (400 MHz, DMSO-d₆) δ 12.08 (1H, br s), 8.94-8.92 (2H, m), 8.09 (1H, d, J=8.7 Hz), 7.93-7.88 (3H, m), 7.83-7.69 (5H, m), 7.59-7.51 (2H, m), 5.86 (1H, dd, J=11.9, 5.0 Hz), 4.02 (1H, dd, J=18.1, 12.0 Hz), 3.38-3.34 (1H, m), 3.11 (1H, dt, J=17.2, 6.7 Hz), 2.99 (1H, dt, J=17.2, 6.6 Hz), 2.53-2.50 (2H, m); HRMS (ESI/Q-TOF) m/z: [M+Na]⁺ calcd for C₂₇H₂₁ClN₄NaO₃, 507.1194; found, 507.1182.

### 4-Oxo-4-(5-(quinoxalin-6-yl)-3-(4′-(trifluoromethyl)-[1,1′-biphenyl]-4-yl)-4,5-dihydro-1H-pyrazol-1-yl)butanoic acid (Compound 20)

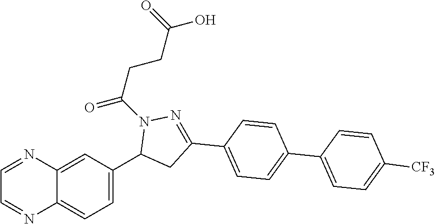 [SMILES: O=C(O)CCC(=O)N1N=C(c2ccc(-c3ccc(C(F)(F)F)cc3)cc2)CC1c1ccc2nccnc2c1]

To a solution of quinoxaline-6-carbaldehyde (158 mg, 1.00 mmol) and 1-(4′-(trifluoromethyl)biphenyl-4-yl)ethanone (264 mg, 1.00 mmol) in EtOH (8 mL) at room temperature was added dropwise 2 M NaOH (2.5 mL, 5.0 mmol), and the reaction mixture was stirred for 3 h. After, the solution was cooled down to 0° C., and the resulting precipitated solids were then collected by filtration, washed with water and dried in vacuo to afford (E)-3-(quinoxalin-6-yl)-1-(4′-(trifluoromethyl)-[1,1′-biphenyl]-4-yl)prop-2-en-1-one as an off-white solid (319 mg), which was used directly in the next step without further purification.

A mixture of (E)-3-(quinoxalin-6-yl)-1-(4′-(trifluoromethyl)-[1,1′-biphenyl]-4-yl)prop-2-en-1-one (319 mg) and hydrazine hydrate (0.15 mL, 50% w/w) in THF (6 mL) was stirred under reflux for 2 h. After, succinic anhydride (237 mg, 2.37 mmol) was added, and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. Purification by flash column chromatography (petroleum ether/EtOAc, 1:1→EtOAc, neat→EtOAc/MeOH, 9:1) afforded Compound 20 as a yellow solid (20 mg, 4% over 2 steps). ¹H NMR (400 MHz, CDCl₃) δ 8.80 (2H, s), 8.10 (1H, d, J=8.7 Hz), 7.98 (1H, s), 7.85 (2H, d, J=8.5 Hz), 7.71-7.65 (7H, m), 5.85 (1H, dd, J=11.9, 4.9 Hz), 3.92 (1H, dd, J=17.7, 11.9 Hz), 3.35-3.15 (3H, m), 2.76 (2H, t, J=6.7 Hz); HRMS (ESI/Q-TOF) m/z: [M+H]⁺ calcd for C₂₈H₂₂F₃N₄O₃, 519.1639; found, 519.1639.

### 4-Oxo-4-(5-(quinoxalin-6-yl)-3-(3′-(trifluoromethoxy)-[1,1′-biphenyl]-4-yl)-4,5-dihydro-1H-pyrazol-1-yl)butanoic acid (Compound 21)

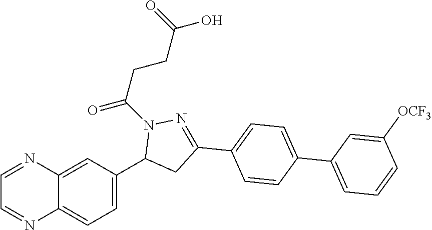 [SMILES: O=C(O)CCC(=O)N1N=C(c2ccc(-c3cccc(OC(F)(F)F)c3)cc2)CC1c1ccc2nccnc2c1]

To a solution of Compound 9 (68 mg, 0.15 mmol) in DMF/water (1.5 mL, 1:1 v/v), was added 3-(trifluoromethoxy)phenylboronic acid (62 mg, 0.30 mmol), potassium phosphate tribasic (127 mg, 0.60 mmol), and palladium acetate (3.4 mg, 10 mol %). The reaction mixture was stirred at 110° C. for 24 h. To the mixture was added water (15 mL) and the suspension was acidified to aprox. pH 2 through the addition of 1 M aq. HCl. The aqueous layer was then extracted with ethyl acetate (2×15 mL) and the combined organic layers were washed with water (3×15 mL), then brine (15 mL), dried over anhydrous Na₂SO₄, filtered through celite and concentrated in vacuo. Purification by flash column chromatography (CH₂Cl₂, neat→CH₂Cl₂/MeOH 9:1) afforded Compound 21 as a brown solid (42 mg, 53%). ¹H NMR (400 MHz, CDCl₃) δ 8.81 (2H, s), 8.11 (1H, d, J=8.7 Hz), 7.99 (1H, s), 7.84 (2H, d, J=8.5 Hz), 7.68 (1H, dd, J=8.7, 1.8 Hz), 7.64 (2H, d, J=8.5 Hz), 7.54 (1H, dt, J=8.0, 1.4 Hz), 7.51-7.42 (2H, m), 7.26-7.23 (1H, m), 5.85 (1H, dd, J=11.9, 4.9 Hz), 3.92 (1H, dd, J=17.7, 12.0 Hz), 3.33-3.15 (3H, m), 2.76 (2H, t, J=6.7 Hz); HRMS (ESI/Q-TOF) m/z: [M+Na]⁺ calcd for C₂₈H₂₁F₃N₄NaO₄, 557.1407; found, 557.1406.

### 4-Oxo-4-(5-(quinoxalin-6-yl)-3-(4′-(trifluoromethoxy)-[1,1′-biphenyl]-4-yl)-4,5-dihydro-1H-pyrazol-1-yl)butanoic acid (Compound 22)

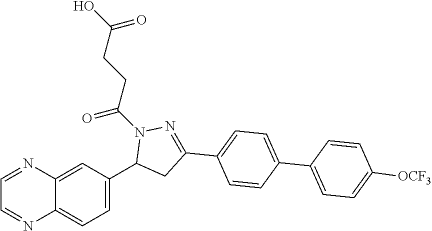 [SMILES: O=C(O)CCC(=O)N1N=C(c2ccc(-c3ccc(OC(F)(F)F)cc3)cc2)CC1c1ccc2nccnc2c1]

To a solution of quinoxaline-6-carbaldehyde (316 mg, 2.00 mmol) and 1-(4′-(trifluoromethoxy) biphenyl-4-yl) ethanone (560 mg, 2.00 mmol) in EtOH (10 mL) at room temperature was added dropwise 2 M NaOH (5.0 mL, 10.0 mmol), and the reaction mixture stirred for 2 h. After, the mixture was cooled down to 0° C., and the resulting precipitated solids were then collected by filtration, washed with water and dried in vacuo to afford (E)-3-(quinoxalin-6-yl)-1-(4′-(trifluoromethoxy)-[1,1′-biphenyl]-4-yl)prop-2-en-1-one as an off-white solid (841 mg), which was used directly in the next step without further purification.

To a stirred solution of (E)-3-(quinoxalin-6-yl)-1-(4′-(trifluoromethoxy)-[1,1′-biphenyl]-4-yl)prop-2-en-1-one (300 mg) in EtOH (5 mL) was added hydrazine hydrate (0.13 mL, 50% w/w), and the reaction mixture heated under reflux for 2 h. The solvent was then removed in vacuo, the residue poured onto ice water and the resulting precipitated solids were collected by filtration and dried in vacuo to afford 6-(3-(4′-(trifluoromethoxy)-[1,1′-biphenyl]-4-yl)-4,5-dihydro-1H-pyrazol-5-yl)quinoxaline as a brown solid (310 mg), which was used in the next step without further purification.

In a sealed vial, a mixture of 6-(3-(4′-(trifluoromethoxy)-[1,1′-biphenyl]-4-yl)-4,5-dihydro-1H-pyrazol-5-yl)quinoxaline (310 mg) and succinic anhydride (143 mg, 1.43 mmol) in THF (4 mL) was subjected to microwave irradiation at 120° C. for 45 min. The resulting reaction mixture was diluted with CH₂Cl₂ (20 mL), washed with water (20 mL) and the separated aqueous layer further extracted with CH₂Cl₂ (3×30 mL). The combined organic layers were washed with water (3×30 mL), then brine (30 mL), dried over anhydrous Na₂SO₄, and concentrated in vacuo. Purification by flash column chromatography (CH₂Cl/MeOH, 20:1) afforded Compound 22 as a pale-brown solid (50 mg, 13% over 3 steps). ¹H NMR (400 MHz, CDCl₃) δ 8.81 (2H, s), 8.11 (1H, d, J=8.7 Hz), 7.99 (1H, d, J=1.6 Hz), 7.85-7.82 (2H, m), 7.69-7.61 (5H, m), 7.32-7.29 (2H, m), 5.85 (1H, dd, J=11.9, 4.9 Hz), 3.92 (1H, dd, J=17.7, 11.9 Hz), 3.33-3.13 (3H, m), 2.76 (2H, t, J=6.9 Hz); HRMS (ESI/Q-TOF) m/z: [M+Na]⁺ calcd for C₂₈H₂₁F₃N₄NaO₄, 557.1407; found, 557.1397.

### 4-(3-(4′-Methyl-[1,1′-biphenyl]-4-yl)-5-(quinoxalin-6-yl)-4,5-dihydro-1H-pyrazol-1-yl)-4-oxobutanoic acid (Compound 23)

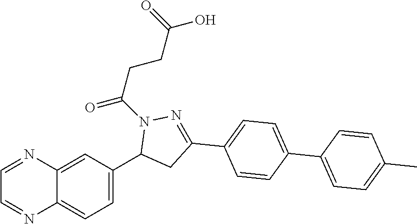 [SMILES: Cc1ccc(-c2ccc(C3=NN(C(=O)CCC(=O)O)C(c4ccc5nccnc5c4)C3)cc2)cc1]

Compound 23 was prepared using a method similar to that described for the preparation of Compound 21: Compound 9 (50 mg, 0.11 mmol), 4-methylphenylboronic acid (30 mg, 0.22 mmol), potassium phosphate tribasic (93 mg, 0.44 mmol), and palladium acetate (2.5 mg, 10 mol %) in DMF/water (1 mL, 1:1 v/v). Purification by flash column chromatography (petroleum ether/EtOAc, 1:1→EtOAc, neat→CH₂Cl₂/MeOH, 9:1) afforded Compound 23 as an orange solid (42 mg, 82%). ¹H NMR (400 MHz, CDCl₃) δ 8.81 (2H, s), 8.10 (1H, d, J=8.7 Hz), 7.99 (1H, d, J=1.9 Hz), 7.81 (2H, d, J=8.6 Hz), 7.68 (1H, dd, J=8.7, 1.9 Hz), 7.64 (2H, d, J=8.5 Hz), 7.52 (2H, d, J=8.2 Hz), 7.28-7.26 (2H, m), 5.84 (1H, dd, J=11.9, 4.9 Hz), 3.91 (1H, dd, J=17.7, 11.9 Hz), 3.33-3.15 (3H, m), 2.75 (2H, t, J=6.8 Hz), 2.40 (3H, s); ¹³C NMR (101 MHz, CDCl₃) δ 176.2 (C), 170.4 (C), 154.3 (C), 145.3 (CH), 145.1 (CH), 143.9 (C), 143.5 (C), 143.0 (C), 142.6 (C), 138.1 (C), 137.2 (C), 130.6 (CH), 129.8 (2×CH), 129.6 (C), 128.2 (CH), 127.4 (2×CH), 127.3 (2×CH), 127.0 (2×CH), 125.9 (CH), 60.2 (CH), 42.4 (CH₂), 29.2 (CH₂), 28.9 (CH₂), 21.3 (CH₃); HRMS (ESI/Q-TOF) m/z: [M+Na]⁺ calcd for C₂₈H₂₄N₄NaO₃, 487.1741; found, 487.1740.

### 4-(3-(4′-Isopropyl-[1,1′-biphenyl]-4-yl)-5-(quinoxalin-6-yl)-4,5-dihydro-1H-pyrazol-1-yl)-4-oxobutanoic acid (Compound 24)

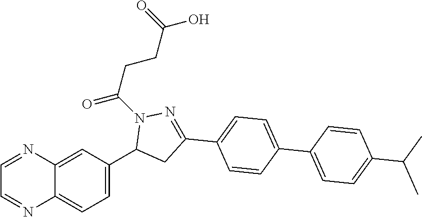 [SMILES: CC(C)c1ccc(-c2ccc(C3=NN(C(=O)CCC(=O)O)C(c4ccc5nccnc5c4)C3)cc2)cc1]

Compound 24 was prepared using a method similar to that described for the preparation of Compound 21: Compound 9 (100 mg, 0.22 mmol), 4-isopropylphenylboronic acid (72 mg, 0.44 mmol), potassium phosphate tribasic (187 mg, 0.88 mmol), and palladium acetate (4.9 mg, 10 mol %) in DMF/water (2 mL, 1:1 v/v). Purification by flash column chromatography (petroleum ether/EtOAc, 1:1→EtOAc, neat→CH₂Cl₂/MeOH, 9:1) afforded Compound 24 as an orange solid (74 mg, 68%). ¹H NMR (400 MHz, CDCl₃) δ 8.82-8.79 (2H, m), 8.10 (1H, d. J=8.7 Hz), 7.99 (1H, d, J=1.8 Hz), 7.81 (2H, d, J=8.6 Hz), 7.70-7.62 (3H, m), 7.56 (2H, d. J=8.3 Hz), 7.33 (2H, d, J=8.1 Hz), 5.84 (1H, dd, J=11.8, 4.9 Hz), 3.91 (1H, dd, J=17.7, 11.9 Hz), 3.33-3.14 (3H, m), 2.96 (1H, hept, J=6.9 Hz), 2.75 (2H, t, J=6.8 Hz), 1.29 (6H, d, J=6.9 Hz); ¹³C NMR (101 MHz, CDCl₃) δ 176.5 (C), 170.4 (C), 154.4 (C), 149.0 (C), 145.3 (CH), 145.1 (CH), 143.8 (C), 143.5 (C), 143.0 (C), 142.6 (C), 137.6 (C), 130.6 (CH), 129.6 (C), 128.2 (CH), 127.4 (4×CH), 127.2 (2×CH), 127.1 (2×CH), 125.9 (CH), 60.2 (CH), 42.4 (CH₂), 34.0 (CH), 29.2 (CH₂), 29.0 (CH₂), 24.1 (2×CH₃); HRMS (ESI/Q-TOF) m/z: [M+H]⁺ calcd for C₃₀H₂₉N₄O₃, 493.2234; found, 493.2234.

### 4-(3-(4′-Methoxy-[1,1′-biphenyl]-4-yl)-5-(quinoxalin-6-yl)-4,5-dihydro-1H-pyrazol-1-yl)-4-oxobutanoic acid (Compound 25)

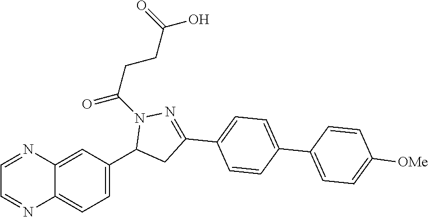 [SMILES: COc1ccc(-c2ccc(C3=NN(C(=O)CCC(=O)O)C(c4ccc5nccnc5c4)C3)cc2)cc1]

Compound 25 was prepared using a method similar to that described for the preparation of Compound 21: Compound 9 (68 mg, 0.15 mmol), 4-methoxyphenylboronic acid (46 mg, 0.30 mmol), potassium phosphate tribasic (127 mg, 0.60 mmol), and palladium acetate (3.4 mg, 10 mol %) in DMF/water (1.5 mL, 1:1 v/v). Purification by flash column chromatography (CH₂Cl₂, neat→CH₂Cl₂/MeOH 9:1) afforded Compound 25 as a yellow solid (32 mg, 44%). ¹H NMR (400 MHz, DMSO-d₆) δ 12.07 (1H, br s), 8.94-8.92 (2H, m), 8.08 (1H, d, J=8.7 Hz), 7.91 (1H, d, J=1.9 Hz), 7.86 (2H, d, J=8.5 Hz), 7.75-7.66 (5H, m), 7.04 (2H, d, J=8.9 Hz), 5.84 (1H, dd, J=11.9, 5.0 Hz), 4.00 (1H, dd, J=18.1, 12.0 Hz), 3.80 (3H, s), 3.36-3.30 (1H, m), 3.11 (1H, dt, J=17.2, 6.7 Hz), 2.98 (1H, dt, J=17.1, 6.6 Hz), 2.53-2.49 (2H, m); ¹³C NMR (101 MHz, DMSO-d₆) δ 173.8 (C), 169.2 (C), 159.4 (C), 154.2 (C), 146.0 (CH), 145.6 (CH), 144.2 (C), 142.2 (C), 141.59 (C), 141.55 (C), 131.4 (C), 129.8 (CH), 129.2 (C), 128.1 (CH), 127.9 (2×CH), 127.4 (2×CH), 126.3 (2×CH), 125.3 (CH), 114.5 (2×CH), 59.6 (CH), 55.2 (CH₃), 41.8 (CH₂), 28.8 (CH₂), 28.4 (CH₂); HRMS (ESI/Q-TOF) m/z: [M+H]⁺ calcd for C₂₈H₂₅N₄O₄, 481.1870; found, 481.1870.

### 4-(3-(2′,4′-Dichloro-[1,1′-biphenyl]-4-yl)-5-(quinoxalin-6-yl)-4,5-dihydro-1H-pyrazol-1-yl)-4-oxobutanoic acid (Compound 26)

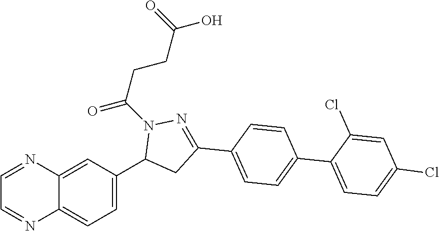 [SMILES: O=C(O)CCC(=O)N1N=C(c2ccc(-c3ccc(Cl)cc3Cl)cc2)CC1c1ccc2nccnc2c1]

Compound 26 was prepared over 2 steps using a method similar to that described for the preparation of Compound 20-step 1: quinoxaline-6-carbaldehyde (158 mg, 1.00 mmol), 1-(2′,4′-dichlorobiphenyl-4-yl) ethanone (265 mg, 1.00 mmol) and 2 M NaOH (2.5 mL, 5.00 mmol) in EtOH (8 mL) to afford (E)-1-(2′,4′-dichloro-[1,1′-biphenyl]-4-yl)-3-(quinoxalin-6-yl)prop-2-en-1-one as a colourless solid (344 mg); step 2: (E)-1-(2′,4′-dichloro-[1,1′-biphenyl]-4-yl)-3-(quinoxalin-6-yl)prop-2-en-1-one (344 mg) and hydrazine hydrate (0.16 mL, 50% w/w) in THF (7 mL), followed by the addition of succinic anhydride (255 mg, 2.55 mmol). Purification by flash column chromatography (petroleum ether/EtOAc, 1:1→EtOAc, neat→EtOAc/MeOH, 9:1) afforded Compound 26 as a yellow solid (93 mg, 18% over 2 steps). ¹H NMR (400 MHz, DMSO-d₆) δ 12.09 (1H, s), 8.94-8.92 (2H, m), 8.08 (1H, d, J=8.7 Hz), 7.92-7.90 (3H, m), 7.76 (1H, d, J=2.1 Hz), 7.73 (1H, dd, J=8.7, 2.0 Hz), 7.55-7.52 (3H, m), 7.47 (1H, d, J=8.3 Hz), 5.86 (1H, dd, J=11.9, 4.9 Hz), 4.02 (1H, dd, J=18.2, 12.0), 3.39-3.33 (1H, m), 3.11 (1H, dt, J=17.1, 6.7 Hz), 2.99 (1H, dt, J=17.2, 6.6 Hz), 2.52-2.49 (2H, m); ¹³C NMR (101 MHz, DMSO-d₆) δ 173.7 (C), 169.3 (C), 154.0 (C), 146.0 (CH), 145.6 (CH), 144.1 (C), 142.1 (C), 141.6 (C), 139.4 (C), 138.0 (C), 133.3 (C), 132.6 (C), 132.3 (CH), 130.7 (C), 129.8 (CH), 129.7 (2×CH), 129.4 (CH), 128.1 (CH), 127.8 (CH), 126.7 (2×CH), 125.4 (CH), 59.7 (CH), 41.7 (CH₂), 28.8 (CH₂), 28.3 (CH₂); HRMS (ESI/Q-TOF) m/z: [M+Na]⁺ calcd for C₂₇H₂₀C₁₂N₄NaO₃, 541.0805; found, 541.0806.

### 4-(3-(3′,4′-Dichloro-[1,1′-biphenyl]-4-yl)-5-(quinoxalin-6-yl)-4,5-dihydro-1H-pyrazol-1-yl)-4-oxobutanoic acid (Compound 27)

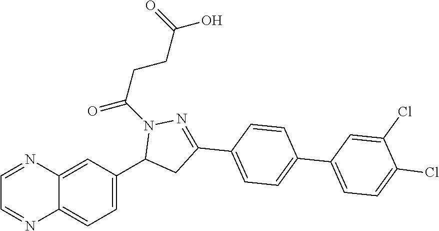 [SMILES: O=C(O)CCC(=O)N1N=C(c2ccc(-c3ccc(Cl)c(Cl)c3)cc2)CC1c1ccc2nccnc2c1]

Compound 27 was prepared over 2 steps using a method similar to that described for the preparation of Compound 20-step 1: quinoxaline-6-carbaldehyde (237 mg, 1.50 mmol), 1-(3′,4′-dichlorobiphenyl-4-yl) ethanone (398 mg, 1.50 mmol) and 2 M NaOH (3.8 mL, 7.50 mmol) in EtOH (12 mL) to afford (E)-1-(3′,4′-dichloro-[1,1′-biphenyl]-4-yl)-3-(quinoxalin-6-yl)prop-2-en-1-one as an off-white solid (486 mg); step 2: (E)-1-(3′,4′-dichloro-[1,1′-biphenyl]-4-yl)-3-(quinoxalin-6-yl)prop-2-en-1-one (270 mg) and hydrazine hydrate (0.12 mL, 50% w/w) in THF (10 mL), followed by the addition of succinic anhydride (200 mg, 2.00 mmol). Purification by flash column chromatography (petroleum ether/EtOAc, 1:1→EtOAc, neat→CH₂Cl₂/MeOH, 9:1) afforded Compound 27 as a yellow solid (97 mg, 22% over 2 steps). ¹H NMR (400 MHz, DMSO-d₆) δ 12.09 (1H, s), 8.94-8.92 (2H, m), 8.08 (1H, d, J=8.7 Hz), 8.02 (1H, s), 7.92-7.90 (3H, m), 7.85 (2H, d, J=8.6 Hz), 7.74-7.71 (3H, m), 5.86 (1H, dd, J=11.9, 5.0 Hz), 4.02 (1H, dd, J=18.2, 12.0 Hz), 3.36 (1H, dd, J=18.3, 5.2 Hz), 3.12 (1H, dt, J=17.1, 6.7 Hz), 2.99 (1H, dt, J=17.2, 6.6 Hz), 2.53-2.50 (2H, m); ¹³C NMR (101 MHz, DMSO-d₆) δ 173.7 (C), 169.3 (C), 154.0 (C), 146.0 (CH), 145.6 (CH), 144.1 (C), 142.1 (C), 141.6 (C), 139.7 (C), 139.0 (C), 131.9 (C), 131.1 (CH), 130.9 (C), 130.7 (C), 129.8 (CH), 128.5 (CH), 128.1 (CH), 127.4 (2×CH), 127.1 (2×CH), 126.9 (CH), 125.4 (CH), 59.6 (CH), 41.7 (CH₂), 28.8 (CH₂), 28.3 (CH₂); HRMS (ESI/Q-TOF) m/z: [M+H]⁺ calcd for C₂₇H₂₁C₁₂N₄O₃, 519.0985; found, 519.0965.

### 4-(3-(3′,5′-Dichloro-[1,1′-biphenyl]-4-yl)-5-(quinoxalin-6-yl)-4,5-dihydro-1H-pyrazol-1-yl)-4-oxobutanoic acid (Compound 28)

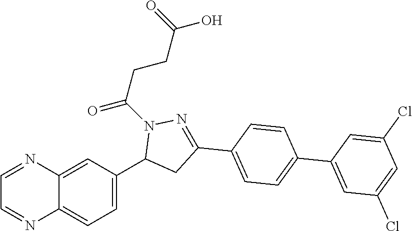 [SMILES: O=C(O)CCC(=O)N1N=C(c2ccc(-c3cc(Cl)cc(Cl)c3)cc2)CC1c1ccc2nccnc2c1]

Compound 28 was prepared over 2 steps using a method similar to that described for the preparation of Compound 20-step 1: quinoxaline-6-carbaldehyde (158 mg, 1.00 mmol), 1-(3′,5′-dichlorobiphenyl-4-yl) ethanone (265 mg, 1.00 mmol) and 2 M NaOH (2.5 mL, 5.00 mmol) in EtOH (8 mL) to afford (E)-1-(3′,5′-dichloro-[1,1′-biphenyl]-4-yl)-3-(quinoxalin-6-yl)prop-2-en-1-one as a yellow solid (353 mg); step 2: (E)-1-(3′,5′-dichloro-[1,1′-biphenyl]-4-yl)-3-(quinoxalin-6-yl)prop-2-en-1-one (353 mg) and hydrazine hydrate (0.16 mL, 50% w/w) in THF (7 mL), followed by the addition of succinic anhydride (261 mg, 2.61 mmol). Purification by flash column chromatography (petroleum ether/EtOAc, 1:1→EtOAc, neat→EtOAc/MeOH, 9:1) afforded Compound 28 as a yellow solid (88 mg, 17% over 2 steps). ¹H NMR (400 MHz, CDCl₃) δ 8.82-8.81 (2H, m), 8.11 (1H, d, J=8.7 Hz), 7.99 (1H, d, J=1.9 Hz), 7.83 (2H, d, J=8.6 Hz), 7.68 (1H, dd, J=8.7, 2.0 Hz), 7.59 (2H, d, J=8.6 Hz), 7.47 (2H, d, J=1.9 Hz), 7.36 (1H, t, J=1.8 Hz), 5.85 (1H, dd, J=11.9, 4.9 Hz), 3.91 (1H, dd, J=17.7, 12.0 Hz), 3.34-3.14 (3H, m), 2.76 (2H, t, J=6.7 Hz); ¹³C NMR (101 MHz, CDCl₃) δ 176.7 (C), 170.4 (C), 153.8 (C), 145.4 (CH), 145.1 (CH), 143.8 (C), 143.1 (C), 143.0 (C), 142.6 (C), 140.6 (C), 135.7 (2× C), 131.2 (C), 130.6 (CH), 128.2 (CH), 127.9 (CH), 127.6 (4× CH), 125.9 (CH), 125.7 (2×CH), 60.3 (CH), 42.3 (CH₂), 29.2 (CH₂), 28.9 (CH₂); HRMS (ESI/Q-TOF) m/z: [M+Na]⁺ calcd for C₂₇H₂₀C₁₂N₄NaO₃, 541.0805; found, 541.0790.

### 4-Oxo-4-(3-(5-phenylpyridin-2-yl)-5-(quinoxalin-6-yl)-4,5-dihydro-1H-pyrazol-1-yl)butanoic acid (Compound 29)

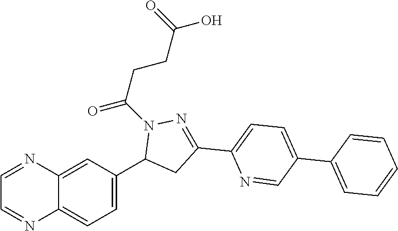 [SMILES: O=C(O)CCC(=O)N1N=C(c2ccc(-c3ccccc3)cn2)CC1c1ccc2nccnc2c1]

To a solution of quinoxaline-6-carbaldehyde (193 mg, 1.22 mmol) and 1-(5-phenylpyridin-2-yl)ethanone (240 mg, 1.22 mmol) in EtOH (12 mL) at 0° C. was added dropwise 2 M NaOH (3.0 mL, 6.10 mmol), and the reaction mixture stirred for 3 h (while gradually warming to room temperature). The resulting precipitated solids were then collected by filtration, washed with water and dried in vacuo to afford (E)-1-(5-phenylpyridin-2-yl)-3-(quinoxalin-6-yl)prop-2-en-1-one as a beige solid (320 mg), which was used directly in the next step without further purification.

To a stirred solution of (E)-1-(5-phenylpyridin-2-yl)-3-(quinoxalin-6-yl)prop-2-en-1-one (320 mg) in EtOH (7 mL) was added hydrazine hydrate (0.18 mL, 50% w/w), and the reaction mixture heated under reflux for 2 h. The solvent was then removed in vacuo, the residue poured onto ice water and the resulting precipitated solids were collected by filtration and dried in vacuo to afford 6-(3-(5-phenylpyridin-2-yl)-4,5-dihydro-1H-pyrazol-5-yl)quinoxaline as a yellow solid (300 mg), which was used in the next step without further purification.

In a sealed vial, a mixture of 6-(3-(5-phenylpyridin-2-yl)-4,5-dihydro-1H-pyrazol-5-yl)quinoxaline (300 mg) and succinic anhydride (171 mg, 1.71 mmol) in THF (10 mL) was subjected to microwave irradiation at 120° C. for 45 min. The resulting reaction mixture was diluted with EtOAc (20 mL), washed with water (20 mL) and the separated aqueous layer further extracted with EtOAc (3×30 mL). The combined organic layers were washed with water (3×30 mL), then brine (30 mL), dried over anhydrous Na₂SO₄, and concentrated in vacuo. Purification by flash column chromatography (CH-Ch/MeOH, 20:1) afforded Compound 29 as an off-white solid (110 mg, 20% over 3 steps). ¹H NMR (400 MHz, DMSO-d₆) δ 12.13 (1H, br s), 8.97-8.92 (3H, m), 8.25-8.18 (2H, m), 8.08 (1H, d, J=8.7 Hz), 7.92 (1H, d, J=1.4 Hz), 7.80-7.73 (3H, m), 7.55-7.45 (3H, m), 5.87 (1H, dd, J=12.1, 5.2 Hz), 4.07 (1H, dd, J=18.7, 12.1 Hz), 3.33 (1H, dd, J=18.7, 5.2 Hz), 3.18-3.00 (2H, m), 2.54-2.50 (2H, m); ¹³C NMR (101 MHz, DMSO-d₆) δ 173.7 (C), 169.6 (C), 155.1 (C), 148.8 (C), 147.5 (CH), 146.0 (CH), 145.6 (CH), 144.0 (C), 142.1 (C), 141.6 (C), 136.3 (C), 136.2 (C), 134.7 (CH), 129.9 (CH), 129.2 (2×CH), 128.6 (CH), 128.1 (CH), 126.9 (2×CH), 125.4 (CH), 121.2 (CH), 59.8 (CH), 41.9 (CH₂), 28.7 (CH₂), 28.3 (CH₂); HRMS (ESI/Q-TOF) m/z: [M+Na]⁺ calcd for C₂₆H₂₁N₅NaO₃, 474.1537; found, 474.1520.

### 4-Oxo-4-(3-(4-(pyridin-3-yl)phenyl)-5-(quinoxalin-6-yl)-4,5-dihydro-1H-pyrazol-1-yl)butanoic acid (Compound 30)

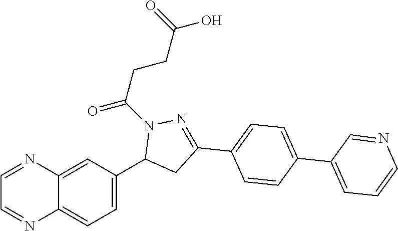 [SMILES: O=C(O)CCC(=O)N1N=C(c2ccc(-c3cccnc3)cc2)CC1c1ccc2nccnc2c1]

To a solution of quinoxaline-6-carbaldehyde (237 mg, 1.50 mmol) and 1-(4-(pyridin-3-yl)phenyl) ethanone (296 mg, 1.50 mmol) in EtOH (12 mL) at room temperature was added dropwise 2 M NaOH (3.8 mL, 7.50 mmol), and the reaction mixture stirred at room temperature for 3 h. After, the solution was cooled down to 0° C., and the resulting precipitated solids were then collected by filtration, washed with water and dried in vacuo to afford (E)-1-(4-(pyridin-3-yl)phenyl)-3-(quinoxalin-6-yl)prop-2-en-1-one as an off-white solid (299 mg), which was used directly in the next step without further purification.

To a stirred solution of (E)-1-(4-(pyridin-3-yl)phenyl)-3-(quinoxalin-6-yl)prop-2-en-1-one (299 mg) in EtOH (7 mL) was added hydrazine hydrate (0.11 mL, 50% w/w), and the reaction mixture heated under reflux for 2 h. The solvent was then removed in vacuo and the resulting residue was poured onto ice water, collected by filtration and dried in vacuo to afford 6-(3-(4-(pyridin-3-yl)phenyl)-4,5-dihydro-1H-pyrazol-5-yl)quinoxaline as an orange wax (199 mg), which was used in the next step without further purification.

In a scaled vial, a mixture of 6-(3-(4-(pyridin-3-yl)phenyl)-4,5-dihydro-1H-pyrazol-5-yl)quinoxaline (199 mg) and succinic anhydride (113 mg, 1.13 mmol) in THF (5 mL) was subjected to microwave irradiation at 120° C. for 45 min. The resulting reaction mixture was concentrated in vacuo. Purification by flash column chromatography (EtOAc, neat→CH₂Cl/MeOH, 9:1) afforded Compound 30 as a yellow solid (68 mg, 10% over 3 steps). ¹H NMR (400 MHz, DMSO-d₆) δ 12.12 (1H, br s), 8.96 (1H, br s), 8.94-8.92 (2H, m), 8.60 (1H, br s), 8.14 (1H, dt, J=8.0, 2.0 Hz), 8.09 (1H, d, J=8.7 Hz), 7.95-7.85 (3H, m), 7.86 (2H, d, J=8.6 Hz), 7.73 (1H, dd, J=8.7, 2.0 Hz), 7.51 (1H, dd, J=7.9, 4.8 Hz), 5.86 (1H, dd, J=11.9, 5.0 Hz), 4.02 (1H, dd, J=18.2, 12.0 Hz), 3.39-3.33 (1H, m), 3.12 (1H, dt, J=17.3, 6.8 Hz), 2.99 (1H, dt, J=17.1, 6.6 Hz), 2.53-2.49 (2H, m); HRMS (ESI/Q-TOF) m/z: [M+Na]⁺ calcd for C₂₆H₂₁N₅NaO₃, 474.1537; found, 474.1554.

### 4-(3-(Naphthalen-2-yl)-5-(quinoxalin-6-yl)-4,5-dihydro-1H-pyrazol-1-yl)-4-oxobutanoic acid (Compound 31)

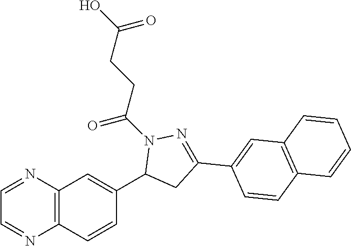 [SMILES: O=C(O)CCC(=O)N1N=C(c2ccc3ccccc3c2)CC1c1ccc2nccnc2c1]

Compound 31 was prepared over 3 steps using a method similar to that described for the preparation of Compound 3-step 1: quinoxaline-6-carbaldehyde (200 mg, 1.26 mmol), 2-acetonaphthone (215 mg, 1.26 mmol) and 2 M NaOH (3.2 mL, 6.32 mmol) in EtOH (12 mL) to afford (E)-1-(naphthalen-2-yl)-3-(quinoxalin-6-yl)prop-2-en-1-one as a colourless solid (300 mg); step 2: (E)-1-(naphthalen-2-yl)-3-(quinoxalin-6-yl)prop-2-en-1-one (250 mg) and hydrazine hydrate (0.10 mL, 50% w/w) in EtOH (10 mL) to afford 6-(3-(naphthalen-2-yl)-4,5-dihydro-1H-pyrazol-5-yl)quinoxaline as a yellow solid (224 mg); and step 3:6-(3-(naphthalen-2-yl)-4,5-dihydro-1H-pyrazol-5-yl)quinoxaline (150 mg) and succinic anhydride (93 mg, 0.93 mmol) in THF (4 mL). Purification by flash column chromatography (EtOAc, neat) afforded Compound 31 as a colourless solid (149 mg, 50%). ¹H NMR (500 MHz, CDCl₃) δ 8.84-8.82 (2H, m), 8.12 (1H, d, J=8.6 Hz), 8.10 (1H, dd, J=8.5, 1.7 Hz), 8.02 (1H, d, J=1.9 Hz), 7.96 (1H, s), 7.90 (1H, d, J=8.7 Hz), 7.87 (1H, d, J=7.9 Hz), 7.84 (1H, d, J=7.6 Hz), 7.71 (1H, dd, J=8.8, 2.0 Hz), 7.55 (1H, td, J=6.9, 1.4 Hz), 7.52 (1H, td, J=6.9, 1.4 Hz), 5.89 (1H, dd, J=11.8, 4.8 Hz), 4.03 (1H, dd, J=17.4, 11.8 Hz), 3.41 (1H, dd, J=17.6, 5.0 Hz), 3.36 (1H, dt, J=17.6, 6.7 Hz), 3.22 (1H, dt, J=17.6, 6.9 Hz), 2.78 (2H, t, J=6.6 Hz); ¹³C NMR (125 MHz, CDCl₃) δ 175.4 (C), 170.6 (C), 154.9 (C), 145.4 (CH), 145.1 (CH), 143.7 (C), 143.1 (C), 142.6 (C), 134.5 (C), 133.1 (C), 130.7 (CH), 128.9 (CH), 128.61 (CH), 128.56 (C), 128.2 (CH), 128.1 (CH), 127.68 (CH), 127.65 (CH), 127.1 (CH), 126.0 (CH), 123.4 (CH), 60.4 (CH), 42.4 (CH₂), 29.2 (CH₂), 29.0 (CH₂).

### 4-(3-(4-Chlorophenyl)-5-(quinoxalin-2-yl)-4,5-dihydro-1H-pyrazol-1-yl)-4-oxobutanoic acid (Compound 32)

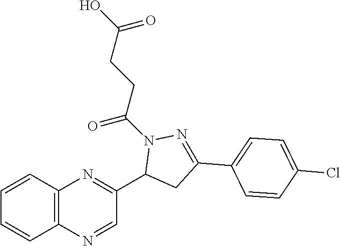 [SMILES: O=C(O)CCC(=O)N1N=C(c2ccc(Cl)cc2)CC1c1cnc2ccccc2n1]

Compound 32 was prepared over 3 steps using a method similar to that described for the preparation of Compound 3-step 1: quinoxaline-2-carbaldehyde (200 mg, 1.26 mmol), 4-chloroacetonophenone (0.16 mL, 1.26 mmol) and 2 M NaOH (3.2 mL, 6.32 mmol) in EtOH (12 mL) to afford (E)-1-(4-chlorophenyl)-3-(quinoxalin-2-yl)prop-2-en-1-one as a colourless solid (300 mg); step 2: (E)-1-(4-chlorophenyl)-3-(quinoxalin-2-yl)prop-2-en-1-one (300 mg) and hydrazine hydrate (0.13 mL, 50% w/w) in EtOH (10 mL) to afford 2-(3-(4-chlorophenyl)-4,5-dihydro-1H-pyrazol-5-yl)quinoxaline as a yellow solid (250 mg); and step 3:2-(3-(4-chlorophenyl)-4,5-dihydro-1H-pyrazol-5-yl)quinoxaline (250 mg) and succinic anhydride (162 mg, 1.62 mmol) in THF (8 mL). Purification by flash column chromatography (EtOAc, neat) afforded Compound 32 as a colourless solid (63 mg, 12%). ¹H NMR (500 MHz, CDCl₃) δ 8.98 (1H, s), 8.11-8.08 (1H, m), 8.02-7.99 (1H, m), 7.75-7.71 (4H, m), 7.42 (2H, d, J=8.7 Hz), 5.91 (1H, dd, J=11.8, 5.9 Hz), 3.80 (1H, dd, J=17.6, 11.7 Hz), 3.68 (1H, dd, J=17.7, 6.0 Hz), 3.18-3.14 (2H, m), 2.73-2.70 (2H, m); ¹³C NMR (125 MHz, CDCl₃) δ 176.5 (C), 170.6 (C), 154.3 (C), 154.1 (C), 144.6 (CH), 142.2 (C), 142.1 (C), 136.8 (C), 130.4 (CH), 130.2 (CH), 129.6 (C), 129.32 (CH), 129.27 (CH), 129.2 (2×CH), 128.2 (2×CH), 60.0 (CH), 39.7 (CH₂), 29.1 (CH₂), 28.8 (CH₂).

### 4-(3-(4-Chlorophenyl)-5-(quinoxalin-5-yl)-4,5-dihydro-1H-pyrazol-1-yl)-4-oxobutanoic acid (Compound 33)

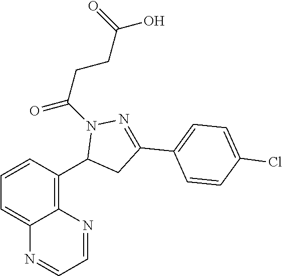 [SMILES: O=C(O)CCC(=O)N1N=C(c2ccc(Cl)cc2)CC1c1cccc2nccnc12]

To a solution of quinoxaline-5-carbaldehyde (237 mg, 1.50 mmol) and 4-chloroacetophenone (0.19 mL, 1.50 mmol) in EtOH (12 mL) at room temperature was added dropwise 2 M NaOH (3.8 mL, 7.50 mmol), and the reaction mixture stirred at room temperature for 2 h. After, the solution was cooled down to 0° C., and the resulting precipitated solids were then collected by filtration, washed with water and dried in vacuo to afford (E)-1-(4-chlorophenyl)-3-(quinoxalin-5-yl)prop-2-en-1-one as a yellow solid (424 mg), which was used directly in the next step without further purification.

To a stirred solution of (E)-1-(4-chlorophenyl)-3-(quinoxalin-5-yl)prop-2-en-1-one (200 mg) in EtOH (5 mL) was added hydrazine hydrate (85 μL, 50% w/w), and the reaction mixture heated under reflux for 2 h. The solvent was then removed in vacuo and the resulting residue was poured onto ice water, collected by filtration and dried in vacuo to afford 5-(3-(4-chlorophenyl)-4,5-dihydro-1H-pyrazol-5-yl)quinoxaline as an orange solid (210 mg), which was used in the next step without further purification.

In a sealed vial, a mixture of 5-(3-(4-chlorophenyl)-4,5-dihydro-1H-pyrazol-5-yl)quinoxaline (210 mg) and succinic anhydride (136 mg, 1.36 mmol) in THF (5 mL) was subjected to microwave irradiation at 120° C. for 45 min. The resulting mixture was diluted in EtOAc (50 mL) and washed with water (2×30 mL), then brine (30 mL), dried over anhydrous Na₂SO₄, filtered, and concentrated in vacuo. (CH₂Cl₂, neat→CH₂Cl₂/MeOH, 95:5) afforded Compound 33 as a brown solid (46 mg, 16% over 3 steps). ¹H NMR (400 MHz, DMSO-d₆) δ 12.20 (1H, br s), 9.03-9.01 (2H, m), 8.02 (1H, dd, J=8.3, 1.3 Hz), 7.80-7.75 (3H, m), 7.52-7.48 (3H, m), 6.52 (1H, dd, J=12.0, 4.9 Hz), 4.03 (1H, dd, J=18.1, 12.1 Hz), 3.22-3.14 (2H, m), 2.96 (1H, dt, J=17.0, 6.5 Hz), 2.55-2.50 (2H, m); HRMS (ESI/Q-TOF) m/z: [M+Na]⁺ calcd for C₂₁H₁₇ClN₄NaO₃, 431.0881; found, 431.0887.

### 4-(3-(4-Chlorophenyl)-5-(quinolin-7-yl)-4,5-dihydro-1H-pyrazol-1-yl)-4-oxobutanoic acid (Compound 34)

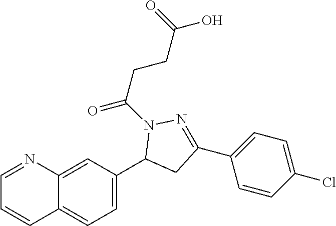 [SMILES: O=C(O)CCC(=O)N1N=C(c2ccc(Cl)cc2)CC1c1ccc2cccnc2c1]

Compound 34 was prepared over 3 steps using a method similar to that described for the preparation of Compound 3-step 1: quinoline-7-carbaldehyde (200 mg, 1.27 mmol), 4-chloroacetonophenone (0.16 mL, 1.27 mmol) and 2 M NaOH (3.2 mL, 6.36 mmol) in EtOH (12 mL) to afford (E)-1-(4-chlorophenyl)-3-(quinolin-7-yl)prop-2-en-1-one as an off-white solid (374 mg); step 2: (E)-1-(4-chlorophenyl)-3-(quinolin-7-yl)prop-2-en-1-one (374 mg) and hydrazine hydrate (0.16 mL, 50% w/w) in EtOH (13 mL) to afford 7-(3-(4-chlorophenyl)-4,5-dihydro-1H-pyrazol-5-yl) quinoline as an off-white solid (392 mg); and step 3:7-(3-(4-chlorophenyl)-4,5-dihydro-1H-pyrazol-5-yl) quinoline (392 mg) and succinic anhydride (255 mg, 2.55 mmol) in THF (13 mL). Purification by flash column chromatography (EtOAc, neat→EtOAc/MeOH, 9:1) afforded Compound 34 as an off-white solid (229 mg, 44% over 3 steps). ¹H NMR (400 MHz, DMSO-d₆) δ 12.09 (1H, br s), 8.88 (1H, dd, J=4.2, 1.8 Hz), 8.33 (1H, d, J=8.3 Hz), 7.95 (1H, d, J=8.5 Hz), 7.84-7.81 (3H, m), 7.56-7.49 (3H, m), 7.45 (1H, dd. J=8.5, 1.8 Hz), 5.79 (1H, dd, J=11.9, 5.0 Hz), 3.96 (1H, dd, J=18.2, 12.0 Hz), 3.27 (1H, dd, J=18.2, 5.0 Hz), 3.11-2.94 (2H, m), 2.55-2.46 (2H, m); ¹³C NMR (101 MHz, DMSO-d₆) δ 173.7 (C), 169.2 (C), 153.4 (C), 150.8 (CH), 147.6 (C), 143.4 (C), 135.7 (CH), 134.9 (C), 129.9 (C), 128.9 (2×CH), 128.8 (CH), 128.5 (2×CH), 127.1 (C), 125.1 (CH), 124.4 (CH), 121.4 (CH), 59.9 (CH), 41.8 (CH₂), 28.8 (CH₂) 28.4 (CH₂); HRMS (ESI/Q-TOF) m/z: [M+H]⁺ calcd for C₂₂H₁₉ClN₃O₃, 408.1109; found, 408.1105.

### 4-(5-(Benzo[c][1,2,5]oxadiazol-5-yl)-3-(4-chlorophenyl)-4,5-dihydro-1H-pyrazol-1-yl)-4-oxobutanoic acid (Compound 35)

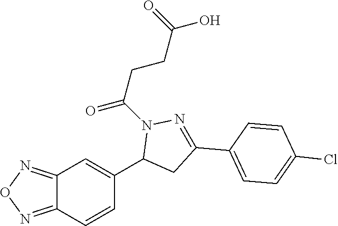 [SMILES: O=C(O)CCC(=O)N1N=C(c2ccc(Cl)cc2)CC1c1ccc2nonc2c1]

To a solution of 2,1,3-benzoxadiazole-5-carbaldehyde (222 mg, 1.50 mmol) and 4-chloroacetonophenone (0.19 mL, 1.50 mmol) in EtOH (12 mL) at room temperature was added dropwise 2 M NaOH (3.8 mL, 7.5 mmol), and the reaction mixture was stirred for 2 h. After, the solution was cooled down to 0° C., and the resulting precipitated solids were then collected by filtration, washed with water and dried in vacuo to afford (E)-3-(benzo[c][1,2,5]oxadiazol-5-yl)-1-(4-chlorophenyl)prop-2-en-1-one as a brown solid (256 mg), which was used directly in the next step without further purification.

A mixture of (E)-3-(benzo[c][1,2,5]oxadiazol-5-yl)-1-(4-chlorophenyl)prop-2-en-1-one (256 mg) and hydrazine hydrate (0.17 mL, 50% w/w) in THF (7 mL) was stirred under reflux for 5 h. After, succinic anhydride (270 mg, 2.70 mmol) was added, and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. Purification by flash column chromatography (petroleum ether/EtOAc, 1:1→EtOAc, neat→EtOAc/MeOH, 9:1) afforded Compound 35 as a brown solid (24 mg, 4% over 2 steps). ¹H NMR (400 MHz, CDCl₃) δ 7.81 (1H, d, J=9.4 Hz), 7.69-7.67 (3H, m), 7.41 (2H, d, J=8.6 Hz), 7.23 (1H, dd, J=9.3, 1.5 Hz), 5.67 (1H, dd, J=12.0, 5.2 Hz), 3.83 (1H, dd, J=17.8, 12.1 Hz), 3.31-3.01 (3H, m), 2.79-2.66 (2H, m); ¹³C NMR (101 MHz, CDCl₃) δ 177.3 (C), 170.4 (C), 153.5 (C), 149.2 (C), 148.9 (C), 144.4 (C), 137.1 (C), 130.1 (CH), 129.3 (2×CH), 129.2 (C), 128.1 (2×CH), 118.1 (CH), 112.8 (CH), 60.3 (CH), 41.3 (CH₂), 29.0 (CH₂) 28.6 (CH₂); HRMS (ESI/Q-TOF) m/z: [M+Na]⁺ calcd for C₁₉H₁₅ClN₄NaO₄, 421.0674; found, 421.0670.

### 4-(5-(Benzo[c][1,2,5]thiadiazol-5-yl)-3-(4-chlorophenyl)-4,5-dihydro-1H-pyrazol-1-yl)-4-oxobutanoic acid (Compound 36)

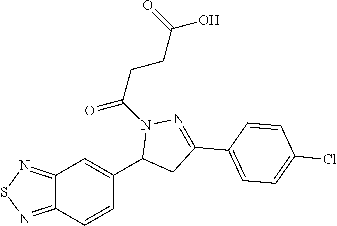 [SMILES: O=C(O)CCC(=O)N1N=C(c2ccc(Cl)cc2)CC1c1ccc2nsnc2c1]

To a solution of 2,1,3-benzothiadiazole-5-carbaldehyde (246 mg, 1.50 mmol) and 4-chloroacetonophenone (0.19 mL, 1.50 mmol) in EtOH (12 mL) at room temperature was added dropwise 2 M NaOH (3.8 mL, 7.5 mmol), and the reaction mixture was stirred for 24 h. After, the solution was cooled down to 0° C., and the resulting precipitated solids were then collected by filtration, washed with water and dried in vacuo to afford (E)-3-(benzo[c][1,2,5]thiadiazol-5-yl)-1-(4-chlorophenyl)prop-2-en-1-one as an off-white solid (248 mg), which was used directly in the next step without further purification.

A mixture of (E)-3-(benzo[c][1,2,5]thiadiazol-5-yl)-1-(4-chlorophenyl)prop-2-en-1-one (248 mg) and hydrazine hydrate (0.10 mL, 50% w/w) in THF (7 mL) was stirred under reflux for 5 h. After, succinic anhydride (248 mg, 2.48 mmol) was added, and the reaction mixture was further stirred under reflux for 1 h. The reaction mixture was then concentrated in vacuo, and purified flash column chromatography (petroleum ether/EtOAc, 1:1→EtOAc, neat). The resulting solid was solubilized in EtOAc (30 mL) and extracted with sat. aq. NaHCO₃ (3× 15 mL). The combined aqueous layers were then washed with EtOAc (15 mL) and acidified to aprox. pH 3 through the addition of 6 M aq. HCl. The aqueous layer was then extracted with EtOAc (3×15 mL), and the combined organic layers were washed with brine (15 mL), dried over anhydrous Na₂SO₄, filtered, and concentrated in vacuo to give Compound 36 as an off-white solid (56 mg, 9% over 2 steps). ¹H NMR (400 MHz, CDCl₃) δ 7.97 (1H, d, J=8.9 Hz), 7.86 (1H, s), 7.69 (2H, d, J=8.7 Hz), 7.44 (1H, dd, J=9.1, 1.8 Hz), 7.41 (2H, d, J=8.7 Hz), 5.76 (1H, dd, J=11.9, 5.0 Hz), 3.84 (1H, dd, J=17.8, 12.0 Hz), 3.27-3.09 (3H, m), 2.73 (2H, t, J=6.7 Hz); ¹³C NMR (101 MHz, CDCl₃) δ 177.4 (C), 170.2 (C), 155.0 (C), 154.5 (C), 153.5 (C), 142.8 (C), 137.0 (C), 129.5 (CH), 129.3 (2×CH), 128.1 (2×CH), 127.7 (C), 122.7 (CH), 118.1 (CH), 60.4 (CH), 42.0 (CH₂), 29.1 (CH₂) 28.7 (CH₂); HRMS (ESI/Q-TOF) m/z: [M+Na]⁺ calcd for C₁₉H₁₅ClN₄NaO₃S, 437.0446; found, 437.0436.

### 4-(5-(benzo[d][1,3]dioxol-5-yl)-3-(4-chlorophenyl)-4,5-dihydro-1H-pyrazol-1-yl)-4-oxobutanoic acid (Compound 37)

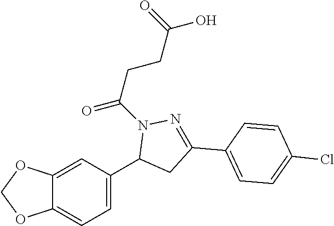 [SMILES: O=C(O)CCC(=O)N1N=C(c2ccc(Cl)cc2)CC1c1ccc2c(c1)OCO2]

To a solution of piperonal (200 mg, 1.33 mmol) and 4-chloroacetophenone (0.17 mL, 1.33 mmol) in EtOH (11 mL) at room temperature was added dropwise 2 M NaOH (3.3 mL, 6.6 mmol), and the reaction mixture was stirred for 2 h. After, the solution was cooled down to 0° C., and the resulting precipitated solids were then collected by filtration, washed with water and dried in vacuo to afford (E)-3-(benzo[d][1,3]dioxol-5-yl)-1-(4-chlorophenyl)prop-2-en-1-one as a colourless solid (305 mg), which was used directly in the next step without further purification.

To a stirred solution of (E)-3-(benzo[d][1,3]dioxol-5-yl)-1-(4-chlorophenyl)prop-2-en-1-one (305 mg) in EtOH (8 mL) was added hydrazine hydrate (0.20 mL, 50% w/w), and the reaction mixture heated under reflux for 2 h. The solvent was then removed in vacuo and the resulting residue was diluted in EtOAc (50 mL) and washed with water (2×30 mL), then brine (30 mL), dried over anhydrous Na₂SO₄, filtered, and concentrated in vacuo to afford 5-(benzo[d][1,3]dioxol-5-yl)-3-(4-chlorophenyl)-4,5-dihydro-1H-pyrazole as an off-white solid (314 mg), which was used in the next step without further purification.

In a sealed vial, a mixture of 5-(benzo[d][1,3]dioxol-5-yl)-3-(4-chlorophenyl)-4,5-dihydro-1H-pyrazole (157 mg) and succinic anhydride (104 mg, 1.04 mmol) in THF (4 mL) was subjected to microwave irradiation at 120° C. for 45 min. The reaction mixture was concentrated in vacuo and purified by flash column chromatography (petroleum ether/EtOAc, 1:1→petroleum ether/EtOAc, 7:3). The resulting solid was solubilized in CHCl₃ (30 mL) and washed with water (2×15 mL), then brine (15 mL), dried over anhydrous Na₂SO₄, filtered, and concentrated in vacuo to afford Compound 37 as an off-white solid (126 mg, 47% over 3 steps). ¹H NMR (400 MHz, DMSO-d₆) δ 12.12 (1H, s), 7.80 (2H, d, J=8.7 Hz), 7.53 (2H, d, J=8.7 Hz), 6.83 (1H, d, J=8.0 Hz), 6.73 (1H, d, J=1.7 Hz), 6.68 (1H, dd, J=8.1, 1.7 Hz), 6.02-5.93 (2H, m), 5.47 (1H, dd, J=11.8, 4.7 Hz), 3.81 (1H, dd, J=18.1, 11.8 Hz), 3.12 (1H, dd, J=18.1, 4.7 Hz), 3.06-2.84 (2H, m), 2.50-2.45 (2H, m); HRMS (ESI/Q-TOF) m/z: [M+Na]⁺ calcd for C₂₀H₁₇ClN₂NaO₅, 423.0718; found, 423.0707.

### 4-(3-(4-Chlorophenyl)-5-(2,2-difluorobenzo[d][1,3]dioxol-5-yl)-4,5-dihydro-1H-pyrazol-1-yl)-4-oxobutanoic acid (Compound 38)

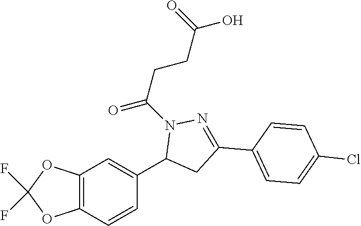 [SMILES: O=C(O)CCC(=O)N1N=C(c2ccc(Cl)cc2)CC1c1ccc2c(c1)OC(F)(F)O2]

To a solution of 2,2-difluoro-1,3-benzodioxole-5-carboxaldehyde (372 mg, 2.00 mmol), 4-chloroacetophenone (0.26 mL, 2.00 mmol) in EtOH (16 mL) at room temperature was added dropwise 2 M NaOH (5.0 mL, 10.0 mmol), and the reaction mixture was stirred for 3 h. After, the solution was cooled down to 0° C., and the resulting precipitated solids were then collected by filtration, washed with water and dried in vacuo to afford (E)-1-(4-chlorophenyl)-3-(2,2-difluorobenzo[d][1,3]dioxol-5-yl)prop-2-en-1-one as a colourless solid (432 mg), which was used directly in the next step without further purification.

A mixture of (E)-1-(4-chlorophenyl)-3-(2,2-difluorobenzo[d][1,3]dioxol-5-yl)prop-2-en-1-one (432 mg) and hydrazine hydrate (0.17 mL, 50% w/w) in THF (11 mL) was stirred under reflux for 2 h. After, succinic anhydride (402 mg, 4.02 mmol) was added, and the reaction mixture was further stirred under reflux for 1 h. The reaction mixture was concentrated in vacuo and purified by flash column chromatography (petroleum ether/EtOAc, 1:1→EtOAc, neat). The resulting solid was solubilized in CHCl₃ (50 mL) and washed with water (2×30 mL), then brine (30 mL), dried over anhydrous Na₂SO₄, filtered, and concentrated in vacuo to afford Compound 38 as a colourless solid (402 mg, 46% over 2 steps). ¹H NMR (400 MHz, CDCl₃) δ 7.67 (2H, d, J=8.7 Hz), 7.41 (2H, d, J=8.7 Hz), 7.00-6.92 (3H, m), 5.56 (1H, dd, J=11.9, 4.9 Hz), 3.75 (1H, dd, J=17.8, 11.9 Hz), 3.24-3.03 (3H, m), 2.73 (2H, t, J=6.7 Hz); HRMS (ESI/Q-TOF) m/z: [M+Na]⁺ calcd for C₂₀H₁₅ClF₂N₂NaO₅, 459.0530; found, 459.0528.

### 4-(3-(4-Chlorophenyl)-5-(2,3-dihydrobenzo[b][1,4]dioxin-6-yl)-4,5-dihydro-1H-pyrazol-1-yl)-4-oxobutanoic acid (Compound 39)

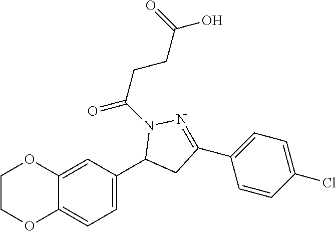 [SMILES: O=C(O)CCC(=O)N1N=C(c2ccc(Cl)cc2)CC1c1ccc2c(c1)OCCO2]

To a solution of 1,4-benzodioxan-6-carboxaldehyde (400 mg, 2.44 mmol) and 4-chloroacetophenone (0.32 mL, 2.44 mmol) in EtOH (24 mL) at 0° C. was added dropwise 2 M NaOH (6.1 mL, 12.2 mmol), and the reaction mixture stirred for 24 h (while gradually warming to room temperature). The resulting precipitated solids were then collected by filtration, washed with water and dried in vacuo to afford (E)-1-(4-chlorophenyl)-3-(2,3-dihydrobenzo[b][1,4]dioxin-6-yl)prop-2-en-1-one as a yellow solid (616 mg), which was used directly in the next step without further purification.

To a stirred solution of (E)-1-(4-chlorophenyl)-3-(2,3-dihydrobenzo[b][1,4]dioxin-6-yl)prop-2-en-1-one (350 mg) in EtOH (12 mL) was added hydrazine hydrate (0.15 mL, 50% w/w), and the reaction mixture heated under reflux for 3 h. The solvent was then removed in vacuo and the resulting residue was poured onto ice water, collected by filtration and dried in vacuo to afford 3-(4-chlorophenyl)-5-(2,3-dihydrobenzo[b][1.4]dioxin-6-yl)-4,5-dihydro-1H-pyrazole as a yellow solid (256 mg), which was used in the next step without further purification.

In a sealed vial, a mixture of 3-(4-chlorophenyl)-5-(2,3-dihydrobenzo[b][1.4]dioxin-6-yl)-4,5-dihydro-1H-pyrazole (200 mg) and succinic anhydride (127 mg, 1.27 mmol) in THF (5 mL) was subjected to microwave irradiation at 120° C. for 45 min. The reaction mixture was concentrated in vacuo and purified by flash column chromatography (petroleum ether/EtOAc, 1:1→EtOAc, neat). The resulting solid was solubilized in CHCl₃ (30 mL) and washed with water (2×15 mL), then brine (15 mL), dried over anhydrous Na₂SO₄, filtered, and concentrated in vacuo to afford Compound 39 as a yellow solid (161 mg, 36% over 3 steps). ¹H NMR (400 MHz. DMSO-d₆) δ 12.10 (1H, br s), 7.79 (2H, d, J=8.6 Hz), 7.53 (2H, d, J=8.7 Hz), 6.77 (1H, d, J=8.3 Hz), 6.65-6.62 (2H, m), 5.43 (1H, dd, J=11.7, 4.5 Hz), 4.19 (4H, s), 3.78 (1H, dd, J=18.1, 11.8 Hz), 3.10 (1H, dd, J=18.1, 4.6 Hz), 3.02-2.87 (2H, m), 2.50-2.46 (2H, m); ¹³C NMR (101 MHz, DMSO-d₆) δ 173.8 (C), 169.0 (C), 153.3 (C), 143.2 (C), 142.5 (C), 135.4 (C), 134.9 (C), 130.1 (C), 128.9 (2×CH), 128.4 (2×CH), 118.2 (CH), 117.2 (CH), 114.2 (CH), 64.1 (CH₂), 64.0 (CH₂), 59.2 (CH), 41.8 (CH₂), 28.8 (CH₂) 28.4 (CH₂); HRMS (ESI/Q-TOF) m/z: [M+Na]⁺ calcd for C₂₁H₁₉ClN₂NaO₅, 437.0875; found, 437.0866.

### 4-(3-([1,1′-Biphenyl]-4-yl)-5-(2,3-dihydrobenzo[b][1,4]dioxin-6-yl)-4,5-dihydro-1H-pyrazol-1-yl)-4-oxobutanoic acid (Compound 40)

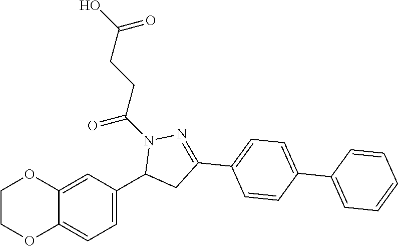 [SMILES: O=C(O)CCC(=O)N1N=C(c2ccc(-c3ccccc3)cc2)CC1c1ccc2c(c1)OCCO2]

To a solution of 1,4-benzodioxan-6-carbaldehyde (492 mg, 3.00 mmol) and 4-acetylbiphenyl (589 mg, 3.00 mmol) in EtOH (10 mL) at 0° C. was added dropwise 2 M NaOH (7.5 mL, 15.0 mmol), and the reaction mixture stirred for 5 h (while gradually warming to room temperature). The resulting precipitated solids were then collected by filtration, washed with water and dried in vacuo to afford (E)-1-([1,1′-biphenyl]-4-yl)-3-(2,3-dihydrobenzo[b][1,4]dioxin-6-yl)prop-2-en-1-one as a yellow solid (1.03 g), which was used directly in the next step without further purification.

To a stirred solution of (E)-1-([1,1′-biphenyl]-4-yl)-3-(2,3-dihydrobenzo[b][1,4]dioxin-6-yl)prop-2-en-1-one (500 mg) in EtOH (8 mL) was added hydrazine hydrate (0.45 mL, 50% w/w), and the reaction mixture heated under reflux for 2 h. The solvent was then removed in vacuo, the residue poured onto ice water and the resulting precipitated solids were collected by filtration and dried in vaCuo 9 afford 3-([1,1′-biphenyl]-4-yl)-5-(2,3-dihydrobenzo[b][1,4]dioxin-6-yl)-4,5-dihydro-1H-pyrazole as a yellow solid (350 mg), which was used in the next step without further purification.

In a sealed vial, a mixture of 3-([1,1′-biphenyl]-4-yl)-5-(2,3-dihydrobenzo[b][1,4]dioxin-6-yl)-4,5-dihydro-1H-pyrazole (350 mg) and succinic anhydride (196 mg, 1.96 mmol) in THF (10 mL) was subjected to microwave irradiation at 120° C. for 45 min. The resulting reaction mixture was diluted with EtOAc (20 mL), washed with water (20 mL) and the separated aqueous layer further extracted with EtOAc (3×30 mL). The combined organic layers were washed with water (3×30 mL), then brine (30 mL), dried over anhydrous Na₂SO₄, and concentrated in vacuo. Purification by flash column chromatography (CH₂Cl₂/MeOH, 50:1) afforded Compound 40 as a yellow solid (153 mg, 23% over 3 steps). ¹H NMR (400 MHz, CDCl₃) δ 7.80 (2H, d, J=8.4 Hz), 7.66-7.62 (4H, m), 7.47 (2H, t, J=7.5 Hz), 7.41-7.36 (1H, m), 6.82-6.80 (1H, m), 6.74-6.71 (2H, m), 5.50 (1H, dd, J=11.6, 4.5 Hz), 4.20 (4H, s), 3.73 (1H, dd, J=17.6, 11.7 Hz), 3.20-3.13 (3H, m), 2.81-2.70 (2H, m); ¹³C NMR (101 MHz, CDCl₃) δ 177.6 (C), 170.0 (C), 154.6 (C), 143.8 (C), 143.3 (C), 143.2 (C), 140.2 (C), 135.1 (C), 130.2 (C), 129.0 (2×CH), 128.0 (CH), 127.4 (2×CH), 127.3 (2×CH), 127.2 (2×CH), 118.7 (CH), 117.8 (CH), 114.5 (CH), 64.4 (2×CH₂), 59.9 (CH), 42.4 (CH₂), 29.2 (CH₂), 29.1 (CH₂); HRMS (ESI/Q-TOF) m/z: [M+H]⁺ calcd for C₂₇H₂₅N₂O₅, 457.1758; found, 457.1770.

### 4-(3-(4-Chlorophenyl)-5-(3-methoxyphenyl)-4,5-dihydro-1H-pyrazol-1-yl)-4-oxobutanoic acid (Compound 41)

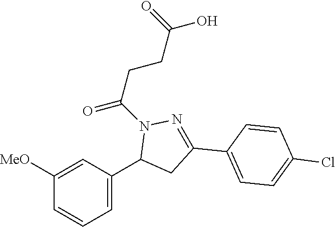 [SMILES: COc1cccc(C2CC(c3ccc(Cl)cc3)=NN2C(=O)CCC(=O)O)c1]

To a solution of 3-methoxybenzaldehyde (200 mg, 1.47 mmol) and 4-chloroacetophenone (0.19 mL, 1.47 mmol) in EtOH (12 mL) at room temperature was added dropwise 2 M NaOH (3.7 mL, 7.4 mmol), and the reaction mixture was stirred for 3 h. After, the solution was cooled down to 0° C., and the resulting precipitated solids were then collected by filtration, washed with water and dried in vacuo to afford (E)-1-(4-chlorophenyl)-3-(3-methoxyphenyl)prop-2-en-1-one as a colourless solid (345 mg), which was used directly in the next step without further purification.

To a stirred solution of (E)-1-(4-chlorophenyl)-3-(3-methoxyphenyl)prop-2-en-1-one (345 mg) in EtOH (10 mL) was added hydrazine hydrate (0.24 mL, 50% w/w), and the reaction mixture heated under reflux for 2 h. The solvent was then removed in vacuo and the resulting residue was diluted in EtOAc (50 mL) and washed with water (2×30 mL), then brine (30 mL), dried over anhydrous Na₂SO₄, filtered, and concentrated in vacuo to afford 3-(4-chlorophenyl)-5-(3-methoxyphenyl)-4,5-dihydro-1H-pyrazole as a yellow solid (363 mg), which was used in the next step without further purification.

In a sealed vial, a mixture of 3-(4-chlorophenyl)-5-(3-methoxyphenyl)-4,5-dihydro-1H-pyrazole (363 mg) and succinic anhydride (253 mg, 2.53 mmol) in THF (10 mL) was subjected to microwave irradiation at 120° C. for 45 min. The resulting reaction mixture was concentrated in vacuo, diluted in EtOAc (30 mL) and extracted with sat. aq. NaHCO₃ (3×30 mL). The combined aqueous layers were washed with EtOAc (30 mL) and acidified to aprox. pH 3 through the addition of 6 M aq. HCl. The aqueous layer was then extracted with EtOAc (3×30 mL), and the combined organic layers were washed with brine (30 mL), dried over anhydrous Na₂SO₄, filtered, and concentrated in vacuo. Purification by flash column chromatography (petroleum ether/EtOAc, 1:1→EtOAc, neat) afforded Compound 41 as a colourless solid (34 mg, 6% over 3 steps). ¹H NMR (400 MHz, CDCl₃) δ 7.67 (2H, d, J=8.7 Hz), 7.40 (2H, d, J=8.7 Hz), 7.25-7.20 (1H, m), 6.79 (2H, dd, J=8.0, 2.1 Hz), 6.73 (1H, t, J=2.1 Hz), 5.55 (1H, dd, J=11.8, 4.7 Hz), 3.77 (3H, s), 3.73 (1H, dd, J=17.7, 11.8 Hz), 3.23-3.09 (3H, m), 2.79-2.66 (2H, m); HRMS (ESI/Q-TOF) m/z: [M+H]⁺ calcd for C₂₀H₂₀ClN₂O₄, 387.1106; found, 387.1103.

### 4-(3-(4-Chlorophenyl)-5-(2,4-dimethoxyphenyl)-4,5-dihydro-1H-pyrazol-1-yl)-4-oxobutanoic acid (Compound 42)

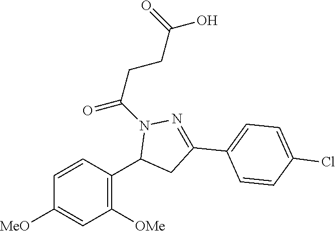 [SMILES: COc1ccc(C2CC(c3ccc(Cl)cc3)=NN2C(=O)CCC(=O)O)c(OC)c1]

To a solution of 2,4-dimethoxybenzaldehyde (299 mg, 1.80 mmol) and 4-chloroacetophenone (0.23 mL, 1.80 mmol) in ⁱPrOH (14 mL) at room temperature was added dropwise 2 M NaOH (2.7 mL, 5.40 mmol), and the reaction mixture was stirred for 6 h. Next, hydrazine hydrate (0.34 mL, 50% w/w) was added and reaction mixture was stirred under reflux for 24 h. After, succinic anhydride (540 mg, 5.40 mmol) was added, and the reaction mixture was further stirred under reflux for 1 h. The excess of ⁱPrOH was removed in vacuo and the residue was then diluted in EtOAc (50 mL) and washed with water (2×30 mL), then brine (30 mL), dried over anhydrous Na₂SO₄, filtered, and concentrated in vacuo. Purification by flash column chromatography (petroleum ether/EtOAc, 1:1→EtOAc, neat) afforded Compound 42 as a yellow solid (158 mg, 21%). ¹H NMR (400 MHz, CDCl₃) δ 7.66 (2H, d, J=8.7 Hz), 7.38 (2H, d, J=8.7 Hz), 6.95 (1H, d, J=8.4 Hz), 6.45 (1H, d, J=2.3 Hz), 6.41 (1H, dd, J=8.4, 2.4 Hz), 5.74 (1H, dd, J=11.7, 4.7 Hz), 3.77 (3H, s), 3.76 (3H, s), 3.64 (1H, dd, J=17.6, 11.7 Hz), 3.23 (1H, dt, J=17.4, 6.7 Hz), 3.12 (1H, dt, J=17.4, 6.7 Hz), 3.03 (1H, dd, J=17.6, 4.7 Hz), 2.74 (2H, t, J=6.7 Hz); HRMS (ESI/Q-TOF) m/z: [M+H]⁺ calcd for C₂₁H₂₂ClN₂O₅, 417.1212; found, 417.1200.

### 4-(3-(4-Chlorophenyl)-5-(3,4-dimethoxyphenyl)-4,5-dihydro-1H-pyrazol-1-yl)-4-oxobutanoic acid (Compound 43)

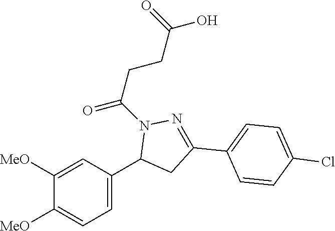 [SMILES: COc1ccc(C2CC(c3ccc(Cl)cc3)=NN2C(=O)CCC(=O)O)cc1OC]

To a solution of 3,4-dimethoxybenzaldehyde (100 mg, 0.60 mmol) and 4-chloroacetophenone (78 μL, 0.60 mmol) in ⁱPrOH (5 mL) at room temperature was added dropwise 2 M NaOH (1.5 mL, 3.00 mmol), and the solution was stirred for 22 h. Next, hydrazine hydrate (0.15 mL, 50% w/w) and succinic anhydride (240 mg, 2.40 mmol) was added, and reaction mixture was stirred under reflux for 5 h. A second portion of hydrazine hydrate (0.15 mL, 50% w/w) and succinic anhydride (240 mg, 2.40 mmol) was added, and the mixture was further stirred under reflux for 3 h. The excess of ⁱPrOH was removed in vacuo and the residue was then diluted in EtOAc (30 mL) and extracted with sat. aq. NaHCO₃ (3×30 mL). The combined aqueous layers were washed with EtOAc (30 mL) and acidified to aprox. pH 3 through the addition of 6 M aq. HCl. The aqueous layer was then extracted with EtOAc (3×30 mL), and the combined organic layers were washed with brine (30 mL), dried over anhydrous Na₂SO₄, filtered, and concentrated in vacuo. Purification by flash column chromatography (petroleum ether/EtOAc, 1:1→EtOAc, neat) afforded Compound 43 as an off-white solid (43 mg, 17%). ¹H NMR (400 MHz, CDCl₃) δ 7.68 (2H, d, J=8.7 Hz), 7.40 (2H, d, J=8.7 Hz), 6.80 (1H, d, J=8.2 Hz), 6.77-6.72 (2H, m), 5.53 (1H, dd, J=11.7, 4.8 Hz), 3.84 (3H, s), 3.83 (3H, s), 3.72 (1H, dd, J=17.7, 11.8 Hz), 3.22-3.08 (3H, m), 2.73 (2H, t, J=7.0 Hz); HRMS (ESI/Q-TOF) m/z: [M+H]⁺ calcd for C₂₁H₂₂ClN₂O₅, 417.1212; found, 417.1204.

### 4-(3-(4-Chlorophenyl)-5-(3-hydroxy-4-methoxyphenyl)-4,5-dihydro-1H-pyrazol-1-yl)-4-oxobutanoic acid (Compound 44)

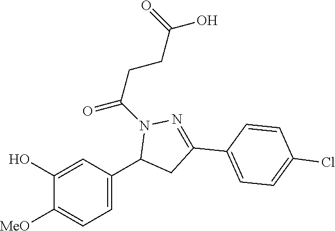 [SMILES: COc1ccc(C2CC(c3ccc(Cl)cc3)=NN2C(=O)CCC(=O)O)cc1O]

To a solution of isovanillin (304 mg, 2.00 mmol) and 4-chloroacetophenone (0.26 mL, 2.00 mmol) in EtOH (16 mL) at room temperature was added dropwise 2 M NaOH (5.0 mL, 10.0 mmol), and the reaction mixture was stirred for 24 h. After, the excess of EtOH was removed in vacuo and water (10 mL) was added, the solution neutralized through the addition of 6 M aq. HCl, and the resulting precipitated solids were then collected by filtration, washed with water and dried in vacuo to afford (E)-1-(4-chlorophenyl)-3-(3-hydroxy-4-methoxyphenyl)prop-2-en-1-one as a yellow solid (485 mg), which was used directly in the next step without further purification.

To a stirred solution of (E)-1-(4-chlorophenyl)-3-(3-hydroxy-4-methoxyphenyl)prop-2-en-1-one (485 mg) in EtOH (13 mL) was added hydrazine hydrate (0.31 mL, 50% w/w), and the reaction mixture heated under reflux for 2 h. The solvent was then removed in vacuo and the resulting residue was diluted in EtOAc (50 mL) and washed with water (2×30 mL), then brine (30 mL), dried over anhydrous Na₂SO₄, filtered, and concentrated in vacuo to afford 5-(3-(4-chlorophenyl)-4,5-dihydro-1H-pyrazol-5-yl)-2-methoxyphenol as a brown solid (478 mg), which was used in the next step without further purification.

In a sealed vial, a mixture of 5-(3-(4-chlorophenyl)-4,5-dihydro-1H-pyrazol-5-yl)-2-methoxyphenol (478 mg) and succinic anhydride (158 mg, 1.58 mmol) in THF (12 mL) was subjected to microwave irradiation at 120° C. for 45 min. The resulting mixture was diluted in EtOAc (50 mL) and washed with water (2×30 mL), then brine (30 mL), dried over anhydrous Na₂SO₄, filtered, and concentrated in vacuo. Purification by flash column chromatography (petroleum ether/EtOAc, 1:1→EtOAc, neat) afforded Compound 44 as a yellow solid (226 mg, 28% over 3 steps). ¹H NMR (400 MHz, CDCl₃) δ 7.66 (2H, d, J=8.6 Hz), 7.39 (2H, d, J=8.6 Hz), 6.79-6.73 (3H, m), 5.50 (1H, dd, J=11.7, 4.6 Hz), 3.84 (3H, s), 3.69 (1H, dd, J=17.7, 11.7 Hz), 3.19-3.08 (3H, m), 2.78-2.64 (2H, m); HRMS (ESI/Q-TOF) m/z: [M+H]⁺ calcd for C₂₀H₂₀ClN₂O₅, 403.1055; found, 403.1049.

### 4-(3-(4-Chlorophenyl)-5-(4-hydroxy-3-methoxyphenyl)-4,5-dihydro-1H-pyrazol-1-yl)-4-oxobutanoic acid (Compound 45)

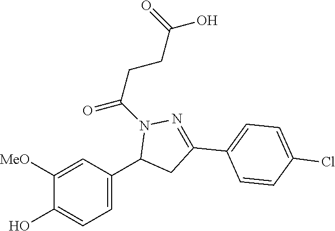 [SMILES: COc1cc(C2CC(c3ccc(Cl)cc3)=NN2C(=O)CCC(=O)O)ccc1O]

To a solution of vanillin (304 mg, 2.00 mmol) and 4-chloroacetophenone (0.26 mL, 2.00 mmol) in EtOH (2 mL) at 0° C. was added dropwise SOCl₂ (0.10 mL, 1.40 mmol), and the reaction mixture stirred at room temperature for 2 h. Next, the mixture was quenched with water (30 mL), neutralized with sat. aq. NaHCO₃, and extracted with CH₂Cl₂ (3× 15 mL). The combined organic layers were then dried over anhydrous Na₂SO₄, filtered, and concentrated in vacuo to afford (E)-1-(4-chlorophenyl)-3-(4-hydroxy-3-methoxyphenyl)prop-2-en-1-one as a dark oil (577 mg), which was used directly in the next step without further purification.

To a stirred solution of (E)-1-(4-chlorophenyl)-3-(4-hydroxy-3-methoxyphenyl)prop-2-en-1-one (577 mg) in EtOH (16 mL) was added hydrazine hydrate (0.37 mL, 50% w/w), and the reaction mixture heated under reflux for 4 h. The solvent was then removed in vacuo and the resulting residue was diluted in EtOAc (50 mL) and washed with water (2×30 mL), then brine (30 mL), dried over anhydrous Na₂SO₄, filtered, and concentrated in vacuo to afford 4-(3-(4-chlorophenyl)-4,5-dihydro-1H-pyrazol-5-yl)-2-methoxyphenol as a brown oil (606 mg), which was used in the next step without further purification.

In a sealed vial, a mixture of 4-(3-(4-chlorophenyl)-4,5-dihydro-1H-pyrazol-5-yl)-2-methoxyphenol (606 mg) and succinic anhydride (400 mg, 4.00 mmol) in THF (16 mL) was subjected to microwave irradiation at 120° C. for 45 min. The reaction mixture was concentrated in vacuo and purified by flash column chromatography (petroleum ether/EtOAc, 1:1→EtOAc, neat). The resulting solid was solubilized in CHCl₃ (30 mL) and washed with water (2×15 mL), then brine (15 mL), dried over anhydrous Na₂SO₄, filtered, and concentrated in vacuo to afford Compound 45 as a brown solid (81 mg, 10% over 3 steps). ¹H NMR (400 MHz, DMSO-d₆) δ 12.12 (1H, br s), 8.89 (1H, br s), 7.79 (2H, d, J=8.7 Hz), 7.53 (2H, d, J=8.7 Hz), 6.73 (1H, d, J=2.0 Hz), 6.68 (1H, d, J=8.1 Hz), 6.55 (1H, dd, J=8.2, 2.0 Hz), 5.45 (1H, dd, J=11.7, 4.6 Hz), 3.79 (1H, dd, J=18.0, 11.8 Hz), 3.72 (3H, s), 3.11 (1H, dd, J=18.1, 4.7 Hz), 3.02 (1H, dt, J=17.0, 6.8 Hz), 2.89 (1H, dt, J=17.0, 6.5 Hz), 2.51-2.47 (2H, m); HRMS (ESI/Q-TOF) m/z: [M+H]⁺ calcd for C₂₀H₂₀ClN₂O₅, 403.1055; found, 403.1047.

### 4-(3-([1,1′-Biphenyl]-4-yl)-5-(4-hydroxy-3-methoxyphenyl)-4,5-dihydro-1H-pyrazol-1-yl)-4-oxobutanoic acid (Compound 46)

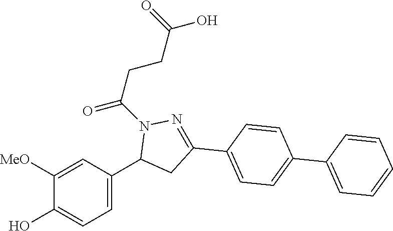 [SMILES: COc1cc(C2CC(c3ccc(-c4ccccc4)cc3)=NN2C(=O)CCC(=O)O)ccc1O]

To a solution of vanillin (304 mg, 2.00 mmol) and 4-acetylbiphenyl (392 mg, 2.00 mmol) in EtOH (16 mL) at 0° C. was added dropwise SOCl₂ (0.10 mL, 1.40 mmol), and the reaction mixture stirred at room temperature for 24 h. After, the solution was cooled down to 0° C., and water (10 mL) was added to the solution, and the resulting precipitated solids were then collected by filtration, washed with water and dried in vacuo to afford (E)-1-([1,1′-biphenyl]-4-yl)-3-(4-hydroxy-3-methoxyphenyl)prop-2-en-1-one as an orange solid (562 g), which was used directly in the next step without further purification.

A mixture of (E)-1-([1,1′-biphenyl]-4-yl)-3-(4-hydroxy-3-methoxyphenyl)prop-2-en-1-one (562 mg) and hydrazine hydrate (0.21 mL, 50% w/w) in THF (14 mL) was stirred under reflux for 2 h. After, succinic anhydride (510 mg, 5.10 mmol) was added and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. Purification by flash column chromatography (petroleum ether/EtOAc, 1:1→EtOAc, neat) afforded Compound 46 as an off-white solid (320 mg, 36% over 2 steps). ¹H NMR (400 MHz, DMSO-d₆) δ 12.08 (1H, s), 8.88 (1H, s), 7.87 (2H, d, J=8.5 Hz), 7.78 (2H, d, J=8.6 Hz), 7.73 (2H, d, J=7.2 Hz), 7.50 (2H, t, J=7.5 Hz), 7.40 (1H, t, J=7.3 Hz), 6.76 (1H, d, J=2.0 Hz), 6.70 (1H, d, J=8.1 Hz), 6.58 (1H, dd, J=8.2, 2.0 Hz), 5.47 (1H, dd, J=11.6, 4.5 Hz), 3.84 (1H, dd, J=18.0, 11.7 Hz), 3.74 (3H, s), 3.16 (1H, dd, J=18.0, 4.6 Hz), 3.06 (1H, dt, J=17.0, 6.8 Hz), 2.92 (1H, dt, J=17.1, 6.9 Hz), 2.54-2.50 (2H, m); HRMS (ESI/Q-TOF) m/z: [M+Na]⁺ calcd for C₂₆H₂₄N₂NaO₅, 467.1577; found, 467.1573.

### 4-(3-(4-Chlorophenyl)-5-(3-ethoxy-4-hydroxyphenyl)-4,5-dihydro-1H-pyrazol-1-yl)-4-oxobutanoic acid (Compound 47)

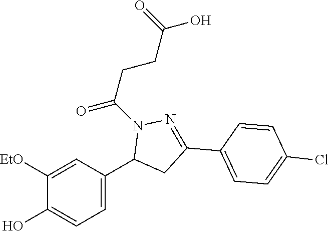 [SMILES: CCOc1cc(C2CC(c3ccc(Cl)cc3)=NN2C(=O)CCC(=O)O)ccc1O]

To a solution of ethyl vanillin (332 mg, 2.00 mmol) and 4-chloroacetophenone (0.26 mL, 2.00 mmol) in EtOH (16 mL) at 0° C. was added dropwise SOCl₂ (0.10 mL, 1.40 mmol), and the reaction mixture stirred at room temperature for 48 h. After, the solution was cooled down to 0° C., and water (16 mL) was added to the solution, and the resulting precipitated solids were then collected by filtration, washed with water and dried in vacuo to afford (E)-1-(4-chlorophenyl)-3-(3-ethoxy-4-hydroxyphenyl)prop-2-en-1-one as an off-white solid (374 mg), which was used directly in the next step without further purification.

A mixture of (E)-1-(4-chlorophenyl)-3-(3-ethoxy-4-hydroxyphenyl)prop-2-en-1-one (374 mg) and hydrazine hydrate (0.15 mL, 50% w/w) in THF (10 mL) was stirred under reflux for 2 h. After, succinic anhydride (354 mg, 3.54 mmol) was added and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. Purification by flash column chromatography (petroleum ether/EtOAc, 1:1→EtOAc, neat) afforded Compound 47 as a colourless solid (342 mg, 41% over 2 steps). ¹H NMR (400 MHz, DMSO-d₆) δ 12.07 (1H, br s), 8.80 (1H, br s), 7.80 (2H, d, J=8.6 Hz), 7.53 (2H, d, J=8.6 Hz), 6.72 (1H, d, J=1.8 Hz), 6.70 (1H, d, J=8.1 Hz), 6.56 (1H, dd, J=8.1, 1.8 Hz), 5.44 (1H, dd, J=11.7, 4.5 Hz), 3.97 (2H, q. J=7.0 Hz), 3.79 (1H, dd, J=18.1, 11.8 Hz), 3.12 (1H, dd, J=18.1, 4.6 Hz), 3.01 (1H, dt, J=17.0, 6.8 Hz), 2.91 (1H, dt, J=17.1, 6.6 Hz), 2.51-2.48 (2H, m), 1.30 (3H, t, J=7.0 Hz); HRMS (ESI/Q-TOF) m/z: [M+Na]⁺ calcd for C₂₁H₂₁ClN₂NaO₅, 439.1031; found, 439.1025.

### 4-(3-(4-Chlorophenyl)-5-(4-fluoro-3-methoxyphenyl)-4,5-dihydro-1H-pyrazol-1-yl)-4-oxobutanoic acid (Compound 48)

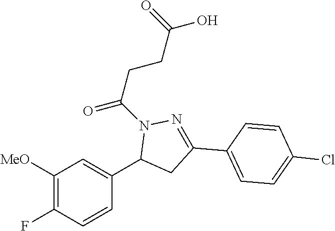 [SMILES: COc1cc(C2CC(c3ccc(Cl)cc3)=NN2C(=O)CCC(=O)O)ccc1F]

Compound 48 was prepared over 2 steps using a method similar to that described for the preparation of Compound 20-step 1:4-fluoro-3-methoxybenzaldehyde (308 mg, 2.00 mmol), 4-chloroacetophenone (0.26 mL, 2.00 mmol) and 2 M NaOH (5.0 mL, 10.0 mmol) in EtOH (16 mL) to afford (E)-1-(4-chlorophenyl)-3-(4-fluoro-3-methoxyphenyl)prop-2-en-1-one as an off-white solid (494 mg); step 2: (E)-1-(4-chlorophenyl)-3-(4-fluoro-3-methoxyphenyl)prop-2-en-1-one (494 mg) and hydrazine hydrate (0.32 mL, 50% w/w) in THF (14 mL), followed by the addition of succinic anhydride (511 mg, 5.10 mmol). Purification by flash column chromatography (petroleum ether/EtOAc, 1:1→EtOAc, neat→CH₂Cl₂/MeOH, 9:1) afforded Compound 48 as a yellow solid (97 mg, 12% over 2 steps); ¹H NMR (400 MHz, CDCl₃) δ 7.68 (2H, d, J=8.7 Hz), 7.40 (2H, d, J=8.7 Hz), 7.00 (1H, dd, J=11.0, 8.3 Hz), 6.82 (1H, dd, J=7.9, 2.1 Hz), 6.72 (1H, ddd, J=8.3, 4.1, 2.2 Hz), 5.53 (1H, dd, J=11.8, 4.9 Hz), 3.85 (3H, s), 3.73 (1H, dd, J=17.7, 11.8 Hz), 3.22-3.05 (3H, m), 2.73 (2H, t, J=6.7 Hz); HRMS (ESI/Q-TOF) m/z: [M+H]⁺ calcd for C₂₀H₁₉ClFN₂O₄, 405.1012; found, 405.1010.

### 3-(3-(4-Chlorophenyl)-5-(quinoxalin-6-yl)-4,5-dihydro-1H-pyrazol-1-yl)-3-oxopropanoic acid (Compound 49)

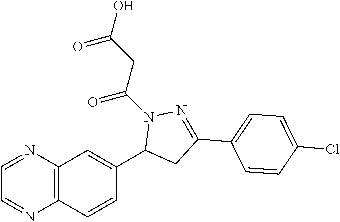 [SMILES: O=C(O)CC(=O)N1N=C(c2ccc(Cl)cc2)CC1c1ccc2nccnc2c1]

To a stirred solution of 3-methoxy-3-oxopropanoic acid (46 mg, 0.39 mmol) in CH₂Cl₂ (5 mL) was added oxalyl chloride (35 μL, 0.41 mmol), followed by DMF (1 drop). The reaction mixture was stirred at room temperature for 4 h before concentrating in vacuo. The residue was taken up in CH₂Cl₂ (5 mL) and to this was added 6-(3-(4-chlorophenyl)-4,5-dihydro-1H-pyrazol-5-yl)quinoxaline (100 mg, 0.32 mmol), followed by pyridine (32 μL, 0.40 mmol), and the reaction mixture stirred at room temperature for 18 h. The reaction was then quenched with sat. aq. NaHCO₃ (5 mL) and the aqueous layer was separated and extracted with CH₂Cl₂. The combined organic layers were dried over anhydrous Na₂SO₄, filtered and concentrated in vacuo. Purification by flash column chromatography (petroleum ether/EtOAc, 1:1) afforded the corresponding methyl ester of Compound 49 as a pale-red solid (77 mg, 59%). ¹H NMR (400 MHz, CDCl₃) δ 8.84-8.82 (2H, m), 8.12 (1H, d, J=8.7 Hz), 8.02 (1H, d, J=2.0 Hz), 7.70 (1H, dd, J=8.7, 2.1 Hz), 7.65 (2H, d, J=8.8 Hz), 7.41 (2H, d, J=8.8 Hz), 5.86 (1H, dd, J=11.9, 4.9 Hz), 3.94-3.85 (3H, m), 3.78 (3H, s), 3.26 (1H, dd, J=17.8, 4.9 Hz).

To a stirred solution of the above methyl ester (40 mg, 0.10 mmol) in THF/H₂O (2.50 mL, 4:1 v/v) was added LiOH·H₂O (8.0 mg, 0.19 mmol), and the reaction mixture stirred at room temperature for 18 h. The reaction mixture was then diluted with H₂O (10 mL) and acidified to aprox. pH 1 through the addition of 2 M aq. HCl, then extracted with EtOAc (2×10 mL). The combined organic layers were dried over anhydrous Na₂SO₄, filtered and concentrated in vacuo to afford Compound 49 as a yellow solid (29 mg, 73%). ¹H NMR (400 MHz, CDCl₃) δ 8.87-8.85 (2H, m), 8.15 (1H, d, J=8.6 Hz), 7.99 (1H, d, J=2.1 Hz), 7.71 (2H, d, J=8.8 Hz), 7.67 (1H, dd, J=8.7, 2.1 Hz), 7.45 (2H, d, J=8.7 Hz), 5.86 (1H, dd, J=11.8, 4.7 Hz), 3.99-3.91 (3H, m), 3.34 (1H, dd, J=18.0, 4.8 Hz).

### 5-(3-(4-Chlorophenyl)-5-(quinoxalin-6-yl)-4,5-dihydro-1H-pyrazol-1-yl)-5-oxopentanoic acid (Compound 50)

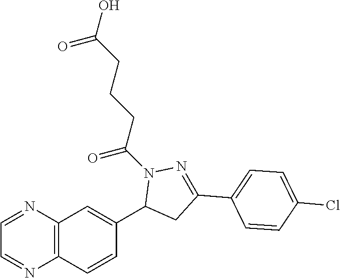 [SMILES: O=C(O)CCCC(=O)N1N=C(c2ccc(Cl)cc2)CC1c1ccc2nccnc2c1]

To a stirred solution of 6-(3-(4-chlorophenyl)-4,5-dihydro-1H-pyrazol-5-yl)quinoxaline (50 mg, 0.16 mmol) in THF (5 mL) was added glutaric anhydride (37 mg, 0.32 mmol), and the reaction mixture heated at 50° C. for 18 h. The resulting reaction mixture was concentrated in vacuo. Purification by flash column chromatography (EtOAc, neat→CH₂Cl₂/MeOH, 9:1) afforded Compound 50 as a pale-yellow solid (52 mg, 77%). ¹H NMR (400 MHz, DMSO-d₆) δ 8.95-8.93 (2H, m), 8.09 (1H, d, J=8.7 Hz), 7.87 (1H, d, J=2.0 Hz), 7.82 (2H, d, J=8.7 Hz), 7.70 (1H, dd, J=8.7, 2.0 Hz), 7.54 (2H, d, J=8.7 Hz), 5.84 (1H, dd, J=12.0, 5.1 Hz), 3.96 (1H, dd, J=18.2, 12.1 Hz), 3.34-3.27 (1H, m), 2.89-2.75 (2H, m), 2.27 (2H, t, J=7.4 Hz), 1.83-1.75 (2H, m).

### (E)-4-(3-(4-Chlorophenyl)-5-(quinoxalin-6-yl)-4,5-dihydro-1H-pyrazol-1-yl)-4-oxobut-2-enoic acid (Compound 51)

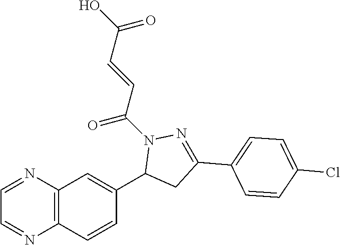 [SMILES: O=C(O)/C=C/C(=O)N1N=C(c2ccc(Cl)cc2)CC1c1ccc2nccnc2c1]

To a stirred solution of mono-ethyl fumarate (70 mg, 0.49 mmol) in CH₂Cl₂ (5 mL) was added oxalyl chloride (45 μL, 0.52 mmol) followed by DMF (1 drop). The reaction mixture was stirred at room temperature for 4 h before concentrating in vacuo. The residue was taken up in CH₂Cl₂ (5 mL) and to this was added 6-(3-(4-chlorophenyl)-4,5-dihydro-1H-pyrazol-5-yl)quinoxaline (100 mg, 0.32 mmol), followed by pyridine (32 μL, 0.40 mmol), and the reaction mixture stirred at room temperature for 18 h. The reaction was quenched with sat. aq. NaHCO₃ (5 mL) and the aqueous layer was separated and extracted with CH₂Cl₂. The combined organic layers were dried over anhydrous Na₂SO₄, filtered, and concentrated in vacuo. Purification by flash column chromatography (petroleum ether/EtOAc, 1:1) afforded the corresponding ethyl ester of Compound 51 as a pale-yellow solid (103 mg, 74%). ¹H NMR (400 MHz, DMSO-d₆) δ 8.83-8.82 (2H, m), 8.11 (1H, d, J=8.7 Hz), 8.04 (1H, d, J=15.5 Hz), 7.98 (1H, d, J=2.0 Hz), 7.74 (2H, d, J=8.6 Hz), 7.68 (1H, dd, J=8.7, 2.1 Hz), 7.44 (2H, d, J=8.5 Hz), 6.89 (1H, d, J=15.6 Hz), 5.91 (1H, dd, J=11.8, 4.9 Hz), 4.29 (2H, q. J=7.1 Hz), 3.92 (1H, dd, J=17.8, 11.8 Hz), 3.29 (1H, dd, J=17.9, 4.9 Hz), 1.34 (3H, t, J=7.0 Hz).

To a stirred solution of above ethyl ester (60 mg, 0.14 mmol) in THF/H₂O (2.50 mL, 4:1 v/v) was added LiOH·H₂O (12 mg, 0.28 mmol). The reaction mixture was stirred at room temperature for 18 h before diluting with H₂O (10 mL). The reaction mixture was acidified to aprox. pH 1 through the addition of 2 M aq. HCl, and the aqueous layer was extracted with EtOAc (2×10 mL). The combined organic layers were dried over anhydrous Na₂SO₄, filtered and concentrated in vacuo to afford Compound 51 as a yellow solid (29 mg, 51%). ¹H NMR (400 MHz, CDCl₃) δ 8.84 (2H, s), 8.15-8.10 (2H, m), 8.00 (1H, d, J=1.9 Hz), 7.75 (2H, d, J=8.6 Hz), 7.69 (1H, dd, J=8.9, 2.0 Hz), 7.44 (2H, d, J=8.6 Hz), 6.91 (1H, d, J=16.0 Hz), 5.92 (1H, dd, J=11.7, 4.8 Hz), 3.93 (1H, dd, J=17.8, 11.8 Hz), 3.31 (1H, dd, J=17.9, 5.0 Hz).

### (Z)-4-(3-(4-Chlorophenyl)-5-(quinoxalin-6-yl)-4,5-dihydro-1H-pyrazol-1-yl)-4-oxobut-2-enoic acid (Compound 52)

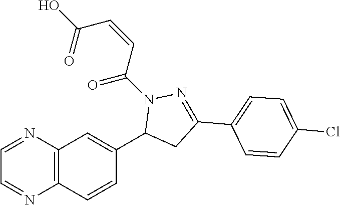 [SMILES: O=C(O)/C=C\C(=O)N1N=C(c2ccc(Cl)cc2)CC1c1ccc2nccnc2c1]

To a stirred solution of 6-(3-(4-chlorophenyl)-4,5-dihydro-1H-pyrazol-5-yl)quinoxaline (50 mg, 0.16 mmol) in THF (5 mL) was added maleic anhydride (32 mg, 0.32 mmol), and the reaction mixture heated at 50° C. for 18 h. The resulting solids were collected by filtration and washed with THF to afford Compound 52 as a pale-yellow solid (55 mg, 83%). ¹H NMR (400 MHz, DMSO-d₆) δ 8.95-8.93 (2H, m), 8.08 (1H, d, J=9.1 Hz), 8.03-7.99 (1H, m), 7.82-7.77 (3H, m), 7.54 (2H, d, J=8.4 Hz), 6.29-6.25 (1H, m), 5.90-5.85 (1H, m), 4.03-3.94 (1H, m), 3.34-3.26 (1H, m).

### 4-(3-(4-Chlorophenyl)-5-(quinoxalin-6-yl)-4,5-dihydro-1H-pyrazol-1-yl)butanoic acid (Compound 53)

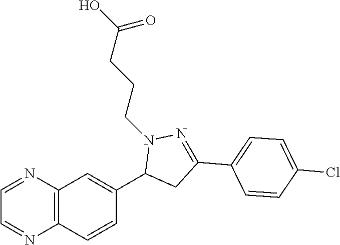 [SMILES: O=C(O)CCCN1N=C(c2ccc(Cl)cc2)CC1c1ccc2nccnc2c1]

To a solution of 6-(3-(4-chlorophenyl)-4,5-dihydro-1H-pyrazol-5-yl)quinoxaline (200 mg, 0.647 mmol) and ethyl 4-oxobutanoate (101 mg, 0.777 mmol) in 1,2-dichloroethane (10 mL) at room temperature was added sodium triacetoxyborohydride (165 mg, 0.777 mmol), and the reaction mixture stirred for 72 h. The reaction mixture was then diluted with CH₂Cl₂ (10 mL) and washed with brine (10 mL). The organic layer was dried over anhydrous MgSO₄, filtered and concentrated in vacuo. Purification by flash column chromatography (petroleum ether/EtOAc, 7:3) afforded the corresponding ethyl ester of Compound 53 as a yellow oil (40 mg, 15%). ¹H NMR (500 MHz, CDCl₃) δ 8.87-8.86 (2H, m), 8.14 (1H, d, J=8.7 Hz), 8.11 (1H, d, J=1.9 Hz), 7.97 (1H, dd, J=8.7, 1.9 Hz), 7.59 (2H, d, J=8.6 Hz), 7.34 (2H, d, J=8.6 Hz), 4.55 (1H, dd, J=13.9, 10.4 Hz), 4.05-3.99 (2H, m), 3.53 (1H, dd, J=16.0, 10.3 Hz), 3.04 (1H, dd, J=16.0, 13.7 Hz), 3.05-2.93 (2H, m), 2.48-2.32 (2H, m), 2.15-1.98 (2H, m), 1.15 (3H, t, J=7.1 Hz); ¹³C NMR (125 MHz, CDCl₃) δ 173.6 (C), 148.2 (C), 145.5 (CH), 145.2 (CH), 143.3 (C), 143.2 (C), 143.0 (C), 134.6 (C), 131.4 (C), 130.3 (CH), 129.4 (CH), 128.9 (2×CH), 128.4 (CH), 127.1 (2×CH), 71.4 (CH), 60.4 (CH₂), 53.3 (CH₂), 43.0 (CH₂), 32.0 (CH₂), 23.3 (CH₂), 14.3 (CH₃).

To a solution of the above ethyl ester (40 mg, 0.095 mmol) in EtOH/H₂O (4 mL, 3:1 v/v) was added lithium hydroxide (8.0 mg, 0.19 mmol), and the reaction stirred at room temperature for 18 h. The resultant reaction mixture was then concentrated in vacuo and diluted with ethyl acetate (10 mL). The organic layer was washed with water (10 mL) then acidified to aprox. pH 1 through the addition of 1 M aq. HCl. The organic layer was then concentrated in vacuo to afford Compound 53 as a yellow solid (28 mg, 75%). ¹H NMR (500 MHz, CDCl₃) δ 8.86-8.84 (2H, m), 8.14 (1H, d, J=8.7 Hz), 8.12 (1H, d, J=1.9 Hz), 7.96 (1H, dd, J=8.7, 1.9 Hz), 7.59 (2H, d, J=8.6 Hz), 7.35 (2H, d, J=8.6 Hz), 4.56 (1H, dd, J=13.9, 10.4 Hz), 3.55 (1H, dd, J=16.0, 10.3 Hz), 3.05 (1H, dd, J=16.0, 13.7 Hz), 3.07-2.95 (2H, m), 2.58-2.43 (2H, m), 2.18-1.96 (2H, m); ¹³C NMR (125 MHz, CDCl₃) δ 176.4 (C), 148.7 (C), 145.4 (CH), 145.2 (CH), 143.1 (C), 143.0 (C), 142.9 (C), 134.8 (C), 131.2 (C), 130.3 (CH), 129.4 (CH), 129.0 (2×CH), 128.3 (CH), 127.2 (2×CH), 71.5 (CH), 52.8 (CH₂), 42.9 (CH₂), 31.4 (CH₂), 23.2 (CH₂).

### Ethyl 4-(3-(4-chlorophenyl)-5-(quinoxalin-6-yl)-4,5-dihydro-1H-pyrazol-1-yl)-4-oxobutanoate

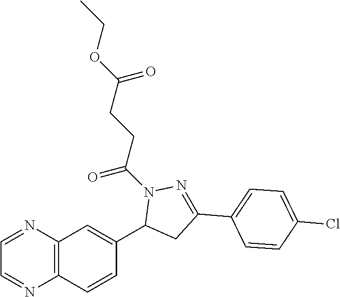 [SMILES: CCOC(=O)CCC(=O)N1N=C(c2ccc(Cl)cc2)CC1c1ccc2nccnc2c1]

To a stirred solution of ethyl succinyl chloride (94 μL, 0.66 mmol) in CH₂Cl₂ (5 mL) was added 6-(3-(4-chlorophenyl)-4,5-dihydro-1H-pyrazol-5-yl)quinoxaline (200 mg, 0.648 mmol), followed by pyridine (63 μL, 0.78 mmol), and the reaction mixture stirred at room temperature for 18 h. The reaction mixture was then quenched with sat. aq. NH₄Cl (5 mL) and the aqueous layer was separated and then extracted with CH₂Cl₂. The combined organic layers were dried over anhydrous Na₂SO₄, filtered, and concentrated in vacuo. Purification by flash column chromatography (EtOAc, neat) afforded the titled compound as a colourless solid (220 mg, 78%). ¹H NMR (400 MHz, CDCl₃) δ 8.83-8.81 (2H, m), 8.10 (1H, d, J=8.7 Hz), 7.96 (1H, d, J=2.0 Hz), 7.71-7.65 (3H, m), 7.41 (2H, d, J=8.8 Hz), 5.82 (1H, dd, J=12.0, 5.0 Hz), 4.12 (2H, q, J=7.1 Hz), 3.85 (1H, dd, J=17.7, 12.0 Hz), 3.24-3.17 (3H, m), 2.74-2.62 (2H, m), 1.22 (3H, t, J=7.1 Hz).

### (Pivaloyloxy)methyl 4-(3-(4-chlorophenyl)-5-(quinoxalin-6-yl)-4,5-dihydro-1H-pyrazol-1-yl)-4-oxobutanoate (Compound 54)

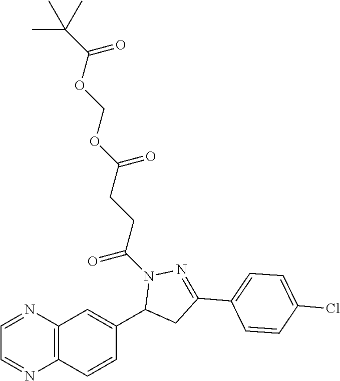 [SMILES: CC(C)(C)C(=O)OCOC(=O)CCC(=O)N1N=C(c2ccc(Cl)cc2)CC1c1ccc2nccnc2c1]

To a stirred mixture of Compound 1 (50 mg, 0.12 mmol), chloromethyl pivalate (38 mg, 0.25 mmol) and NaI (38 mg, 0.25 mmol) in acetone (5 mL) at room temperature was added DIPEA (25 μL, 0.14 mmol), and the reaction mixture stirred at room temperature for 18 h before adding sat. aq. NH₄Cl (20 mL) and EtOAc (20 mL). The aqueous layer was then separated and extracted with EtOAc, and the combined organic layers were dried over anhydrous Na₂SO₄, filtered and concentrated in vacuo. Purification by flash column chromatography (petroleum ether/EtOAc, 1:1) afforded Compound 54 as a yellow residue (43 mg, 68%). ¹H NMR (400 MHz, CDCl₃) δ 8.83-8.82 (2H, m), 8.10 (1H, d, J=8.7 Hz), 7.95 (1H, d, J=2.0 Hz), 7.69 (2H, d, J=8.6 Hz), 7.66 (1H, dd, J=8.7, 2.0 Hz), 7.41 (2H, d, J=8.6 Hz), 5.81 (1H, dd, J=12.0, 5.0 Hz), 5.76-5.72 (2H, m), 3.87 (1H, dd, J=17.7, 12.0 Hz), 3.25-3.18 (3H, m), 2.81-2.66 (2H, m), 1.17 (9H, s).

### 4-(3-(4-Chlorophenyl)-5-(quinoxalin-6-yl)-4,5-dihydro-1-pyrazol-1-yl)-N-(methylsulfonyl)-4-oxobutanamide (Compound 55)

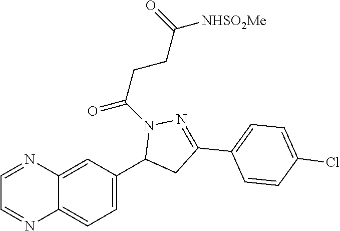

A solution of Compound 1 (0.10 g, 0.24 mmol), methanesulfonamide (37 mg, 0.39 mmol), N-(3-dimethylaminopropyl)-N′-ethylcarbodiimide hydrochloride (EDCI) (74 mg, 0.39 mmol) and 4-(dimethylamino)pyridine (DMAP) (35 mg, 0.29 mmol) in CH₂Cl₂ (2 mL) was stirred at room temperature for 16 h. The reaction mixture was diluted with CH₂Cl₂ (20 mL), washed with 0.2 M aq. HCl (2×20 mL), water (2×10 mL), then brine (10 mL), dried over anhydrous sodium sulfate and concentrated in vacuo. Purification by flash column chromatography (CH₂Cl₂/MeOH, 25:1) afforded Compound 55 as a pale-brown solid (50 mg, 42%). ¹H NMR (400 MHz, CDCl₃) δ 8.82-8.81 (2H, m), 8.10 (1H, d, J=8.7 Hz), 7.94 (1H, d, J=2.0 Hz), 7.70-7.63 (3H, m), 7.43-7.40 (2H, m), 5.85 (1H, dd, J=11.8, 4.7 Hz), 3.87 (1H, dd, J=17.8, 11.9 Hz), 3.32-3.17 (3H, m), 3.14 (3H, s), 2.68-2.53 (2H, m); ¹³C NMR (101 MHz, CDCl₃) δ 171.7 (C), 170.5 (C), 154.5 (C), 145.6 (CH), 145.4 (CH), 143.2 (C), 143.0 (C), 142.7 (C), 137.2 (C), 130.9 (CH), 129.3 (2×CH), 129.3 (C), 128.2 (2×CH), 128.0 (CH), 126.0 (CH), 60.3 (CH), 42.3 (CH₂), 41.4 (CH₃), 31.4 (CH₂), 29.1 (CH₂); HRMS (ESI/Q-TOF) m/z: [M+Na]⁺ calcd for C₂₂H₂₀ClN₅NaO₄S, 508.0817; found, 508.0815.

### 4-(3-([1,1′-Biphenyl]-4-yl)-5-(quinoxalin-6-yl)-4,5-dihydro-1H-pyrazol-1-yl)-N-(methylsulfonyl)-4-oxobutanamide (Compound 57)

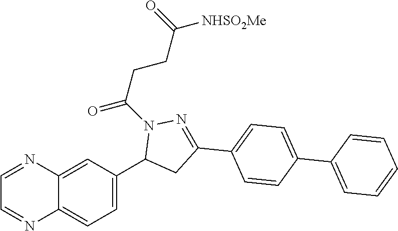

Compound 57 was prepared following a method similar to that described for the preparation of Compound 55 using Compound 15 (0.15 g, 0.33 mmol), methanesulfonamide (60 mg, 0.67 mmol), N-(3-dimethylaminopropyl)-N′-ethylcarbodiimide hydrochloride (EDCI) (0.13 g, 0.67 mmol) and 4-(dimethylamino)pyridine (DMAP) (50 mg, 0.40 mmol) in CH₂Cl₂ (3 mL). Purification by flash column chromatography (CH₂Cl/MeOH, 20:1) afforded Compound 57 as a colourless solid (65 mg, 38%). ¹H NMR (400 MHz, DMSO-d₆) δ 11.72 (1H, br s), 8.94-8.93 (2H, m), 8.09 (1H, d, J=8.6 Hz), 7.92-7.90 (3H, m), 7.80-7.72 (5H, m), 7.51-7.38 (3H, m), 5.86 (1H, dd, J=11.8, 4.8 Hz), 4.02 (1H, dd, J=18.1, 12.0 Hz), 3.39-3.32 (1H, m), 3.20-3.00 (5H, m), 2.60 (2H, t, J=6.5 Hz); ¹³C NMR (101 MHz, DMSO-d₆) δ 172.0 (C), 169.0 (C), 154.3 (C), 146.0 (CH), 145.6 (CH), 144.1 (C), 142.1 (C), 141.9 (C), 141.6 (C), 139.1 (C), 130.0 (C), 129.8 (CH), 129.0 (2×CH), 128.1 (CH), 128.0 (CH), 127.4 (2×CH), 127.0 (2×CH), 126.7 (2×CH), 125.3 (CH), 59.6 (CH), 41.8 (CH₂), 40.8 (CH₃), 30.1 (CH₂), 28.1 (CH₂); HRMS (ESI/Q-TOF) m/z: [M+Na]⁺ calcd for C₂₈H₂₅N₅NaO₄S, 550.1519; found, 550.1507.

### 4-(3-(4-Chlorophenyl)-5-(quinoxalin-6-yl)-4,5-dihydro-1H-pyrazol-1-yl)-N—(N,N-dimethylsulfamoyl)-4-oxobutanamide (Compound 59)

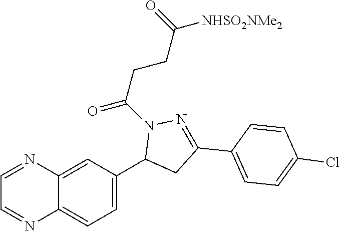

Compound 59 was prepared following a method similar to that described for the preparation of Compound 55 using Compound 1 (0.10 g, 0.24 mmol), N,N-dimethylsulfamide (45 mg, 0.36 mmol), N-(3-dimethylaminopropyl)-N′-ethylcarbodiimide hydrochloride (EDCI) (69 mg, 0.36 mmol) and 4-(dimethylamino)pyridine (DMAP) (35 mg, 0.29 mmol) in CH₂Cl₂ (2 mL). Purification by flash column chromatography (petroleum ether/EtOAc, 1:1→EtOAc, neat) afforded Compound 59 as a colourless solid (98 mg, 79%). ¹H NMR (400 MHz, CDCl₃) δ 9.00 (1H, s), 8.82 (2H, s), 8.10 (1H, d, J=8.7 Hz), 7.95 (1H, d, J=2.0 Hz), 7.68 (2H, d, J=8.7 Hz), 7.65 (1H, dd, J=8.7, 2.0 Hz), 7.41 (2H, d, J=8.7 Hz), 5.84 (1H, dd, J=11.9, 4.8 Hz), 3.87 (1H, dd, J=17.8, 11.9 Hz), 3.34-3.13 (3H, m), 2.76 (6H, s), 2.67-2.54 (2H, m); 13C NMR (101 MHz, CDCl₃) δ 171.0 (C), 170.3 (C), 154.1 (C), 145.6 (CH), 145.3 (CH), 143.4 (C), 143.1 (C), 142.7 (C), 137.1 (C), 130.8 (CH), 129.4 (C), 129.3 (2×CH), 128.2 (2×CH), 128.1 (CH), 126.1 (CH), 60.3 (CH), 42.3 (CH₂), 38.2 (2×CH₃), 30.9 (CH₂), 29.3 (CH₂); HRMS (ESI/Q-TOF) m/z: [M+Na]⁺ calcd for C₂₃H₂₃ClN₆NaO₄S, 537.1082; found, 537.1083.

### 3-(3-(4-Chlorophenyl)-5-(quinoxalin-6-yl)-4,5-dihydro-1H-pyrazol-1-yl)benzoic acid (Compound 60)

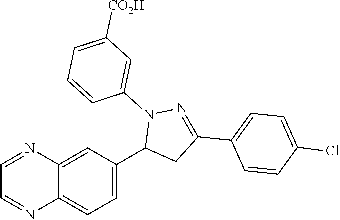 [SMILES: O=C(O)c1cccc(N2N=C(c3ccc(Cl)cc3)CC2c2ccc3nccnc3c2)c1]

To a suspension of 3-aminobenzoic acid (1.00 g, 7.29 mmol) in concentrated HCl (10 mL) at −5° C. was added dropwise sodium nitrite (601 mg, 8.75 mmol) in water (2.5 mL), and the reaction mixture stirred at −5° C. for 1 h. A solution of tin (II) chloride dihydrate (4.93 g, 21.9 mmol) in water (2.5 mL) was then added dropwise at −5° C., and the reaction mixture was further stirred at −5° C. for 2 h. The resulting precipitated solids were collected by filtration, washed with water (10 mL), then diethyl ether (10 mL) and dried in vacuo to afford 3-hydrazinobenzoic acid as a colourless solid (754 mg, 68%). ¹H NMR (400 MHz, DMSO-d₆) δ 10.78 (3H, s), 8.43 (1H, s), 7.55 (1H, s), 7.48 (1H, d, J=7.5 Hz), 7.38 (1H, t, J=7.7 Hz), 7.21 (1H, d, J=7.2 Hz); ¹³C NMR (101 MHz, DMSO-d₆) δ 167.06 (C), 146.28 (C), 131.43 (C), 129.04 (C), 121.74 (CH), 118.44 (CH), 114.45 (CH).

A suspension of (E)-1-(4-chlorophenyl)-3-(quinoxalin-6-yl)prop-2-en-1-one (0.20 g, 0.68 mmol) and 3-hydrazinobenzoic acid (0.10 g, 0.68 mmol) in AcOH/ⁿBuOH (1:2 v/v, 15 mL) was stirred at 120° C. for 4 h (before returning to room temperature). The reaction mixture was then concentrated in vacuo and the resulting residue was purified by flash column chromatography (CH₂Cl₂/MeOH, 20:1) to afford Compound 60 as a brown solid (60 mg, 21%). ¹H NMR (400 MHz, CDCl₃) δ 9.94 (1H, br s), 8.82 (2H, s), 8.10 (2H, d, J=8.5 Hz), 7.81 (1H, s), 7.67 (3H, m), 7.50 (1H, d, J=6.7 Hz), 7.35-7.20 (4H, m), 5.58 (1H, dd, J=11.5, 6.3 Hz), 3.92 (1H, dd, J=16.7, 12.8 Hz), 3.19 (1H, dd, J=17.1, 6.4 Hz); ¹³C NMR (101 MHz, CDCl₃) δ 171.3 (C), 146.5 (C), 145.5 (CH), 145.2 (CH), 144.4 (C), 144.2 (C), 143.0 (C), 142.5 (C), 135.0 (C), 131.0 (CH), 130.8 (2×C), 129.3 (2×CH), 129.0 (CH), 128.2 (CH), 127.2 (2× CH), 126.4 (CH), 121.3 (CH), 118.3 (CH), 114.8 (CH), 64.0 (CH), 43.4 (CH₂); HRMS (ESI/Q-TOF) m/z: [M+H]⁺ calcd for C₂₄H₁₈ClN₄O₂, 429.1113; found, 429.1123.

### 3-(3-([1,1′-Biphenyl]-4-yl)-5-(quinoxalin-6-yl)-4,5-dihydro-1H-pyrazol-1-yl)benzoic acid (Compound 61)

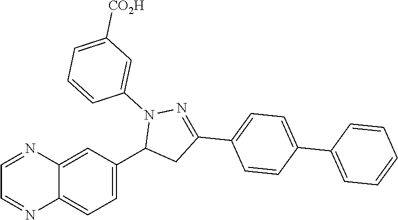 [SMILES: O=C(O)c1cccc(N2N=C(c3ccc(-c4ccccc4)cc3)CC2c2ccc3nccnc3c2)c1]

A suspension of (E)-1-([1,1′-biphenyl]-4-yl)-3-(quinoxalin-6-yl)prop-2-en-1-one (0.30 g, 0.90 mmol) and 3-hydrazinobenzoic acid (0.14 g, 0.90 mmol) in AcOH/ⁿBuOH (1:2 v/v, 18 mL) was stirred at 120° C. for 4 h. A second portion of 3-hydrazinobenzoic acid was added (0.14 g, 0.90 mmol) and the mixture was further stirred at 120° C. for 2 h. The reaction mixture was then concentrated in vacuo and the resulting residue was diluted in EtOAc (50 mL) and washed with water (2×30 mL), then brine (30 mL), dried over anhydrous Na₂SO₄, filtered, and concentrated in vacuo. Purification by flash column chromatography (petroleum ether/EtOAc, 1:1→EtOAc, neat→CH₂Cl₂/MeOH, 9:1) afforded Compound 61 as a yellow solid (68 mg, 16%). ¹H NMR (400 MHz, DMSO-d₆) δ 12.83 (1H, br s), 8.95-8.93 (2H, m), 8.13 (1H, d, J=8.7 Hz), 8.04 (1H, d, J=2.0 Hz), 7.90 (2H, d, J=8.5 Hz), 7.81-7.71 (6H, m), 7.54-7.46 (2H, m), 7.42-7.38 (1H, m), 7.37-7.23 (3H, m), 5.95 (1H, dd, J=12.2, 5.8 Hz), 4.11 (1H, dd, J=17.6, 12.3 Hz), 3.40-3.34 (1H, m); HRMS (ESI/Q-TOF) m/z: [M+H]⁺ calcd for C₃₀H₂₃N₄O₂, 471.1816; found, 471.1812.

### 5-(2-(3-(4-Chlorophenyl)-5-(quinoxalin-6-yl)-4,5-dihydro-1H-pyrazol-1-yl)-2-oxoethyl)thiazolidine-2,4-dione (Compound 63)

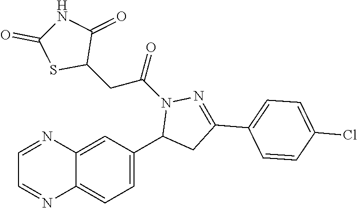 [SMILES: O=C1NC(=O)C(CC(=O)N2N=C(c3ccc(Cl)cc3)CC2c2ccc3nccnc3c2)S1]

A solution of maleic anhydride (10.0 g, 102 mmol) and thiourea (7.76 g, 102 mmol) in concentrated HCl (30 mL) was heated at reflux for 5 h (before returning to room temperature). The precipitates solids were collected by filtration, washed with water (2×20 mL) and dried in vacuo to afford 2-(2,4-dioxothiazolidin-5-yl) acetic acid as a clear crystal solid (10.6 g, 59%). ¹H NMR (400 MHz, DMSO-d₆) δ 12.72 (1H, s), 11.99 (1H, s), 4.65 (1H, dd, J=7.7, 4.7 Hz), 3.01 (2H, m); ¹³C NMR (101 MHz, DMSO-d₆) δ 175.68 (C), 172.51 (C), 171.56 (C), 46.66 (CH), 35.96 (CH₂).

A solution of 2-(2,4-dioxothiazolidin-5-yl) acetic acid (3.50 g, 20.0 mmol) and SOCl₂ (3.00 mL, 41.3 mmol) in dry 1,4-dioxane (12 mL) was heated at reflux for 5 h (before returning to room temperature). Hexane (50 mL) was then added to the reaction mixture and the resulting precipitated solids were collected by filtration, washed with hexane (50 mL) and dried in vacuo to afford 2-(2,4-dioxothiazolidin-5-yl) acetyl chloride (3.02 g, 78%) as an orange solid, which was used without further purifications.

To a solution of 6-(3-(4-chlorophenyl)-4,5-dihydro-1H-pyrazol-5-yl)quinoxaline (420 mg, 1.36 mmol) in THF (13 mL) was added 2-(2,4-dioxothiazolidin-5-yl) acetyl chloride (527 mg, 2.72 mmol), followed by pyridine (0.16 mL, 2.04 mmol) in a single portion and the reaction mixture heated at reflux for 2 h (before returning to room temperature). Water (50 mL) was then added and the resulting precipitated solids were collected by filtration, washed with CH₂CL/MeOH (1:1, 10 mL), CH₂Cl₂ (10 mL), EtOAc (10 mL) then petroleum ether (10 mL), and dried in vacuo to afford Compound 63 as an off-white solid (150 mg, 24%). ¹H NMR (400 MHz, DMSO-d₆) δ 11.98 (1H, s), 8.93 (2H, d, J=4.9 Hz), 8.09 (1H, dd, J=8.6, 6.2 Hz), 7.94-7.83 (3H, m), 7.75-7.69 (1H, m), 7.55 (2H, d, J=8.5 Hz), 5.87 (1H, m), 4.69 (1H, dd, J=9.7, 3.3 Hz), 4.04-3.96 (1H, m), 3.85-3.71 (1H, m), 3.54-3.37 (2H, m); ¹³C NMR (101 MHz, DMSO-d₆) δ 176.0 (C), 172.8 (C), 172.7* (C), 167.1 (C), 154.7 (C), 146.1* (CH), 146.0 (CH), 145.7* (CH), 145.6 (CH), 143.6 (C), 143.5* (C), 142.1 (C), 141.6 (C), 135.3 (C), 130.0 (CH), 129.9* (CH), 129.6 (C), 129.5* (C), 128.9 (2×CH), 128.7 (2×CH), 128.2 (CH), 128.1* (CH), 125.6 (CH), 125.4* (CH), 59.9 (CH), 59.8* (CH), 46.6* (CH), 46.5 (CH), 41.9 (CH₂), 41.8* (CH₂), 36.6 (CH₂), 36.4* (CH₂). * denotes the second stereoisomer. HRMS (ESI/Q-TOF) m/z: [M+H]⁺ calcd for C₂₂H₁₇ClN₅O₃S, 466.0735; found, 466.0719.

### 4-(3-(4′-Isopropoxy-[1,1′-biphenyl]-4-yl)-5-(quinoxalin-6-yl)-4,5-dihydro-1H-pyrazol-1-yl)-4-oxobutanoic acid (Compound 64)

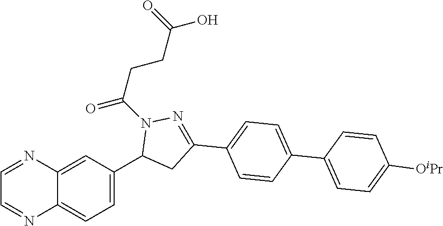 [SMILES: CC(C)Oc1ccc(-c2ccc(C3=NN(C(=O)CCC(=O)O)C(c4ccc5nccnc5c4)C3)cc2)cc1]

Compound 64 was prepared over 2 steps using a method similar to that described for the preparation of Compound 20-step 1: quinoxaline-6-carbaldehyde (158 mg, 1.00 mmol), 1-(4′-isopropoxy-[1,1′-biphenyl]-4-yl)ethan-1-one (254 mg, 1.00 mmol) and 2 M NaOH (2.5 mL, 5.00 mmol) in EtOH (8 mL) to afford (E)-1-(4′-isopropoxy-[1,1′-biphenyl]-4-yl)-3-(quinoxalin-6-yl)prop-2-en-1-one as a pale yellow solid (296 mg); step 2: (E)-1-(4′-isopropoxy-[1,1′-biphenyl]-4-yl)-3-(quinoxalin-6-yl)prop-2-en-1-one (296 mg) and hydrazine hydrate (93 μL, 50% w/w) in THF (6 mL), followed by the addition of succinic anhydride (225 mg, 2.25 mmol). Purification by flash column chromatography (petroleum ether/EtOAc, 1:1→EtOAc, neat) afforded Compound 64 as a yellow solid (153 mg, 30% over 2 steps). ¹H NMR (400 MHz, CDCl₃) δ 8.81-8.80 (2H, m), 8.10 (1H, d, J=8.7 Hz), 7.99 (1H, d, J=1.9 Hz), 7.79 (2H, d, J=8.5 Hz), 7.68 (1H, dd, J=8.7, 2.0 Hz), 7.61 (2H, d, J=8.5 Hz), 7.54 (2H, d, J=8.8 Hz), 6.97 (2H, d, J=8.8 Hz), 5.84 (1H, dd, J=11.9, 4.8 Hz), 4.60 (1H, hept, J=6.1 Hz), 3.90 (1H, dd, J=17.7, 11.9 Hz), 3.34-3.24 (2H, m), 3.17 (1H, dt, J=17.3, 6.8 Hz), 2.75 (2H, t, J=6.8 Hz), 1.37 (6H, d, J=6.1 Hz); ¹³C NMR (101 MHz, CDCl₃) δ 176.4 (C), 170.3 (C), 158.2 (C), 154.4 (C), 145.3 (CH), 145.1 (CH), 143.9 (C), 143.2 (C), 143.0 (C), 142.6 (C), 132.3 (C), 130.6 (CH), 129.2 (C), 128.3 (2×CH), 128.2 (CH), 127.4 (2×CH), 127.0 (2×CH), 125.9 (CH), 116.4 (2×CH), 70.2 (CH), 60.2 (CH), 42.4 (CH₂), 29.2 (CH₂), 29.0 (CH₂), 22.2 (2×CH₃); HRMS (ESI/Q-TOF) m/z: [M+H]⁺ calcd for C₃₀H₂₉N₄O₄, 509.2183; found, 509.2184.

### 4-(3-(4′-(Difluoromethoxy)-[1,1′-biphenyl]-4-yl)-5-(quinoxalin-6-yl)-4,5-dihydro-1H-pyrazol-1-yl)-4-oxobutanoic acid (Compound 65)

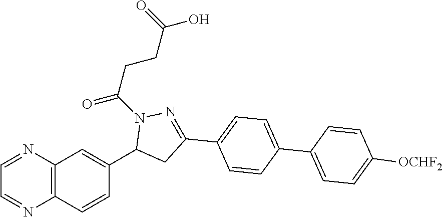 [SMILES: O=C(O)CCC(=O)N1N=C(c2ccc(-c3ccc(OC(F)F)cc3)cc2)CC1c1ccc2nccnc2c1]

To a solution of Compound 9 (77 mg, 0.17 mmol) in DMF/water (3.0 mL, 1:1 v/v), was added 4-(difluoromethoxy)phenylboronic acid (41 mg, 0.22 mmol), potassium phosphate tribasic (93 mg, 0.44 mmol), and palladium acetate (2.5 mg, 10 mol %). The reaction mixture was stirred at room temperature for 16 h. To the mixture was added water (15 mL) and the suspension was acidified to aprox. pH 2 through the addition of 1 M aq. HCl. The aqueous layer was then extracted with ethyl acetate (2×15 mL) and the combined organic layers were washed with water (3×15 mL), then brine (15 mL), dried over anhydrous Na₂SO₄, filtered through celite and concentrated in vacuo. Purification by flash column chromatography (petroleum ether/EtOAc, 1:1→petroleum ether/EtOAc, 8:2→CH₂Cl₂, neat→CH₂Cl₂/MeOH, 9:1) afforded Compound 65 as an off-white solid (25 mg, 28%).

¹H NMR (400 MHz, CDCl₃) δ 8.82-8.81 (2H, m), 8.11 (1H, d, J=8.7 Hz), 7.98 (1H, d, J=2.0 Hz), 7.83 (2H, d, J=8.5 Hz), 7.68 (1H, dd, J=8.7, 2.0 Hz), 7.64-7.59 (4H, m), 7.22 (2H, d. J=9.0 Hz), 6.56 (1H, t, J=73.7 Hz), 5.85 (1H, dd, J=11.9, 4.8 Hz), 3.92 (1H, dd, J=17.7, 11.9 Hz), 3.36-3.26 (2H, m), 3.19 (1H, dt, J=17.4, 6.6 Hz), 2.76 (2H, t, J=6.6 Hz); ¹³C NMR (75 MHz, CDCl₃) δ 174.0 (C), 170.3 (C), 154.6 (C), 152.3 (C), 146.6 (CH), 146.2 (CH), 145.4 (C), 143.7 (C), 143.2 (C), 142.3 (C), 137.9 (C), 131.6 (C), 130.9 (CH), 129.3 (CH), 128.9 (CH), 128.2 (CH), 127.9 (CH), 126.8 (CH), 120.3 (CH), 117.3 (CH, t, J_{C-F}=257.4 Hz), 61.0 (CH), 42.7 (CH₂), 29.8 (CH₂), 28.9 (CH₂).

### 4-(3-(3′,5′-Difluoro-[1,1′-biphenyl]-4-yl)-5-(quinoxalin-6-yl)-4,5-dihydro-1H-pyrazol-1-yl)-4-oxobutanoic acid (Compound 66)

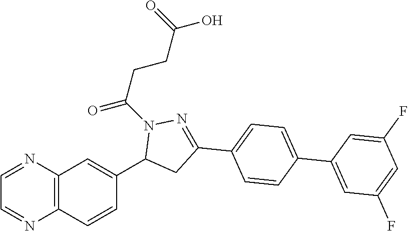 [SMILES: O=C(O)CCC(=O)N1N=C(c2ccc(-c3cc(F)cc(F)c3)cc2)CC1c1ccc2nccnc2c1]

To a solution of quinoxaline-6-carbaldehyde (237 mg, 1.50 mmol) and 1-(3′,5′-difluoro-[1,1′-biphenyl]-4-yl) ethan-1-one (348 mg, 1.50 mmol) in EtOH (12 mL) at room temperature was added dropwise 2 M NaOH (3.8 mL, 7.50 mmol), and the reaction mixture was stirred for 3 h. After, the solution was cooled down to 0° C., and the resulting precipitated solids were then collected by filtration, washed with water and dried in vacuo to afford (E)-1-(3′,5′-difluoro-[1,1′-biphenyl]-4-yl)-3-(quinoxalin-6-yl)prop-2-en-1-one as a pale-yellow solid (492 mg), which was used directly in the next step without further purification.

A mixture of (E)-1-(3′,5′-difluoro-[1,1′-biphenyl]-4-yl)-3-(quinoxalin-6-yl)prop-2-en-1-one (300 mg) and hydrazine hydrate (0.10 mL, 50% w/w) in THF (6 mL) was stirred under reflux for 2 h. After, succinic anhydride (242 mg, 2.42 mmol) was added, and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. The resulting solid was washed with CH₂Cl₂ (10 mL) and isolated by filtration. Purification by flash column chromatography (CH₂Cl₂, neat→CH₂Cl₂/MeOH, 9:1) afforded Compound 66 as an off-white solid (184 mg, 41% over 2 steps). ¹H NMR (400 MHz, DMSO-d₆) δ 12.08 (1H, s), 8.94-8.92 (2H, m), 8.08 (1H, d, J=8.7 Hz), 7.92-7.86 (5H, m), 7.72 (1H, dd, J=8.7, 2.0 Hz), 7.56-7.49 (2H, m), 7.26 (1H, tt, J=9.3, 2.3 Hz), 5.86 (1H, dd, J=11.9, 5.0 Hz), 4.02 (1H, dd, J=18.2, 12.0 Hz), 3.36 (1H, dd, J=18.2, 5.1 Hz), 3.12 (1H, dt, J=17.1, 6.7 Hz), 2.99 (1H, dt, J=17.2, 6.5 Hz), 2.53-2.50 (2H, m); ¹³C NMR (101 MHz, DMSO-d₆) δ 173.7 (C), 169.3 (C), 162.9 (2× C, dd, J_{C-F}=245.8, 13.7 Hz), 153.9 (C), 146.0 (CH), 145.6 (CH), 144.1 (C), 142.7 (C, t, J_{C-F}=9.8 Hz), 142.1 (C), 141.6 (C), 139.1 (C), 131.2 (C), 129.8 (CH), 128.1 (CH), 127.4 (2×CH), 127.2 (2×CH), 125.4 (CH), 109.8 (2×CH, dd, J_{C-F}=18.8, 7.0 Hz), 103.2 (CH, t. J_{C-F}=25.9 Hz), 59.7 (CH), 41.7 (CH₂), 28.8 (CH₂), 28.3 (CH₂); HRMS (ESI/Q-TOF) m/z: [M+H]⁺ calcd for C₂₇H₂₁F₂N₄O₃, 487.1576; found, 487.1576.

### 4-(3-(3′-Fluoro-4′-(trifluoromethoxy)-[1,1′-biphenyl]-4-yl)-5-(quinoxalin-6-yl)-4,5-dihydro-1H-pyrazol-1-yl)-4-oxobutanoic acid (Compound 67)

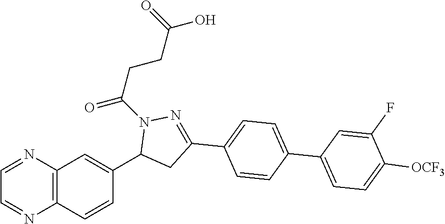 [SMILES: O=C(O)CCC(=O)N1N=C(c2ccc(-c3ccc(OC(F)(F)F)c(F)c3)cc2)CC1c1ccc2nccnc2c1]

Compound 67 was prepared over 2 steps using a method similar to that described for the preparation of Compound 20-step 1: quinoxaline-6-carbaldehyde (316 mg, 2.00 mmol), 1-(3′-fluoro-4′-(trifluoromethoxy)-[1,1′-biphenyl]-4-yl) ethan-1-one (596 mg, 2.00 mmol) and 2 M NaOH (5.0 mL, 10.0 mmol) in EtOH (16 mL) to afford (E)-1-(3′-fluoro-4′-(trifluoromethoxy)-[1,1′-biphenyl]-4-yl)-3-(quinoxalin-6-yl)prop-2-en-1-one as an off-white solid (798 mg); step 2: (E)-1-(3′-fluoro-4′-(trifluoromethoxy)-[1,1′-biphenyl]-4-yl)-3-(quinoxalin-6-yl)prop-2-en-1-one (400 mg) and hydrazine hydrate (0.11 mL, 50% w/w) in THF (7 mL), followed by the addition of succinic anhydride (274 mg, 2.74 mmol). Purification by flash column chromatography (CH₂Cl₂, neat→CH₂Cl₂/MeOH, 95:5) afforded Compound 67 as an off-white solid (171 mg, 31% over 2 steps). ¹H NMR (400 MHz, CDCl₃) δ 8.82 (2H, s), 8.11 (1H, d, J=8.7 Hz), 7.99 (1H, s), 7.85 (2H, d, J=8.2 Hz), 7.68 (1H, dd, J=8.8, 2.0 Hz), 7.61 (2H, d, J=8.1 Hz), 7.46-7.40 (3H, m), 5.86 (1H, dd, J=11.9, 4.9 Hz), 3.92 (1H, dd, J=17.7, 12.0 Hz), 3.33-3.25 (2H, m), 3.19 (1H, dt, J=17.3, 6.8 Hz), 2.76 (2H, t, J=6.8 Hz); ¹³C NMR (101 MHz, CDCl₃) δ 176.8 (C), 170.3 (C), 154.8 (C, d, J_{C-F}=253.0 Hz), 153.8 (C), 145.3 (CH), 145.1 (CH), 143.8 (C), 143.0 (C), 142.6 (C), 140.84 (C, d, J_{C-F}=2.0 Hz), 140.83 (C, d, J_{C-F}=6.7 Hz), 136.2 (C, dq, J_{C-F}=12.7, 2.2 Hz), 131.0 (C), 130.6 (CH), 128.2 (CH), 127.6 (2× CH), 127.5 (2×CH), 125.8 (CH), 124.2 (CH), 123.2 (CH, d, J_{C-F}=3.6 Hz), 120.6 (C, q, J_{C-F}=258.8 Hz), 116.0 (CH, d, J_{C-F}=19.4 Hz), 60.3 (CH), 42.3 (CH₂), 29.2 (CH₂), 28.9 (CH₂); HRMS (ESI/Q-TOF) m/z: [M+H]⁺ calcd for C₂₈H₂₁F₄N₄O₄, 553.1493; found, 553.1494.

### 4-(3-([1,1′-Biphenyl]-3-yl)-5-(quinoxalin-6-yl)-4,5-dihydro-1H-pyrazol-1-yl)-N-(methylsulfonyl)-4-oxobutanamide (Compound 68)

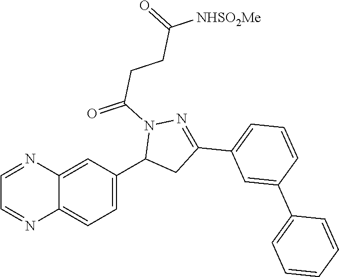

Compound 68 was prepared following a method similar to that described for the preparation of Compound 55 using Compound 13 (45 mg, 0.10 mmol), methanesulfonamide (14 mg, 0.15 mmol), N-(3-dimethylaminopropyl)-N′-ethylcarbodiimide hydrochloride (EDCI) (29 mg, 0.15 mmol), and 4-(dimethylamino)pyridine (DMAP) (15 mg, 0.12 mmol) in CH₂Cl₂ (1.0 mL). Purification by flash column chromatography (petroleum ether/EtOAc, 1:1→EtOAc, neat) afforded Compound 68 as a yellow solid (14 mg, 26%). ¹H NMR (400 MHz, DMSO-d₆) δ 11.71 (1H, s), 8.94-8.92 (2H, m), 8.09 (1H, d, J=8.7 Hz), 8.02 (1H, s), 7.92 (1H, d, J=1.8 Hz), 7.87 (1H, d, J=7.9 Hz), 7.78 (1H, d, J=7.8 Hz), 7.75-7.72 (3H, m), 7.58 (1H, t, J=7.8 Hz), 7.48 (2H, t, J=7.5 Hz), 7.39 (1H, t, J=7.3 Hz), 5.87 (1H, dd, J=12.0, 5.0 Hz), 4.05 (1H, dd, J=18.3, 12.0 Hz), 3.47 (1H, dd, J=18.3, 5.0 Hz), 3.19 (1H, dt, J=17.2, 6.6 Hz), 3.14 (3H, s), 3.05 (1H, dt, J=17.2, 6.8 Hz), 2.60 (2H, t, J=6.6 Hz); ¹³C NMR (101 MHz, DMSO-d₆) δ 172.0 (C), 169.1 (C), 154.7 (C), 146.0 (CH), 145.6 (CH), 144.1 (C), 142.1 (C), 141.6 (C), 140.7 (C), 139.4 (C), 131.6 (C), 129.8 (CH), 129.4 (CH), 129.0 (2×CH), 128.7 (CH), 128.2 (CH), 127.8 (CH), 126.9 (2×CH), 125.6 (CH), 125.3 (CH), 125.2 (CH), 59.6 (CH), 41.8 (CH₂), 40.8 (CH₃), 30.1 (CH₂), 28.0 (CH₂); HRMS (ESI/Q-TOF) m/z: [M+H]⁺ calcd for C₂₈H₂₆N₅O₄S, 528.1700; found, 528.1706.

### 4-(3-([1,1′-Biphenyl]-4-yl)-5-(quinoxalin-6-yl)-4,5-dihydro-1H-pyrazol-1-yl)-N-(isopropylsulfonyl)-4-oxobutanamide (Compound 69)

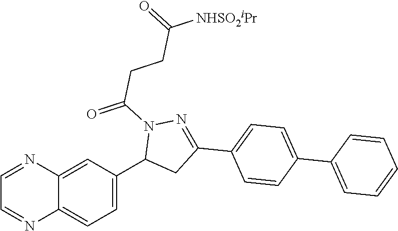

Compound 69 was prepared following a method similar to that described for the preparation of Compound 55 using Compound 15 (40 mg, 0.090 mmol), propane-2-sulfonamide (17 mg, 0.14 mmol), N-(3-dimethylaminopropyl)-N′-ethylcarbodiimide hydrochloride (EDCI) (26 mg, 0.14 mmol), and 4-(dimethylamino)pyridine (DMAP) (13 mg, 0.11 mmol) in CH₂Cl₂ (0.9 mL). Purification by flash column chromatography (petroleum ether/EtOAc, 1:1→EtOAc, neat) afforded Compound 69 as a colourless solid (19 mg, 38%). ¹H NMR (400 MHz, CDCl₃) δ 8.84-8.83 (2H, m), 8.12 (1H, d, J=8.7 Hz), 7.97 (1H, d, J=2.0 Hz), 7.83 (2H, d, J=8.7 Hz), 7.69-7.62 (5H, m), 7.49-7.45 (2H, m), 7.42-7.37 (1H, m), 5.86 (1H, dd, J=11.8, 4.7 Hz), 3.94 (1H, dd, J=17.7, 11.8 Hz), 3.65 (1H, hept, J=6.9 Hz), 3.38 (1H, ddd, J=18.1, 7.8, 4.5 Hz), 3.31 (1H, dd, J=17.8, 4.7 Hz), 3.23 (1H, ddd, J=18.1, 8.2, 4.6 Hz), 2.74-2.61 (2H, m), 1.31 (3H, d, J=6.9 Hz), 1.17 (3H, d, J=6.9 Hz); ¹³C NMR (101 MHz, CDCl₃) δ 171.6 (C), 170.4 (C), 155.2 (C), 145.5 (CH), 145.3 (CH), 143.8 (C), 143.5 (C), 143.1 (C), 142.7 (C), 140.1 (C), 130.8 (CH), 129.6 (C), 129.1 (2×CH), 128.2 (CH), 128.1 (CH), 127.6 (2×CH), 127.5 (2× CH), 127.2 (2×CH), 126.1 (CH), 60.2 (CH), 53.8 (CH), 42.4 (CH₂), 31.6 (CH₂), 29.4 (CH₂), 16.0 (CH₃), 15.7 (CH₃); HRMS (ESI/Q-TOF) m/z: [M+H]⁺ calcd for C₃₀H₃₀N₅O₄S, 556.2013; found, 556.2027.

### 4-(3-(4-Chlorophenyl)-5-(quinoxalin-6-yl)-4,5-dihydro-1H-pyrazol-1-yl)-4-oxo-N-(phenylsulfonyl) butanamide (Compound 70)

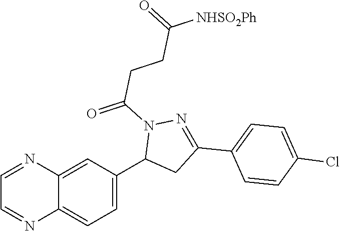

Compound 70 was prepared following a method similar to that described for the preparation of Compound 55 using Compound 1 (0.10 g, 0.24 mmol), benzenesulfonamide (75 mg, 0.48 mmol), N-(3-dimethylaminopropyl)-N′-ethylcarbodiimide hydrochloride (EDCI) (92 mg, 0.48 mmol), and 4-(dimethylamino)pyridine (DMAP) (35 mg, 0.29 mmol) in CH₂Cl₂ (2.4 mL). Purification by flash column chromatography (petroleum ether/EtOAc, 1:1→EtOAc, neat) afforded Compound 70 as a colourless solid (74 mg, 56%). ¹H NMR (400 MHz, DMSO-d₆) δ 12.11 (1H, s), 8.95-8.93 (2H, m), 8.06 (1H, d, J=8.7 Hz), 7.89-7.87 (3H, m), 7.80 (2H, d, J=8.7 Hz), 7.66 (1H, dd, J=8.7, 2.0 Hz), 7.64-7.60 (1H, m), 7.54-7.50 (4H, m) 5.78 (1H, dd, J=11.9, 5.1 Hz), 3.94 (1H, dd, J=18.3, 12.0 Hz), 3.29 (1H, dd, J=18.3, 5.2 Hz), 3.04-3.89 (2H, m), 2.59-2.51 (2H, m); ¹³C NMR (101 MHz, DMSO-d₆) δ 170.8 (C), 168.9 (C), 153.6 (C), 146.0 (CH), 145.6 (CH), 144.0 (C), 142.1 (C), 141.6 (C), 139.5 (C), 135.0 (C), 133.4 (CH), 129.81 (CH), 129.79 (C), 129.0 (2×CH), 128.9 (2×CH), 128.5 (2×CH), 128.1 (CH), 127.3 (2×CH), 125.4 (CH), 59.7 (CH), 41.6 (CH₂), 30.0 (CH₂), 27.8 (CH₂); HRMS (ESI/Q-TOF) m/z: [M+H]⁺ calcd for C₂₇H₂₃ClN₅O₄S, 548.1154; found, 548.1154.

### 4-(3-([1,1′-Biphenyl]-4-yl)-5-(quinoxalin-6-yl)-4,5-dihydro-1H-pyrazol-1-yl)-N—(N,N-dimethylsulfamoyl)-4-oxobutanamide (Compound 71)

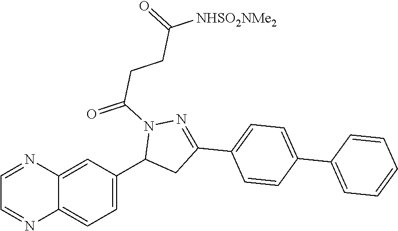

Compound 71 was prepared following a method similar to that described for the preparation of Compound 55 using Compound 15 (40 mg, 0.090 mmol), N,N-dimethylsulfamide (17 mg, 0.14 mmol), N-(3-dimethylaminopropyl)-N′-ethylcarbodiimide hydrochloride (EDCI) (26 mg, 0.14 mmol), and 4-(dimethylamino)pyridine (DMAP) (13 mg, 0.11 mmol) in CH₂Cl₂ (0.9 mL). Purification by flash column chromatography (petroleum ether/EtOAc, 1:1→EtOAc, neat) afforded Compound 71 as a colourless solid (22 mg, 44%). ¹H NMR (300 MHz, DMSO-d₆) δ 11.36 (1H, s), 8.94-8.92 (2H, m), 8.08 (1H, d, J=8.7 Hz), 7.93-7.90 (3H, m), 7.80 (2H, d, J=8.4 Hz), 7.75-7.72 (3H, m), 7.50 (2H, t, J=7.4 Hz), 7.40 (1H, t, J=7.3 Hz), 5.85 (1H, dd, J=11.8, 4.8 Hz), 4.02 (1H, dd, J=18.2, 12.0 Hz), 3.37 (1H, dd, J=18.3, 5.0 Hz), 3.18 (1H, dt, J=17.0, 6.5 Hz), 3.00 (1H, dt, J=17.2, 6.2 Hz), 2.62 (6H, s), 2.59-2.53 (2H, m); ¹³C NMR (75 MHz, DMSO-d₆) δ 171.2 (C), 169.0 (C), 154.2 (C), 145.9 (CH), 145.6 (CH), 144.1 (C), 142.1 (C), 141.9 (C), 141.6 (C), 139.1 (C), 130.0 (C), 129.8 (CH), 129.0 (2×CH), 128.2 (CH), 128.0 (CH), 127.4 (2×CH), 126.9 (2×CH), 126.7 (2×CH), 125.4 (CH), 59.6 (CH), 41.7 (CH₂), 37.7 (2×CH₃), 29.6 (CH₂), 28.3 (CH₂); HRMS (ESI/Q-TOF) m/z: [M+H]⁺ calcd for C₂₉H₂₉N₆O₄S, 557.1966; found, 557.1964.

### 3-(3-(4-Chlorophenyl)-5-(quinoxalin-6-yl)-4,5-dihydro-1H-pyrazol-1-yl)-N-(methylsulfonyl)benzamide (Compound 72)

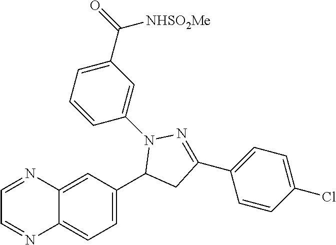

Compound 72 was prepared following a method similar to that described for the preparation of Compound 55 using compound 25312 (21 mg, 0.050 mmol), methanesulfonamide (9.5 mg, 0.10 mmol), N-(3-dimethylaminopropyl)-N′-ethylcarbodiimide hydrochloride (EDCI) (19 mg, 0.10 mmol), and 4-(dimethylamino)pyridine (DMAP) (7.3 mg, 0.060 mmol) in CH₂Cl₂ (0.7 mL). Purification by flash column chromatography (petroleum ether/EtOAc, 1:1→EtOAc, neat) afforded Compound 72 as a yellow solid (9.6 mg, 38%). ¹H NMR (400 MHz, CDCl₃) δ 8.83 (2H, s), 8.10 (1H, d, J=8.7 Hz), 8.00 (1H, d, J=1.7 Hz), 7.69-7.65 (4H, m), 7.37 (2H, d, J=8.6 Hz), 7.24-7.13 (3H, m), 5.59 (1H, dd, J=12.3, 6.4 Hz), 3.97 (1H, dd, J=17.2, 12.4 Hz), 3.39 (3H, s), 3.23 (1H, dd, J=17.3, 6.4 Hz); ¹³C NMR (101 MHz, CDCl₃) δ 165.7 (C), 147.4 (C), 145.7 (CH), 145.4 (CH), 144.8 (C), 143.5 (C), 143.2 (C), 142.8 (C), 135.4 (C), 132.2 (C), 131.4 (CH), 130.5 (C), 129.9 (CH), 129.1 (2×CH), 127.8 (CH), 127.4 (2×CH), 126.5 (CH), 118.3 (CH), 118.0 (CH), 112.8 (CH), 63.9 (CH), 43.5 (CH₂), 41.9 (CH₃); HRMS (ESI/Q-TOF) m/z: [M+H]⁺ calcd for C₂₅H₂₁ClNO₃S, 506.1048; found, 506.1057.

### 4-(7-Chloro-3-(quinoxalin-6-yl)-3,3a,4,5-tetrahydro-2H-benzo[g]indazol-2-yl)-4-oxobutanoic acid (Compound 73)

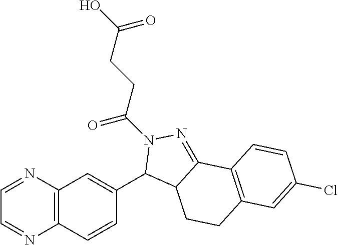 [SMILES: O=C(O)CCC(=O)N1N=C2c3ccc(Cl)cc3CCC2C1c1ccc2nccnc2c1]

Compound 73 was prepared over 3 steps using a method similar to that described for the preparation of Compound 3-step 1: quinoxaline-6-carbaldehyde (200 mg, 1.26 mmol), 6-chloro-1-tetralone (220 mg, 1.22 mmol) and 2 M NaOH (3.2 mL, 6.32 mmol) in EtOH (12 mL) to afford (E)-6-chloro-2-(quinoxalin-6-ylmethylene)-3,4-dihydronaphthalen-1(2H)-one as a beige solid (360 mg); step 2: (E)-6-chloro-2-(quinoxalin-6-ylmethylene)-3,4-dihydronaphthalen-1(2H)-one (360 mg) and hydrazine hydrate (0.14 mL, 50% w/w) in EtOH (8 mL) to afford 7-chloro-3-(quinoxalin-6-yl)-3,3a,4,5-tetrahydro-2H-benzo[g]indazole as a pale brown solid (120 mg); and step 3:7-chloro-3-(quinoxalin-6-yl)-3,3a,4,5-tetrahydro-2H-benzo[g]indazole (120 mg) and succinic anhydride (72 mg, 0.72 mmol) in THF (5 mL). Purification by flash column chromatography (EtOAc, neat→CH₂Cl₂/MeOH, 9:1) afforded Compound 73 as a colourless solid (22 mg, 4% over 3 steps). ¹H NMR (400 MHz, CDCl₃) δ 8.84-8.83 (2H, m), 8.14 (1H, d, J=8.7 Hz), 8.04 (1H, d, J=2.0 Hz), 7.94 (1H, d, J=8.4 Hz), 7.73 (1H, dd, J=8.7, 2.1 Hz), 7.30-7.21 (2H, m), 5.22 (1H, d, J=9.6 Hz), 3.35-3.15 (3H, m), 2.96-2.92 (2H, m), 2.72 (2H, t, J=6.6 Hz), 2.41-2.36 (1H, m), 2.15-2.04 (1H, m).

### 4-Oxo-4-(8-phenyl-3-(quinoxalin-6-yl)-3,3a,4,5-tetrahydro-2H-benzo[g]indazol-2-yl)butanoic acid (Compound 74)

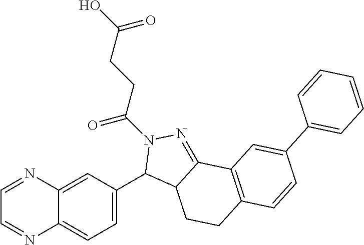 [SMILES: O=C(O)CCC(=O)N1N=C2c3cc(-c4ccccc4)ccc3CCC2C1c1ccc2nccnc2c1]

To a solution of quinoxaline-6-carbaldehyde (200 mg, 1.26 mmol) and 7-phenyl-1-tetralone (280 mg, 1.26 mmol) in EtOH (12 mL) at room temperature was added dropwise 2 M NaOH (3.2 mL, 6.32 mmol), and the reaction mixture stirred for 2 h. The resulting precipitated solids were then collected by filtration, washed with H₂O and dried in vacuo to afford (E)-7-phenyl-2-(quinoxalin-6-ylmethylene)-3,4-dihydronaphthalen-1(2H)-one as a pale-brown solid (420 mg), which was used directly in the next step without further purification.

To a stirred solution of (E)-7-phenyl-2-(quinoxalin-6-ylmethylene)-3,4-dihydronaphthalen-1(2H)-one (100 mg) in EtOH (5 mL) was added hydrazine hydrate (0.10 mL, 50% w/w), and the reaction mixture heated under reflux for 3 h. The solvent was then removed in vacuo, the residue poured onto ice water and the resulting precipitated solids were collected by filtration and dried in vacuo to afford 8-phenyl-3-(quinoxalin-6-yl)-3,3a,4,5-tetrahydro-2H-benzo[g]indazole as a yellow solid (100 mg), which was used in the next step without further purification.

In a sealed vial, a mixture of 8-phenyl-3-(quinoxalin-6-yl)-3,3a,4,5-tetrahydro-2H-benzo[g]indazole (100 mg) and succinic anhydride (200 mg, 2.00 mmol) in THF (4 mL) was subjected to microwave irradiation at 120° C. for 45 min. The resulting reaction mixture was diluted with CH-Ch (20 mL), washed with water (20 mL) and the separated aqueous layer further extracted with CH₂Cl (3×30 mL). The combined organic layers were washed with water (3×30 mL), then brine (30 mL), dried over anhydrous Na₂SO₄, and concentrated in vacuo. Purification by flash column chromatography (CH₂Ch/MeOH, 25:1) afforded Compound 74 as a pale-brown solid (30 mg, 21% over 3 steps). ¹H NMR (400 MHz, CDCl₃) δ 8.83-8.81 (2H, m), 8.22 (1H, d, J=1.9 Hz), 8.14 (1H, d, J=8.7 Hz), 8.06 (1H, d, J=1.8 Hz), 7.75 (1H, dd, J=8.7, 1.9 Hz), 7.66-7.63 (2H, m), 7.59 (1H, dd, J=8.0, 2.0 Hz), 7.47 (2H, t, J=7.6 Hz), 7.40-7.35 (1H, m), 7.26 (1H, d, J=8.0 Hz), 5.24 (1H, d, J=9.6 Hz), 3.36-3.29 (1H, m), 3.27-3.14 (2H, m), 3.05-2.91 (2H, m), 2.78-2.70 (2H, m), 2.42-2.37 (1H, m), 2.14-2.03 (1H, m); ¹³C NMR (101 MHz, CDCl₃) δ 176.7 (C), 171.6 (C), 155.8 (C), 145.2 (CH), 144.8 (CH), 143.6 (C), 143.0 (C), 142.4 (C), 140.1 (C), 140.0 (C), 138.3 (C), 130.4 (CH), 129.6 (CH), 129.5 (CH), 128.9 (CH), 127.9 (2×CH), 127.7 (CH), 127.4 (C), 127.1 (2×CH), 126.0 (CH), 123.4 (CH), 67.6 (CH), 55.6 (CH), 29.3 (CH₂), 29.0 (CH₂), 28.8 (CH₂), 27.9 (CH₂); HRMS (ESI/Q-TOF) m/z: [M+Na]⁺ calcd for C₂₉H₂₄N₄NaO₃, 499.1741; found, 499.1740.

### 4-Oxo-4-(7-phenyl-3-(quinoxalin-6-yl)-3,3a,4,5-tetrahydro-2H-benzo[g]indazol-2-yl)butanoic acid (Compound 75)

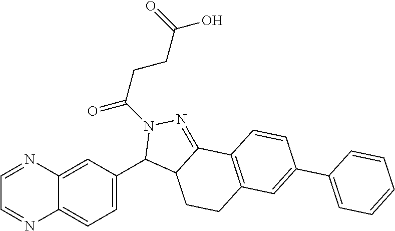 [SMILES: O=C(O)CCC(=O)N1N=C2c3ccc(-c4ccccc4)cc3CCC2C1c1ccc2nccnc2c1]

To a solution of quinoxaline-6-carbaldehyde (200 mg, 1.26 mmol) and 6-phenyl-1-tetralone (280 mg, 1.26 mmol) in EtOH (10 mL) at room temperature was added dropwise 2 M NaOH (3.2 mL, 6.32 mmol), and the reaction mixture stirred at room temperature for 3 h. After, the solution was cooled down to 0° C., and the resulting precipitated solids were then collected by filtration, washed with water and dried in vacuo to afford (E)-6-phenyl-2-(quinoxalin-6-ylmethylene)-3,4-dihydronaphthalen-1(2H)-one as an off-white solid (315 mg), which was used directly in the next step without further purification.

To a stirred solution of (E)-6-phenyl-2-(quinoxalin-6-ylmethylene)-3,4-dihydronaphthalen-1(2H)-one (315 mg) in EtOH (7 mL) was added hydrazine hydrate (0.16 mL, 50% w/w), and the reaction mixture heated under reflux for 2 h. The solvent was then removed in vacuo and the resulting residue was poured onto ice water, collected by filtration and dried in vacuo to afford 7-phenyl-3-(quinoxalin-6-yl)-3,3a,4,5-tetrahydro-2H-benzo[g]indazole as an off-white solid (262 mg), which was used in the next step without further purification.

In a sealed vial, a mixture of 7-phenyl-3-(quinoxalin-6-yl)-3,3a,4,5-tetrahydro-2H-benzo[g]indazole (262 mg) and succinic anhydride (139 mg, 1.39 mmol) in THF (5 mL) was subjected to microwave irradiation at 120° C. for 45 min. The resulting reaction mixture was diluted with CH₂Cl (20 mL), washed with H₂O (20 mL) and the separated aqueous layer further extracted with CH-Ch (3×30 mL). The combined organic layers were washed with H₂O (3×30 mL), then brine (30 mL), dried over anhydrous Na₂SO₄, and concentrated in vacuo. Purification by flash column chromatography (EtOAc, neat→CH₂Cl₂/MeOH, 9:1) afforded Compound 75 as an off-white solid (192 mg, 32% over 3 steps). ¹H NMR (400 MHz, DMSO-d₆) δ 12.09 (1H, br s), 8.98-8.95 (2H, m), 8.11 (1H, d, J=8.7 Hz), 8.05 (1H, d, J=2.0 Hz), 8.01 (1H, d, J=8.1 Hz), 7.85 (1H, dd, J=8.7, 2.0 Hz), 7.74-7.71 (2H, m), 7.66 (1H, dd, J=8.2, 1.9 Hz), 7.61 (1H, d, J=1.8 Hz), 7.50 (2H, t, J=7.5 Hz), 7.43-7.39 (1H, m), 5.31 (1H, d, J=10.1 Hz), 3.45 (1H, ddd, J=13.1, 10.1, 4.9 Hz), 3.12-2.95 (4H, m), 2.52-2.48 (2H, m), 2.37-2.29 (1H, m), 2.13-2.02 (1H, m); ¹³C NMR (101 MHz, DMSO-d₆) δ 173.8 (C), 170.7 (C), 155.0 (C), 145.9 (CH), 145.4 (CH), 143.9 (C), 142.2 (C), 141.9 (C), 141.6 (C), 140.5 (C), 139.3 (C), 129.5 (CH), 129.0 (2×CH), 128.3 (CH), 128.0 (CH), 127.3 (CH), 126.7 (2×CH), 126.0 (C), 125.7 (CH), 125.03 (CH), 124.98 (CH), 66.7 (CH), 54.5 (CH), 29.0 (CH₂), 28.7 (CH₂), 28.4 (CH₂), 27.0 (CH₂); HRMS (ESI/Q-TOF) m/z: [M+Na]⁺ calcd for C₂₉H₂₄N₄NaO₃, 499.1741; found, 499.1728.

### 4-Oxo-4-(3-(quinoxalin-6-yl)-7-(4-(trifluoromethoxy)phenyl)-3,3a,4,5-tetrahydro-2H-benzo[g]indazol-2-yl)butanoic acid (Compound 76)

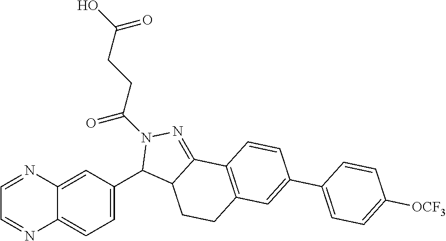 [SMILES: O=C(O)CCC(=O)N1N=C2c3ccc(-c4ccc(OC(F)(F)F)cc4)cc3CCC2C1c1ccc2nccnc2c1]

Compound 76 was prepared over 3 steps using a method similar to that described for the preparation of Compound 75-step 1: quinoxaline-6-carbaldehyde (237 mg, 1.50 mmol), 6-(4-(trifluoromethoxy)phenyl)-1-tetralone (459 mg, 1.50 mmol) and 2 M NaOH (3.8 mL, 7.50 mmol) in EtOH (12 mL) to afford (E)-2-(quinoxalin-6-ylmethylene)-6-(4-(trifluoromethoxy)phenyl)-3,4-dihydronaphthalen-1(2H)-one as a beige solid (462 mg); step 2: (E)-2-(quinoxalin-6-ylmethylene)-6-(4-(trifluoromethoxy)phenyl)-3,4-dihydronaphthalen-1(2H)-one (300 mg) and hydrazine hydrate (0.12 mL, 50% w/w) in EtOH (6 mL) to afford 3-(quinoxalin-6-yl)-7-(4-(trifluoromethoxy)phenyl)-3,3a,4,5-tetrahydro-2H-benzo[g]indazole as an yellow solid (310 mg); and step 3:3-(quinoxalin-6-yl)-7-(4-(trifluoromethoxy)phenyl)-3,3a,4,5-tetrahydro-2H-benzo[g]indazole (310 mg) and succinic anhydride (100 mg, 1.00 mmol) in THF (4 mL). Purification by flash column chromatography (CH₂Cl₂/MeOH, 20:1) afforded Compound 76 as a beige solid (150 mg, 28% over 3 steps). ¹H NMR (400 MHz, CDCl₃) δ 8.81 (2H, s), 8.15-8.07 (3H, m), 7.75 (1H, d, J=8.7 Hz), 7.62-7.59 (2H, m), 7.50 (1H, dd, J=8.1, 1.4 Hz), 7.38 (1H, s), 7.29 (2H, d, J=8.1 Hz), 5.24 (1H, d, J=9.7 Hz), 3.36-3.12 (3H, m), 3.05-2.94 (2H, m), 2.73 (2H, s), 2.43-2.39 (1H, m), 2.15-2.04 (1H, m); HRMS (ESI/Q-TOF) m/z: [M+Na]⁺ calcd for C₃₀H₂₃F₃N₄NaO₄, 583.1564; found, 583.1564.

### 4-(5-(4-Chlorophenyl)-3-(quinoxalin-6-yl)-4,5-dihydro-1H-pyrazol-1-yl)-4-oxobutanoic acid (Compound 77)

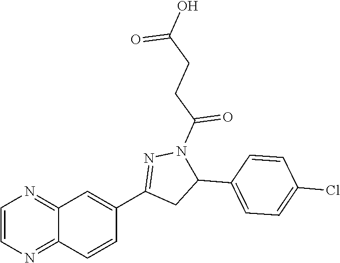 [SMILES: O=C(O)CCC(=O)N1N=C(c2ccc3nccnc3c2)CC1c1ccc(Cl)cc1]

Compound 77 was prepared over 3 steps using a method similar to that described for the preparation of Compound 3-step 1:4-chlorobenzaldehyde (163 mg, 1.16 mmol), 1-(quinoxalin-6-yl) ethanone (200 mg, 1.16 mmol) and 2 M NaOH (3.2 mL, 6.32 mmol) in EtOH (12 mL) to afford (E)-3-(4-chlorophenyl)-1-(quinoxalin-6-yl)prop-2-en-1-one as a colourless solid (200 mg); step 2: (E)-3-(4-chlorophenyl)-1-(quinoxalin-6-yl)prop-2-en-1-one as a colourless solid (200 mg) and hydrazine hydrate (85 μL, 50% w/w) in EtOH (8 mL) to afford 6-(5-(4-chlorophenyl)-4,5-dihydro-1H-pyrazol-3-yl)quinoxaline as a yellow solid (150 mg); and step 3:6-(5-(4-chlorophenyl)-4,5-dihydro-1H-pyrazol-3-yl)quinoxaline (150 mg) and succinic anhydride (97 mg, 0.97 mmol) in THF (5 mL). Purification by flash column chromatography (EtOAc, neat→CH₂Cl₂/MeOH, 9:1) afforded Compound 77 as a colourless solid (22 mg, 5% over 3 steps). ¹H NMR (400 MHz, DMSO-d₆) δ 12.15 (1H, br s), 9.00-8.98 (2H, m), 8.41 (1H, dd, J=8.8, 1.9 Hz), 8.30 (1H, d, J=1.8 Hz), 8.17 (1H, d, J=8.8 Hz), 7.38 (2H, d, J=8.5 Hz), 7.28 (2H, d, J=8.5 Hz), 5.63 (1H, dd, J=12.0, 5.0 Hz), 4.07-3.99 (1H, m), 3.37 (1H, dd, J=18.2, 5.0 Hz), 3.12-2.95 (2H, m), 2.54-2.50 (2H, m).

### 4-Oxo-4-(3-(4′-propoxy-[1,1′-biphenyl]-4-yl)-5-(quinoxalin-6-yl)-4,5-dihydro-1H-pyrazol-1-yl)butanoic acid (Compound 78)

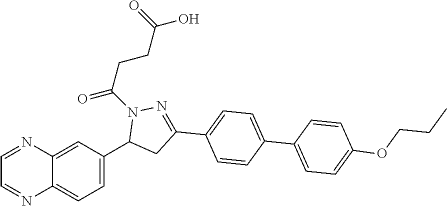 [SMILES: CCCOc1ccc(-c2ccc(C3=NN(C(=O)CCC(=O)O)C(c4ccc5nccnc5c4)C3)cc2)cc1]

Compound 78 was prepared over 2 steps using a method similar to that described for the preparation of Compound 1A-step 1: quinoxaline-6-carbaldehyde (100 mg, 0.632 mmol), 1-(4′-propoxy-[1,1′-biphenyl]-4-yl) ethan-1-one (161 mg, 0.632 mmol) and 2 M NaOH (1.6 mL, 3.16 mmol) in EtOH (5 mL) to afford (E)-1-(4′-propoxy-[1,1′-biphenyl]-4-yl)-3-(quinoxalin-6-yl)prop-2-en-1-one as a yellow solid (249 mg); step 2: (E)-1-(4′-propoxy-[1,1′-biphenyl]-4-yl)-3-(quinoxalin-6-yl)prop-2-en-1-one (249 mg) and hydrazine hydrate (79 μL, 50% w/w) in THF (5 mL), followed by the addition of succinic anhydride (190 mg, 1.90 mmol). Purification by flash column chromatography (petroleum ether/EtOAc, 1:1→EtOAc, neat→CH₂Cl/MeOH 9:1) afforded Compound 78 as a yellow solid (83.6 mg, 26% over 2 steps). ¹H NMR (400 MHz, CDCl₃) δ 8.81-8.80 (2H, m), 8.10 (1H, d, J=8.7 Hz), 7.99 (1H, d, J=2.0 Hz), 7.79 (2H, d, J=8.5 Hz), 7.67 (1H, dd, J=8.8, 2.0 Hz), 7.61 (2H, d, J=8.5 Hz), 7.55 (2H, d, J=8.8 Hz), 6.98 (2H, d, J=8.8 Hz), 5.83 (1H, dd, J=11.9, 4.9 Hz), 3.97 (2H, t, J=6.6 Hz), 3.90 (1H, dd, J=17.7, 11.9 Hz), 3.35-3.23 (2H, m), 3.18 (1H, dt, J=17.3, 6.8 Hz), 2.75 (2H, t, J=6.7 Hz), 1.84 (2H, sext, J=7.1 Hz), 1.06 (3H, t, J=7.4 Hz); ¹³C NMR (101 MHz, CDCl₃) § 176.4 (C), 170.4 (C), 159.5 (C), 154.5 (C), 145.4 (CH), 145.1 (CH), 143.9 (C), 143.3 (C), 143.0 (C), 142.6 (C), 132.4 (C), 130.6 (CH), 129.2 (C), 128.24 (2×CH), 128.19 (CH), 127.4 (2×CH), 127.0 (2× CH), 125.9 (CH), 115.1 (2×CH), 69.8 (CH₂), 60.2 (CH), 42.4 (CH₂), 29.2 (CH₂), 29.0 (CH₂), 22.7 (CH₂), 10.7 (CH₃); HRMS (ESI/Q-TOF) m/z: [M+Na]⁺ calcd for C₃₀H₂₈N₄NaO₄, 531.2003; found, 531.2003.

### 4-(3-(4′-Ethoxy-[1,1′-biphenyl]-4-yl)-5-(quinoxalin-6-yl)-4,5-dihydro-1H-pyrazol-1-yl)-4-oxobutanoic acid (Compound 79)

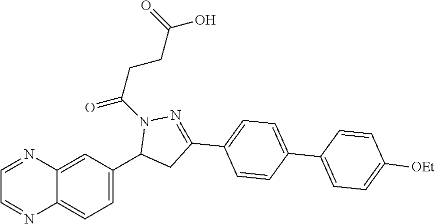 [SMILES: CCOc1ccc(-c2ccc(C3=NN(C(=O)CCC(=O)O)C(c4ccc5nccnc5c4)C3)cc2)cc1]

Compound 79 was prepared over 2 steps using a method similar to that described for the preparation of Compound 20-step 1: quinoxaline-6-carbaldehyde (79 mg, 0.5 mmol), 1-(4′-ethoxy-[1,1′-biphenyl]-4-yl)ethan-1-one (113 mg, 0.5 mmol) and 2 M NaOH (1.0 mL, 2.0 mmol) in EtOH (4 mL) to afford (E)-1-(4′-ethoxy-[1,1′-biphenyl]-4-yl)-3-(quinoxalin-6-yl)prop-2-en-1-one as a colourless solid; step 2: The crude (E)-1-(4′-ethoxy-[1,1′-biphenyl]-4-yl)-3-(quinoxalin-6-yl)prop-2-en-1-one and hydrazine hydrate (25 μL, 50% w/w) in THF (2 mL), followed by the addition of succinic anhydride (50 mg, 0.5 mmol). Purification by flash column chromatography (EtOAc→DCM/MeOH 10:1) afforded Compound 79 as a colourless solid (6 mg, 3% over 2 steps); ¹H NMR (400 MHz, CDCl₃) δ 8.83-8.80 (2H, m), 8.11 (1H, d, J=8.7 Hz), 7.99 (1H, d, J=1.7 Hz), 7.84-7.79 (2H, m), 7.70-7.66 (1H, m), 7.64-7.60 (2H, m), 7.58-7.53 (2H, m), 7.05-6.96 (2H, m), 5.84 (1H, dd, J=11.8, 4.1 Hz), 4.12-4.04 (2H, m), 3.91 (1H, dd, J=17.5, 11.8 Hz), 3.35-3.25 (2H, m), 3.22-3.14 (1H, m), 2.75 (2H, t, J=6.8 Hz), 1.45 (3H, t, J=7.1 Hz); ¹³C NMR (101 MHz, CDCl₃) δ 175.6 (C), 170.4 (C), 159.2 (C), 154.6 (C), 145.3 (CH), 145.1 (CH), 143.7 (C), 143.2 (C), 143.0 (C), 142.6 (C), 132.3 (C), 130.6 (CH), 129.1 (C), 128.2 (2×CH), 128.1 (CH), 127.3 (2×CH), 126.9 (2×CH), 125.5 (CH), 115.0 (2×CH), 63.7 (CH₂), 60.1 (CH), 42.3 (CH₂), 29.8 (CH₂), 29.1 (CH₂), 14.9 (CH₃).

### 4-(3-(4′-Butoxy-[1,1′-biphenyl]-4-yl)-5-(quinoxalin-6-yl)-4,5-dihydro-1H-pyrazol-1-yl)-4-oxobutanoic acid (Compound 80)

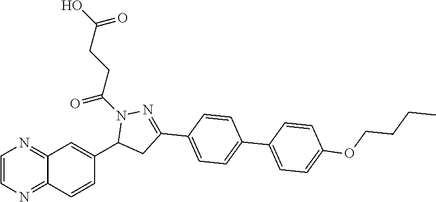 [SMILES: CCCCOc1ccc(-c2ccc(C3=NN(C(=O)CCC(=O)O)C(c4ccc5nccnc5c4)C3)cc2)cc1]

Compound 80 was prepared over 2 steps using a method similar to that described for the preparation of Compound 20-step 1: quinoxaline-6-carbaldehyde (210 mg, 1.33 mmol), 1-(4′-butoxy-[1,1′-biphenyl]-4-yl)ethan-1-one (357 mg, 1.33 mmol) and 2 M NaOH (3.3 mL, 6.65 mmol) in EtOH (10.5 mL) to afford (E)-1-(4′-butoxy-[1,1′-biphenyl]-4-yl)-3-(quinoxalin-6-yl)prop-2-en-1-one as a pale yellow solid without further purification (464 mg); step 2: (E)-1-(4′-butoxy-[1,1′-biphenyl]-4-yl)-3-(quinoxalin-6-yl)prop-2-en-1-one (464 mg) and hydrazine hydrate (150 μL, 50% w/w, 2.28 mmol) in THF (9 mL), followed by the addition of succinic anhydride (342 mg, 3.42 mmol). Purification by flash column chromatography (petroleum ether/EtOAc, 1:1→EtOAc, neat→EtOAc/MeOH, 19:1) afforded Compound 80 as a yellow solid (179 mg, 26% over 2 steps). ¹H NMR (400 MHz, CDCl₃) δ 8.82-8.81 (2H, m), 8.11 (1H, d, J=8.7 Hz), 7.99 (1H, d, J=1.9 Hz), 7.80 (2H, dt, J=8.8, 1.9 Hz), 7.68 (1H, dt, J=8.7, 2.0 Hz), 7.62 (2H, dt, J=8.6, 1.9 Hz), 7.55 (2H, dt, J=9.4, 2.5 Hz), 6.98 (2H, dt, J=9.4, 2.5 Hz), 5.84 (1H, dd, J=11.9, 4.8 Hz), 4.01 (2H, t, J=6.5 Hz), 3.92 (1H, dd, J=17.7, 11.8 Hz), 3.35-3.15 (3H, m), 2.76 (2H, t, J=6.7 Hz), 1.83-1.76 (2H, m), 1.56-1.47 (2H, m), 0.99 (3H, t, J=7.4 Hz); ¹³C NMR (101 MHz, CDCl₃) δ 175.8 (C), 170.5 (C), 159.5 (C), 154.6 (C), 145.4 (CH), 145.1 (CH), 143.8 (C), 143.3 (C), 143.1 (C), 142.7 (C), 132.3 (C), 130.7 (CH), 129.1 (C), 128.25 (2×CH), 128.17 (CH), 127.4 (2×CH), 127.0 (2×CH), 125.9 (CH), 115.1 (2×CH), 68.2 (CH₂), 60.2 (CH), 42.4 (CH₂), 31.5 (CH₂), 29.2 (CH₂), 29.0 (CH₂), 19.4 (CH₂), 14.0 (CH₃); HRMS (ESI/Q-TOF) m/z: [M+Na]⁺ calcd for C₃₁H₃₀N₄NaO₄, 545.2159; found, 545.2160.

### 4-(3-(4′-(tert-Butoxy)-[1,1′-biphenyl]-4-yl)-5-(quinoxalin-6-yl)-4,5-dihydro-1H-pyrazol-1-yl)-4-oxobutanoic acid (Compound 81)

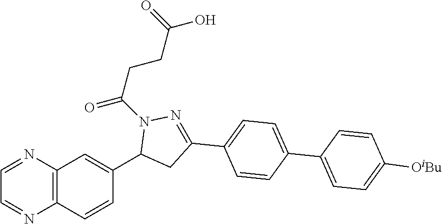

Compound 81 was prepared over 2 steps using a method similar to that described for the preparation of Compound 20-step 1: quinoxaline-6-carbaldehyde (200 mg, 1.26 mmol), 1-(4′-(tert-butoxy)-[1,1′-biphenyl]-4-yl) ethan-1-one (339 mg, 1.26 mmol) and 2 M NaOH (3.2 mL, 6.32 mmol) in EtOH (10 mL) to afford (E)-1-(4′-(tert-butoxy)-[1,1′-biphenyl]-4-yl)-3-(quinoxalin-6-yl)prop-2-en-1-one as an off-white solid (422 mg); step 2: (E)-1-(4′-(tert-butoxy)-[1,1′-biphenyl]-4-yl)-3-(quinoxalin-6-yl)prop-2-en-1-one (422 mg) and hydrazine hydrate (0.13 mL, 50% w/w) in THF (8 mL), followed by the addition of succinic anhydride (310 mg, 3.10 mmol). Purification by flash column chromatography (petroleum ether/EtOAc, 1:1→EtOAc, neat) afforded Compound 81 as a yellow solid (151 mg, 23% over 2 steps). ¹H NMR (400 MHz, CDCl₃) δ 8.81-8.79 (2H, m), 8.10 (1H, d, J=8.7 Hz), 7.99 (1H, d, J=2.1 Hz), 7.80 (2H, d, J=8.5 Hz), 7.67 (1H, dd, J=8.7, 2.0 Hz), 7.63 (2H, d, J=8.5 Hz), 7.52 (2H, d. J=8.6 Hz), 7.07 (2H, d, J=8.6 Hz), 5.83 (1H, dd, J=11.9, 4.9 Hz), 3.90 (1H, dd, J=17.7, 11.9 Hz), 3.32-3.23 (2H, m), 3.18 (1H, dt, J=17.2, 6.8 Hz), 2.75 (2H, t, J=6.8 Hz), 1.39 (9H, s); ¹³C NMR (101 MHz, CDCl₃) δ 176.8 (C), 170.3 (C), 155.8 (C), 154.3 (C), 145.3 (CH), 145.1 (CH), 143.9 (C), 143.1 (C), 143.0 (C), 142.5 (C), 135.0 (C), 130.6 (CH), 129.5 (C), 128.2 (CH), 127.7 (2×CH), 127.4 (2×CH), 127.2 (2×CH), 125.9 (CH), 124.5 (2×CH), 79.0 (C), 60.2 (CH), 42.3 (CH₂), 29.2 (CH₂), 29.03 (3×CH₃), 28.96 (CH₂); HRMS (ESI/Q-TOF) m/z: [M+Na]⁺ calcd for C₃₁H₃₀N₄NaO₄, 545.2159; found, 545.2154.

### 4-(3-(4′-Isobutoxy-[1,1′-biphenyl]-4-yl)-5-(quinoxalin-6-yl)-4,5-dihydro-1H-pyrazol-1-yl)-4-oxobutanoic acid (Compound 82)

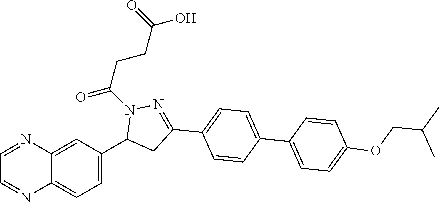 [SMILES: CC(C)COc1ccc(-c2ccc(C3=NN(C(=O)CCC(=O)O)C(c4ccc5nccnc5c4)C3)cc2)cc1]

Compound 82 was prepared over 2 steps using a method similar to that described for the preparation of Compound 20-step 1: quinoxaline-6-carbaldehyde (79 mg, 0.5 mmol), 1-(4′-cyclopropoxy-[1,1′-biphenyl]-4-yl) ethan-1-one (134 mg, 0.5 mmol) and 2 M NaOH (1.0 mL, 2.0 mmol) in EtOH (4 mL) to afford (E)-1-(4′-isobutoxy-[1,1′-biphenyl]-4-yl)-3-(quinoxalin-6-yl)prop-2-en-1-one as a colourless solid; step 2: The crude (E)-1-(4′-isobutoxy-[1,1′-biphenyl]-4-yl)-3-(quinoxalin-6-yl)prop-2-en-1-one and hydrazine hydrate (25 μL, 50% w/w) in THF (2 mL), followed by the addition of succinic anhydride (50 mg, 0.5 mmol). Purification by flash column chromatography (EtOAc→DCM/MeOH 10:1) afforded Compound 82 as a colourless solid (60 mg, 23% over 2 steps); ¹H NMR (400 MHz, CDCl₃) δ 8.83-8.79 (2H, m), 8.12 (1H, d, J=8.7 Hz), 7.99 (1H, d, J=1.7 Hz), 7.81-7.78 (2H, m), 7.67 (1H, dd, J=8.8, 1.9 Hz), 7.63-7.59 (2H, m), 7.56-7.52 (2H, m), 7.04-6.92 (2H, m), 5.84 (1H, dd, J=11.5, 4.8 Hz), 3.90 (1H, dd, J=17.5, 12.0 Hz), 3.77 (2H, d, J=6.4 Hz), 3.36-3.12 (3H, m), 2.75 (2H, t, J=6.7 Hz), 2.17-2.06 (1H, m), 1.04 (6H, d, J=6.5 Hz); ¹³C NMR (101 MHz, CDCl₃) δ 176.4 (C), 170.3 (C), 159.5 (C), 154.3 (C), 145.3 (CH), 145.0 (CH), 143.8 (C), 143.2 (C), 142.9 (C), 142.5 (C), 132.2 (C), 130.5 (CH), 129.1 (C), 128.09 (2×CH), 128.05 (CH), 127.3 (2×CH), 126.9 (2×CH), 125.8 (CH), 115.1 (2×CH), 74.6 (CH₂), 60.1 (CH), 42.3 (CH₂), 29.1 (CH₂), 28.9 (CH₂), 28.3 (CH), 19.3 (2×CH₃); HRMS (ESI/Q-TOF) m/z: [M+Na]⁺ calcd for C₃₁H₃₀N₄NaO₄, 545.2159; found, 545.2160.

### 4-(3-(4′-(sec-Butoxy)-[1,1′-biphenyl]-4-yl)-5-(quinoxalin-6-yl)-4,5-dihydro-1H-pyrazol-1-yl)-4-oxobutanoic acid (Compound 83)

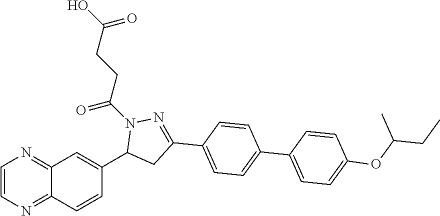 [SMILES: CCC(C)Oc1ccc(-c2ccc(C3=NN(C(=O)CCC(=O)O)C(c4ccc5nccnc5c4)C3)cc2)cc1]

Compound 83 was prepared over 2 steps using a method similar to that described for the preparation of Compound 20-step 1: quinoxaline-6-carbaldehyde (184 mg, 1.16 mmol), 1-(4′-(sec-butoxy)-[1,1′-biphenyl]-4-yl) ethan-1-one (312 mg, 1.16 mmol) and 2 M NaOH (2.9 mL, 5.80 mmol) in EtOH (9 mL) to afford (E)-1-(4′-(sec-butoxy)-[1,1′-biphenyl]-4-yl)-3-(quinoxalin-6-yl)prop-2-en-1-one as a colourless solid without further purification (400 mg); step 2: (E)-1-(4′-(sec-butoxy)-[1,1′-biphenyl]-4-yl)-3-(quinoxalin-6-yl)prop-2-en-1-one (400 mg) and hydrazine hydrate (122 μL, 50% w/w, 1.96 mmol) in THF (8 mL), followed by the addition of succinic anhydride (294 mg, 2.94 mmol). Purification by flash column chromatography (petroleum ether/EtOAc, 1:1→EtOAc, neat→EtOAc/MeOH, 9:1) afforded Compound 83 as a beige solid (147 mg, 24% over 2 steps). ¹H NMR (400 MHz, CDCl₃) δ 8.82-8.81 (2H, m), 8.11 (1H, d, J=8.7 Hz), 7.99 (1H, d, J=1.9 Hz), 7.80-7.78 (2H, m), 7.68 (1H, dd, J=8.7, 2.1 Hz), 7.63-7.60 (2H, m), 7.54 (2H, dt, J=9.4, 2.5 Hz), 6.97 (2H, dt, J=9.4, 2.5 Hz), 5.84 (1H, dd, J=11.9, 4.8 Hz), 4.35 (1H, sext, J=6.0 Hz), 3.91 (1H, dd, J=17.7, 11.9 Hz), 3.34-3.14 (3H, m), 2.75 (2H, t, J=6.7 Hz), 1.81-1.73 (1H, m), 1.70-1.61 (1H, m), 1.33 (3H, d, J=6.1 Hz), 1.00 (3H, t, J=7.4 Hz); ¹³C NMR (101 MHz, CDCl₃) δ 176.2 (C), 170.4 (C), 158.6 (C), 154.5 (C), 145.3 (CH), 145.1 (CH), 143.9 (C), 143.3 (C), 143.0 (C), 142.6 (C), 132.2 (C), 130.6 (CH), 129.1 (C), 128.3 (2×CH), 128.2 (CH), 127.4 (2×CH), 127.0 (2× CH), 125.9 (CH), 116.4 (2×CH), 75.3 (CH), 60.2 (CH), 42.4 (CH₂), 29.3 (CH₂), 29.2 (CH₂), 29.0 (CH₂), 19.4 (CH₃), 9.9 (CH₃); HRMS (ESI/Q-TOF) m/z: [M+Na]⁺ calcd for C₃₁H₃₀N₄NaO₄, 545.2159; found, 545.2160.

### 4-(3-(4′-Cyclopropoxy-[1,1′-biphenyl]-4-yl)-5-(quinoxalin-6-yl)-4,5-dihydro-1H-pyrazol-1-yl)-4-oxobutanoic acid (Compound 84)

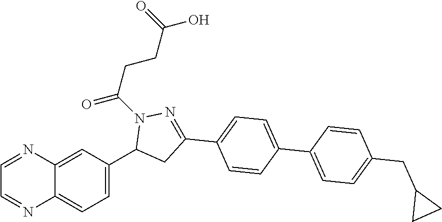 [SMILES: O=C(O)CCC(=O)N1N=C(c2ccc(-c3ccc(CC4CC4)cc3)cc2)CC1c1ccc2nccnc2c1]

Compound 84 was prepared over 2 steps using a method similar to that described for the preparation of Compound 20-step 1: quinoxaline-6-carbaldehyde (79 mg, 0.5 mmol), 1-(4′-cyclopropoxy-[1,1′-biphenyl]-4-yl) ethan-1-one (102 mg, 0.4 mmol) and 2 M NaOH (1.0 mL, 2.0 mmol) in EtOH (4 mL) to afford (E)-1-(4′-cyclopropoxy-[1,1′-biphenyl]-4-yl)-3-(quinoxalin-6-yl)prop-2-en-1-one as a colourless solid; step 2: The crude (E)-1-(4′-cyclopropoxy-[1,1′-biphenyl]-4-yl)-3-(quinoxalin-6-yl)prop-2-en-1-one and hydrazine hydrate (25 μL, 50% w/w) in THF (2 mL), followed by the addition of succinic anhydride (50 mg, 0.5 mmol). Purification by flash column chromatography (EtOAc→DCM/MeOH 10:1) afforded Compound 84 as a colourless solid (42 mg, 21% over 2 steps); ¹H NMR (400 MHz, CDCl₃) ¿ 8.86-8.78 (2H, m), 8.11 (1H, d, J=8.7 Hz), 7.98 (1H, d, J=1.7 Hz), 7.82-7.72 (2H, m), 7.68 (1H, dd, J=8.8, 2.1 Hz), 7.64-7.60 (2H, m), 7.58-7.54 (2H, m), 7.17-7.11 (2H, m), 5.84 (1H, dd, J=11.5, 4.8 Hz), 3.92 (1H, dd, J=17.5, 12.0 Hz), 3.34-3.12 (1H, m), 3.36-3.11 (3H, m), 2.76 (2H, t, J=6.5 Hz), 0.83-0.78 (4H, m); ¹³C NMR (101 MHz, CDCl₃) δ 176.1 (C), 170.3 (C), 159.2 (C), 154.4 (C), 145.3 (CH), 145.1 (CH), 143.7 (C), 143.2 (C), 143.0 (C), 142.6 (C), 132.8 (C), 130.6 (CH), 129.2 (C), 128.1 (3×CH), 127.3 (2×CH), 127.0 (2×CH), 125.9 (CH), 115.6 (2×CH), 60.1 (CH), 51.1 (CH), 42.3 (CH₂), 29.2 (CH₂), 28.9 (CH₂), 6.3 (2×CH₂); HRMS (ESI/Q-TOF) m/z: [M+Na]⁺ calcd for C₃₀H₂₆N₄NaO₄, 529.1846; found, 529.1847.

### 4-(3-(4′-(Cyclopentyloxy)-[1,1′-biphenyl]-4-yl)-5-(quinoxalin-6-yl)-4,5-dihydro-1H-pyrazol-1-yl)-4-oxobutanoic acid (Compound 85)

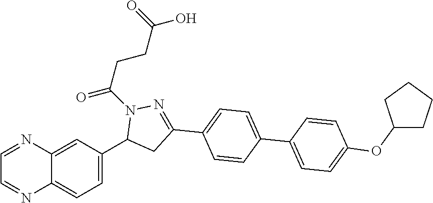 [SMILES: O=C(O)CCC(=O)N1N=C(c2ccc(-c3ccc(OC4CCCC4)cc3)cc2)CC1c1ccc2nccnc2c1]

Compound 85 was prepared over 2 steps using a method similar to that described for the preparation of Compound 20-step 1: quinoxaline-6-carbaldehyde (79 mg, 0.5 mmol), 1-(4′-(cyclopentyloxy)-[1,1′-biphenyl]-4-yl) ethan-1-one (140 mg, 0.5 mmol) and 2 M NaOH (1.0 mL, 2.0 mmol) in EtOH (4 mL) to afford (E)-1-(4′-(cyclopentyloxy)-[1,1′-biphenyl]-4-yl)-3-(quinoxalin-6-yl)prop-2-en-1-one as a colourless solid; step 2: The crude (E)-1-(4′-(cyclopentyloxy)-[1,1′-biphenyl]-4-yl)-3-(quinoxalin-6-yl)prop-2-en-1-one and hydrazine hydrate (25 μL, 50% w/w) in THF (2 mL), followed by the addition of succinic anhydride (50 mg, 0.5 mmol). Purification by flash column chromatography (EtOAc→DCM/MeOH 10:1) afforded Compound 85 as a colourless solid (56 mg, 21% over 2 steps); ¹H NMR (400 MHz, DMSO-d₆) δ 8.79-8.73 (2H, m), 8.01 (1H, d, J=8.7 Hz), 7.99 (1H, d, J=1.7 Hz), 7.88-7.82 (2H, m), 7.72-7.61 (3H, m), 7.61-7.54 (2H, m), 6.97-6.92 (2H, m), 5.82 (1H, dd, J=11.5, 4.8 Hz), 4.87-4.79 (m, 1H), 3.96 (1H, dd, J=17.5, 12.0 Hz), 3.29 (1H, dd, J=17.8, 5.4 Hz), 3.22-3.02 (2H, m), 2.57 (2H, t, J=6.7 Hz), 1.97-1.68 (6H, m), 1.68-1.57 (2H, m); ¹³C NMR (101 MHz. DMSO-d₆) δ 173.7 (C), 169.2 (C), 157.7 (C), 154.2 (C), 145.9 (CH), 145.5 (CH), 144.2 (C), 142.1 (C), 141.6 (2×C), 131.0 (C), 129.8 (CH), 129.1 (C), 128.1 (CH), 127.8 (2×CH), 127.3 (2×CH), 126.2 (2×CH), 125.3 (CH), 115.9 (2×CH), 78.7 (CH), 59.5 (CH), 41.7 (CH₂), 32.2 (2×CH₂), 28.7 (CH₂), 28.3 (CH₂), 23.6 (2×CH₂); HRMS (ESI/Q-TOF) m/z: [M+Na]⁺ calcd for C₃₂H₃₀N₄NaO₄, 557.2159; found, 557.2159.

### 4-(3-(4′-Isobutyl-[1,1′-biphenyl]-4-yl)-5-(quinoxalin-6-yl)-4,5-dihydro-1H-pyrazol-1-yl)-4-oxobutanoic acid (Compound 86)

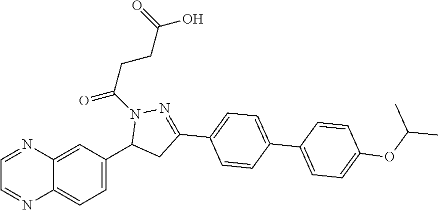 [SMILES: CC(C)Oc1ccc(-c2ccc(C3=NN(C(=O)CCC(=O)O)C(c4ccc5nccnc5c4)C3)cc2)cc1]

Compound 86 was prepared over 2 steps using a method similar to that described for the preparation of Compound 20-step 1: quinoxaline-6-carbaldehyde (158 mg, 1.00 mmol), 1-(4′-isobutyl-[1,1′-biphenyl]-4-yl)ethan-1-one (252 mg, 1.00 mmol) and 2 M NaOH (2.5 mL, 5.0 mmol) in EtOH (8 mL) to afford (E)-1-(4′-isobutyl-[1,1′-biphenyl]-4-yl)-3-(quinoxalin-6-yl)prop-2-en-1-one as an colourless solid; step 2: The crude (E)-3-(quinoxalin-6-yl)-1-(4-(trifluoromethyl)phenyl)prop-2-en-1-one and hydrazine hydrate (54 μL, 50% w/w) in THF (4 mL), followed by the addition of succinic anhydride (400 mg, 4.00 mmol). Purification by flash column chromatography (EtOAc, neat) afforded Compound 86 as a colourless solid (151 mg, 30% over 2 steps); ¹H NMR (400 MHz, CDCl₃) δ 8.81 (2H, s), 8.11 (1H, d, J=8.7 Hz), 7.99 (1H, d, J=1.7 Hz), 7.84-7.79 (2H, m), 7.71-7.63 (3H, m), 7.56-7.51 (2H, m), 7.25-7.21 (2H, m), 5.84 (1H, dd, J=11.8, 4.1 Hz), 3.92 (1H, dd, J=17.5, 11.8 Hz), 3.37-3.11 (3H, m), 2.76 (2H, t, J=6.8 Hz), 2.52 (2H, d, J=7.3 Hz), 1.98-1.84 (1H, m), 0.94 (6H, d, J=6.7 Hz); 13C NMR (101 MHz, CDCl₃) § 176.2 (C), 170.3 (C), 154.4 (C), 145.2 (CH), 145.0 (CH), 143.8 (C), 143.5 (C), 142.9 (C), 142.5 (C), 141.9 (C), 137.4 (C), 130.6 (CH), 129.8 (2×CH), 129.4 (C), 128.2 (CH), 127.3 (2×CH), 127.29 (2×CH), 126.8 (2×CH), 125.8 (CH), 60.1 (CH), 45.1 (CH₂), 42.3 (CH₂), 30.3 (CH), 29.1 (CH₂), 28.9 (CH₂), 22.5 (2×CH₃); HRMS (ESI/Q-TOF) m/z: [M+Na]⁺ calcd for C₃₁H₃₀N₄NaO₃, 529.2210; found, 529.2209.

### 4-(3-(4′-(tert-Butyl)-[1,1′-biphenyl]-4-yl)-5-(quinoxalin-6-yl)-4,5-dihydro-1H-pyrazol-1-yl)-4-oxobutanoic acid (Compound 87)

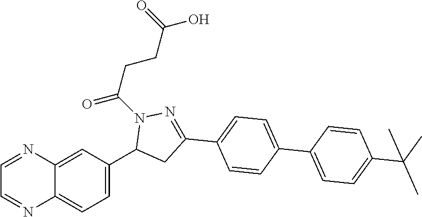 [SMILES: CC(C)(C)c1ccc(-c2ccc(C3=NN(C(=O)CCC(=O)O)C(c4ccc5nccnc5c4)C3)cc2)cc1]

Compound 87 was prepared over 2 steps using a method similar to that described for the preparation of Compound 20-step 1: quinoxaline-6-carbaldehyde (158 mg, 1.00 mmol), 1-(4′-(tert-butyl)-[1,1′-biphenyl]-4-yl)ethan-1-one (252 mg, 1.00 mmol) and 2 M NaOH (2.5 mL, 5.0 mmol) in EtOH (8 mL) to afford (E)-1-(4′-(tert-butyl)-[1,1′-biphenyl]-4-yl)-3-(quinoxalin-6-yl)prop-2-en-1-one as a colourless solid; step 2: The crude (E)-1-(4′-(tert-butyl)-[1,1′-biphenyl]-4-yl)-3-(quinoxalin-6-yl)prop-2-en-1-one and hydrazine hydrate (54 μL, 50% w/w) in THF (4 mL), followed by the addition of succinic anhydride (129 mg, 1.29 mmol). Purification by flash column chromatography (EtOAc→DCM/MeOH 10:1) afforded Compound 87 as a colourless solid (120 mg, 24% over 2 steps); ¹H NMR (400 MHz, CDCl₃) δ 8.83-8.79 (2H, m), 8.11 (1H, d, J=8.7 Hz), 7.99 (1H, d, J=1.7 Hz), 7.85-7.78 (2H, m), 7.69-7.64 (3H, m), 7.59-7.55 (2H, m), 7.51-7.47 (2H, m), 5.84 (1H, dd, J=11.5, 4.8 Hz), 3.91 (1H, dd, J=17.5, 12.0 Hz), 3.36-3.12 (3H, m), 2.76 (2H, t, J=6.5 Hz), 1.37 (9H, m); 13C NMR (101 MHz, CDCl₃) δ 176.3 (C), 170.3 (C), 154.4 (C), 151.3 (C), 145.3 (CH), 145.0 (CH), 143.8 (C), 143.4 (C), 143.0 (C), 142.5 (C), 137.1 (C), 130.6 (CH), 129.5 (C), 128.1 (CH), 127.3 (4×CH), 126.8 (2×CH), 126.0 (2×CH), 125.9 (CH), 60.2 (CH), 42.3 (CH₂), 34.7 (C), 31.4 (3×CH₃), 29.1 (CH₂), 28.9 (CH₂); HRMS (ESI/Q-TOF) m/z: [M+Na]⁺ calcd for C₃₁H₃₀N₄NaO₃, 529.2210; found, 529.2208.

### 4-Oxo-4-(5-(quinoxalin-6-yl)-3-(4′-(2,2,2-trifluoroethyl)-[1,1′-biphenyl]-4-yl)-4,5-dihydro-1H-pyrazol-1-yl)butanoic acid (Compound 88)

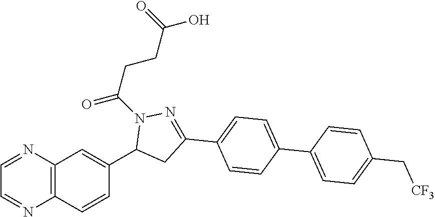 [SMILES: O=C(O)CCC(=O)N1N=C(c2ccc(-c3ccc(CC(F)(F)F)cc3)cc2)CC1c1ccc2nccnc2c1]

Compound 88 was prepared over 2 steps using a method similar to that described for the preparation of Compound 20-step 1: quinoxaline-6-carbaldehyde (103 mg, 0.650 mmol), 1-(4′-(2,2,2-trifluoroethyl)-[1,1′-biphenyl]-4-yl) ethan-1-one (181 mg, 0.650 mmol) and 2 M NaOH (1.6 mL, 3.25 mmol) in EtOH (5 mL) to afford (E)-3-(quinoxalin-6-yl)-1-(4′-(2,2,2-trifluoroethyl)-[1,1′-biphenyl]-4-yl)prop-2-en-1-one as an off-white solid (242 mg); step 2: (E)-3-(quinoxalin-6-yl)-1-(4′-(2,2,2-trifluoroethyl)-[1,1′-biphenyl]-4-yl)prop-2-en-1-one (242 mg) and hydrazine hydrate (72 μL, 50% w/w) in THF (5 mL), followed by the addition of succinic anhydride (174 mg, 1.74 mmol). Purification by flash column chromatography (petroleum ether/EtOAc, 1:1→EtOAc, neat) afforded Compound 88 as a yellow solid (180 mg, 52% over 2 steps). ¹H NMR (400 MHz, CDCl₃) δ 8.81-8.80 (2H, m), 8.10 (1H, d, J=8.8 Hz), 7.99 (1H, d, J=2.0 Hz), 7.83 (2H, d, J=8.6 Hz), 7.68 (1H, dd, J=8.8, 2.0 Hz), 7.65 (1H, d, J=8.6 Hz), 7.61 (2H, d, J=8.3 Hz), 7.39 (2H, d, J=8.0 Hz), 5.84 (1H, dd, J=11.9, 4.9 Hz), 3.90 (1H, dd, J=17.7, 11.9 Hz), 3.42 (2H, q. J=10.8 Hz), 3.32-3.15 (3H, m), 2.76 (2H, t, J=6.9 Hz); ¹³C NMR (101 MHz, CDCl₃) δ 176.9 (C), 170.3 (C), 154.1 (C), 145.3 (CH), 145.1 (CH), 143.9 (C), 143.0 (C), 142.7 (C), 142.5 (C), 140.0 (C), 130.9 (2×CH), 130.6 (CH), 130.2 (C), 130.0 (C, q, J_{C-F}=2.9 Hz), 128.2 (CH), 127.5 (2×CH), 127.5 (2×CH), 127.4 (2×CH), 125.9 (CH), 125.8 (C, q, J_{C-F}=277.0 Hz), 60.2 (CH), 42.3 (CH₂), 40.0 (CH₂, q. J_{C-F}=29.7 Hz), 29.2 (CH₂), 28.9 (CH₂); HRMS (ESI/Q-TOF) m/z: [M+Na]⁺ calcd for C₂₉H₂₃F₃N₄NaO₃, 555.1614; found, 555.1615.

### 4-(3-(4′-(Methoxymethyl)-[1,1′-biphenyl]-4-yl)-5-(quinoxalin-6-yl)-4,5-dihydro-1H-pyrazol-1-yl)-4-oxobutanoic acid (Compound 89)

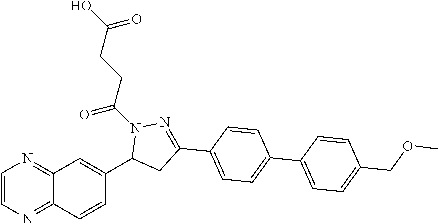 [SMILES: COCc1ccc(-c2ccc(C3=NN(C(=O)CCC(=O)O)C(c4ccc5nccnc5c4)C3)cc2)cc1]

Compound 89 was prepared over 2 steps using a method similar to that described for the preparation of Compound 20-step 1: quinoxaline-6-carbaldehyde (303 mg, 1.91 mmol), 1-(4′-(methoxymethyl)-[1,1′-biphenyl]-4-yl) ethan-1-one (460 mg, 1.91 mmol) and 2 M NaOH (4.80 mL, 9.60 mmol) in EtOH (15 mL) to afford (E)-1-(4′-(methoxymethyl)-[1,1′-biphenyl]-4-yl)-3-(quinoxalin-6-yl)prop-2-en-1-one as a colourless solid without further purification (550 mg); step 2: (E)-1-(4′-(methoxymethyl)-[1,1′-biphenyl]-4-yl)-3-(quinoxalin-6-yl)prop-2-en-1-one (550 mg) and hydrazine hydrate (180 μL, 50% w/w, 2.89 mmol) in THF (12 mL), followed by the addition of succinic anhydride (434 mg, 4.34 mmol). Purification by flash column chromatography (EtOAc/petroleum ether, 4:1→EtOAc, neat→EtOAc/MeOH, 9:1) afforded Compound 89 as a yellow solid (280 mg, 30% over 2 steps). ¹H NMR (400 MHz, CDCl₃) δ 8.82-8.81 (2H, m), 8.11 (1H, d, J=8.7 Hz), 7.99 (1H, d, J=1.8 Hz), 7.83 (2H, d, J=8.4 Hz), 7.69-7.60 (5H, m), 7.43 (2H, d, J=8.1 Hz), 5.85 (1H, dd, J=11.9, 4.8 Hz), 4.51 (2H, s), 3.92 (1H, dd, J=17.7, 11.9 Hz), 3.43 (3H, s), 3.34-3.15 (3H, m), 2.76 (2H, t, J=6.7 Hz); ¹³C NMR (101 MHz, CDCl₃) δ 175.9 (C), 170.4 (C), 154.4 (C), 145.4 (CH), 145.1 (CH), 143.8 (C), 143.3 (C), 143.1 (C), 142.7 (C), 139.5 (C), 138.3 (C), 130.7 (CH), 129.9 (C), 128.4 (2×CH), 128.2 (CH), 127.5 (2×CH), 127.4 (2×CH), 127.2 (2×CH), 125.9 (CH), 74.5 (CH₂), 60.3 (CH), 58.4 (CH₃), 42.4 (CH₂), 29.2 (CH₂), 29.0 (CH₂); HRMS (ESI/Q-TOF) m/z: [M+Na]⁺ calcd for C₂₉H₂₆N₄NaO₄, 517.1846; found, 517.1845.

### 4-(3-(3′-Methoxy-[1,1′-biphenyl]-4-yl)-5-(quinoxalin-6-yl)-4,5-dihydro-1H-pyrazol-1-yl)-4-oxobutanoic acid (Compound 90)

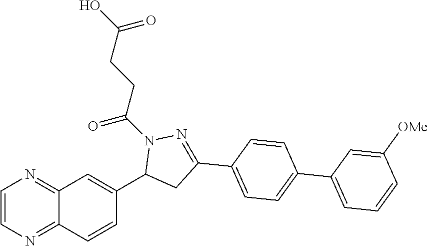 [SMILES: COc1cccc(-c2ccc(C3=NN(C(=O)CCC(=O)O)C(c4ccc5nccnc5c4)C3)cc2)c1]

Compound 90 was prepared over 2 steps using a method similar to that described for the preparation of Compound 20-step 1: quinoxaline-6-carbaldehyde (158 mg, 1.00 mmol), 1-(3′-methoxy-[1,1′-biphenyl]-4-yl) ethan-1-one (226 mg, 1.00 mmol) and 2 M NaOH (2.5 mL, 5.00 mmol) in EtOH (8 mL) to afford (E)-1-(3′-methoxy-[1,1′-biphenyl]-4-yl)-3-(quinoxalin-6-yl)prop-2-en-1-one as an off-white solid without further purification (340 mg); step 2: (E)-1-(3′-methoxy-[1,1′-biphenyl]-4-yl)-3-(quinoxalin-6-yl)prop-2-en-1-one (340 mg) and hydrazine hydrate (115 μL, 50% w/w, 1.86 mmol) in THF (7 mL), followed by the addition of succinic anhydride (278 mg, 2.78 mmol). Purification by flash column chromatography (petroleum ether/EtOAc, 1:1→EtOAc, neat) afforded Compound 90 as a colourless solid (104 mg, 22% over 2 steps). ¹H NMR (400 MHz, CDCl₃) δ 8.81-8.80 (2H, m), 8.10 (1H, d, J=8.7 Hz), 7.99 (1H, d, J=1.9 Hz), 7.82 (2H, d, J=8.4 Hz), 7.69-7.64 (3H, m), 7.38 (1H, t, J=8.0 Hz), 7.20 (1H, dt, J=7.9, 2.0 Hz), 7.14 (1H, t, J=2.0 Hz), 6.94-6.92 (1H, m), 5.84 (1H, dd, J=11.8, 4.9 Hz), 3.95-3.86 (4H, m), 3.34-3.13 (3H, m), 2.75 (2H, t, J=6.7 Hz); ¹³C NMR (101 MHz, CDCl₃) δ 176.3 (C), 170.4 (C), 160.2 (C), 154.3 (C), 145.4 (CH), 145.1 (CH), 143.8 (C), 143.4 (C), 143.0 (C), 142.6 (C), 141.7 (C), 130.7 (CH), 130.11 (CH), 130.06 (C), 128.2 (CH), 127.6 (2×CH), 127.4 (2×CH), 125.9 (CH), 119.7 (CH), 113.4 (CH), 113.1 (CH), 60.2 (CH), 55.5 (CH₃), 42.4 (CH₂), 29.2 (CH₂), 29.0 (CH₂); HRMS (ESI/Q-TOF) m/z: [M+Na]⁺ calcd for C₂₈H₂₄N₄NaO₄, 503.1690; found, 503.1688.

### 4-(3-(4′-(2-Methoxyethoxy)-[1,1′-biphenyl]-4-yl)-5-(quinoxalin-6-yl)-4,5-dihydro-1H-pyrazol-1-yl)-4-oxobutanoic acid (Compound 91)

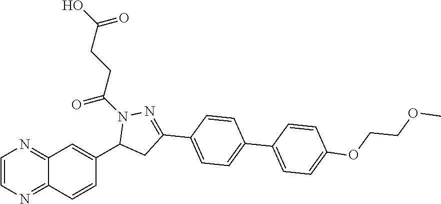 [SMILES: COCCOc1ccc(-c2ccc(C3=NN(C(=O)CCC(=O)O)C(c4ccc5nccnc5c4)C3)cc2)cc1]

Compound 91 was prepared over 2 steps using a method similar to that described for the preparation of Compound 20-step 1: quinoxaline-6-carbaldehyde (261 mg, 1.65 mmol), 1-(4′-(2-methoxyethoxy)-[1,1′-biphenyl]-4-yl) ethan-1-one (445 mg, 1.65 mmol) and 2 M NaOH (4.15 mL, 8.30 mmol) in EtOH (13 mL) to afford (E)-1-(4′-(2-methoxyethoxy)-[1,1′-biphenyl]-4-yl)-3-(quinoxalin-6-yl)prop-2-en-1-one as a beige solid without further purification (480 mg); step 2: (E)-1-(4′-(2-methoxyethoxy)-[1,1′-biphenyl]-4-yl)-3-(quinoxalin-6-yl)prop-2-en-1-one (480 mg) and hydrazine hydrate (145 μL, 50% w/w, 2.34 mmol) in THF (10 mL), followed by the addition of succinic anhydride (351 mg, 3.51 mmol). Purification by flash column chromatography (EtOAc, neat→EtOAc/MeOH, 9:1→acetone/MeOH, 9:1) afforded Compound 91 as a dark yellow solid (147 mg, 17% over 2 steps). ¹H NMR (400 MHz, CDCl₃) δ 8.82 (2H, s), 8.11 (1H, d, J=8.7 Hz), 7.99 (1H, s), 7.80 (2H, d, J=8.2 Hz), 7.68 (1H, dd, J=8.7, 1.4 Hz), 7.62 (2H, d, J=8.2 Hz), 7.56 (2H, d, J=8.6 Hz), 7.02 (2H, d, J=8.6 Hz), 5.84 (1H, dd, J=11.8, 4.7 Hz), 4.18 (2H, t, J=4.6 Hz), 3.92 (1H, dd, J=17.7, 11.9 Hz), 3.78 (2H, t, J=4.6 Hz), 3.47 (3H, s), 3.35-3.16 (3H, m), 2.75 (2H, t, J=6.6 Hz); ¹³C NMR (101 MHz, CDCl₃) δ 175.3 (C), 170.5 (C), 159.1 (C), 154.7 (C), 145.4 (CH), 145.2 (CH), 143.7 (C), 143.2 (C), 143.1 (C), 142.7 (C), 132.8 (C), 130.7 (CH), 129.2 (C), 128.3 (2×CH), 128.1 (CH), 127.4 (2×CH), 127.1 (2×CH), 126.0 (CH), 115.2 (2×CH), 71.2 (CH₂), 67.6 (CH₂), 60.3 (CH), 59.4 (CH₃), 42.4 (CH₂), 29.2 (CH₂), 29.0 (CH₂); HRMS (ESI/Q-TOF) m/z: [M+Na]⁺ calcd for C₃₀H₂₈N₄NaO₅, 547.1952; found, 547.1954.

### 4-Oxo-4-(5-(quinoxalin-6-yl)-3-(4′-(2,2,2-trifluoroethoxy)-[1,1′-biphenyl]-4-yl)-4,5-dihydro-1H-pyrazol-1-yl)butanoic acid (Compound 92)

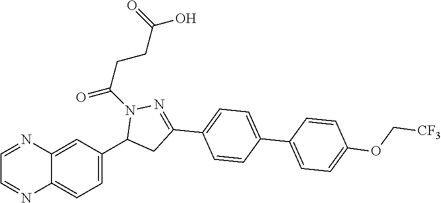 [SMILES: O=C(O)CCC(=O)N1N=C(c2ccc(-c3ccc(OCC(F)(F)F)cc3)cc2)CC1c1ccc2nccnc2c1]

Compound 92 was prepared over 2 steps using a method similar to that described for the preparation of Compound 20-step 1: quinoxaline-6-carbaldehyde (158 mg, 1.00 mmol), 1-(4′-(2,2,2-trifluoroethoxy)-[1,1′-biphenyl]-4-yl)ethan-1-one (294 mg, 1.00 mmol) and 2 M NaOH (2.5 mL, 5.0 mmol) in EtOH (8 mL) to afford (E)-3-(quinoxalin-6-yl)-1-(4′-(2,2,2-trifluoroethoxy)-[1,1′-biphenyl]-4-yl)prop-2-en-1-one as an colourless solid; step 2: The crude (E)-3-(quinoxalin-6-yl)-1-(4′-(2,2,2-trifluoroethoxy)-[1,1′-biphenyl]-4-yl)prop-2-en-1-one and hydrazine hydrate (54 μL, 50% w/w) in THF (4 mL), followed by the addition of succinic anhydride (400 mg, 4.00 mmol). Purification by flash column chromatography (EtOAc→DCM/MeOH 10:1) afforded Compound 92 as a colourless solid (186 mg, 34% over 2 steps); ¹H NMR (400 MHz, CDCl₃) δ 8.80-8.77 (2H, m), 8.11 (1H, d, J=8.7 Hz), 7.99 (1H, d, J=1.7 Hz), 7.84-7.79 (2H, m), 7.68-7.54 (5H, m), 7.05-6.99 (2H, m), 5.84 (1H, dd, J=11.8, 4.1 Hz), 4.43-4.34 (2H, m), 3.87 (1H, dd, J=17.5, 11.8 Hz), 3.33-3.11 (3H, m), 2.75 (2H, t, J=6.8 Hz); ¹³C NMR (101 MHz, CDCl₃) δ 176.8 (C), 170.2 (C), 157.4 (C), 154.0 (C), 145.2 (CH), 144.9 (CH), 143.9 (C), 142.8 (C), 142.4 (C), 142.3 (C), 134.4 (C), 130.4 (CH), 129.6 (C), 128.4 (2×CH), 128.1 (CH), 127.3 (2×CH), 127.0 (2×CH), 125.7 (CH), 123.4 (C, q, J_{C-F}=268 Hz), 115.4 (2×CH), 66.0 (CH₂, q. J_{C-F}=36 Hz), 60.1 (CH), 42.2 (CH₂), 29.2 (CH₂), 28.8 (CH₂); HRMS (ESI/Q-TOF) m/z: [M+Na]⁺ calcd for C₂₉H₂₃F₃N₄NaO, 571.1564; found, 571.1563.

### 4-(3-(4′-(Isopropylthio)-[1,1′-biphenyl]-4-yl)-5-(quinoxalin-6-yl)-4,5-dihydro-1H-pyrazol-1-yl)-4-oxobutanoic acid (Compound 93)

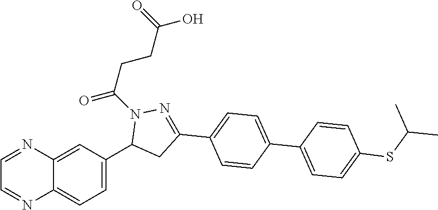 [SMILES: CC(C)Sc1ccc(-c2ccc(C3=NN(C(=O)CCC(=O)O)C(c4ccc5nccnc5c4)C3)cc2)cc1]

Compound 93 was prepared over 2 steps using a method similar to that described for the preparation of Compound 20-step 1: quinoxaline-6-carbaldehyde (158 mg, 1.00 mmol), 1-(4′-(isopropylthio)-[1,1′-biphenyl]-4-yl)ethan-1-one (270 mg, 1.00 mmol) and 2 M NaOH (2.5 mL, 5.0 mmol) in EtOH (8 mL) to afford (E)-1-(4′-(isopropylthio)-[1,1′-biphenyl]-4-yl)-3-(quinoxalin-6-yl)prop-2-en-1-one as a colourless solid; step 2: The crude (E)-1-(4′-(isopropylthio)-[1,1′-biphenyl]-4-yl)-3-(quinoxalin-6-yl)prop-2-en-1-one and hydrazine hydrate (54 μL, 50% w/w) in THF (4 mL), followed by the addition of succinic anhydride (129 mg, 1.29 mmol). Purification by flash column chromatography (EtOAc→DCM/MeOH 10:1) afforded Compound 93 as a colourless solid (100 mg, 19% over 2 steps); ¹H NMR (400 MHz, CDCl₃) δ 8.83-8.79 (2H, m), 8.11 (1H, d, J=8.7 Hz), 7.99 (1H, d, J=1.7 Hz), 7.85-7.78 (2H, m), 7.69-7.61 (3H, m), 7.57-7.55 (2H, m), 7.48-7.42 (2H, m), 5.84 (1H, dd, J=11.5, 4.8 Hz), 3.90 (1H, dd, J=17.5, 12.0 Hz), 3.50-3.38 (1H, m), 3.36-3.12 (3H, m), 2.76 (2H, t, J=6.7 Hz), 1.33 (6H, d, J=6.5 Hz); ¹³C NMR (101 MHz, CDCl₃) δ 176.7 (C), 170.2 (C), 154.1 (C), 145.2 (CH), 145.0 (CH), 143.8 (C), 142.9 (C), 142.6 (C), 142.5 (C), 138.2 (C), 135.9 (C), 131.9 (2×CH), 130.5 (CH), 129.9 (C), 128.1 (CH), 127.4 (2×CH), 127.3 (2×CH), 127.2 (2×CH), 125.8 (CH), 60.1 (CH), 42.2 (CH₂), 38.1 (CH), 29.2 (CH₂), 28.9 (CH₂), 23.2 (2×CH₃); HRMS (ESI/Q-TOF) m/z: [M+Na]⁺ calcd for C₃₀H₂₈N₄NaO₃S, 547.1774; found, 547.1769.

### 4-Oxo-4-(5-(quinoxalin-6-yl)-3-(4′-((trifluoromethyl)thio)-[1,1′-biphenyl]-4-yl)-4,5-dihydro-1H-pyrazol-1-yl)butanoic acid (Compound 94)

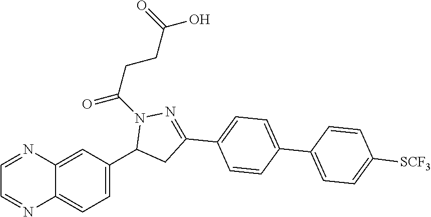

Compound 94 was prepared over 2 steps using a method similar to that described for the preparation of Compound 20-step 1: quinoxaline-6-carbaldehyde (158 mg, 1.00 mmol), 1-(4′-((trifluoromethyl)thio)-[1,1′-biphenyl]-4-yl) ethan-1-one (296 mg, 1.00 mmol) and 2 M NaOH (2.5 mL, 5.0 mmol) in EtOH (8 mL) to afford (E)-3-(quinoxalin-6-yl)-1-(4′-((trifluoromethyl)thio)-[1,1′-biphenyl]-4-yl)prop-2-en-1-one as an yellow solid; step 2: The crude (E)-3-(quinoxalin-6-yl)-1-(4-(trifluoromethyl)phenyl)prop-2-en-1-one and hydrazine hydrate (54 μL, 50% w/w) in THF (4 mL), followed by the addition of succinic anhydride (400 mg, 4.00 mmol). Purification by flash column chromatography (EtOAc→DCM/MeOH 10:1) afforded Compound 94 as a colourless solid (186 mg, 34% over 2 steps); ¹H NMR (400 MHz, CDCl₃) δ 8.79 (2H, s), 8.10 (1H, d, J=8.7 Hz), 7.99 (1H, d, J=1.7 Hz), 7.87-7.81 (2H, m), 7.76-7.61 (7H, m), 5.85 (1H, dd, J=11.8, 4.1 Hz), 3.34-3.12 (3H, m), 2.75 (2H, t, J=6.8 Hz); ¹³C NMR (101 MHz, CDCl₃) δ 176.8 (C), 170.3 (C), 153.8 (C), 145.2 (CH), 145.0 (CH), 143.8 (C), 142.9 (C), 142.6 (C), 142.4 (C), 141.7 (C), 136.8 (2×CH), 131.1 (CF₃), 130.8 (C), 130.5 (CH), 128.2 (CH), 128.1 (2×CH), 127.6 (2×CH), 127.4 (2×CH), 125.7 (CH), 123.9 (C), 60.1 (CH), 42.2 (CH₂), 29.2 (CH₂), 28.8 (CH₂); HRMS (ESI/Q-TOF) m/z: [M+Na]⁺ calcd for C₂₈H₂₁F₃N₄NaO₃S, 573.1179; found, 573.1162.

### 4-(3-(4′-(Dimethylamino)-[1,1′-biphenyl]-4-yl)-5-(quinoxalin-6-yl)-4,5-dihydro-1H-pyrazol-1-yl)-4-oxobutanoic acid (Compound 95)

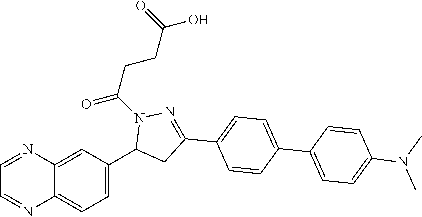 [SMILES: CN(C)c1ccc(-c2ccc(C3=NN(C(=O)CCC(=O)O)C(c4ccc5nccnc5c4)C3)cc2)cc1]

To a solution of Compound 9 (80 mg, 0.18 mmol) in DMF/water (2.0 mL, 1:1 v/v), was added (4-(dimethylamino)phenyl) boronic acid (59 mg, 0.36 mmol), potassium phosphate tribasic (0.19 g, 0.90 mmol), and palladium acetate (4.0 mg, 10 mol %). The reaction mixture was stirred at 80° C. for 16 h. The reaction mixture was then diluted with EtOAc (10 mL), neutralized with 0.5 M aq. HCl and the separated aqueous layer further extracted with EtOAc (2×10 mL). The combined organic layers were washed with brine (20 mL), dried over anhydrous Na₂SO₄, filtered and concentrated in vacuo. Purification by flash column chromatography (DCM/MeOH, 10:1) afforded Compound 95 as a yellow solid (30 mg, 34%). ¹H NMR (400 MHz, CDCl₃) δ 8.84 (2H, br s), 8.13 (1H, d, J=8.7 Hz), 8.00 (1H, d, J=1.9 Hz), 7.82 (2H, d, J=8.4 Hz), 7.69 (1H, dd, J=8.6, 2.0 Hz), 7.66-7.62 (4H, m), 7.27 (2H, d, J=8.4 Hz), 5.85 (1H, dd, J=11.9, 4.7 Hz), 3.92 (1H, dd, J=17.6, 11.8 Hz), 3.36-3.16 (3H, m), 3.13 (6H, s), 2.76 (2H, t, J=6.6 Hz).

### 4-Oxo-4-(3-(4′-(piperidin-1-yl)-[1,1′-biphenyl]-4-yl)-5-(quinoxalin-6-yl)-4,5-dihydro-1H-pyrazol-1-yl)butanoic acid (Compound 96)

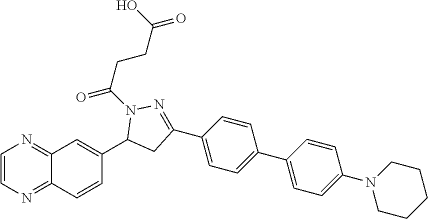 [SMILES: O=C(O)CCC(=O)N1N=C(c2ccc(-c3ccc(N4CCCCC4)cc3)cc2)CC1c1ccc2nccnc2c1]

Compound 96 was prepared over 2 steps using a method similar to that described for the preparation of Compound 20-step 1: quinoxaline-6-carbaldehyde (261 mg, 1.65 mmol), 1-(4′-(piperidin-1-yl)-[1,1′-biphenyl]-4-yl) ethan-1-one (461 mg, 1.65 mmol) and 2 M NaOH (4.15 mL, 8.30 mmol) in EtOH (13 mL) to afford (E)-1-(4′-(piperidin-1-yl)-[1,1′-biphenyl]-4-yl)-3-(quinoxalin-6-yl)prop-2-en-1-one as a colourless solid without further purification (605 mg); step 2: (E)-1-(4′-(piperidin-1-yl)-[1,1′-biphenyl]-4-yl)-3-(quinoxalin-6-yl)prop-2-en-1-one (605 mg) and hydrazine hydrate (180 μL, 50% w/w, 2.89 mmol) in THF (12 mL), followed by the addition of succinic anhydride (434 mg, 4.34 mmol). Purification by flash column chromatography (EtOAc/petroleum ether, 4:1→EtOAc, neat→EtOAc/MeOH, 9:1) afforded Compound 96 as a yellow solid (320 mg, 36% over 2 steps). ¹H NMR (400 MHz, CDCl₃) δ 8.81 (2H, s), 8.11 (1H, d, J=8.6 Hz), 7.99 (1H, d, J=2.0 Hz), 7.78 (2H, d, J=8.4 Hz), 7.67 (1H, dd, J=8.7, 1.9 Hz), 7.63 (2H, d, J=8.4 Hz), 7.54 (2H, d, J=8.8 Hz), 7.00 (2H, d, J=8.8 Hz), 5.83 (1H, dd, J=11.9, 4.7 Hz), 3.91 (1H, dd, J=17.6, 11.8 Hz), 3.36-3.15 (7H, m), 2.75 (2H, t, J=6.7 Hz), 1.75-1.68 (4H, m), 1.64-1.58 (2H, m); ¹³C NMR (101 MHz, CDCl₃) δ 175.9 (C), 170.4 (C), 154.7 (C), 152.0 (C), 145.4 (CH), 145.1 (CH), 143.8 (C), 143.4 (C), 143.1 (C), 142.6 (C), 130.7 (CH), 130.0 (C), 128.7 (C), 128.2 (CH), 127.8 (2×CH), 127.4 (2× CH), 126.6 (2×CH), 125.9 (CH), 116.4 (2×CH), 60.2 (CH), 50.2 (2×CH₂), 42.4 (CH₂), 29.2 (CH₂), 29.1 (CH₂), 25.8 (2×CH₂), 24.5 (CH₂); HRMS (ESI/Q-TOF) m/z: [M+Na]⁺ calcd for C₃₂H₃₁N₅NaO₃, 556.2319; found, 556.2319.

### 4-(3-(3′-Methoxy-[1,1′-biphenyl]-3-yl)-5-(quinoxalin-6-yl)-4,5-dihydro-1H-pyrazol-1-yl)-4-oxobutanoic acid (Compound 97)

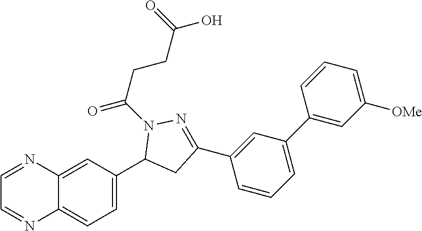 [SMILES: COc1cccc(-c2cccc(C3=NN(C(=O)CCC(=O)O)C(c4ccc5nccnc5c4)C3)c2)c1]

Compound 97 was prepared over 2 steps using a method similar to that described for the preparation of Compound 20-step 1: quinoxaline-6-carbaldehyde (79 mg, 0.5 mmol), 1-(3′-methoxy-[1,1′-biphenyl]-3-yl) ethan-1-one (113 mg, 0.5 mmol) and 2 M NaOH (1.0 mL, 2.0 mmol) in EtOH (4 mL) to afford (E)-1-(3′-methoxy-[1,1′-biphenyl]-3-yl)-3-(quinoxalin-6-yl)prop-2-en-1-one as a colourless solid; step 2: The crude (E)-1-(3′-methoxy-[1,1′-biphenyl]-3-yl)-3-(quinoxalin-6-yl)prop-2-en-1-one and hydrazine hydrate (25 μL, 50% w/w) in THF (2 mL), followed by the addition of succinic anhydride (50 mg, 0.5 mmol). Purification by flash column chromatography (EtOAc→DCM/MeOH, 10:1) afforded Compound 97 as a colourless solid (65 mg, 27% over 2 steps); ¹H NMR (400 MHz, CDCl₃) δ 8.83-8.78 (2H, m), 8.12 (1H, d, J=8.7 Hz), 7.98 (1H, d, J=1.8 Hz), 7.94 (1H, t, J=1.3 Hz), 7.73-7.68 (3H, m), 7.49 (1H, t, J=7.8 Hz), 7.37 (1H, t, J=7.8 Hz), 7.20-7.11 (2H, m), 6.95-6.90 (m, 1H), 5.85 (1H, dd, J=11.5, 4.8 Hz), 4.01-3.89 (m, 1H), 3.82 (3H, s), 3.37-3.25 (2H, m), 3.23-3.16 (1H, m), 2.75 (2H, t, J=6.6 Hz); ¹³C NMR (101 MHz, CDCl₃) δ 176.4 (C), 170.4 (C), 160.1 (C), 154.4 (C), 145.3 (CH), 145.0 (CH), 143.8 (C), 142.9 (C), 142.5 (C), 141.94 (C), 141.92 (C), 131.5 (C), 130.6 (CH), 130.0 (CH), 129.6 (CH), 129.3 (CH), 128.1 (CH), 125.8 (CH), 125.76 (CH), 125.6 (CH), 119.8 (CH), 113.2 (CH), 113.1 (CH), 60.1 (CH₃), 55.5 (CH), 42.4 (CH₂), 29.1 (CH₂), 28.8 (CH₂); HRMS (ESI/Q-TOF) m/z: [M+Na]⁺ calcd for C₂₈H₂₄N₄NaO₄, 503.1690; found, 503.1688.

### 4-Oxo-4-(5-(quinoxalin-6-yl)-3-(3′-(trifluoromethoxy)-[1,1′-biphenyl]-3-yl)-4,5-dihydro-1H-pyrazol-1-yl)butanoic acid (Compound 98)

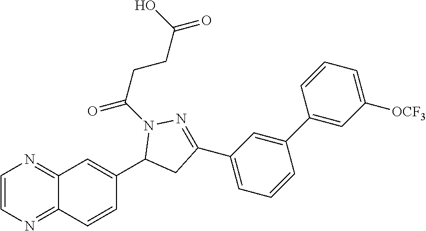 [SMILES: O=C(O)CCC(=O)N1N=C(c2cccc(-c3cccc(OC(F)(F)F)c3)c2)CC1c1ccc2nccnc2c1]

Compound 98 was prepared over 2 steps using a method similar to that described for the preparation of Compound 20-step 1: quinoxaline-6-carbaldehyde (158 mg, 1.00 mmol), 1-(3′-(trifluoromethoxy)-[1,1′-biphenyl]-3-yl) ethan-1-one (280 mg, 1.00 mmol) and 2 M NaOH (2.5 mL, 5.00 mmol) in EtOH (8 mL) to afford (E)-3-(quinoxalin-6-yl)-1-(3′-(trifluoromethoxy)-[1,1′-biphenyl]-3-yl)prop-2-en-1-one as a colourless solid without further purification (400 mg); step 2: (E)-3-(quinoxalin-6-yl)-1-(3′-(trifluoromethoxy)-[1,1′-biphenyl]-3-yl)prop-2-en-1-one (400 mg) and hydrazine hydrate (120 μL, 50% w/w, 1.90 mmol) in THF (8 mL), followed by the addition of succinic anhydride (286 mg, 2.85 mmol). Purification by flash column chromatography (petroleum ether/EtOAc, 1:1→EtOAc, neat) afforded Compound 98 as beige crystals (105 mg, 20% over 2 steps). ¹H NMR (400 MHz, CDCl₃) δ 8.81 (2H, s), 8.11 (1H, d, J=8.6 Hz), 7.99 (1H, s), 7.93 (1H, s), 7.76 (1H, d, J=7.5 Hz), 7.68 (1H, d, J=8.7 Hz), 7.63 (1H, d, J=7.4 Hz), 7.54-7.43 (4H, m), 7.24 (1H, d, J=7.8 Hz), 5.86 (1H, dd, J=11.7, 4.5 Hz), 3.94 (1H, dd, J=17.6, 12.0 Hz), 3.33-3.14 (3H, m), 2.75 (2H, t, J=6.4 Hz); ¹³C NMR (101 MHz, CDCl₃) δ 176.6 (C), 170.4 (C), 154.2 (C), 149.9 (C), 145.4 (CH), 145.1 (CH), 143.8 (C), 143.0 (C), 142.6 (C), 140.6 (C), 131.8 (C), 130.7 (CH), 130.4 (CH), 129.61 (CH), 129.57 (CH), 128.2 (CH), 126.4 (CH), 125.9 (CH), 125.8 (CH), 125.5 (CH), 120.7 (C, q, J_{C-F}=257.2 Hz), 120.2 (CH), 120.0 (CH), 60.3 (CH), 42.4 (CH₂), 29.2 (CH₂), 28.9 (CH₂); HRMS (ESI/Q-TOF) m/z: [M+Na]⁺ calcd for C₂₈H₂₁F₃N₄NaO₄, 557.1407; found, 557.1407.

### 4-(3-(4′-Methoxy-[1,1′-biphenyl]-3-yl)-5-(quinoxalin-6-yl)-4,5-dihydro-1H-pyrazol-1-yl)-4-oxobutanoic acid (Compound 99)

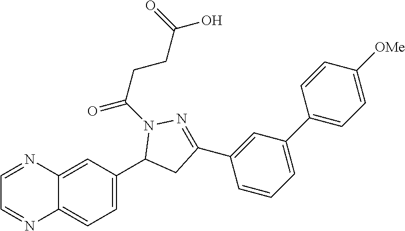 [SMILES: COc1ccc(-c2cccc(C3=NN(C(=O)CCC(=O)O)C(c4ccc5nccnc5c4)C3)c2)cc1]

Compound 99 was prepared over 2 steps using a method similar to that described for the preparation of Compound 20-step 1: quinoxaline-6-carbaldehyde (79 mg, 0.5 mmol), 1-(4′-methoxy-[1,1′-biphenyl]-3-yl)ethan-1-one (113 mg, 0.5 mmol) and 2 M NaOH (1.0 mL, 2.0 mmol) in EtOH (4 mL) to afford (E)-1-(4′-methoxy-[1,1′-biphenyl]-3-yl)-3-(quinoxalin-6-yl)prop-2-en-1-one as a colourless solid; step 2: The crude (E)-1-(4′-methoxy-[1,1′-biphenyl]-3-yl)-3-(quinoxalin-6-yl)prop-2-en-1-one and hydrazine hydrate (25 μL, 50% w/w) in THF (2 mL), followed by the addition of succinic anhydride (50 mg, 0.5 mmol). Purification by flash column chromatography (EtOAc→DCM/MeOH 10:1) afforded Compound 99 as a colourless solid (74 mg, 31% over 2 steps); ¹H NMR (400 MHz, DMSO-d₆) δ 11.62 (1H, br s), 8.98-8.91 (2H, m), 8.12 (1H, d, J=8.7 Hz), 7.99 (1H, t, J=1.3 Hz), 7.95 (1H, d, J=1.8 Hz), 7.85-7.80 (1H, m), 7.79-7.73 (2H, m), 7.73-7.68 (2H, m), 7.57 (1H, t, J=8.1 Hz), 7.12-6.51 (2H, m), 5.89 (1H, dd, J=11.5, 4.8 Hz), 4.13-4.01 (m, 1H), 3.82 (3H, s), 3.47 (1H, dd, J=17.5, 12.0 Hz), 3.20-2.96 (2H, m), 2.59-2.53 (2H, m); ¹³C NMR (101 MHz, DMSO-d₆) δ 173.7 (C), 169.3 (C), 159.2 (C), 154.6 (C), 145.9 (CH), 145.5 (CH), 144.2 (C), 142.1 (C), 141.5 (C), 140.3 (C), 131.7 (C), 131.5 (C), 129.7 (CH), 129.3 (CH), 128.2 (2×CH), 128.0 (2×CH), 125.3 (CH), 124.8 (CH), 124.6 (CH), 114.4 (2×CH), 59.5 (CH), 55.2 (CH₃), 41.8 (CH₂), 28.7 (CH₂), 28.3 (CH₂); HRMS (ESI/Q-TOF) m/z: [M+Na]⁺ calcd for C₂₈H₂₄N₄NaO₄, 503.1690; found, 503.1688.

### 4-(3-(3′-Fluoro-4′-isopropoxy-[1,1′-biphenyl]-4-yl)-5-(quinoxalin-6-yl)-4,5-dihydro-1H-pyrazol-1-yl)-4-oxobutanoic acid (Compound 100)

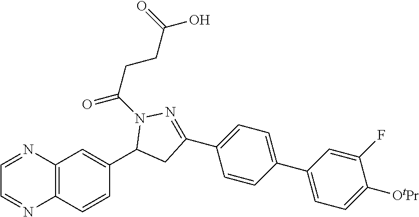

Compound 100 was prepared over 2 steps using a method similar to that described for the preparation of Compound 20-step 1: quinoxaline-6-carbaldehyde (32 mg, 0.20 mmol), 1-(3′-fluoro-4′-isopropoxy-[1,1′-biphenyl]-4-yl) ethan-1-one (55 mg, 0.20 mmol) and 2 M NaOH (0.50 mL, 1.00 mmol) in EtOH (2.0 mL) to afford (E)-1-(3′-fluoro-4′-isopropoxy-[1,1′-biphenyl]-4-yl)-3-(quinoxalin-6-yl)prop-2-en-1-one as an off-white solid (64 mg); step 2: (E)-1-(3′-fluoro-4′-isopropoxy-[1,1′-biphenyl]-4-yl)-3-(quinoxalin-6-yl)prop-2-en-1-one (63.5 mg) and hydrazine hydrate (19 μL, 50% w/w) in THF (1.5 mL), followed by the addition of succinic anhydride (46 mg, 0.46 mmol). Purification by flash column chromatography (petroleum ether/EtOAc, 1:1→EtOAc, neat) afforded Compound 100 as a yellow solid (49 mg, 47% over 2 steps). ¹H NMR (400 MHz, CDCl₃) δ 8.82-8.80 (2H, m), 8.11 (1H, d, J=8.7 Hz), 7.99 (1H, d, J=2.0 Hz), 7.80 (2H, d, J=8.5 Hz), 7.68 (1H, dd, J=8.8, 2.0 Hz), 7.59 (2H, d. J=8.5 Hz), 7.37-7.30 (2H, m), 7.05 (1H, t, J=8.5 Hz), 5.84 (1H, dd, J=11.9, 4.9 Hz), 4.60 (1H, hept, J=6.1 Hz), 3.91 (1H, dd, J=17.7, 11.9 Hz), 3.36-3.24 (2H, m), 3.17 (1H, dt, J=17.3, 6.8 Hz), 2.75 (2H, t, J=6.7 Hz), 1.40 (6H, d, J=6.0 Hz); ¹³C NMR (101 MHz, CDCl₃) δ 176.5 (C), 170.4 (C), 154.2 (C), 154.0 (C, d, J_{C-F}=245.7 Hz), 146.0 (C, d, J_{C-F}=10.6 Hz), 145.4 (CH), 145.1 (CH), 143.8 (C), 143.1 (C), 142.6 (C), 142.0 (C), 133.6 (C, d, J_{C-F}=6.6 Hz), 130.6 (CH), 129.8 (C), 128.2 (CH), 127.5 (2×CH), 127.0 (2×CH), 125.9 (CH), 122.8 (CH, d, J_{C-F}=3.2 Hz), 117.9 (CH, d, J_{C-F}=2.5 Hz), 115.1 (CH, d, J_{C-F}=19.8 Hz), 72.6 (CH), 60.3 (CH), 42.4 (CH₂), 29.2 (CH₂), 29.0 (CH₂), 22.2 (2×CH₃); HRMS (ESI/Q-TOF) m/z: [M+Na]⁺ calcd for C₃₀H₂₇FN₄NaO₄, 549.1909; found, 549.1912.

### 4-(3-(2′-Fluoro-4′-(trifluoromethoxy)-[1,1′-biphenyl]-4-yl)-5-(quinoxalin-6-yl)-4,5-dihydro-1H-pyrazol-1-yl)-4-oxobutanoic acid (Compound 101)

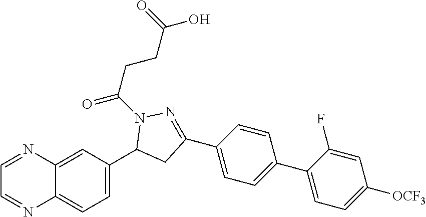 [SMILES: O=C(O)CCC(=O)N1N=C(c2ccc(-c3ccc(OC(F)(F)F)cc3F)cc2)CC1c1ccc2nccnc2c1]

Compound 101 was prepared over 2 steps using a method similar to that described for the preparation of Compound 20-step 1: quinoxaline-6-carbaldehyde (200 mg, 1.26 mmol), 1-(2′-fluoro-4′-(trifluoromethoxy)-[1,1′-biphenyl]-4-yl) ethan-1-one (376 mg, 1.26 mmol) and 2 M NaOH (3.2 mL, 6.30 mmol) in EtOH (10 mL) to afford (E)-1-(2′-fluoro-4′-(trifluoromethoxy)-[1,1′-biphenyl]-4-yl)-3-(quinoxalin-6-yl)prop-2-en-1-one as an off-white solid (492 mg); step 2: (E)-1-(2′-fluoro-4′-(trifluoromethoxy)-[1,1′-biphenyl]-4-yl)-3-(quinoxalin-6-yl)prop-2-en-1-one (250 mg) and hydrazine hydrate (71 μL, 50% w/w) in THF (5 mL), followed by the addition of succinic anhydride (171 mg, 1.71 mmol). Purification by flash column chromatography (petroleum ether/EtOAc, 1:1→EtOAc, neat) afforded Compound 101 as an off-white solid (148 mg, 42% over 2 steps). ¹H NMR (400 MHz, CDCl₃) δ 8.82-8.81 (2H, m), 8.11 (1H, d, J=8.7 Hz), 7.99 (1H, d, J=1.9 Hz), 7.84 (2H, d, J=8.6 Hz), 7.68 (1H, dd, J=8.7, 2.0 Hz), 7.60-7.58 (2H, m), 7.49 (1H, t, J=8.5 Hz), 7.14-7.07 (2H, m), 5.86 (1H, dd, J=11.9, 4.9 Hz), 3.91 (1H, dd, J=17.7, 11.9 Hz), 3.33-3.24 (2H, m), 3.18 (1H, dt, J=17.3, 6.8 Hz), 2.76 (2H, t, J=6.8 Hz); ¹³C NMR (101 MHz, CDCl₃) δ 176.8 (C), 170.4 (C), 159.7 (C, d, J_{C-F}=251.5 Hz), 153.9 (C), 149.6-149.3 (C, m), 145.3 (CH), 145.1 (CH), 143.8 (C), 142.9 (C), 142.5 (C), 136.8 (C, d, J_{C-F}=1.8 Hz), 131.4 (CH, d, J_{C-F}=4.3 Hz), 130.8 (C), 130.6 (CH), 129.4 (2×CH, d, J_{C-F}=2.7 Hz), 128.2 (CH), 127.1 (2×CH), 127.0 (C, d, J_{C-F}=13.4 Hz), 125.8 (CH), 120.5 (C, q, J_{C-F}=258.6 Hz), 117.0 (CH, d, J_{C-F}=4.0 Hz), 109.8 (CH, d, J_{C-F}=26.6 Hz), 60.3 (CH), 42.3 (CH₂), 29.2 (CH₂), 28.9 (CH₂); HRMS (ESI/Q-TOF) m/z: [M+H]⁺ calcd for C₂₈H₂₁F₄N₄O₄, 553.1493; found, 553.1490.

### 4-(3-(3′-Chloro-4′-(trifluoromethoxy)-[1,1′-biphenyl]-4-yl)-5-(quinoxalin-6-yl)-4,5-dihydro-1H-pyrazol-1-yl)-4-oxobutanoic acid (Compound 102)

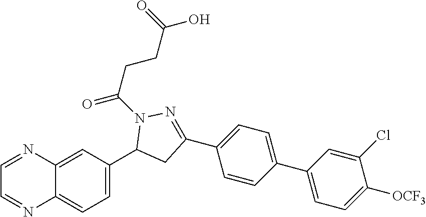 [SMILES: O=C(O)CCC(=O)N1N=C(c2ccc(-c3ccc(OC(F)(F)F)c(Cl)c3)cc2)CC1c1ccc2nccnc2c1]

Compound 102 was prepared over 2 steps using a method similar to that described for the preparation of Compound 20-step 1: quinoxaline-6-carbaldehyde (158 mg, 1.00 mmol), 1-(3′-chloro-4′-(trifluoromethoxy)-[1,1′-biphenyl]-4-yl) ethan-1-one (314 mg, 1.00 mmol) and 2 M NaOH (2.5 mL, 5.0 mmol) in EtOH (8 mL) to afford (E)-1-(3′-chloro-4′-(trifluoromethoxy)-[1,1′-biphenyl]-4-yl)-3-(quinoxalin-6-yl)prop-2-en-1-one as a colourless solid; step 2: The crude (E)-1-(3′-chloro-4′-(trifluoromethoxy)-[1,1′-biphenyl]-4-yl)-3-(quinoxalin-6-yl)prop-2-en-1-one and hydrazine hydrate (54 μL, 50% w/w) in THF (4 mL), followed by the addition of succinic anhydride (129 mg, 1.29 mmol). Purification by flash column chromatography (EtOAc→DCM/MeOH 10:1) afforded Compound 102 as a colourless solid (142 mg, 25% over 2 steps); ¹H NMR (400 MHz, CDCl₃) δ 8.80-8.77 (2H, m), 8.11 (1H, d, J=8.7 Hz), 7.98 (1H, d, J=1.7 Hz), 7.87-7.81 (2H, m), 7.72-7.65 (2H, m), 7.62-7.56 (2H, m), 7.53-7.48 (1H, m), 7.42-7.37 (1H, m), 5.85 (1H, dd, J=12.5, 4.1 Hz), 3.90 (1H, dd, J=17.5, 12.0 Hz), 3.34-3.12 (3H, m), 2.76 (2H, t, J=6.5 Hz); ¹³C NMR (101 MHz, CDCl₃) δ 176.8 (C), 170.2 (C), 153.7 (C), 145.3 (CH), 145.0 (CH), 144.95 (C), 143.7 (C), 142.9 (C), 142.5 (C), 140.6 (C), 140.2 (C), 130.9 (C), 130.5 (CH), 129.4 (C), 128.1 (CH), 128.0 (CH), 127.5 (2×CH), 127.46 (2×CH), 126.5 (CH), 125.8 (CH), 123.4 (C, q, J_{C-F}=268 Hz), 123.0 (CH), 60.2 (CH), 42.2 (CH₂), 29.1 (CH₂), 28.8 (CH₂); HRMS (ESI/Q-TOF) m/z: [M+Na]⁺ calcd for C₂₈H₂₀ClF₃N₄NaO₄, 591.1017; found, 591.1031.

### 4-(3-(4-(Benzo[d][1,3]dioxol-5-yl)phenyl)-5-(quinoxalin-6-yl)-4,5-dihydro-1H-pyrazol-1-yl)-4-oxobutanoic acid (Compound 103)

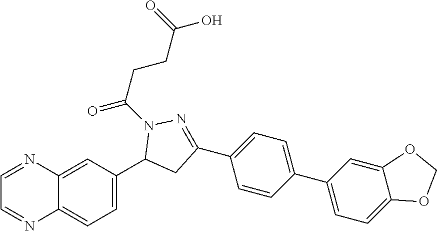 [SMILES: O=C(O)CCC(=O)N1N=C(c2ccc(-c3ccc4c(c3)OCO4)cc2)CC1c1ccc2nccnc2c1]

Compound 103 was prepared over 2 steps using a method similar to that described for the preparation of Compound 20-step 1: quinoxaline-6-carbaldehyde (158 mg, 1.00 mmol), 1-(4-(benzo[d][1,3]dioxol-5-yl)phenyl) ethan-1-one (240 mg, 1.00 mmol) and 2 M NaOH (2.5 mL, 5.0 mmol) in EtOH (8 mL) to afford (E)-1-(4-(benzo[d][1,3]dioxol-5-yl)phenyl)-3-(quinoxalin-6-yl)prop-2-en-1-one as a colourless solid; step 2: The crude (E)-1-(4-(benzo[d][1,3]dioxol-5-yl)phenyl)-3-(quinoxalin-6-yl)prop-2-en-1-one and hydrazine hydrate (54 μL, 50% w/w) in THF (4 mL), followed by the addition of succinic anhydride (129 mg, 1.29 mmol). Purification by flash column chromatography (EtOAc→DCM/MeOH 10:1) afforded Compound 103 as a colourless solid (100 mg, 20% over 2 steps); ¹H NMR (400 MHz, DMSO-d₆) δ 12.09 (1H, s), 8.10 (1H, d, J=9.3 Hz), 7.93 (1H, d, J=1.7 Hz), 7.89-7.84 (2H, m), 7.77-7.71 (3H, m), 7.35-7.33 (1H, d, J=9.3 Hz), 7.25 (1H, dd, J=7.8, 2.0 Hz), 7.04 (1H, d. J=7.8 Hz), 7.51-7.47 (2H, m), 6.09 (2H, s), 5.86 (1H, dd. J=11.5, 4.8 Hz), 4.02 (1H, dd, J=17.8, 12.0 Hz), 3.43-3.30 (3H, m), 3.17-2.94 (2H, m); ¹³C NMR (101 MHz, DMSO-d₆) δ 173.7 (C), 169.2 (C), 154.1 (C), 148.1 (C), 147.3 (C), 146.0 (CH), 145.6 (CH), 144.2 (C), 142.1 (C), 141.5 (C), 133.3 (C), 129.8 (CH), 129.5 (C), 128.1 (CH), 127.3 (2×CH), 127.2 (C), 126.6 (2×CH), 125.3 (CH), 120.5 (CH), 108.7 (CH), 107.0 (CH), 101.3 (CH₂), 59.5 (CH), 41.7 (CH₂), 28.7 (CH₂), 28.3 (CH₂); HRMS (ESI/Q-TOF) m/z: [M+Na]⁺ calcd for C₂₈H₂₂N₄NaO₅, 517.1482; found, 517.1482.

### 4-(3-(4-(2,2-Difluorobenzo[d][1,3]dioxol-5-yl)phenyl)-5-(quinoxalin-6-yl)-4,5-dihydro-1H-pyrazol-1-yl)-4-oxobutanoic acid (Compound 104)

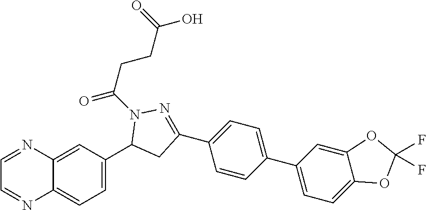 [SMILES: O=C(O)CCC(=O)N1N=C(c2ccc(-c3ccc4c(c3)OC(F)(F)O4)cc2)CC1c1ccc2nccnc2c1]

Compound 104 was prepared over 2 steps using a method similar to that described for the preparation of Compound 20-step 1: quinoxaline-6-carbaldehyde (158 mg, 1.00 mmol), 1-(4-(2,2-difluorobenzo[d][1,3]dioxol-5-yl)phenyl) ethan-1-one (276 mg, 1.00 mmol) and 2 M NaOH (2.5 mL, 5.0 mmol) in EtOH (8 mL) to afford (E)-1-(4-(2,2-difluorobenzo[d][1,3]dioxol-5-yl)phenyl)-3-(quinoxalin-6-yl)prop-2-en-1-one as a colourless solid; step 2: The crude (E)-1-(4-(2,2-difluorobenzo[d][1,3]dioxol-5-yl)phenyl)-3-(quinoxalin-6-yl)prop-2-en-1-one and hydrazine hydrate (54 μL, 50% w/w) in THF (4 mL), followed by the addition of succinic anhydride (129 mg, 1.29 mmol). Purification by flash column chromatography (EtOAc→DCM/MeOH 10:1) afforded Compound 104 as a colourless solid (137 mg, 26% over 2 steps); ¹H NMR (400 MHz, CDCl₃) δ 8.84-8.79 (2H, m), 8.11 (1H, d, J=9.3 Hz), 7.98 (1H, d, J=1.7 Hz), 7.85-7.78 (2H, m), 7.68 (1H, dd, J=8.7, 2.0 Hz), 7.59-7.55 (2H, m), 7.33-7.28 (2H, m), 7.13 (1H, d, J=8.3 Hz), 5.86 (1H, dd, J=11.5, 4.8 Hz), 3.91 (1H, dd, J=17.8, 12.0 Hz), 3.35-3.12 (3H, m), 2.75 (2H, d, J=6.6 Hz); ¹³C NMR (101 MHz, CDCl₃) δ 176.2 (C), 170.3 (C), 154.0 (C), 145.3 (CH), 145.1 (CH), 144.5 (C), 143.7 (C), 143.0 (C), 142.6 (C), 142.1 (C), 136.7 (C), 131.8 (C), 130.6 (CH), 130.3 (C), 128.1 (CH), 127.5 (4×CH), 125.8 (CH), 122.7 (CH), 109.8 (CH), 108.5 (CH), 60.2 (CH), 42.3 (CH₂), 29.1 (CH₂), 28.8 (CH₂); HRMS (ESI/Q-TOF) m/z: [M+Na]⁺ calcd for C₂₈H₂₀F₂N₄NaO₅ 553.1294; found, 553.1292.

### 4-(3-(4-(Benzofuran-5-yl)phenyl)-5-(quinoxalin-6-yl)-4,5-dihydro-1H-pyrazol-1-yl)-4-oxobutanoic acid (Compound 105)

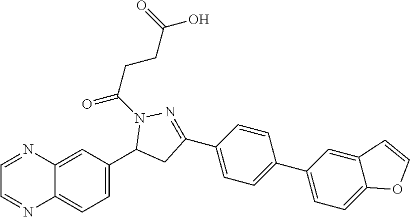 [SMILES: O=C(O)CCC(=O)N1N=C(c2ccc(-c3ccc4occc4c3)cc2)CC1c1ccc2nccnc2c1]

Compound 105 was prepared using a method similar to that described for the preparation of Compound 21: Compound 9 (70 mg, 0.15 mmol), benzofuran-5-boronic acid (50 mg, 0.31 mmol), potassium phosphate tribasic (0.13 g, 0.62 mmol), and palladium acetate (3.5 mg, 10 mol %) in DMF/water (1.6 mL, 1:1 v/v). Purification by flash column chromatography (petroleum ether/EtOAc, 1:1→EtOAc, neat→EtOAc/MeOH, 99:1) afforded Compound 105 as a yellow solid (35 mg, 46%). ¹H NMR (400 MHz, DMSO-d₆) δ 12.07 (1H, s), 8.94-8.92 (2H, m), 8.09 (1H, d, J=8.8 Hz), 8.05 (1H, d, J=2.2 Hz), 8.00 (1H, dd, J=1.9, 0.7 Hz), 7.92-7.89 (3H, m), 7.81 (2H, d, J=8.6 Hz), 7.74-7.65 (3H, m), 7.03 (1H, dd, J=2.2, 0.9 Hz), 5.86 (1H, dd, J=11.9, 5.0 Hz), 4.02 (1H, dd, J=18.1, 12.0 Hz), 3.35 (1H, dd, J=18.2, 5.0 Hz), 3.12 (1H, dt, J=17.1, 6.7 Hz), 3.00 (1H, dt, J=17.1, 6.6 Hz), 2.54-2.50 (2H, m); 13C NMR (101 MHz, DMSO-d₆) δ 173.7 (C), 169.2 (C), 154.2 (C), 154.2 (C), 146.9 (CH), 146.0 (CH), 145.6 (CH), 144.2 (C), 142.3 (C), 142.1 (C), 141.6 (C), 134.5 (C), 129.8 (CH), 129.6 (C), 128.1 (CH), 128.0 (C), 127.4 (2×CH), 127.2 (2×CH), 125.3 (CH), 123.5 (CH), 119.5 (CH), 111.7 (CH), 107.0 (CH), 59.6 (CH), 41.8 (CH₂), 28.8 (CH₂), 28.4 (CH₂); HRMS (ESI/Q-TOF) m/z: [M+Na]⁺ calcd for C₂₉H₂₂N₄NaO₄, 513.1533; found, 513.1532.

### 4-(3-(4-(1H-Indol-5-yl)phenyl)-5-(quinoxalin-6-yl)-4,5-dihydro-1H-pyrazol-1-yl)-4-oxobutanoic acid (Compound 106)

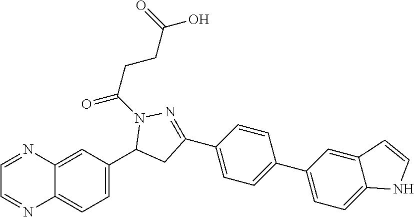 [SMILES: O=C(O)CCC(=O)N1N=C(c2ccc(-c3ccc4[nH]ccc4c3)cc2)CC1c1ccc2nccnc2c1]

Compound 106 was prepared over 2 steps using a method similar to that described for the preparation of Compound 20-step 1: quinoxaline-6-carbaldehyde (158 mg, 1.00 mmol), 1-(4-(1H-indol-5-yl)phenyl)ethan-1-one (235 mg, 1.00 mmol) and 2 M NaOH (2.5 mL, 5.00 mmol) in EtOH (8 mL) to afford (E)-1-(4-(1H-indol-5-yl)phenyl)-3-(quinoxalin-6-yl)prop-2-en-1-one as an green solid (278 mg); step 2: (E)-1-(4-(1H-indol-5-yl)phenyl)-3-(quinoxalin-6-yl)prop-2-en-1-one (150 mg) and hydrazine hydrate (50 μL, 50% w/w) in THF (3 mL), followed by the addition of succinic anhydride (120 mg, 1.20 mmol). Purification by flash column chromatography (petroleum ether/EtOAc, 1:1→EtOAc, neat) afforded Compound 106 as a yellow solid (70 mg, 27% over 2 steps). ¹H NMR (400 MHz, THF-d₈) δ 10.83 (1H, br s), 10.25 (1H, s), 8.78-8.75 (2H, m), 8.01 (1H, d, J=8.7 Hz), 7.97 (1H, d, J=2.1 Hz), 7.88-7.85 (3H, m), 7.73 (2H, d, J=8.6 Hz), 7.70 (1H, dd, J=8.7, 2.1 Hz), 7.42-7.41 (2H, m), 7.24 (1H, dd, J=3.2, 2.4 Hz), 6.50-6.48 (1H, m), 5.82 (1H, dd, J=12.0, 5.1 Hz), 3.96 (1H, dd, J=17.7, 12.1 Hz), 3.31 (1H, dd, J=17.8, 5.1 Hz), 3.17 (1H, dt, J=17.0, 7.1 Hz), 3.06 (1H, dt, J=17.1, 7.1 Hz), 2.58 (2H, t, J=7.1 Hz); ¹³C NMR (101 MHz, THF-d₈) δ 174.2 (C), 170.2 (C), 154.5 (C), 146.5 (CH), 146.1 (CH), 145.75 (C), 145.72 (C), 144.3 (C), 143.7 (C), 137.5 (C), 132.4 (C), 131.0 (CH), 130.5 (C), 130.0 (C), 129.0 (CH), 128.1 (2×CH), 127.9 (2×CH), 127.3 (CH), 126.4 (CH), 121.6 (CH), 119.7 (CH), 112.4 (CH), 103.0 (CH), 61.2 (CH), 42.9 (CH₂), 30.2 (CH₂), 29.2 (CH₂); HRMS (ESI/Q-TOF) m/z: [M+Na]⁺ calcd for C₂₉H₂₃N₅NaO₃, 512.1693; found, 512.1693.

### 4-(3-(4-(6-Isopropoxypyridin-3-yl)phenyl)-5-(quinoxalin-6-yl)-4,5-dihydro-1H-pyrazol-1-yl)-4-oxobutanoic acid (Compound 107)

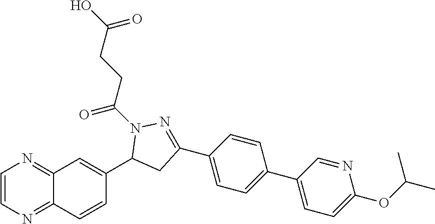 [SMILES: CC(C)Oc1ccc(-c2ccc(C3=NN(C(=O)CCC(=O)O)C(c4ccc5nccnc5c4)C3)cc2)cn1]

Compound 107 was prepared over 2 steps using a method similar to that described for the preparation of Compound 20-step 1: quinoxaline-6-carbaldehyde (294 mg, 1.86 mmol), 1-(4-(6-isopropoxypyridin-3-yl)phenyl)ethan-1-one (475 mg, 1.86 mmol) and 2 M NaOH (4.65 mL, 9.30 mmol) in EtOH (15 mL) to afford (E)-1-(4-(6-isopropoxypyridin-3-yl)phenyl)-3-(quinoxalin-6-yl)prop-2-en-1-one as a colourless solid without further purification (637 mg); step 2: (E)-1-(4-(6-isopropoxypyridin-3-yl)phenyl)-3-(quinoxalin-6-yl)prop-2-en-1-one (637 mg) and hydrazine hydrate (200 μL, 50% w/w, 3.22 mmol) in THF (13 mL), followed by the addition of succinic anhydride (483 mg, 4.83 mmol). Purification by flash column chromatography (EtOAc/petroleum ether, 9:1→EtOAc, neat→EtOAc/MeOH, 9:1→acetone/MeOH, 9:1) afforded Compound 107 as a pale-yellow solid (142 mg, 15% over 2 steps). ¹H NMR (400 MHz, CDCl₃) δ 8.81 (2H, s), 8.41 (1H, d, J=2.5 Hz), 8.11 (1H, d, J=8.7 Hz), 7.99 (1H, d, J=1.7 Hz), 7.84-7.79 (3H, m), 7.68 (1H, dd, J=8.7, 1.9 Hz), 7.59 (2H, d. J=8.3 Hz), 6.77 (1H, d, J=8.7 Hz), 5.85 (1H, dd, J=11.9, 4.9 Hz), 5.34 (1H, hept, J=6.2 Hz), 3.92 (1H, dd, J=17.7, 11.9 Hz), 3.34-3.15 (3H, m), 2.76 (2H, t, J=6.7 Hz), 1.38 (6H, d, J=6.2 Hz); ¹³C NMR (101 MHz, CDCl₃) δ 176.0 (C), 170.4 (C), 163.5 (C), 154.2 (C), 145.4 (CH), 145.3 (CH), 145.2 (CH), 143.8 (C), 143.1 (C), 142.7 (C), 140.5 (C), 137.3 (CH), 130.7 (CH), 129.8 (C), 128.6 (C), 128.2 (CH), 127.6 (2×CH), 126.9 (2×CH), 125.9 (CH), 111.9 (CH), 68.6 (CH), 60.3 (CH), 42.4 (CH₂), 29.2 (CH₂), 28.9 (CH₂), 22.2 (2×CH₃); HRMS (ESI/Q-TOF) m/z: [M+Na]⁺ calcd for C₂₉H₂₇N₅NaO₄, 532.1955; found, 532.1956.

### 4-(3-(4-(Furan-3-yl)phenyl)-5-(quinoxalin-6-yl)-4,5-dihydro-1H-pyrazol-1-yl)-4-oxobutanoic acid (Compound 108)

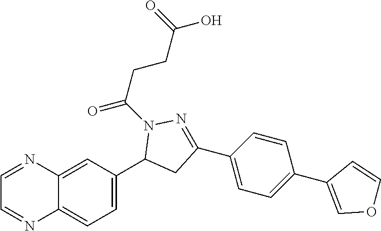 [SMILES: O=C(O)CCC(=O)N1N=C(c2ccc(-c3ccoc3)cc2)CC1c1ccc2nccnc2c1]

Compound 108 was prepared over 2 steps using a method similar to that described for the preparation of Compound 20-step 1: quinoxaline-6-carbaldehyde (158 mg, 1.00 mmol), 1-(4-(furan-3-yl)phenyl) ethan-1-one (186 mg, 1.00 mmol) and 2 M NaOH (2.5 mL, 5.00 mmol) in EtOH (8 mL) to afford (E)-1-(4-(furan-3-yl)phenyl)-3-(quinoxalin-6-yl)prop-2-en-1-one as an yellow solid (251 mg); step 2: (E)-1-(4-(furan-3-yl)phenyl)-3-(quinoxalin-6-yl)prop-2-en-1-one (150 mg) and hydrazine hydrate (57 μL, 50% w/w) in THF (4 mL), followed by the addition of succinic anhydride (138 mg, 1.38 mmol). Purification by flash column chromatography (petroleum ether/EtOAc, 1:1→EtOAc, neat) afforded Compound 108 as a yellow solid (113 mg, 43% over 2 steps). ¹H NMR (400 MHz, DMSO-d₆) δ 12.10 (1H, br s), 8.94-8.92 (2H, m), 8.31 (1H, dd, J=1.6, 0.9 Hz), 8.08 (1H, d, J=8.7 Hz), 7.90 (1H, d, J=2.0 Hz), 7.82 (2H, d, J=8.6 Hz), 7.78 (1H, t, J=1.7 Hz), 7.74-7.70 (3H, m), 7.04 (1H, dd, J=1.9, 0.9 Hz), 5.84 (1H, dd, J=11.9, 5.0 Hz), 3.99 (1H, dd, J=18.2, 12.0 Hz), 3.33 (1H, dd, J=18.1, 5.0 Hz), 3.11 (1H, dt, J=17.1, 6.7 Hz), 2.97 (1H, dt, J=17.2, 6.6 Hz), 2.52-2.50 (2H, m); ¹³C NMR (101 MHz, DMSO-d₆) δ 173.8 (C), 169.2 (C), 154.2 (C), 146.0 (CH), 145.6 (CH), 144.6 (CH), 144.2 (C), 142.2 (C), 141.6 (C), 140.2 (CH), 134.0 (C), 129.8 (CH), 129.4 (C), 128.2 (CH), 127.3 (2×CH), 125.7 (2×CH), 125.3 (CH), 125.2 (C), 108.6 (CH), 59.5 (CH), 41.8 (CH₂), 28.7 (CH₂), 28.3 (CH₂); HRMS (ESI/Q-TOF) m/z: [M+Na]⁺ calcd for C₂₅H₂₀N₄NaO₄, 463.1377; found, 463.1378.

### 4-(3-(3-Fluoro-[1,1′-biphenyl]-4-yl)-5-(quinoxalin-6-yl)-4,5-dihydro-1H-pyrazol-1-yl)-4-oxobutanoic acid (Compound 109)

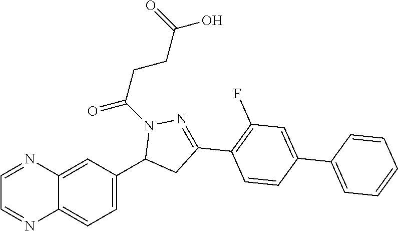 [SMILES: O=C(O)CCC(=O)N1N=C(c2ccc(-c3ccccc3)cc2F)CC1c1ccc2nccnc2c1]

To a solution of quinoxaline-6-carbaldehyde (158 mg, 1.00 mmol) and 1-(3-fluoro-[1,1′-biphenyl]-4-yl) ethan-1-one (214 mg, 1.00 mmol) in EtOH (8 mL) at room temperature was added dropwise 2 M NaOH (2.5 mL, 5.00 mmol), and the reaction mixture was stirred for 3 h. After, the solution was cooled down to 0° C., and the resulting precipitated solids were then collected by filtration, washed with water and dried in vacuo to afford (E)-1-(3-fluoro-[1,1′-biphenyl]-4-yl)-3-(quinoxalin-6-yl)prop-2-en-1-one as an off-white solid (340 mg), which was used directly in the next step without further purification.

A mixture of (E)-1-(3-fluoro-[1,1′-biphenyl]-4-yl)-3-(quinoxalin-6-yl)prop-2-en-1-one (340 mg) and hydrazine hydrate (0.12 mL, 50% w/w) in THF (8 mL) was stirred under reflux for 2 h. After, succinic anhydride (288 mg, 2.88 mmol) was added, and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. The resulting solid was washed with CH₂Cl₂ and isolated by filtration to afford Compound 109 as an off-white solid (328 mg, 70% over 2 steps). ¹H NMR (400 MHz, DMSO-d₆) δ 12.08 (1H, s), 8.95-8.93 (2H, m), 8.09 (1H, d, J=8.7 Hz), 8.01 (1H, t, J=8.3 Hz), 7.92 (1H, d, J=2.0 Hz), 7.79-7.76 (2H, m), 7.74 (1H, dd, J=8.7, 2.0 Hz), 7.70-7.65 (2H, m), 7.52-7.48 (2H, m), 7.45-7.41 (1H, m), 5.83 (1H, dd, J=12.0, 5.1 Hz), 4.09 (1H, ddd, J=18.3, 12.1, 1.9 Hz), 3.35-3.29 (1H, m), 3.11 (1H, dt, J=17.2, 6.7 Hz), 2.98 (1H, dt, J=17.2, 6.5 Hz), 2.53-2.50 (2H, m); ¹³C NMR (101 MHz, DMSO-d₆) δ 173.7 (C), 169.5 (C), 160.7 (C, d, J_{C-F}=252.8 Hz), 150.4 (C, d, J_{C-F}=4.0 Hz), 146.0 (CH), 145.6 (CH), 144.0 (C), 144.0 (C, d, J_{C-F}=8.4 Hz), 142.1 (C), 141.6 (C), 137.7 (C, d, J_{C-F}=1.7 Hz), 129.8 (CH), 129.6 (CH, d, J_{C-F}=3.6 Hz), 129.1 (2×CH), 128.6 (CH), 128.1 (CH), 126.8 (2×CH), 125.4 (CH), 122.8 (CH, d, J_{C-F}=2.8 Hz), 117.8 (C, d. J_{C-F}=11.1 Hz), 114.4 (CH, d, J_{C-F}=23.1 Hz), 59.3 (CH), 43.8 (CH₂, d, J_{C-F}=6.2 Hz), 28.8 (CH₂), 28.3 (CH₂); HRMS (ESI/Q-TOF) m/z: [M+H]⁺ calcd for C₂₇H₂₂FN₄O₃, 469.1670; found, 469.1671.

### 4-(3-(2-Fluoro-[1,1′-biphenyl]-4-yl)-5-(quinoxalin-6-yl)-4,5-dihydro-1H-pyrazol-1-yl)-4-oxobutanoic acid (Compound 110)

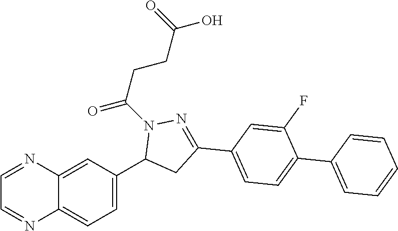 [SMILES: O=C(O)CCC(=O)N1N=C(c2ccc(-c3ccccc3)c(F)c2)CC1c1ccc2nccnc2c1]

A solution of quinoxaline-6-carbaldehyde (158 mg, 1.00 mmol) in EtOH (4 mL) at room temperature was stirred for 10 min, followed by the addition of 2 M NaOH (0.50 mL, 1.00 mmol) at 0° C. Next, 1-(2-fluoro-[1,1′-biphenyl]-4-yl) ethan-1-one (214 mg, 1.00 mmol) was added portion-wise over 30 min, and the reaction mixture was stirred at 0° C. for additional 1 h. After, the resulting precipitated solids were then collected by filtration, washed with water and dried in vacuo to afford (E)-1-(2-fluoro-[1,1′-biphenyl]-4-yl)-3-(quinoxalin-6-yl)prop-2-en-1-one as a brown solid (142 mg), which was used directly in the next step without further purification.

A mixture of (E)-1-(2-fluoro-[1,1′-biphenyl]-4-yl)-3-(quinoxalin-6-yl)prop-2-en-1-one (110 mg) and hydrazine hydrate (39 μL, 50% w/w) in THF (2.5 mL) was stirred under reflux for 2 h. After, succinic anhydride (93 mg, 0.93 mmol) was added, and the reaction mixture was further stirred under reflux for 1 h. The resulting mixture was diluted in EtOAc (30 mL) and washed with water (2×20 mL), then brine (20 mL), dried over anhydrous Na₂SO₄, filtered, and concentrated in vacuo. Purification by flash column chromatography (CH₂Cl₂, neat→CH₂Cl₂/MeOH, 95:5) afforded Compound 110 as a beige solid (25 mg, 7% over 2 steps). ¹H NMR (400 MHz, DMSO-d₆) δ 12.09 (1H, s), 8.95-8.93 (2H, m), 8.09 (1H, d, J=8.6 Hz), 7.92 (1H, d, J=2.0 Hz), 7.77-7.70 (3H, m), 7.66 (1H, t, J=8.1 Hz), 7.61-7.58 (2H, m), 7.53-7.48 (2H, m), 7.46-7.42 (1H, m), 5.87 (1H, dd, J=12.0, 5.1 Hz), 4.01 (1H, dd, J=18.2, 12.0 Hz), 3.37 (1H, dd, J=18.3, 5.1 Hz), 3.11 (1H, dt, J=17.2, 6.7 Hz), 2.99 (1H, dt, J=17.2, 6.6 Hz), 2.53-2.50 (2H, m); ¹³C NMR (101 MHz, DMSO-d₆) δ 173.7 (C), 169.4 (C), 159.0 (C, d, J_{C-F}=246.6 Hz), 153.3 (C), 146.0 (CH), 145.6 (CH), 144.0 (C), 142.1 (C), 141.6 (C), 134.4 (C), 132.3 (C, d, J_{C-F}=8.6 Hz), 131.16 (CH, d, J_{C-F}=3.7 Hz), 129.8 (C, d. J_{C-F}=13.4 Hz), 129.8 (CH), 128.8 (2×CH), 128.7 (2×CH), 128.3 (CH), 128.1 (CH), 125.4 (CH), 123.2 (CH), 114.3 (CH, d, J_{C-F}=24.5 Hz), 59.8 (CH), 41.7 (CH₂), 28.7 (CH₂), 28.3 (CH₂); HRMS (ESI/Q-TOF) m/z: [M+H]⁺ calcd for C₂₇H₂₂FN₄O₃, 469.1670; found, 469.1670.

### 4-(3-([1,1′-Biphenyl]-4-yl)-5-(benzo[c][1,2,5]oxadiazol-5-yl)-4,5-dihydro-1H-pyrazol-1-yl)-4-oxobutanoic acid (Compound 111)

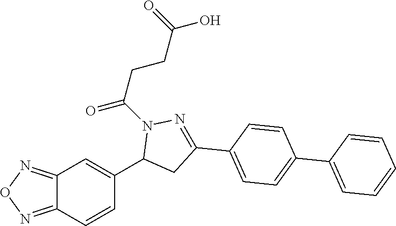 [SMILES: O=C(O)CCC(=O)N1N=C(c2ccc(-c3ccccc3)cc2)CC1c1ccc2nonc2c1]

To a solution of 2,1,3-benzoxadiazole-5-carbaldehyde (296 mg, 2.00 mmol) and 4-acetylbiphenyl (0.33 mL, 2.00 mmol) in EtOH (16 mL) at 0° C. was added dropwise 2 M NaOH (1.0 mL, 2.0 mmol), and the reaction mixture was stirred for 3 h at 0° C. After, the resulting precipitated solids were then collected by filtration, washed with water and dried in vacuo to afford (E)-1-([1,1′-biphenyl]-4-yl)-3-(benzo[c][1,2,5]oxadiazol-5-yl)prop-2-en-1-one as a green solid (535 mg), which was used directly in the next step without further purification.

A mixture of (E)-1-([1,1′-biphenyl]-4-yl)-3-(benzo[c][1,2,5]oxadiazol-5-yl)prop-2-en-1-one (250 mg) and hydrazine hydrate (95 μL, 50% w/w) in THF (6 mL) was stirred under reflux for 2 h. After, succinic anhydride (230 mg, 2.30 mmol) was added, and the reaction mixture was further stirred under reflux for 1 h. The resulting mixture was diluted in EtOAc (50 mL) and washed with water (2×30 mL), then brine (30 mL), dried over anhydrous Na₂SO₄, filtered, and concentrated in vacuo. Purification by flash column chromatography (petroleum ether/EtOAc, 3:7→EtOAc, neat) afforded Compound 111 as an orange solid (300 mg, 73% over 2 steps). ¹H NMR (400 MHz, CDCl₃) δ 7.84-7.81 (3H, m), 7.73-7.72 (1H, m), 7.68 (2H, d, J=8.7 Hz), 7.64-7.61 (2H, m), 7.49-7.45 (2H, m), 7.41-7.37 (1H, m), 7.28-7.26 (1H, m), 5.69 (1H, dd, J=12.0, 5.1 Hz), 3.88 (1H, dd, J=17.8, 12.0 Hz), 3.32-3.23 (1H, m), 3.22 (1H, dd, J=17.7, 5.1 Hz), 3.12 (1H, ddd, J=17.4, 7.2, 5.9 Hz), 2.82-2.69 (2H, m); ¹³C NMR (101 MHz, CDCl₃) δ 177.4 (C), 170.3 (C), 154.3 (C), 149.3 (C), 148.9 (C), 144.6 (C), 143.8 (C), 140.1 (C), 130.2 (CH), 129.6 (C), 129.1 (2×CH), 128.2 (CH), 127.7 (2×CH), 127.4 (2×CH), 127.2 (2×CH), 118.1 (CH), 112.8 (CH), 60.2 (CH), 41.4 (CH₂), 29.1 (CH₂), 28.7 (CH₂); HRMS (ESI/Q-TOF) m/z: [M+Na]⁺ calcd for C₂₅H₂₀N₄NaO₄, 463.1377; found, 463.1377.

### 4-(3-(4-Chlorophenyl)-5-(3,4-difluorophenyl)-4,5-dihydro-1H-pyrazol-1-yl)-4-oxobutanoic acid (Compound 112)

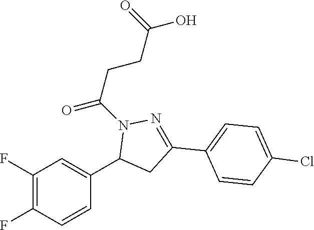 [SMILES: O=C(O)CCC(=O)N1N=C(c2ccc(Cl)cc2)CC1c1ccc(F)c(F)c1]

Compound 112 was prepared over 2 steps using a method similar to that described for the preparation of Compound 20-step 1:3,4-difluorobenzaldehyde (142 mg, 1.00 mmol), 4-chloroacetophenone (0.13 mL, 1.00 mmol) and 2 M NaOH (2.5 mL, 5.00 mmol) in EtOH (8 mL) to afford (E)-1-(4-chlorophenyl)-3-(3,4-difluorophenyl)prop-2-en-1-one as a colourless solid (198 mg); step 2: (E)-1-(4-chlorophenyl)-3-(3,4-difluorophenyl)prop-2-en-1-one (198 mg) and hydrazine hydrate (89 μL, 50% w/w) in THF (6 mL), followed by the addition of succinic anhydride (213 mg, 2.13 mmol). Purification by flash column chromatography (petroleum ether/EtOAc, 1:1→EtOAc, neat) afforded Compound 112 as an off-white solid (106 mg, 27% over 2 steps). ¹H NMR (400 MHz, CDCl₃) δ 7.67 (2H, d, J=8.7 Hz), 7.40 (2H, d, J=8.8 Hz), 7.14-7.07 (1H, m), 7.02 (1H, ddd, J=10.9, 7.4, 2.2 Hz), 6.98-6.94 (1H, m), 5.54 (1H, dd, J=11.9, 4.8 Hz), 3.74 (1H, dd, J=17.8, 11.9 Hz), 3.18 (1H, dt, J=17.4, 6.7 Hz), 3.12-3.04 (2H, m), 2.73 (2H, t, J=6.7 Hz); ¹³C NMR (101 MHz, CDCl₃) δ 177.8 (C), 170.1 (C), 153.4 (C), 151.5 (C, dd, J_{C-F}=71.6, 12.4 Hz), 149.1 (C, dd, J_{C-F}=70.4, 12.7 Hz), 138.5 (C), 136.9 (C), 129.5 (C), 129.3 (2×CH), 128.1 (2×CH), 121.9 (CH, dd, J_{C-F}=5.9, 3.2 Hz), 118.0 (CH, d, J_{C-F}=17.6 Hz), 114.9 (CH, d, J_{C-F}=18.0 Hz), 59.6 (CH), 42.2 (CH₂), 29.0 (CH₂), 28.8 (CH₂); HRMS (ESI/Q-TOF) m/z: [M+Na]⁺ calcd for C₁₉H₁₅ClF₂N₂NaO₃, 415.0631; found, 415.0629.

### 4-(3-(4-Chlorophenyl)-5-(quinoxalin-6-yl)-4,5-dihydro-1H-pyrazol-1-yl)-4-oxobutanamide (Compound 113)

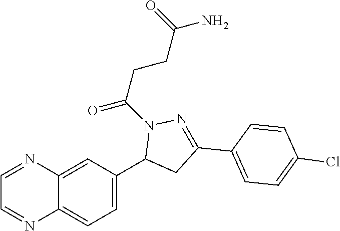 [SMILES: NC(=O)CCC(=O)N1N=C(c2ccc(Cl)cc2)CC1c1ccc2nccnc2c1]

A mixture of (E)-1-(4-chlorophenyl)-3-(quinoxalin-6-yl)prop-2-en-1-one (0.15 g, 0.51 mmol), 4-hydrazino-4-oxobutanamide (67 mg, 0.51 mmol) and NaOH (20 mg, 0.51 mmol) in EtOH (3.0 mL) was heated at 80° C. for 18 h. The resulting reaction mixture was then diluted with EtOAc (20 mL), washed with water (20 mL) and the separated aqueous layer further extracted with EtOAc (3×30 mL). The combined organic layers were washed with water (3×30 mL), then brine (30 mL), dried over anhydrous Na₂SO₄, and concentrated in vacuo. Purification by flash column chromatography (EtOAc, neat→EtOAc/MeOH, 20:1) afforded Compound 113 as a yellow solid (50 mg, 24%). ¹H NMR (300 MHz, CDCl₃) δ 8.82-8.81 (2H, m), 8.10 (1H, d, J=8.7 Hz), 7.95 (1H, d, 1.9 Hz), 7.70-7.63 (3H, m), 7.42-7.38 (2H, m), 5.80 (1H, dd, J=12.3, 6.4 Hz), 3.85 (1H, dd, J=17.2, 12.3 Hz), 3.26-3.17 (3H, m), 2.62-2.52 (2H, m).

### 3-(3-([1,1′-Biphenyl]-3-yl)-5-(quinoxalin-6-yl)-4,5-dihydro-1H-pyrazol-1-yl)benzoic acid (Compound 114)

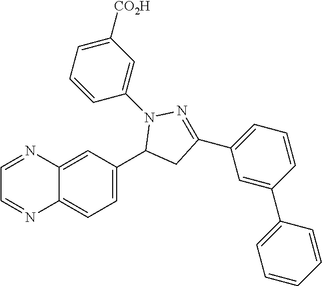 [SMILES: O=C(O)c1cccc(N2N=C(c3cccc(-c4ccccc4)c3)CC2c2ccc3nccnc3c2)c1]

A suspension of (E)-1-([1,1′-biphenyl]-3-yl)-3-(quinoxalin-6-yl)prop-2-en-1-one (200 mg, 0.595 mmol) and 3-hydrazinobenzoic acid (455 mg, 2.97 mmol) in AcOH (4 mL) was stirred under reflux for 6 h. The reaction mixture was poured onto ice water, collected by filtration and dried in vacuo. Purification by flash column chromatography (petroleum ether/EtOAc, 7:3→petroleum ether/EtOAc, 1:1) afforded Compound 114 as a yellow solid (31 mg, 11%, ca. 90% purity). ¹H NMR (400 MHz, CDCl₃) 8.85-8.84 (2H, m), 8.13-8.11 (2H, m), 7.95 (1H, t, J=1.8 Hz), 7.80-7.74 (3H, m), 7.64-7.57 (3H, m), 7.53-7.45 (4H, m), 7.41-7.35 (2H, m), 7.29-7.25 (1H, m), 5.65 (1H, dd, J=12.3, 6.4 Hz), 4.06 (1H, dd, J=17.2, 12.3 Hz), 3.33 (1H, dd. J=17.1, 6.5 Hz); ¹³C NMR (101 MHz, CDCl₃) δ 171.1 (C), 147.7 (C), 145.5 (CH), 145.2 (CH), 144.6 (C), 144.4 (C), 143.1 (C), 142.7 (C), 141.9 (C), 140.9 (C), 132.8 (C), 131.1 (CH), 130.4 (C), 129.3 (CH), 129.2 (CH), 129.0 (2×CH), 128.2 (CH), 128.1 (CH), 127.7 (CH), 127.4 (2×CH), 126.5 (CH), 125.0 (CH), 124.9 (CH), 121.2 (CH), 118.6 (CH), 114.7 (CH), 64.0 (CH), 43.7 (CH₂); HRMS (ESI/Q-TOF) m/z: [M+Na]⁺ calcd for C₃₀H₂₂N₄NaO₂, 493.1635; found, 493.1638.

### 4-(3-(4-Chlorophenyl)-5-(quinoxalin-6-yl)-4,5-dihydro-1H-pyrazol-1-yl)benzoic acid (Compound 115)

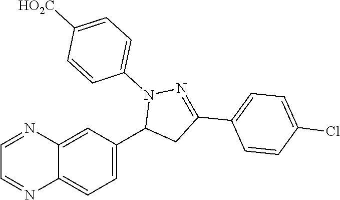 [SMILES: O=C(O)c1ccc(N2N=C(c3ccc(Cl)cc3)CC2c2ccc3nccnc3c2)cc1]

Compound 115 was prepared following a method similar to that described for the preparation of Compound 114 using (E)-1-(4-chlorophenyl)-3-(quinoxalin-6-yl)prop-2-en-1-one (200 mg, 0.679 mmol) and 4-hydrazinobenzoic acid (516 mg, 3.39 mmol) in AcOH (4 mL). Purification by flash column chromatography (petroleum ether/EtOAc, 7:3→petroleum ether/EtOAc, 1:1) afforded Compound 115 as a yellow solid (26 mg, 9%). ¹H NMR (400 MHz, DMSO-d₆) δ 12.32 (1H, br s), 8.92 (2H, s), 8.11 (1H, d, J=8.7 Hz), 7.98 (1H, d, J=2.0 Hz), 7.82 (2H, d, J=8.6 Hz), 7.75-7.71 (3H, m), 7.51 (2H, d, J=8.6 Hz), 7.12 (2H, d, J=9.0 Hz), 5.99 (1H, dd, J=12.2, 5.1 Hz), 4.07 (1H, dd, J=17.8, 12.3 Hz), 3.35 (1H, dd, J=18.0, 5.2 Hz); ¹³C NMR (101 MHz, DMSO-d₆) δ 167.1 (C), 148.9 (C), 146.7 (C), 146.1 (CH), 145.8 (CH), 143.5 (C), 142.1 (C), 141.7 (C), 133.8 (C), 130.9 (2×CH), 130.6 (C), 130.5 (CH), 128.8 (2×CH), 128.0 (CH), 127.8 (2×CH), 125.8 (CH), 120.5 (C), 112.2 (2×CH), 62.2 (CH), 42.5 (CH₂); HRMS (ESI/Q-TOF) m/z: [M+H]⁺ calcd for C₂₄H₁₈ClN₄O₂, 429.1113; found, 429.1112.

### N-(Methylsulfonyl)-4-oxo-4-(5-(quinoxalin-6-yl)-3-(4-(trifluoromethoxy)phenyl)-4,5-dihydro-1H-pyrazol-1-yl) butanamide (Compound 116)

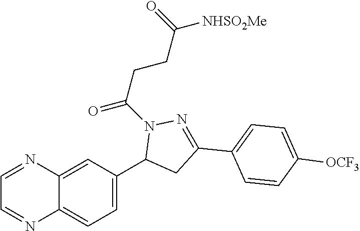

Compound 116 was prepared following a method similar to that described for the preparation of Compound 55 using Compound 8 (0.10 g, 0.22 mmol), methanesulfonamide (31 mg, 0.33 mmol), N-(3-dimethylaminopropyl)-N′-ethylcarbodiimide hydrochloride (EDCI) (63 mg, 0.33 mmol), and 4-(dimethylamino)pyridine (DMAP) (32 mg, 0.26 mmol) in CH₂Cl₂ (2.2 mL). Purification by flash column chromatography (petroleum ether/EtOAc, 1:1→EtOAc, neat) afforded Compound 116 as a colourless solid (74 mg, 63%). ¹H NMR (400 MHz, CDCl₃) δ 9.57 (1H, s), 8.82 (2H, s), 8.11 (1H, d, J=8.7 Hz), 7.95 (1H, d, J=2.0 Hz), 7.79 (2H, d, J=8.9 Hz), 7.64 (1H, dd, J=8.7, 2.0 Hz), 7.28 (2H, d, J=9.2 Hz), 5.87 (1H, dd, J=11.8, 4.7 Hz), 3.88 (1H, dd, J=17.8, 11.8 Hz), 3.33-3.17 (3H, m), 3.15 (3H, s), 2.72-2.52 (2H, m); ¹³C NMR (101 MHz, CDCl₃) δ 171.7 (C), 170.5 (C), 154.2 (C), 151.1 (C), 145.6 (CH), 145.4 (CH), 143.2 (C), 143.0 (C), 142.7 (C), 130.8 (CH), 129.4 (C), 128.6 (2×CH), 128.0 (CH), 126.0 (CH), 121.3 (2×CH), 120.5 (C, q, J_{C-F}=258.4 Hz), 60.3 (CH), 42.4 (CH₂), 41.3 (CH₃), 31.2 (CH₂), 29.0 (CH₂); HRMS (ESI/Q-TOF) m/z: [M+H]⁺ calcd for C₂₃H₂₁F₃N₅O₅S, 536.1210; found, 536.1211.

### 4-(3-(4′-Isopropoxy-[1,1′-biphenyl]-4-yl)-5-(quinoxalin-6-yl)-4,5-dihydro-1H-pyrazol-1-yl)-N-(methylsulfonyl)-4-oxobutanamide (Compound 117)

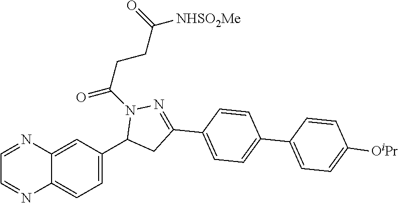

A solution of Compound 64 (41 mg, 0.080 mmol), methanesulfonamide (11 mg, 0.12 mmol), N-(3-dimethylaminopropyl)-N′-ethylcarbodiimide hydrochloride (EDCI) (23 mg, 0.12 mmol), and 4-(dimethylamino)pyridine (DMAP) (12 mg, 0.096 mmol) in CH₂Cl₂ (0.8 mL) was stirred at room temperature for 24 h. The reaction mixture was diluted with CH₂Cl₂ (30 mL), washed with 0.2 M aq. HCl (2×15 mL), water (2×15 mL), then brine (15 mL), dried over anhydrous sodium sulfate and concentrated in vacuo. Purification by flash column chromatography (petroleum ether/EtOAc, 1:1→EtOAc, neat) afforded Compound 117 as an off-white solid (23 mg, 50%). ¹H NMR (400 MHz, CDCl₃) δ 8.81 (2H, s), 8.11 (1H, d, J=8.7 Hz), 7.98 (1H, d, J=2.0 Hz), 7.78 (2H, d, J=8.5 Hz), 7.66 (1H, dd, J=8.7, 2.1 Hz), 7.62 (2H, d. J=8.5 Hz), 7.55 (2H, d, J=8.8 Hz), 6.97 (2H, d, J=8.8 Hz), 5.86 (1H, dd, J=11.7, 4.6 Hz), 4.61 (1H, hept, J=6.1 Hz), 3.90 (1H, dd, J=17.7, 11.8 Hz), 3.35-3.18 (3H, m), 3.15 (3H, $), 2.69-2.52 (2H, m), 1.37 (6H, d, J=6.0 Hz); ¹³C NMR (101 MHz, CDCl₃) δ 171.8 (C), 170.4 (C), 158.3 (C), 155.5 (C), 145.5 (CH), 145.3 (CH), 143.53 (C), 143.46 (C), 143.1 (C), 142.7 (C), 132.2 (C), 130.8 (CH), 128.8 (C), 128.3 (2×CH), 128.1 (CH), 127.5 (2×CH), 127.0 (2×CH), 126.0 (CH), 116.4 (2×CH), 70.2 (CH), 60.2 (CH), 42.4 (CH₂), 41.3 (CH₃), 31.4 (CH₂), 29.2 (CH₂), 22.2 (2×CH₃); HRMS (ESI/Q-TOF) m/z: [M+H]⁺ calcd for C₃₁H₃₂N₅O₅S, 586.2119; found, 586.2119.

### N-(Isopropylsulfonyl)-4-oxo-4-(5-(quinoxalin-6-yl)-3-(4-(trifluoromethoxy)phenyl)-4,5-dihydro-1H-pyrazol-1-yl) butanamide (Compound 118)

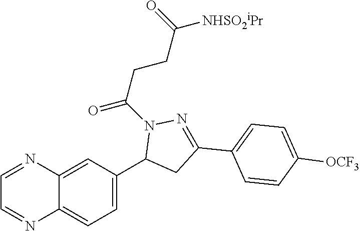

Compound 118 was prepared following a method similar to that described for the preparation of Compound 55 using Compound 8 (46 mg, 0.10 mmol), propane-2-sulfonamide (25 mg, 0.20 mmol), N-(3-dimethylaminopropyl)-N′-ethylcarbodiimide hydrochloride (EDCI) (38 mg, 0.20 mmol), and 4-(dimethylamino)pyridine (DMAP) (15 mg, 0.12 mmol) in CH₂Cl₂ (1.0 mL). Purification by flash column chromatography (petroleum ether/EtOAc, 1:1→EtOAc, neat) afforded Compound 118 as a colourless solid (37 mg, 65%). ¹H NMR (400 MHz, CDCl₃) δ 9.31 (1H, s), 8.81 (2H, s), 8.10 (1H, d, J=8.7 Hz), 7.95 (1H, d, J=2.0 Hz), 7.78 (2H, d, J=8.8 Hz), 7.64 (1H, dd, J=8.7, 2.1 Hz), 7.28 (2H, d, J=9.0 Hz), 5.87 (1H, dd, J=11.8, 4.7 Hz), 3.87 (1H, dd, J=17.8, 11.9 Hz), 3.63 (1H, hept, J=6.9 Hz), 3.33 (1H, dt, J=17.9, 6.4 Hz), 3.22 (1H, dd, J=17.8, 4.7 Hz), 3.20-3.11 (1H, m), 2.65 (2H, t, J=6.4 Hz), 1.27 (3H, d, J=6.9 Hz), 1.11 (3H, d, J=6.8 Hz); ¹³C NMR (101 MHz, CDCl₃) δ 171.6 (C), 170.5 (C), 154.0 (C), 151.1 (C), 145.6 (CH), 145.3 (CH), 143.3 (C), 143.0 (C), 142.6 (C), 130.8 (CH), 129.4 (C), 128.6 (2×CH), 128.0 (CH), 126.0 (CH), 121.2 (2×CH), 120.5 (C, q, J_{C-F}=258.2 Hz), 60.3 (CH), 53.8 (CH), 42.3 (CH₂), 31.3 (CH₂), 29.1 (CH₂), 15.9 (CH₃), 15.7 (CH₃); HRMS (ESI/Q-TOF) m/z: [M+H]⁺ calcd for C₂₅H₂₅F₃NO₅S, 564.1523; found, 564.1524.

### 4-(3-([1,1′-Biphenyl]-3-yl)-5-(quinoxalin-6-yl)-4,5-dihydro-1H-pyrazol-1-yl)-N-(isopropylsulfonyl)-4-oxobutanamide (Compound 119)

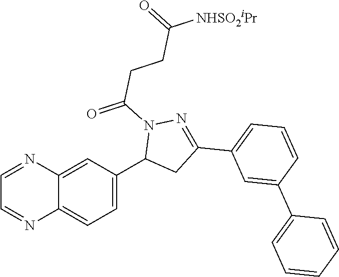

Compound 119 was prepared following a method similar to that described for the preparation of Compound 117 using Compound 13 (68 mg, 0.15 mmol), propane-2-sulfonamide (28 mg, 0.22 mmol), N-(3-dimethylaminopropyl)-N′-ethylcarbodiimide hydrochloride (EDCI) (43 mg, 0.22 mmol), and 4-(dimethylamino)pyridine (DMAP) (22 mg, 0.18 mmol) in CH₂Cl₂ (1.5 mL). Purification by flash column chromatography (petroleum ether/EtOAc, 1:1→EtOAc, neat) afforded Compound 119 as a colourless solid (42 mg, 50%). ¹H NMR (400 MHz, CDCl₃) δ 8.80 (2H, s), 8.10 (1H, d, J=8.7 Hz), 7.98 (1H, d, J=2.0 Hz), 7.95 (1H, t, J=1.8 Hz), 7.72-7.59 (5H, m), 7.52-7.44 (3H, m), 7.38 (1H, t, J=7.3 Hz), 5.88 (1H, dd, J=11.8, 4.6 Hz), 3.92 (1H, dd, J=17.8, 11.8 Hz), 3.64 (1H, hept, J=7.0 Hz), 3.37 (1H, dt, J=17.9, 6.4 Hz), 3.28 (1H, dd, J=17.8, 4.7 Hz), 3.17 (1H, dt, J=17.7, 6.4 Hz), 2.66 (2H, t, J=6.5 Hz), 1.27 (3H, d, J=6.8 Hz), 1.11 (3H, d, J=6.8 Hz); ¹³C NMR (101 MHz, CDCl₃) δ 171.7 (C), 170.5 (C), 155.4 (C), 145.5 (CH), 145.2 (CH), 143.5 (C), 143.0 (C), 142.6 (C), 142.2 (C), 140.3 (C), 131.3 (C), 130.7 (CH), 129.8 (CH), 129.4 (CH), 129.1 (2×CH), 128.1 (CH), 128.0 (CH), 127.3 (2×CH), 126.0 (CH), 125.8 (CH), 125.7 (CH), 60.2 (CH), 53.7 (CH), 42.5 (CH₂), 31.4 (CH₂), 29.2 (CH₂), 15.9 (CH₃), 15.7 (CH₃); HRMS (ESI/Q-TOF) m/z: [M+H]⁺ calcd for C₃₀H₃₀N₅O₄S, 556.2013; found, 556.2009.

### 4-(3-(4′-Isopropoxy-[1,1′-biphenyl]-4-yl)-5-(quinoxalin-6-yl)-4,5-dihydro-1H-pyrazol-1-yl)-N-(isopropylsulfonyl)-4-oxobutanamide (Compound 120)

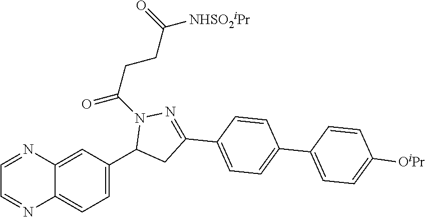

Compound 120 was prepared following a method similar to that described for the preparation of Compound 117 using Compound 64 (46 mg, 0.090 mmol), propane-2-sulfonamide (17 mg, 0.14 mmol), N-(3-dimethylaminopropyl)-N′-ethylcarbodiimide hydrochloride (EDCI) (26 mg, 0.14 mmol), and 4-(dimethylamino)pyridine (DMAP) (13 mg, 0.11 mmol) in CH₂Cl₂ (0.9 mL). Purification by flash column chromatography (petroleum ether/EtOAc, 1:1→EtOAc, neat) afforded Compound 120 as a yellow solid (27 mg, 49%). ¹H NMR (400 MHz, CDCl₃) δ 9.25 (1H, br s), 8.83-8.82 (2H, m), 8.11 (1H, d, J=8.7 Hz), 7.97 (1H, d, J=2.0 Hz), 7.79 (2H, d, J=8.7 Hz), 7.67 (1H, dd, J=8.7, 2.1 Hz), 7.63 (2H, d, J=8.7 Hz), 7.55 (2H, d, J=8.9 Hz), 6.98 (2H, d, J=8.9 Hz), 5.85 (1H, dd, J=11.8, 4.6 Hz), 4.61 (1H, hept, J=6.0 Hz), 3.92 (1H, dd, J=17.7, 11.8 Hz), 3.66 (1H, hept, J=6.8 Hz), 3.41-3.17 (3H, m), 2.73-2.61 (2H, m), 1.37 (6H, d, J=6.1 Hz), 1.30 (3H, d, J=6.9 Hz), 1.15 (3H, d. J=6.8 Hz); ¹³C NMR (101 MHz, CDCl₃) δ 171.6 (C), 170.4 (C), 158.3 (C), 155.3 (C), 145.5 (CH), 145.3 (CH), 143.5 (2× C), 143.1 (C), 142.7 (C), 132.2 (C), 130.8 (CH), 128.9 (C), 128.3 (2×CH), 128.1 (CH), 127.5 (2×CH), 127.0 (2×CH), 126.1 (CH), 116.4 (2×CH), 70.2 (CH), 60.2 (CH), 53.8 (CH), 42.4 (CH₂), 31.7 (CH₂), 29.4 (CH₂), 22.2 (2×CH₃), 16.0 (CH₃), 15.7 (CH₃); HRMS (ESI/Q-TOF) m/z: [M+H]⁺ calcd for C₃₃H₃₆N₅O₅S, 614.2432; found, 614.2432.

### N-(tert-Butylsulfonyl)-4-(3-(4-chlorophenyl)-5-(quinoxalin-6-yl)-4,5-dihydro-1H-pyrazol-1-yl)-4-oxobutanamide (Compound 121)

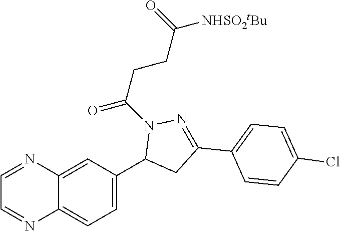

Compound 121 was prepared following a method similar to that described for the preparation of Compound 117 using Compound 1 (0.10 g, 0.24 mmol), tert-butylsulfonamide (49 mg, 0.36 mmol), N-(3-dimethylaminopropyl)-N′-ethylcarbodiimide hydrochloride (EDCI) (69 mg, 0.36 mmol) and 4-(dimethylamino)pyridine (DMAP) (35 mg, 0.29 mmol) in CH₂Cl₂ (2.4 mL). Purification by flash column chromatography (petroleum ether/EtOAc, 1:1→EtOAc, neat) afforded Compound 121 as an off-white solid (66 mg, 52%). ¹H NMR (400 MHz, CDCl₃) δ 8.80 (2H, s), 8.08 (1H, d, J=8.8 Hz), 7.94 (1H, d, J=2.0 Hz), 7.67 (2H, d, J=8.6 Hz), 7.63 (1H, dd, J=8.7, 2.1 Hz), 7.40 (2H, d, J=8.7 Hz), 5.82 (1H, dd, J=11.9, 4.8 Hz), 3.84 (1H, dd, J=17.7, 11.9 Hz), 3.30-3.14 (3H, m), 2.75-2.60 (2H, m), 1.29 (9H, s); 13C NMR (101 MHz, CDCl₃) δ 170.8 (C), 170.4 (C), 154.1 (C), 145.5 (CH), 145.3 (CH), 143.4 (C), 143.1 (C), 142.7 (C), 137.1 (C), 130.8 (CH), 129.4 (C), 129.3 (2×CH), 128.2 (2×CH), 128.1 (CH), 126.0 (CH), 62.1 (C), 60.3 (CH), 42.3 (CH₂), 31.8 (CH₂), 29.3 (CH₂), 24.3 (3×CH₃); HRMS (ESI/Q-TOF) m/z: [M+Na]⁺ calcd for C₂₅H₂₆ClN₅NaO₄S, 550.1286; found, 550.1289.

### 4-(3-([1,1′-Biphenyl]-3-yl)-5-(quinoxalin-6-yl)-4,5-dihydro-1H-pyrazol-1-yl)-4-oxo-N-(phenylsulfonyl) butanamide (Compound 122)

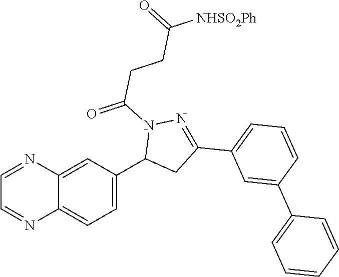

Compound 122 was prepared following a method similar to that described for the preparation of Compound 117 using Compound 13 (68 mg, 0.15 mmol), benzenesulfonamide (35 mg, 0.22 mmol), N-(3-dimethylaminopropyl)-N′-ethylcarbodiimide hydrochloride (EDCI) (43 mg, 0.22 mmol), and 4-(dimethylamino)pyridine (DMAP) (22 mg, 0.18 mmol) in CH₂Cl₂ (1.5 mL). Purification by flash column chromatography (petroleum ether/EtOAc, 1:1→EtOAc, neat) afforded Compound 122 as a colourless solid (50 mg, 56%). ¹H NMR (400 MHz, DMSO-d₆) δ 12.11 (1H, s), 8.95-8.93 (2H, m), 8.07 (1H, d, J=8.7 Hz), 7.99 (1H, t, J=1.8 Hz), 7.90-7.87 (3H, m), 7.83 (1H, dt, J=7.6, 1.3 Hz), 7.77 (1H, ddd, J=7.8, 1.9, 1.1 Hz), 7.73-7.68 (3H, m), 7.63-7.59 (1H, m), 7.58-7.45 (5H, m), 7.41-7.37 (1H, m), 5.81 (1H, dd, J=11.9, 5.0 Hz), 4.02 (1H, dd, J=18.3, 12.0 Hz), 3.43 (1H, dd, J=18.3, 5.0 Hz), 3.08-2.93 (2H, m), 2.61-2.52 (2H, m); ¹³C NMR (101 MHz, DMSO-d₆) δ 170.9 (C), 168.9 (C), 154.6 (C), 146.0 (CH), 145.6 (CH), 144.1 (C), 142.1 (C), 141.6 (C), 140.7 (C), 139.5 (C), 139.4 (C), 133.4 (CH), 131.6 (C), 129.8 (CH), 129.4 (CH), 129.0 (4×CH), 128.7 (CH), 128.1 (CH), 127.8 (CH), 127.3 (2×CH), 126.8 (2×CH), 125.6 (CH), 125.3 (CH), 125.1 (CH), 59.6 (CH), 41.8 (CH₂), 30.0 (CH₂), 27.8 (CH₂); HRMS (ESI/Q-TOF) m/z: [M+H]⁺ calcd for C₃₃H₂₈N₅O₄S, 590.1857; found, 590.1857.

### 4-(3-([1,1′-Biphenyl]-4-yl)-5-(quinoxalin-6-yl)-4,5-dihydro-1H-pyrazol-1-yl)-4-oxo-N-(phenylsulfonyl) butanamide (Compound 123)

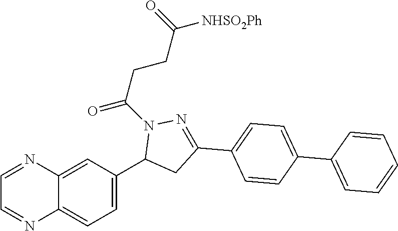

Compound 123 was prepared following a method similar to that described for the preparation of Compound 55 using Compound 15 (59 mg, 0.13 mmol), benzenesulfonamide (31 mg, 0.20 mmol), N-(3-dimethylaminopropyl)-N′-ethylcarbodiimide hydrochloride (EDCI) (37 mg, 0.20 mmol), and 4-(dimethylamino)pyridine (DMAP) (19 mg, 0.16 mmol) in CH₂Cl₂ (1.3 mL). Purification by flash column chromatography (petroleum ether/EtOAc, 1:1→EtOAc, neat) afforded Compound 123 as a yellow solid (25 mg, 33%). ¹H NMR (400 MHz, CDCl₃) δ 9.95 (1H, br s), 8.83 (2H, s), 8.11 (1H, d, J=8.7 Hz), 7.98 (1H, d, J=2.1 Hz), 7.97-7.94 (2H, m), 7.80 (2H, d, J=8.6 Hz), 7.68-7.60 (5H, m), 7.54-7.50 (1H, m), 7.49-7.37 (5H, m), 5.90 (1H, dd, J=11.7, 4.5 Hz), 3.91 (1H, dd, J=17.7, 11.8 Hz), 3.27 (1H, dd, J=17.8, 4.6 Hz), 3.27-3.13 (2H, m), 2.68-2.53 (2H, m); ¹³C NMR (101 MHz, CDCl₃) δ 170.5 (C), 170.4 (C), 155.4 (C), 145.6 (CH), 145.3 (CH), 143.9 (C), 143.4 (C), 143.1 (C), 142.7 (C), 140.1 (C), 139.1 (C), 133.7 (CH), 130.8 (CH), 129.6 (C), 129.1 (2×CH), 128.9 (2×CH), 128.4 (2× CH), 128.2 (CH), 128.1 (CH), 127.6 (2×CH), 127.5 (2×CH), 127.2 (2×CH), 126.0 (CH), 60.2 (CH), 42.5 (CH₂), 31.5 (CH₂), 29.0 (CH₂); HRMS (ESI/Q-TOF) m/z: [M+Na]⁺ calcd for C₃₃H₂₇N₅NaO₄S, 612.1676; found, 612.1673.

### 4-(3-(4-Chlorophenyl)-5-(quinoxalin-6-yl)-4,5-dihydro-1H-pyrazol-1-yl)-4-oxo-N-(o-tolylsulfonyl) butanamide (Compound 124)

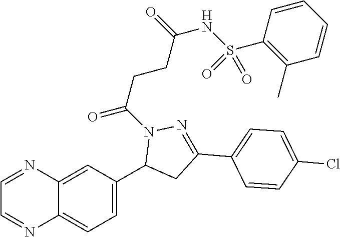 [SMILES: Cc1ccccc1S(=O)(=O)NC(=O)CCC(=O)N1N=C(c2ccc(Cl)cc2)CC1c1ccc2nccnc2c1]

Compound 124 was prepared following a method similar to that described for the preparation of Compound 117 using Compound 1 (0.10 g, 0.24 mmol), o-toluenesulfonamide (62 mg, 0.36 mmol), N-(3-dimethylaminopropyl)-N′-ethylcarbodiimide hydrochloride (EDCI) (69 mg, 0.36 mmol), and 4-(dimethylamino)pyridine (DMAP) (35 mg, 0.29 mmol) in CH₂Cl₂ (2.4 mL). Purification by flash column chromatography (petroleum ether/EtOAc, 1:1→EtOAc, neat) afforded Compound 124 as a colourless solid (89 mg, 66%). ¹H NMR (400 MHz, DMSO-d₆) δ 12.17 (1H, s), 8.94-8.93 (2H, m), 8.04 (1H, d, J=8.7 Hz), 7.91 (1H, dd, J=7.9, 1.4 Hz), 7.87 (1H, d, J=2.0 Hz), 7.79 (2H, d, J=8.6 Hz), 7.65 (1H, dd, J=8.7, 2.1 Hz), 7.52 (2H, d, J=8.6 Hz), 7.47 (1H, td, J=7.5, 1.5 Hz), 7.34-7.29 (2H, m), 5.78 (1H, dd, J=12.0, 5.1 Hz), 3.93 (1H, dd, J=18.3, 12.0 Hz), 3.28 (1H, dd, J=18.3, 5.2 Hz), 3.02-2.89 (2H, m), 2.57-2.55 (2H, m), 2.54 (3H, s); ¹³C NMR (101 MHz, DMSO-d₆) δ 170.7 (C), 168.9 (C), 153.6 (C), 146.0 (CH), 145.6 (CH), 143.9 (C), 142.1 (C), 141.6 (C), 137.5 (C), 136.8 (C), 135.0 (C), 133.2 (CH), 132.2 (CH), 129.9 (CH), 129.80 (CH), 129.79 (C), 128.8 (2×CH), 128.5 (2×CH), 128.1 (CH), 126.0 (CH), 125.4 (CH), 59.7 (CH), 41.6 (CH₂), 29.9 (CH₂), 27.9 (CH₂), 19.4 (CH₃); HRMS (ESI/Q-TOF) m/z: [M+Na]⁺ calcd for C₂₈H₂₄ClN₅NaO₄S, 584.1130; found, 584.1131.

### 4-(3-(4-Chlorophenyl)-5-(quinoxalin-6-yl)-4,5-dihydro-1H-pyrazol-1-yl)-4-oxo-N-tosylbutanamide (Compound 125)

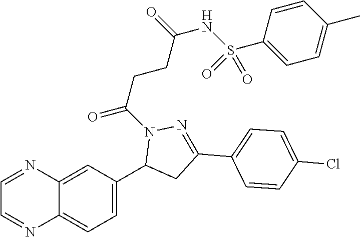 [SMILES: Cc1ccc(S(=O)(=O)NC(=O)CCC(=O)N2N=C(c3ccc(Cl)cc3)CC2c2ccc3nccnc3c2)cc1]

Compound 125 was prepared following a method similar to that described for the preparation of Compound 117 using Compound 1 (0.10 g, 0.24 mmol), p-toluenesulfonamide (62 mg, 0.36 mmol), N-(3-dimethylaminopropyl)-N′-ethylcarbodiimide hydrochloride (EDCI) (69 mg, 0.36 mmol), and 4-(dimethylamino)pyridine (DMAP) (35 mg, 0.29 mmol) in CH₂Cl₂ (2.4 mL). Purification by flash column chromatography (petroleum ether/EtOAc, 1:1→EtOAc, neat) afforded Compound 125 as a colourless solid (76 mg, 56%). ¹H NMR (400 MHz, DMSO-d₆) δ 12.02 (1H, s), 8.95-8.93 (2H, m), 8.06 (1H, d, J=8.7 Hz), 7.88 (1H, d, J=2.0 Hz), 7.80 (2H, d, J=8.7 Hz), 7.76 (2H, d, J=8.4 Hz), 7.66 (1H, dd, J=8.7, 2.0 Hz), 7.53 (2H, d, J=8.7 Hz), 7.32 (2H, d, J=8.7 Hz), 5.79 (1H, dd, J=12.0, 5.1 Hz), 3.94 (1H, dd, J=18.3, 12.1 Hz), 3.28 (1H, dd, J=18.4, 5.2 Hz), 3.00 (1H, dt, J=17.2, 6.6 Hz), 2.92 (1H, dt, J=17.2, 6.7 Hz), 2.54-2.51 (2H, m), 2.34 (3H, s); ¹³C NMR (101 MHz, DMSO-d₆) δ 170.7 (C), 168.9 (C), 153.6 (C), 146.0 (CH), 145.6 (CH), 144.0 (C), 143.9 (C), 142.1 (C), 141.6 (C), 136.6 (C), 135.0 (C), 129.82 (C), 129.79 (CH), 129.4 (2×CH), 128.9 (2×CH), 128.5 (2×CH), 128.0 (CH), 127.4 (2×CH), 125.4 (CH), 59.7 (CH), 41.7 (CH₂), 30.0 (CH₂), 27.8 (CH₂), 21.0 (CH₃); HRMS (ESI/Q-TOF) m/z: [M+Na]⁺ calcd for C₂₈H₂₄ClN₅NaO₄S, 584.1130; found, 584.1131.

### N—(N,N-Dimethylsulfamoyl)-4-oxo-4-(5-(quinoxalin-6-yl)-3-(4-(trifluoromethoxy)phenyl)-4,5-dihydro-1H-pyrazol-1-yl) butanamide (Compound 126)

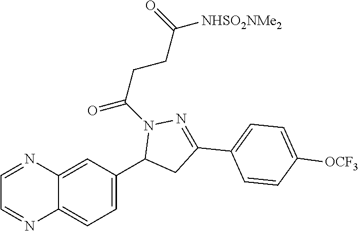

Compound 126 was prepared following a method similar to that described for the preparation of Compound 55 using Compound 8 (46 mg, 0.10 mmol), N,N-dimethylsulfamide (25 mg, 0.20 mmol), N-(3-dimethylaminopropyl)-N′-ethylcarbodiimide hydrochloride (EDCI) (38 mg, 0.20 mmol), and 4-(dimethylamino)pyridine (DMAP) (15 mg, 0.12 mmol) in CH₂Cl₂ (1.0 mL). Purification by flash column chromatography (petroleum ether/EtOAc, 1:1→EtOAc, neat) afforded Compound 126 as a colourless solid (38 mg, 67%). ¹H NMR (400 MHz, CDCl₃) δ 9.02 (1H, s), 8.81 (2H, s), 8.10 (1H, d, J=8.7 Hz), 7.95 (1H, d, J=2.0 Hz), 7.78 (2H, d, J=9.0 Hz), 7.65 (1H, dd, J=8.7, 2.0 Hz), 7.28 (2H, d, J=9.0 Hz), 5.85 (1H, dd, J=11.8, 4.7 Hz), 3.88 (1H, dd, J=17.8, 11.9 Hz), 3.30 (1H, ddd, J=18.1, 7.4, 5.4 Hz), 3.23 (1H, dd, J=17.7, 4.8 Hz), 3.17 (1H, ddd, J=18.0, 7.7, 5.6 Hz), 2.76 (6H, s), 2.67-2.54 (2H, m); ¹³C NMR (101 MHz, CDCl₃) δ 171.0 (C), 170.4 (C), 153.8 (C), 151.0 (C, q. J_{C-F}=1.8 Hz), 145.6 (CH), 145.3 (CH), 143.4 (C), 143.1 (C), 142.7 (C), 130.8 (CH), 129.5 (C), 128.6 (2×CH), 128.0 (CH), 126.1 (CH), 121.3 (2×CH), 120.5 (C, q, J_{C-F}=258.3 Hz), 60.3 (CH), 42.3 (CH₂), 38.2 (2×CH₃), 30.9 (CH₂), 29.3 (CH₂); HRMS (ESI/Q-TOF) m/z: [M+H]⁺ calcd for C₂₄H₂₄F₃N₆O₅S, 565.1475; found, 565.1475.

### 4-(3-([1,1′-Biphenyl]-3-yl)-5-(quinoxalin-6-yl)-4,5-dihydro-1H-pyrazol-1-yl)-N—(N,N-dimethylsulfamoyl)-4-oxobutanamide (Compound 127)

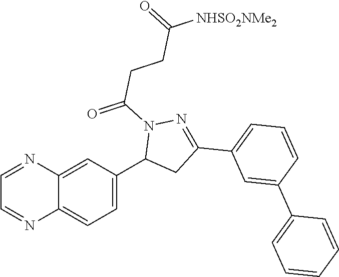

Compound 127 was prepared following a method similar to that described for the preparation of Compound 117 using Compound 13 (68 mg, 0.15 mmol), N,N-dimethylsulfamide (28 mg, 0.22 mmol), N-(3-dimethylaminopropyl)-N′-ethylcarbodiimide hydrochloride (EDCI) (43 mg, 0.22 mmol), and 4-(dimethylamino)pyridine (DMAP) (22 mg, 0.18 mmol) in CH₂Cl₂ (1.5 mL). Purification by flash column chromatography (petroleum ether/EtOAc, 1:1→EtOAc, neat) afforded Compound 127 as a colourless solid (58 mg, 69%). ¹H NMR (400 MHz, CDCl₃) δ 8.81 (2H, s), 8.10 (1H, d, J=8.7 Hz), 7.97 (2H, d, J=11.1 Hz), 7.73-7.60 (5H, m), 7.53-7.45 (3H, m), 7.39 (1H, t, J=7.3 Hz), 5.87 (1H, dd, J=11.8, 4.6 Hz), 3.94 (1H, dd, J=17.8, 11.8 Hz), 3.39-3.28 (2H, m), 3.20 (1H, ddd, J=18.1, 7.5, 5.4 Hz), 2.76 (6H, s), 2.68-2.56 (2H, m); ¹³C NMR (101 MHz, CDCl₃) δ 171.1 (C), 170.4 (C), 155.3 (C), 145.5 (CH), 145.3 (CH), 143.6 (C), 143.1 (C), 142.7 (C), 142.2 (C), 140.4 (C), 131.4 (C), 130.8 (CH), 129.8 (CH), 129.4 (CH), 129.1 (2×CH), 128.1 (CH), 128.0 (CH), 127.3 (2×CH), 126.1 (CH), 125.8 (CH), 125.7 (CH), 60.2 (CH), 42.5 (CH₂), 38.2 (2×CH₃), 31.0 (CH₂), 29.4 (CH₂); HRMS (ESI/Q-TOF) m/z: [M+H]⁺ calcd for C₂₉H₂₉N₆O₄S, 557.1966; found, 557.1965.

### 4-(3-(4-Chlorophenyl)-5-(quinoxalin-6-yl)-4,5-dihydro-1H-pyrazol-1-yl)-4-oxo-N-(pyrrolidin-1-ylsulfonyl) butanamide (Compound 128)

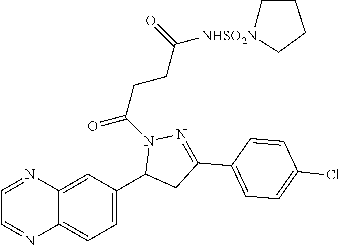

Compound 128 was prepared following a method similar to that described for the preparation of Compound 117 using Compound 1 (0.10 g, 0.24 mmol), pyrrolidine-1-sulfonamide (54 mg, 0.36 mmol), N-(3-dimethylaminopropyl)-N′-ethylcarbodiimide hydrochloride (EDCI) (69 mg, 0.36 mmol), and 4-(dimethylamino)pyridine (DMAP) (35 mg, 0.29 mmol) in CH₂Cl₂ (2.4 mL). Purification by flash column chromatography (petroleum ether/EtOAc, 1:1→EtOAc, neat) afforded Compound 128 as a colourless solid (73 mg, 56%). ¹H NMR (400 MHz, CDCl₃) δ 8.81 (2H, s), 8.09 (1H, d, J=8.7 Hz), 7.95 (1H, d, J=2.1 Hz), 7.68 (2H, d, J=8.9 Hz), 7.64 (1H, dd, J=8.7, 2.0 Hz), 7.40 (2H, d, J=8.9 Hz), 5.83 (1H, dd, J=11.8, 4.8 Hz), 3.85 (1H, dd, J=17.7, 11.9 Hz), 3.43-3.28 (4H, m), 3.28-3.12 (3H, m), 2.66-2.54 (2H, m), 1.68-1.59 (4H, m); ¹³C NMR (101 MHz, CDCl₃) δ 170.8 (C), 170.4 (C), 154.0 (C), 145.5 (CH), 145.3 (CH), 143.5 (C), 143.1 (C), 142.7 (C), 137.1 (C), 130.8 (CH), 129.4 (C), 129.3 (2×CH), 128.2 (2×CH), 128.0 (CH), 126.0 (CH), 60.3 (CH), 48.7 (2×CH₂), 42.3 (CH₂), 30.8 (CH₂), 29.2 (CH₂), 25.7 (2×CH₂); HRMS (ESI/Q-TOF) m/z: [M+Na]⁺ calcd for C₂₅H₂₅ClN₆NaO₄S, 563.1239; found, 563.1239.

### 4-Oxo-4-(6-phenyl-3-(quinoxalin-6-yl)-3,3a,4,5-tetrahydro-2H-benzo[g]indazol-2-yl)butanoic acid (Compound 129)

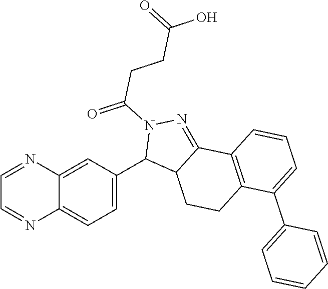 [SMILES: O=C(O)CCC(=O)N1N=C2c3cccc(-c4ccccc4)c3CCC2C1c1ccc2nccnc2c1]

To a solution of quinoxaline-6-carbaldehyde (277 mg, 1.75 mmol) and 5-phenyl-1-tetralone (389 mg, 1.75 mmol) in EtOH (14 mL) at room temperature was added dropwise 2 M NaOH (4.4 mL, 8.75 mmol), and the reaction mixture was stirred for 3 h. After, the solution was cooled down to 0° C., and the resulting precipitated solids were then collected by filtration, washed with water and dried in vacuo to afford (E)-5-phenyl-2-(quinoxalin-6-ylmethylene)-3,4-dihydronaphthalen-1(2H)-one as a colourless solid (520 mg), which was used directly in the next step without further purification.

To a stirred solution of (E)-5-phenyl-2-(quinoxalin-6-ylmethylene)-3,4-dihydronaphthalen-1(2H)-one (300 mg) in EtOH (7 mL) was added hydrazine hydrate (0.10 mL, 50% w/w), and the reaction mixture heated under reflux for 2 h. After, the solution was cooled down to 0° C. and the resulting precipitated solids were then collected by filtration, washed with water and dried in vacuo to afford 6-phenyl-3-(quinoxalin-6-yl)-3,3a,4,5-tetrahydro-2H-benzo[g]indazole as a yellow solid (259 mg), which was used in the next step without further purification.

A mixture of 6-phenyl-3-(quinoxalin-6-yl)-3,3a,4,5-tetrahydro-2H-benzo[g]indazole (259 mg) and succinic anhydride (103 mg, 1.03 mmol) in THF (5 mL) was stirred under reflux for 1 h. The solvent was then removed in vacuo and the crude material was purified by flash column chromatography (petroleum ether/EtOAc, 1:1→EtOAc, neat→CH₂Cl₂/MeOH, 9:1). The resulting solid was washed with CH₂Cl₂ and isolated by filtration to afford Compound 129 as an off-white solid (154 mg, 32% over 3 steps). ¹H NMR (400 MHz, DMSO-d₆) δ 12.07 (1H, s), 8.95-8.93 (2H, m), 8.09 (1H, d, J=8.7 Hz), 8.02-7.99 (2H, m), 7.81 (1H, dd, J=8.7, 2.0 Hz), 7.46-7.35 (4H, m), 7.32-7.29 (3H, m), 5.28 (1H, d, J=9.7 Hz), 3.43 (1H, ddd, J=14.0, 9.7, 4.8 Hz), 3.11-2.84 (3H, m), 2.64-2.59 (1H, m), 2.50-2.47 (2H, m), 2.26-2.23 (1H, m), 1.91 (1H, ddd, J=12.8, 12.8, 4.6 Hz); ¹³C NMR (101 MHz, DMSO-d₆) δ 173.7 (C), 170.6 (C), 155.6 (C), 145.9 (CH), 145.5 (CH), 143.9 (C), 142.2 (C), 142.0 (C), 141.6 (C), 140.4 (C), 137.1 (C), 131.7 (CH), 129.5 (CH), 128.8 (2×CH), 128.33 (2×CH), 128.28 (CH), 127.4 (C), 127.3 (CH), 126.6 (CH), 125.8 (CH), 123.7 (CH), 66.7 (CH), 54.1 (CH), 29.0 (CH₂), 28.4 (CH₂), 27.4 (CH₂), 27.1 (CH₂); HRMS (ESI/Q-TOF) m/z: [M+H]⁺ calcd for C₂₉H₂₅N₄O₃, 477.1921; found, 477.1921.

### In Vitro Studies

The compounds of the present invention were initially screened for activity using an in vitro fully automated incubation system for the measurement of total gas production and gas composition (methane and hydrogen). The system used is described in detail in the paper by Muetzel et al. (2014; Animal Feed Science and Technology 196, 1-11). The system in this specification is referred to as the rumen in vitro system or RIV. The RIV system provides a relatively simple tool for determining the rate and production of methane and also whether the rate and production of methane is inhibited or reduced by a particular compound or feed.

### Rumen In Vitro Assay Method.

Rumen in vitro assays use rumen fluid from donor animals (typically either sheep or cattle) which is typically combined with a buffer and then incubated in sealed fermentation vessels at 39° C. for either 24 or 48 h. These assays systems have been well-characterised and used by the scientific community over the last two decades (Rymer et al. 2005). The system that has been used here to characterise the effects of inhibitors on methane production and gas production, and the formation of H₂, was described by Muetzel et al. (2014). Rumen in vitro assay systems reflect what can occur in the rumen in vivo, but only short-term, due to their short incubation times (typically not exceeding 48 h) which is limited by its buffering capacity and the fact that it is a closed system.

Stock solutions of inhibitors were re-suspended in dimethylformamide (DMF) at concentrations 1000-fold higher than the highest used for the assays. Any further dilutions were also prepared using DMF. The total amount of inhibitor solution added to the rumen fluid-buffer mixture (60 mL) was 60 μl. Incubations used 60 ml of medium containing 12 ml of filtered rumen fluid and 48 ml of buffer in serum bottles for 24-48 h, essentially as described by Muetzel et al. (2014). Two fistulated cattle were used as donor animals for rumen fluid and treatments were incubated in duplicate bottles. Sets of duplicate incubation vials that contained ryegrass (with rumen fluid-buffer mixture), and ryegrass with 30 μM bromoethanesulfonate (BES) were also incubated as negative and positive controls, respectively. One experiment consists of 2 replicates of an inhibitor at any one concentration run at the same time, and a run can contain up to 32 bottle, including positive (30 UM BES) and negative (no inhibitor added) controls.

Compound 1, for example, was tested using the 60 mL rumen in vitro assay system at 50 μM, 10 μM, and 2 μM final concentrations and compared to a positive control with 30 UM BES. At 30 μM, BES inhibition levels were 70.5% inhibition at 10 hours and 55.6% inhibition at 18 hours.

TABLE 1

| Compounds | | | | |
|---|---|---|---|---|
| of Formula I | | RIV (all figures are % | | |
| Compound | | methane reduction +/− SD) | | |
| No | Structure | 50 μM | 10 μM | 2 μM |
| 1 | 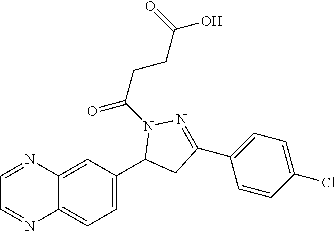 [SMILES: O=C(O)CCC(=O)N1N=C(c2ccc(Cl)cc2)CC1c1ccc2nccnc2c1] | 37.6 ± 1.3 | | |
| 1A | 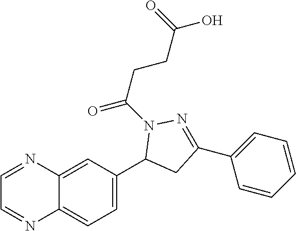 [SMILES: O=C(O)CCC(=O)N1N=C(c2ccccc2)CC1c1ccc2nccnc2c1] | <5 | | |
| 2 | 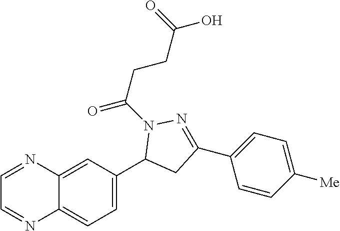 [SMILES: Cc1ccc(C2=NN(C(=O)CCC(=O)O)C(c3ccc4nccnc4c3)C2)cc1] | 13.9 | | |
| 3 | 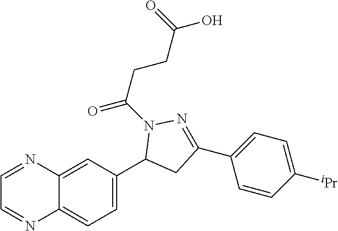 [SMILES: CC(C)c1ccc(C2=NN(C(=O)CCC(=O)O)C(c3ccc4nccnc4c3)C2)cc1] | 78.6 ± 1.1 | 5.5 ± 2.8 | |
| 4 | 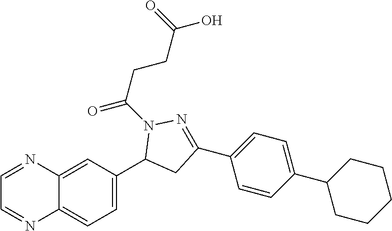 [SMILES: O=C(O)CCC(=O)N1N=C(c2ccc(C3CCCCC3)cc2)CC1c1ccc2nccnc2c1] | 79.5 ± 4.4 | <5 | |
| 5 | 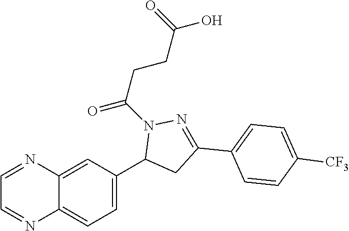 [SMILES: O=C(O)CCC(=O)N1N=C(c2ccc(C(F)(F)F)cc2)CC1c1ccc2nccnc2c1] | 46.5 ± 2.7 | | |
| 6 | 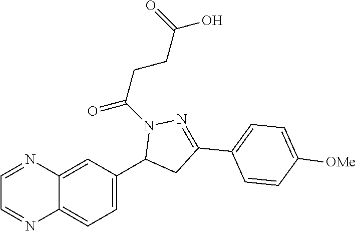 [SMILES: COc1ccc(C2=NN(C(=O)CCC(=O)O)C(c3ccc4nccnc4c3)C2)cc1] | 5.6 ± 2.0 | | |
| 7 | 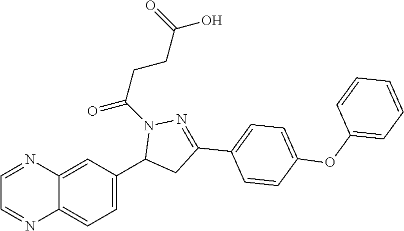 [SMILES: O=C(O)CCC(=O)N1N=C(c2ccc(Oc3ccccc3)cc2)CC1c1ccc2nccnc2c1] | 66.4 ± 14.7 | 30.7 ± 3.6 | |
| 8 | 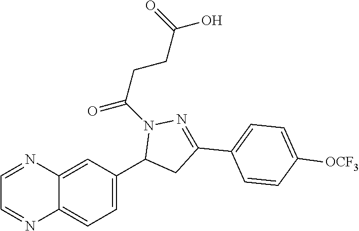 [SMILES: O=C(O)CCC(=O)N1N=C(c2ccc(OC(F)(F)F)cc2)CC1c1ccc2nccnc2c1] | 75.2 ± 1.6 | 22.6 ± 2.8 | |
| 9 | 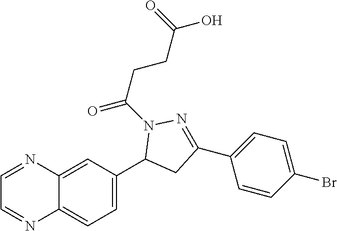 [SMILES: O=C(O)CCC(=O)N1N=C(c2ccc(Br)cc2)CC1c1ccc2nccnc2c1] | 64.9 ± 1.9 | | |
| 10 | 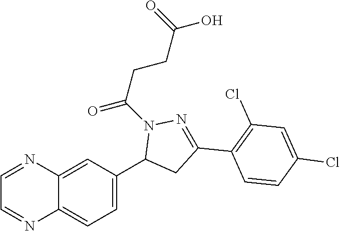 [SMILES: O=C(O)CCC(=O)N1N=C(c2ccc(Cl)cc2Cl)CC1c1ccc2nccnc2c1] | 64.0 ± 0.6 | | |
| 11 | 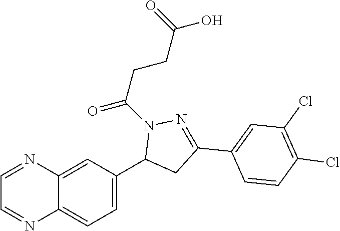 [SMILES: O=C(O)CCC(=O)N1N=C(c2ccc(Cl)c(Cl)c2)CC1c1ccc2nccnc2c1] | 62.8 ± 3.4 | | |
| 12 | 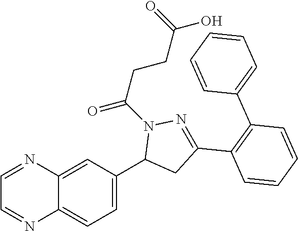 [SMILES: O=C(O)CCC(=O)N1N=C(c2ccccc2-c2ccccc2)CC1c1ccc2nccnc2c1] | 95.9 ± 0.9 | 51.6 ± 1.5 | |
| 13 | 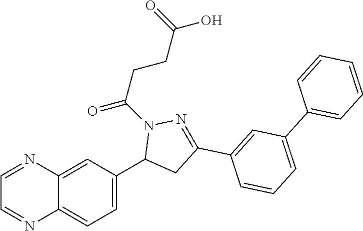 [SMILES: O=C(O)CCC(=O)N1N=C(c2cccc(-c3ccccc3)c2)CC1c1ccc2nccnc2c1] | 97.2 ± 0.1 | 74.4 ± 3.3 | 5.0 ± 0.4 |
| 14 | 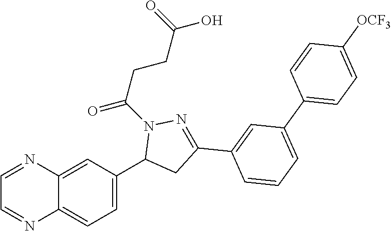 [SMILES: O=C(O)CCC(=O)N1N=C(c2cccc(-c3ccc(OC(F)(F)F)cc3)c2)CC1c1ccc2nccnc2c1] | 95.4 | 72.4 ± 0.8 | <5 |
| 15 | 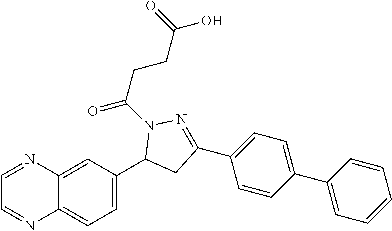 [SMILES: O=C(O)CCC(=O)N1N=C(c2ccc(-c3ccccc3)cc2)CC1c1ccc2nccnc2c1] | 87.8 ± 1.6 | 60.1 ± 1.0 | <5 |
| 16 | 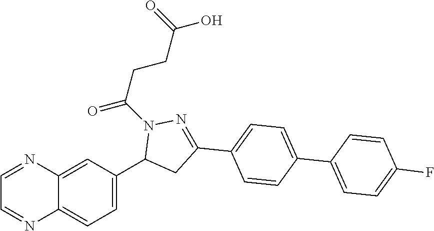 [SMILES: O=C(O)CCC(=O)N1N=C(c2ccc(-c3ccc(F)cc3)cc2)CC1c1ccc2nccnc2c1] | 96.6 ± 0.2 | 77.5 ± 2.1 | <5 |
| 17 | 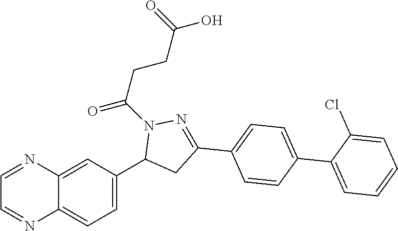 [SMILES: O=C(O)CCC(=O)N1N=C(c2ccc(-c3ccccc3Cl)cc2)CC1c1ccc2nccnc2c1] | 90.0 ± 1.2 | 74.2 ± 7.8 | <5 |
| 18 | 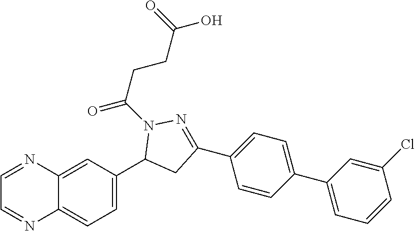 [SMILES: O=C(O)CCC(=O)N1N=C(c2ccc(-c3cccc(Cl)c3)cc2)CC1c1ccc2nccnc2c1] | 94.1 ± 0.1 | 83.6 ± 1.5 | <5 |
| 19 | 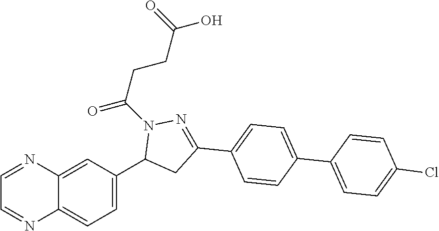 [SMILES: O=C(O)CCC(=O)N1N=C(c2ccc(-c3ccc(Cl)cc3)cc2)CC1c1ccc2nccnc2c1] | 91.1 ± 0.3 | 83.7 ± 0.6 | 5.3 ± 0.6 |
| 20 | 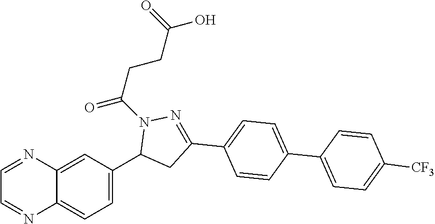 [SMILES: O=C(O)CCC(=O)N1N=C(c2ccc(-c3ccc(C(F)(F)F)cc3)cc2)CC1c1ccc2nccnc2c1] | | 37.2 ± 5.9 | |
| 21 | 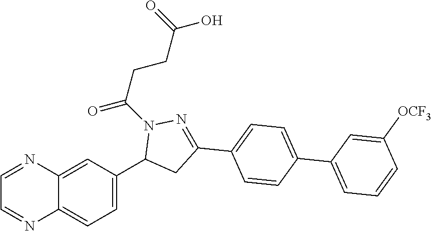 [SMILES: O=C(O)CCC(=O)N1N=C(c2ccc(-c3cccc(OC(F)(F)F)c3)cc2)CC1c1ccc2nccnc2c1] | 97.9 | 91.9 ± 1.5 | 15.8 ± 1.1 |
| 22 | 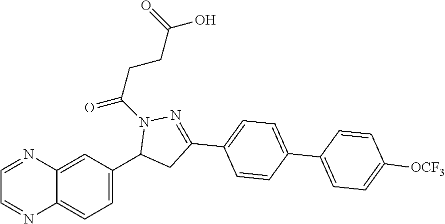 [SMILES: O=C(O)CCC(=O)N1N=C(c2ccc(-c3ccc(OC(F)(F)F)cc3)cc2)CC1c1ccc2nccnc2c1] | 94.7 ± 0.8 | 85.1 ± 0.8 | 23.8 ± 1.1 |
| 23 | 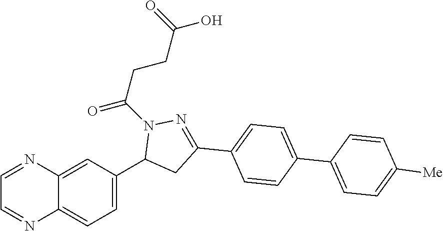 [SMILES: Cc1ccc(-c2ccc(C3=NN(C(=O)CCC(=O)O)C(c4ccc5nccnc5c4)C3)cc2)cc1] | 94.8 ± 0.4 | 69.0 ± 3.7 | |
| 24 | 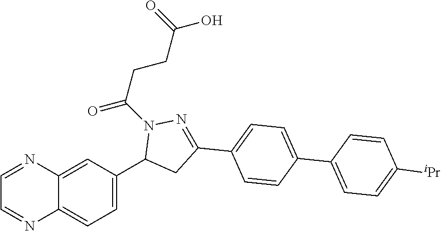 [SMILES: CC(C)c1ccc(-c2ccc(C3=NN(C(=O)CCC(=O)O)C(c4ccc5nccnc5c4)C3)cc2)cc1] | 97.8 ± 0.05 | 94.1 ± 0.3 | 21.8 |
| 25 | 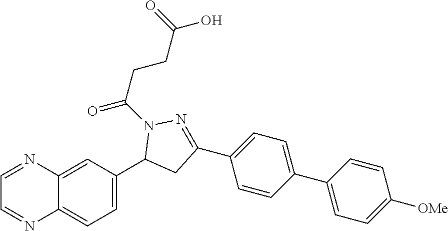 [SMILES: COc1ccc(-c2ccc(C3=NN(C(=O)CCC(=O)O)C(c4ccc5nccnc5c4)C3)cc2)cc1] | | 83.7 ± 0.4 | <5 |
| 26 | 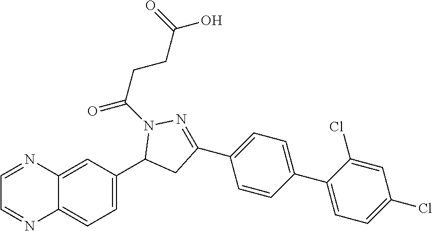 [SMILES: O=C(O)CCC(=O)N1N=C(c2ccc(-c3ccc(Cl)cc3Cl)cc2)CC1c1ccc2nccnc2c1] | 96.0 ± 1.1 | 65.8 ± 3.7 | |
| 27 | 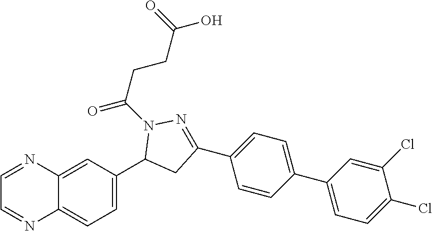 [SMILES: O=C(O)CCC(=O)N1N=C(c2ccc(-c3ccc(Cl)c(Cl)c3)cc2)CC1c1ccc2nccnc2c1] | 92.1 ± 1.1 | 48.3 ± 7.5 | |
| 28 | 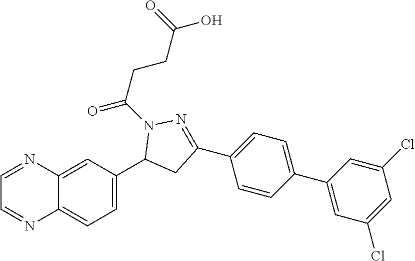 [SMILES: O=C(O)CCC(=O)N1N=C(c2ccc(-c3cc(Cl)cc(Cl)c3)cc2)CC1c1ccc2nccnc2c1] | 96.1 ± 0.1 | 84.0 ± 0.8 | 8.0 ± 2.1 |
| 29 | 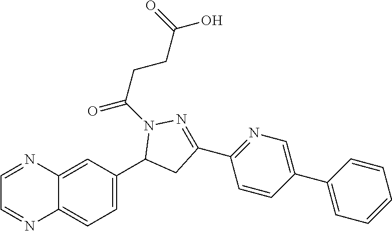 [SMILES: O=C(O)CCC(=O)N1N=C(c2ccc(-c3ccccc3)cn2)CC1c1ccc2nccnc2c1] | 33.3 ± 0.6 | | |
| 30 | 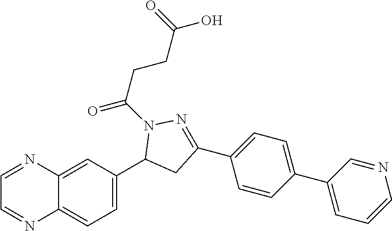 [SMILES: O=C(O)CCC(=O)N1N=C(c2ccc(-c3cccnc3)cc2)CC1c1ccc2nccnc2c1] | 9.3 ± 2.8 | | |
| 31 | 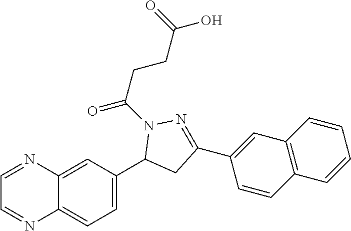 [SMILES: O=C(O)CCC(=O)N1N=C(c2ccc3ccccc3c2)CC1c1ccc2nccnc2c1] | 56.6 ± 6.9 | | |
| 32 | 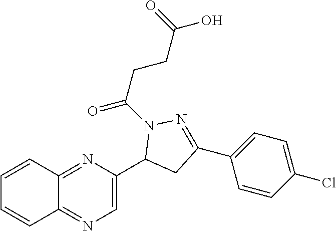 [SMILES: O=C(O)CCC(=O)N1N=C(c2ccc(Cl)cc2)CC1c1cnc2ccccc2n1] | 81.0 ± 2.0 | <5 | |
| 33 | 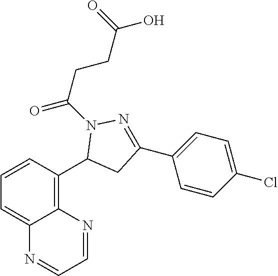 [SMILES: O=C(O)CCC(=O)N1N=C(c2ccc(Cl)cc2)CC1c1cccc2nccnc12] | 18.4 ± 3.7 | | |
| 34 | 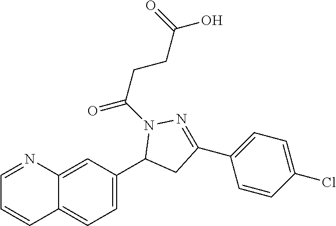 [SMILES: O=C(O)CCC(=O)N1N=C(c2ccc(Cl)cc2)CC1c1ccc2cccnc2c1] | 22.0 ± 2.1 | | |
| 35 | 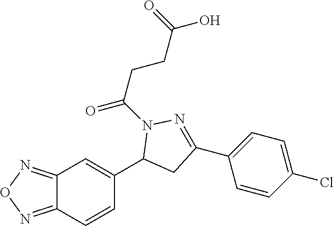 [SMILES: O=C(O)CCC(=O)N1N=C(c2ccc(Cl)cc2)CC1c1ccc2nonc2c1] | 91.3 ± 2.1 | 43.6 ± 6.6 | |
| 36 | 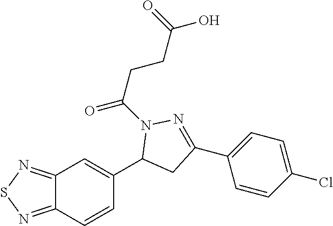 [SMILES: O=C(O)CCC(=O)N1N=C(c2ccc(Cl)cc2)CC1c1ccc2nsnc2c1] | 91.3 ± 0.2 | 33.4 ± 2.3 | |
| 37 | 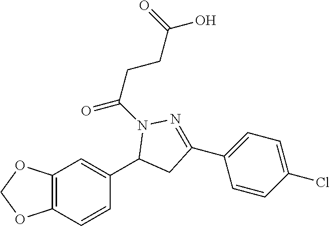 [SMILES: O=C(O)CCC(=O)N1N=C(c2ccc(Cl)cc2)CC1c1ccc2c(c1)OCO2] | 15.6 ± 2.0 | | |
| 38 | 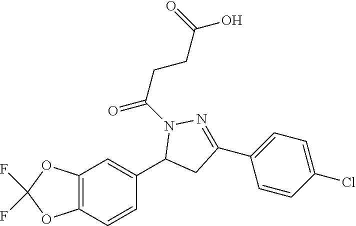 [SMILES: O=C(O)CCC(=O)N1N=C(c2ccc(Cl)cc2)CC1c1ccc2c(c1)OC(F)(F)O2] | 93.4 ± 0.5 | 55.8 ± 1.8 | |
| 39 | 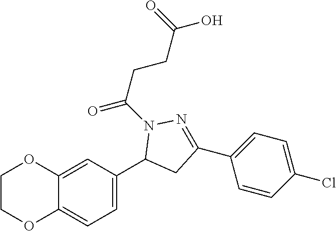 [SMILES: O=C(O)CCC(=O)N1N=C(c2ccc(Cl)cc2)CC1c1ccc2c(c1)OCCO2] | 79.2 ± 0.0 | 20.0 ± 5.1 | |
| 40 | 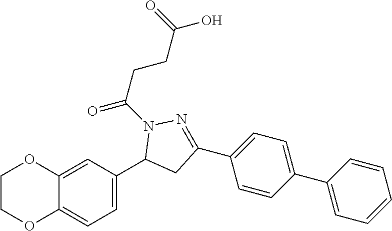 [SMILES: O=C(O)CCC(=O)N1N=C(c2ccc(-c3ccccc3)cc2)CC1c1ccc2c(c1)OCCO2] | 89.5 ± 1.4 | 39.8 ± 1.3 | |
| 41 | 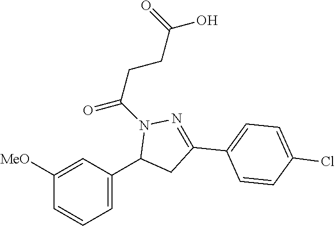 [SMILES: COc1cccc(C2CC(c3ccc(Cl)cc3)=NN2C(=O)CCC(=O)O)c1] | 13.4 ± 3.2 | | |
| 42 | 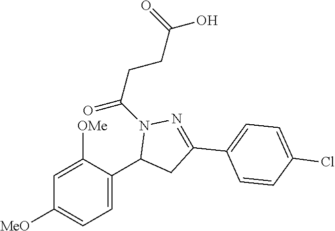 [SMILES: COc1ccc(C2CC(c3ccc(Cl)cc3)=NN2C(=O)CCC(=O)O)c(OC)c1] | 80.0 ± 4.7 | 10.5 ± 3.5 | |
| 43 | 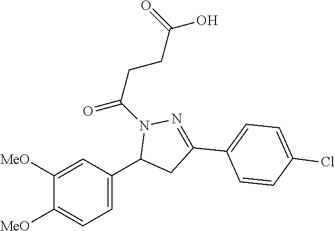 [SMILES: COc1ccc(C2CC(c3ccc(Cl)cc3)=NN2C(=O)CCC(=O)O)cc1OC] | 39.7 ± 7.2 | | |
| 44 | 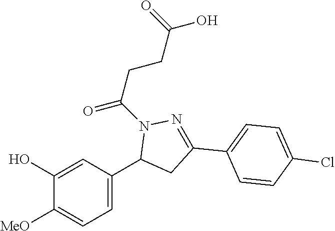 [SMILES: COc1ccc(C2CC(c3ccc(Cl)cc3)=NN2C(=O)CCC(=O)O)cc1O] | 31.3 ± 0.8 | | |
| 45 | 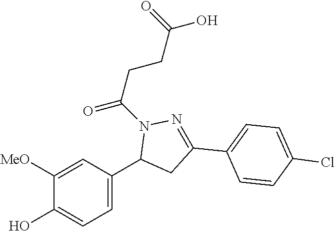 [SMILES: COc1cc(C2CC(c3ccc(Cl)cc3)=NN2C(=O)CCC(=O)O)ccc1O] | 73.1 ± 8.6 | 9.9 ± 1.8 | |
| 46 | 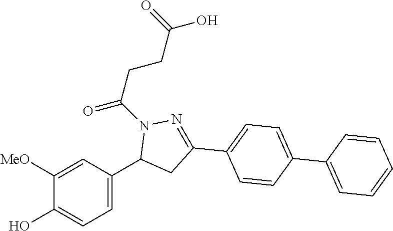 [SMILES: COc1cc(C2CC(c3ccc(-c4ccccc4)cc3)=NN2C(=O)CCC(=O)O)ccc1O] | 48.6 ± 1.7 | | |
| 47 | 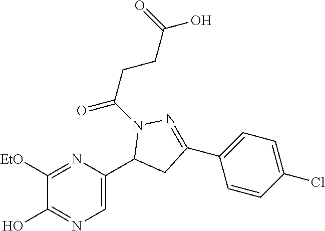 [SMILES: CCOc1nc(C2CC(c3ccc(Cl)cc3)=NN2C(=O)CCC(=O)O)cnc1O] | 68.3 ± 3.9 | | |
| 48 | 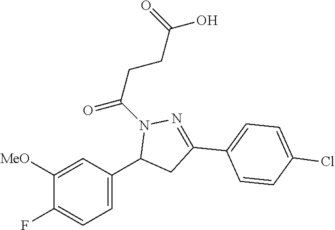 [SMILES: COc1cc(C2CC(c3ccc(Cl)cc3)=NN2C(=O)CCC(=O)O)ccc1F] | 43.6 ± 4.5 | | |
| 49 | 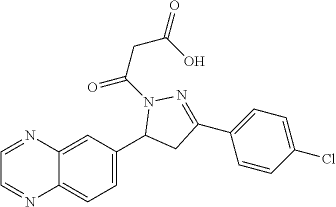 [SMILES: O=C(O)CC(=O)N1N=C(c2ccc(Cl)cc2)CC1c1ccc2nccnc2c1] | 8.1 ± 7.7 | | |
| 50 | 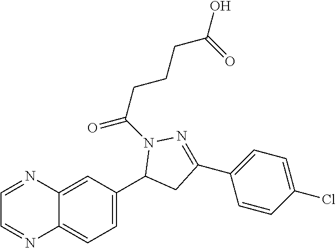 [SMILES: O=C(O)CCCC(=O)N1N=C(c2ccc(Cl)cc2)CC1c1ccc2nccnc2c1] | 66.4 ± 2.9 | | |
| 51 | 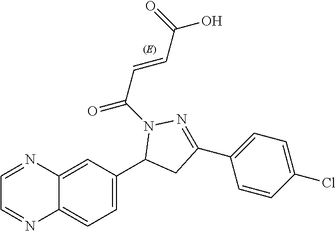 [SMILES: O=C(O)/C=C/C(=O)N1N=C(c2ccc(Cl)cc2)CC1c1ccc2nccnc2c1] | 57.1 ± 7.5 | | |
| 52 | 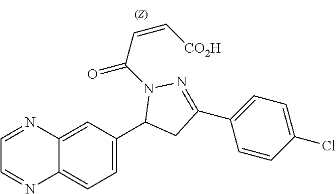 [SMILES: O=C(O)/C=C\C(=O)N1N=C(c2ccc(Cl)cc2)CC1c1ccc2nccnc2c1] | 34.1 ± 1.3 | | |
| 53 | 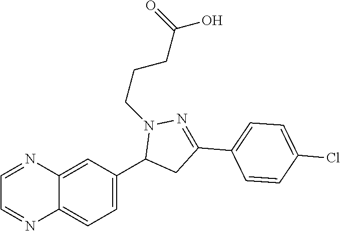 [SMILES: O=C(O)CCCN1N=C(c2ccc(Cl)cc2)CC1c1ccc2nccnc2c1] | 82.1 ± 1.2 | 19.4 ± 1.9 | |
| 54 | 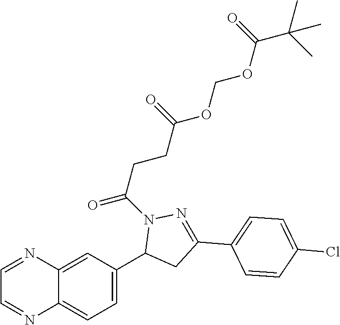 [SMILES: CC(C)(C)C(=O)OCOC(=O)CCC(=O)N1N=C(c2ccc(Cl)cc2)CC1c1ccc2nccnc2c1] | 23.8 ± 9.4 | | |
| 55 | 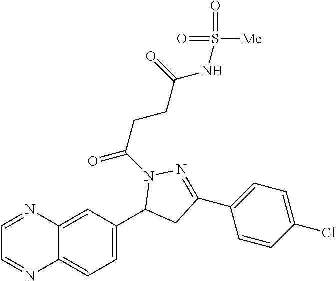 [SMILES: CS(=O)(=O)NC(=O)CCC(=O)N1N=C(c2ccc(Cl)cc2)CC1c1ccc2nccnc2c1] | 96.1 ± 0.5 | 70.0 ± 4.5 | <5 |
| 56 | 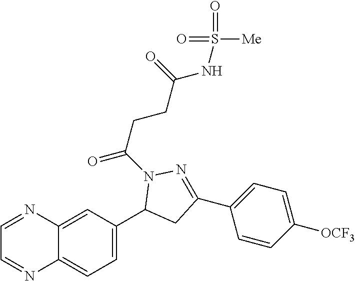 [SMILES: CS(=O)(=O)NC(=O)CCC(=O)N1N=C(c2ccc(OC(F)(F)F)cc2)CC1c1ccc2nccnc2c1] | 98.6 | 72.4 ± 10.6 | <5 |
| 57 | 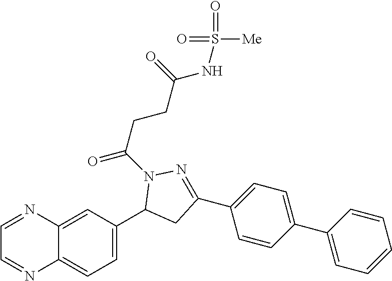 [SMILES: CS(=O)(=O)NC(=O)CCC(=O)N1N=C(c2ccc(-c3ccccc3)cc2)CC1c1ccc2nccnc2c1] | 89.7 ± 0.5 | 62.7 ± 4.4 | |
| 58 | 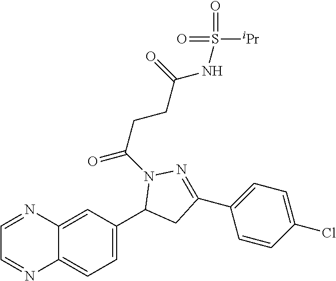 [SMILES: CC(C)S(=O)(=O)NC(=O)CCC(=O)N1N=C(c2ccc(Cl)cc2)CC1c1ccc2nccnc2c1] | 93.9 ± 4.7 | 80.4 ± 6.3 | 6.0 ± 1.6 |
| 59 | 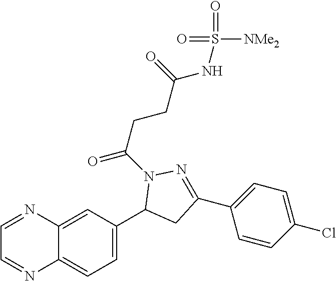 [SMILES: CN(C)S(=O)(=O)NC(=O)CCC(=O)N1N=C(c2ccc(Cl)cc2)CC1c1ccc2nccnc2c1] | 94.4 ± 4.0 | 86.7 ± 7.0 | 10.6 ± 2.5 |
| 60 | 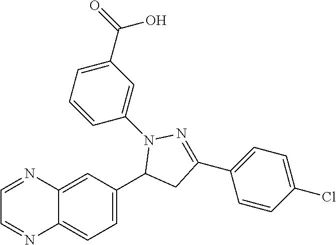 [SMILES: O=C(O)c1cccc(N2N=C(c3ccc(Cl)cc3)CC2c2ccc3nccnc3c2)c1] | 97.7 ± 0.6 | 90.9 ± 1.2 | 16.1 ± 0.5 |
| 61 | 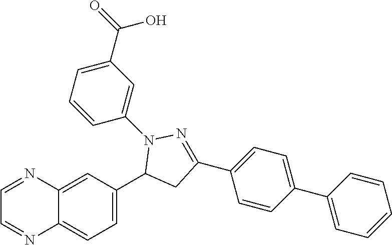 [SMILES: O=C(O)c1cccc(N2N=C(c3ccc(-c4ccccc4)cc3)CC2c2ccc3nccnc3c2)c1] | 95.9 ± 0.6 | 88.3 ± 0.9 | 13.1 |
| 62 | 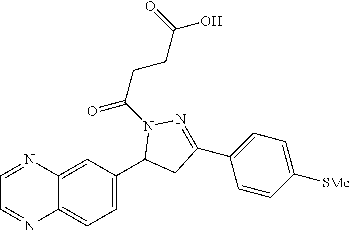 [SMILES: CSc1ccc(C2=NN(C(=O)CCC(=O)O)C(c3ccc4nccnc4c3)C2)cc1] | 31.8 | | |
| 63 | 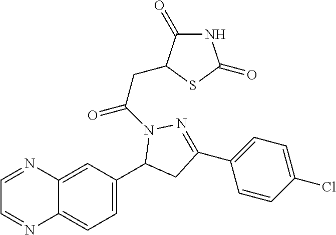 [SMILES: O=C1NC(=O)C(CC(=O)N2N=C(c3ccc(Cl)cc3)CC2c2ccc3nccnc3c2)S1] | 43.6 ± 2.9 | | |
| 64 | 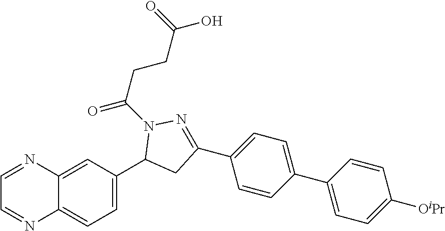 [SMILES: CC(C)Oc1ccc(-c2ccc(C3=NN(C(=O)CCC(=O)O)C(c4ccc5nccnc5c4)C3)cc2)cc1] | | 97.5 | 81.0 |
| 65 | 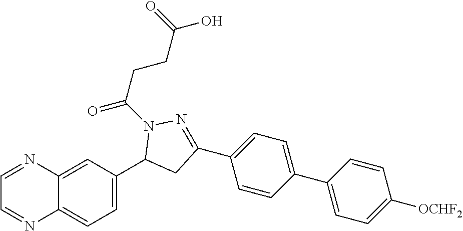 [SMILES: O=C(O)CCC(=O)N1N=C(c2ccc(-c3ccc(OC(F)F)cc3)cc2)CC1c1ccc2nccnc2c1] | | 90.2 | 26.2 |
| 66 | 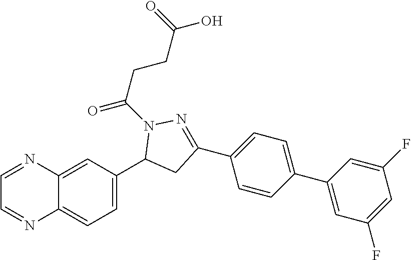 [SMILES: O=C(O)CCC(=O)N1N=C(c2ccc(-c3cc(F)cc(F)c3)cc2)CC1c1ccc2nccnc2c1] | | 85.4 | 17.5 |
| 67 | 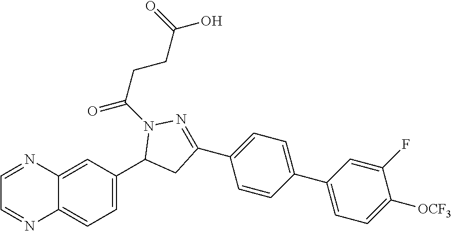 [SMILES: O=C(O)CCC(=O)N1N=C(c2ccc(-c3ccc(OC(F)(F)F)c(F)c3)cc2)CC1c1ccc2nccnc2c1] | | 92.4 ± 1.5 | 51.8 ± 8.4 |
| 68 | 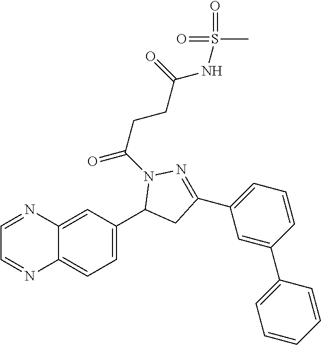 [SMILES: CS(=O)(=O)NC(=O)CCC(=O)N1N=C(c2cccc(-c3ccccc3)c2)CC1c1ccc2nccnc2c1] | | 85.4 ± 0.5 | 20.5 ± 1.4 |
| 69 | 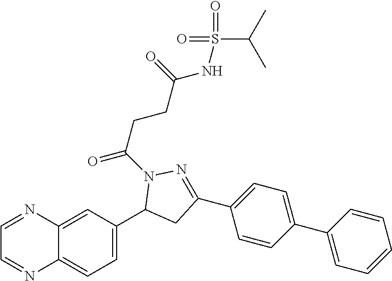 [SMILES: CC(C)S(=O)(=O)NC(=O)CCC(=O)N1N=C(c2ccc(-c3ccccc3)cc2)CC1c1ccc2nccnc2c1] | | 92.8 ± 1.8 | 37.6 ± 5.6 |
| 70 | 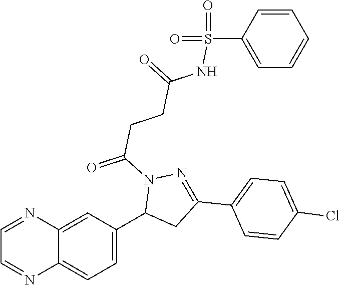 [SMILES: O=C(CCC(=O)N1N=C(c2ccc(Cl)cc2)CC1c1ccc2nccnc2c1)NS(=O)(=O)c1ccccc1] | | 88.9 ± 1.5 | 27.1 ± 13.1 |
| 71 | 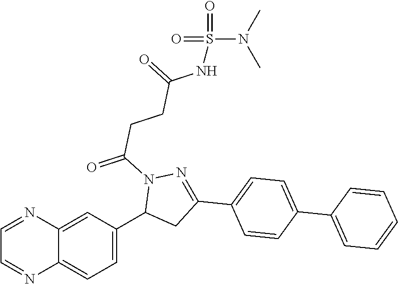 [SMILES: CN(C)S(=O)(=O)NC(=O)CCC(=O)N1N=C(c2ccc(-c3ccccc3)cc2)CC1c1ccc2nccnc2c1] | | 70.7 | <5 |
| 72 | 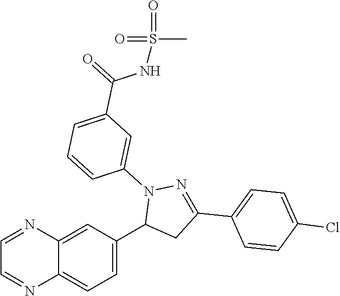 [SMILES: CS(=O)(=O)NC(=O)c1cccc(N2N=C(c3ccc(Cl)cc3)CC2c2ccc3nccnc3c2)c1] | | 82.5 ± 2.5 | 14.3 ± 4.7 |
| Compound of Formula II 73 | 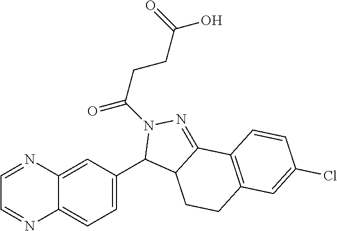 [SMILES: O=C(O)CCC(=O)N1N=C2c3ccc(Cl)cc3CCC2C1c1ccc2nccnc2c1] | 78.0 ± 1.9 | 18.8 ± 1.6 | |
| Compound of Formula II 74 | 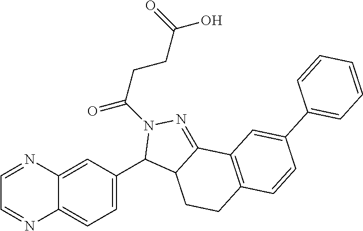 [SMILES: O=C(O)CCC(=O)N1N=C2c3cc(-c4ccccc4)ccc3CCC2C1c1ccc2nccnc2c1] | 97.2 ± 0.5 | 87.8 ± 0.7 | 9.0 ± 1.9 |
| Compound of Formula II 75 | 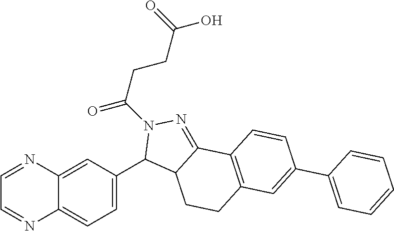 [SMILES: O=C(O)CCC(=O)N1N=C2c3ccc(-c4ccccc4)cc3CCC2C1c1ccc2nccnc2c1] | 92.8 ± 0.5 | 87.5 ± 0.6 | 15.8 ± 4.5 |
| Compound of Formula II 76 | 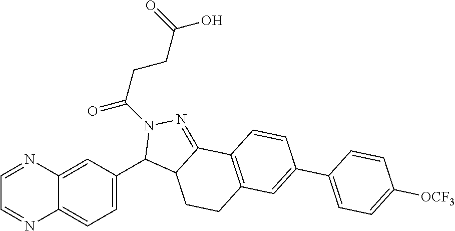 [SMILES: O=C(O)CCC(=O)N1N=C2c3ccc(-c4ccc(OC(F)(F)F)cc4)cc3CCC2C1c1ccc2nccnc2c1] | 96.5 | 77.3 ± 1.3 | 21.1 ± 3.2 |
| Compound of Formula IV 77 | 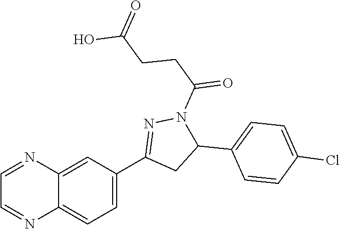 [SMILES: O=C(O)CCC(=O)N1N=C(c2ccc3nccnc3c2)CC1c1ccc(Cl)cc1] | 77.0 ± 0.4 | | |
| 78 | 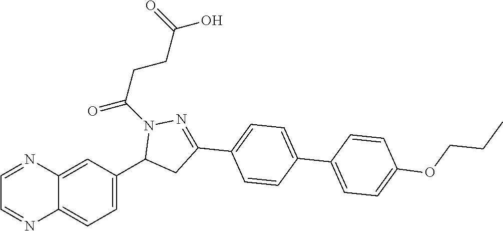 [SMILES: CCCOc1ccc(-c2ccc(C3=NN(C(=O)CCC(=O)O)C(c4ccc5nccnc5c4)C3)cc2)cc1] | | 88.8 ± 2.0 | 39.8 ± 18.5 |
| 79 | 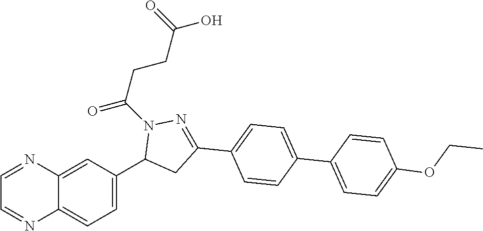 [SMILES: CCOc1ccc(-c2ccc(C3=NN(C(=O)CCC(=O)O)C(c4ccc5nccnc5c4)C3)cc2)cc1] | | 81.5 ± 0.1 | 25.2 ± 2.5 |
| 80 | 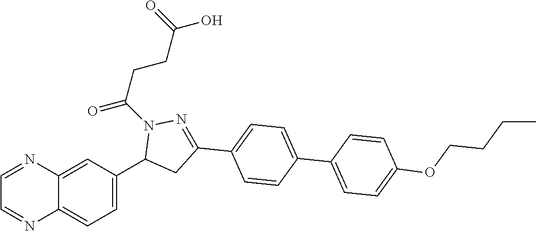 [SMILES: CCCCOc1ccc(-c2ccc(C3=NN(C(=O)CCC(=O)O)C(c4ccc5nccnc5c4)C3)cc2)cc1] | | 89.4 ± 2.5 | 21.9 ± 4.3 |
| 81 | 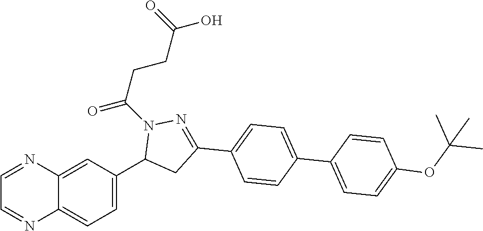 [SMILES: CC(C)(C)Oc1ccc(-c2ccc(C3=NN(C(=O)CCC(=O)O)C(c4ccc5nccnc5c4)C3)cc2)cc1] | | 29.8 ± 17.8 | |
| 82 | 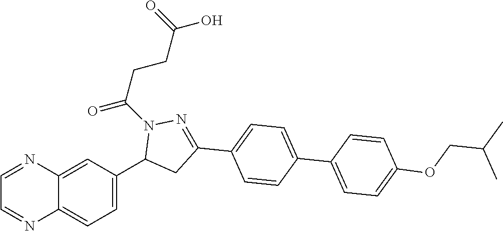 [SMILES: CC(C)COc1ccc(-c2ccc(C3=NN(C(=O)CCC(=O)O)C(c4ccc5nccnc5c4)C3)cc2)cc1] | | 75.7 ± 4.6 | 9.3 ± 7.6 |
| 83 | 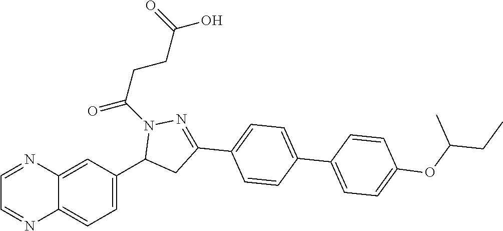 [SMILES: CCC(C)Oc1ccc(-c2ccc(C3=NN(C(=O)CCC(=O)O)C(c4ccc5nccnc5c4)C3)cc2)cc1] | | 69.2 ± 1.8 | <5 |
| 84 | 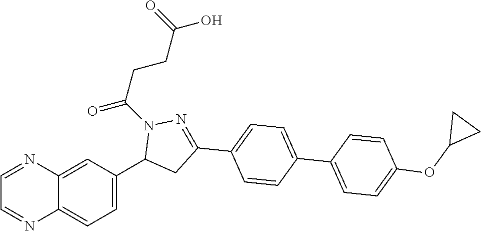 [SMILES: O=C(O)CCC(=O)N1N=C(c2ccc(-c3ccc(OC4CC4)cc3)cc2)CC1c1ccc2nccnc2c1] | | 75.1 ± 4.2 | 9.8 ± 1.0 |
| 85 | 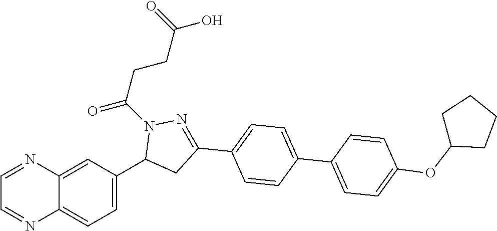 [SMILES: O=C(O)CCC(=O)N1N=C(c2ccc(-c3ccc(OC4CCCC4)cc3)cc2)CC1c1ccc2nccnc2c1] | | 71.8 ± 4.5 | 17.9 ± 7.3 |
| 86 | 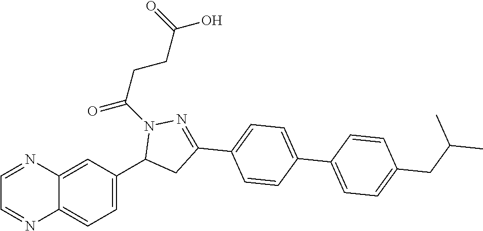 [SMILES: CC(C)Cc1ccc(-c2ccc(C3=NN(C(=O)CCC(=O)O)C(c4ccc5nccnc5c4)C3)cc2)cc1] | | 29.0 ± 8.6 | |
| 87 | 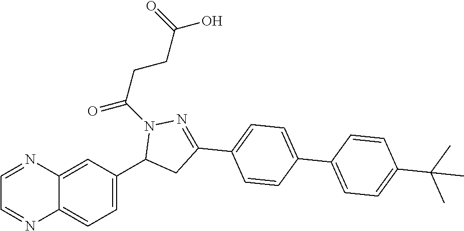 [SMILES: CC(C)(C)c1ccc(-c2ccc(C3=NN(C(=O)CCC(=O)O)C(c4ccc5nccnc5c4)C3)cc2)cc1] | | 6.0 ± 0.5 | |
| 88 | 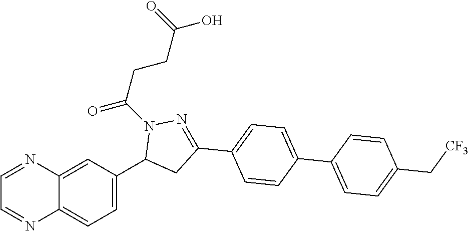 [SMILES: O=C(O)CCC(=O)N1N=C(c2ccc(-c3ccc(CC(F)(F)F)cc3)cc2)CC1c1ccc2nccnc2c1] | | 83.0 ± 1.1 | <5 |
| 89 | 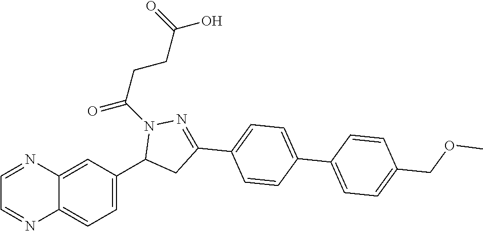 [SMILES: COCc1ccc(-c2ccc(C3=NN(C(=O)CCC(=O)O)C(c4ccc5nccnc5c4)C3)cc2)cc1] | 85.0 ± 1.7 | 21.9 ± 7.0 | |
| 90 | 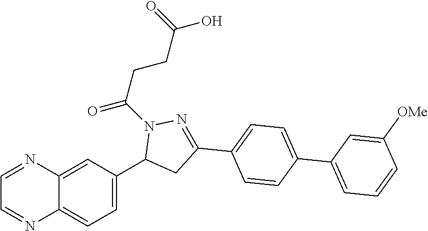 [SMILES: COc1cccc(-c2ccc(C3=NN(C(=O)CCC(=O)O)C(c4ccc5nccnc5c4)C3)cc2)c1] | 84.2 | 6.9 ± 2.2 | |
| 91 | 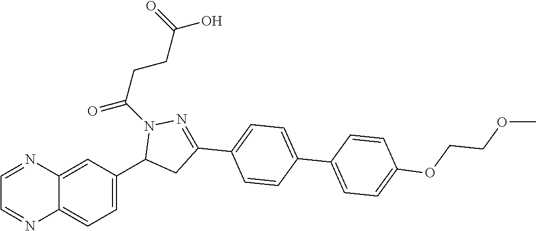 [SMILES: COCCOc1ccc(-c2ccc(C3=NN(C(=O)CCC(=O)O)C(c4ccc5nccnc5c4)C3)cc2)cc1] | | 73.2 ± 2.8 | <5 |
| 92 | 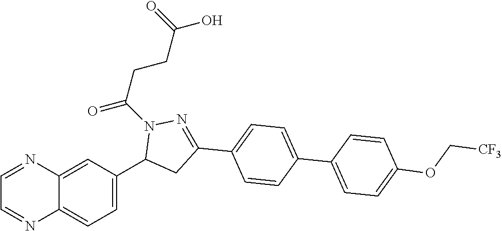 [SMILES: O=C(O)CCC(=O)N1N=C(c2ccc(-c3ccc(OCC(F)(F)F)cc3)cc2)CC1c1ccc2nccnc2c1] | | 81.4 ± 3.1 | 32.1 ± 0.8 |
| 93 | 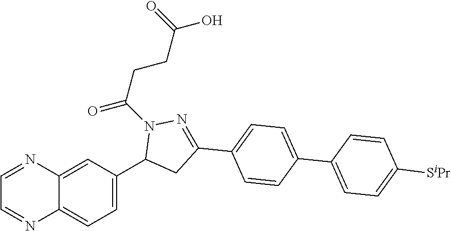 | | 49.1 ± 10.8 | |
| 94 | 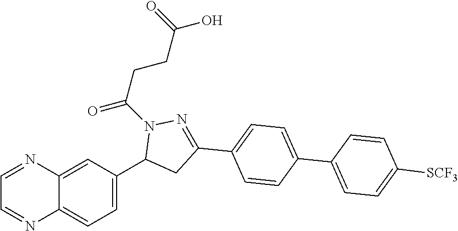 | | 32.7 ± 1.2 | |
| 95 | 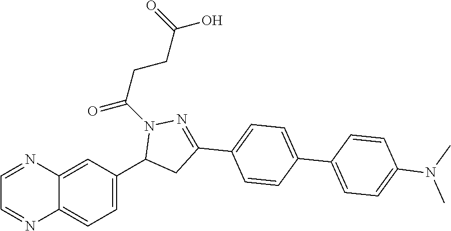 [SMILES: CN(C)c1ccc(-c2ccc(C3=NN(C(=O)CCC(=O)O)C(c4ccc5nccnc5c4)C3)cc2)cc1] | | 68.3 ± 2.1 | |
| 96 | 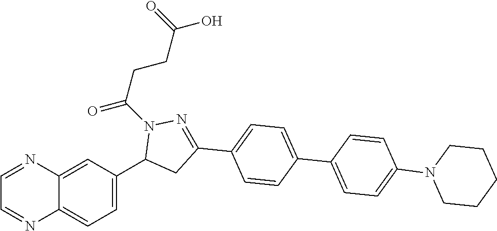 [SMILES: O=C(O)CCC(=O)N1N=C(c2ccc(-c3ccc(N4CCCCC4)cc3)cc2)CC1c1ccc2nccnc2c1] | 73.3 ± 12.1 | 8.2 ± 2.5 | |
| 97 | 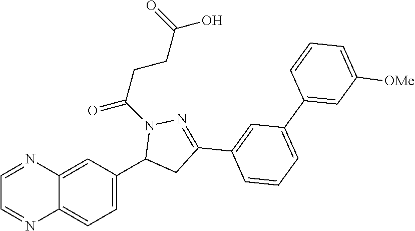 [SMILES: COc1cccc(-c2cccc(C3=NN(C(=O)CCC(=O)O)C(c4ccc5nccnc5c4)C3)c2)c1] | 76.0 ± 1.4 | 5.4 ± 0.2 | |
| 98 | 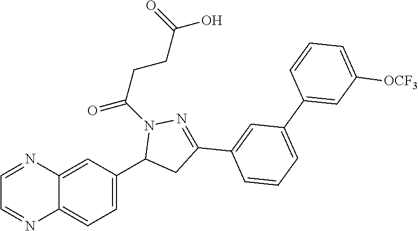 [SMILES: O=C(O)CCC(=O)N1N=C(c2cccc(-c3cccc(OC(F)(F)F)c3)c2)CC1c1ccc2nccnc2c1] | 91.0 ± 2.7 | 15.6 ± 0.6 | |
| 99 | 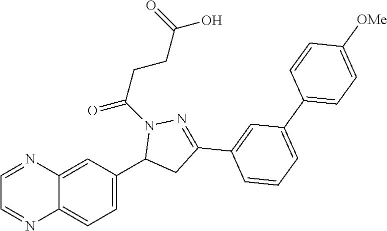 [SMILES: COc1ccc(-c2cccc(C3=NN(C(=O)CCC(=O)O)C(c4ccc5nccnc5c4)C3)c2)cc1] | 71.8 | <5 | |
| 100 | 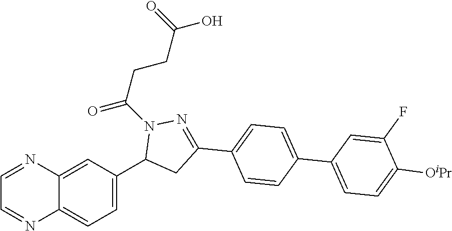 [SMILES: CC(C)Oc1ccc(-c2ccc(C3=NN(C(=O)CCC(=O)O)C(c4ccc5nccnc5c4)C3)cc2)cc1F] | | 89.2 ± 3.0 | 25.0 ± 7.8 |
| 101 | 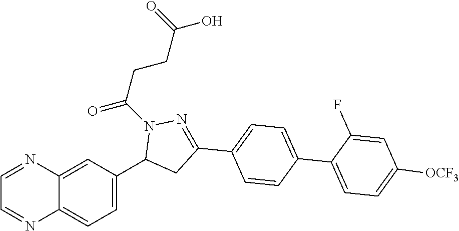 [SMILES: O=C(O)CCC(=O)N1N=C(c2ccc(-c3ccc(OC(F)(F)F)cc3F)cc2)CC1c1ccc2nccnc2c1] | | 65.7 ± 2.9 | |
| 102 | 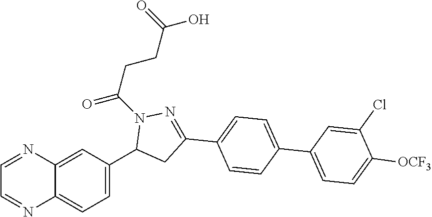 [SMILES: O=C(O)CCC(=O)N1N=C(c2ccc(-c3ccc(OC(F)(F)F)c(Cl)c3)cc2)CC1c1ccc2nccnc2c1] | | 52.8 ± 2.5 | |
| 103 | 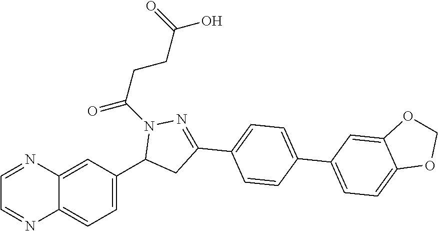 [SMILES: O=C(O)CCC(=O)N1N=C(c2ccc(-c3ccc4c(c3)OCO4)cc2)CC1c1ccc2nccnc2c1] | | 24.8 ± 6.2 | |
| 104 | 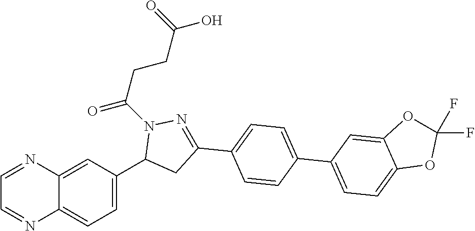 [SMILES: O=C(O)CCC(=O)N1N=C(c2ccc(-c3ccc4c(c3)OC(F)(F)O4)cc2)CC1c1ccc2nccnc2c1] | | 41.9 ± 8.9 | |
| 105 | 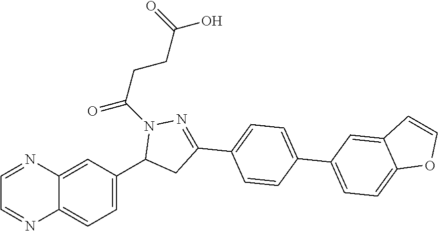 [SMILES: O=C(O)CCC(=O)N1N=C(c2ccc(-c3ccc4occc4c3)cc2)CC1c1ccc2nccnc2c1] | | 32.4.4 ± 12.1 | |
| 106 | 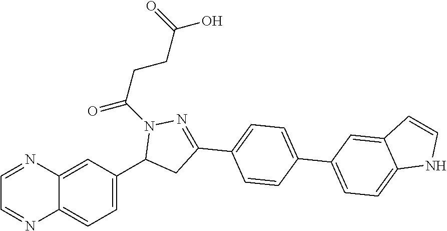 [SMILES: O=C(O)CCC(=O)N1N=C(c2ccc(-c3ccc4[nH]ccc4c3)cc2)CC1c1ccc2nccnc2c1] | 26.0 ± 2.5 | <5 | |
| 107 | 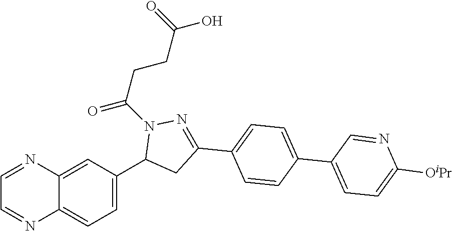 [SMILES: CC(C)Oc1ccc(-c2ccc(C3=NN(C(=O)CCC(=O)O)C(c4ccc5nccnc5c4)C3)cc2)cn1] | | 33.4 ± 2.1 | |
| 108 | 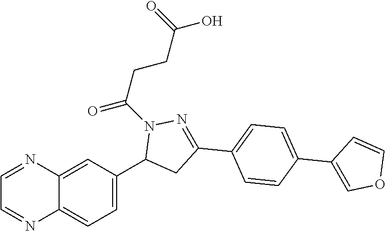 [SMILES: O=C(O)CCC(=O)N1N=C(c2ccc(-c3ccoc3)cc2)CC1c1ccc2nccnc2c1] | 48.3 ± 2.8 | 6.3 ± 6.1 | |
| 109 | 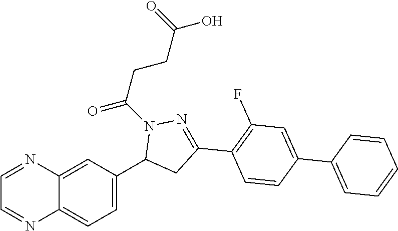 [SMILES: O=C(O)CCC(=O)N1N=C(c2ccc(-c3ccccc3)cc2F)CC1c1ccc2nccnc2c1] | | 40.4 ± 8.1 | |
| 110 | 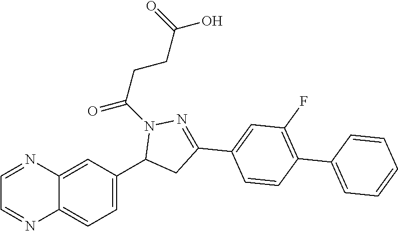 [SMILES: O=C(O)CCC(=O)N1N=C(c2ccc(-c3ccccc3)c(F)c2)CC1c1ccc2nccnc2c1] | | 40.4 ± 7.6 | |
| 111 | 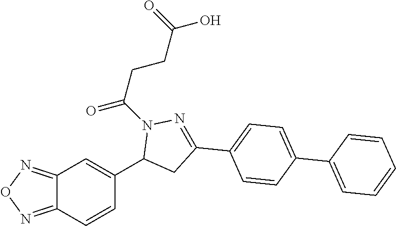 [SMILES: O=C(O)CCC(=O)N1N=C(c2ccc(-c3ccccc3)cc2)CC1c1ccc2nonc2c1] | | 43.3 ± 9.0 | |
| 112 | 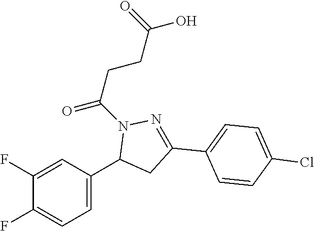 [SMILES: O=C(O)CCC(=O)N1N=C(c2ccc(Cl)cc2)CC1c1ccc(F)c(F)c1] | 21.7 ± 0.9 | | |
| 113 | 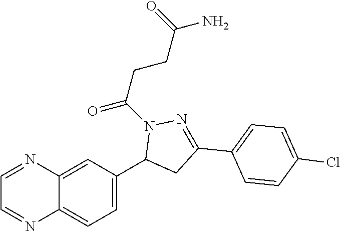 [SMILES: NC(=O)CCC(=O)N1N=C(c2ccc(Cl)cc2)CC1c1ccc2nccnc2c1] | 15.6 ± 0.4 | | |
| 114 | 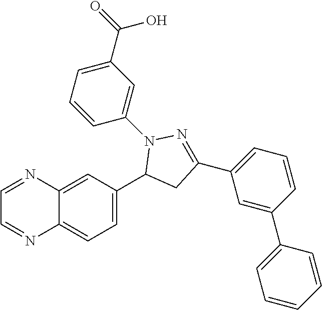 [SMILES: O=C(O)c1cccc(N2N=C(c3cccc(-c4ccccc4)c3)CC2c2ccc3nccnc3c2)c1] | | 58.3 ± 16.6 | |
| 115 | 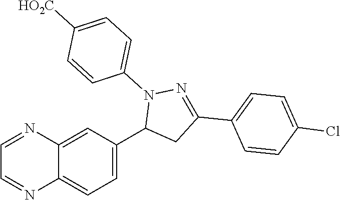 [SMILES: O=C(O)c1ccc(N2N=C(c3ccc(Cl)cc3)CC2c2ccc3nccnc3c2)cc1] | 84.0 ± 10.6 | 20.1 ± 1.1 | |
| 116 | 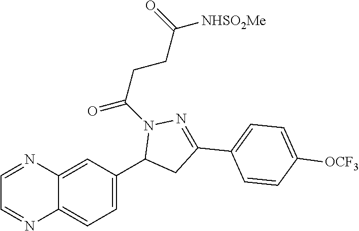 | 93.9 ± 4.7 | 72.4 ± 10.6 | <5 |
| 117 | 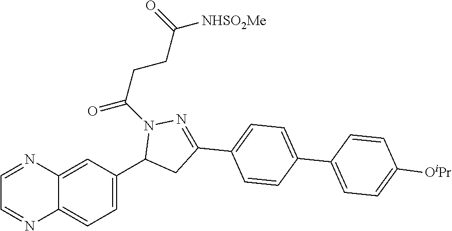 | | 72.5 ± 4.0 | 9.1 ± 2.4 |
| 118 | 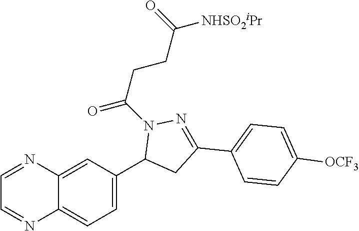 | | 29.7 ± 6.0 | |
| 119 | 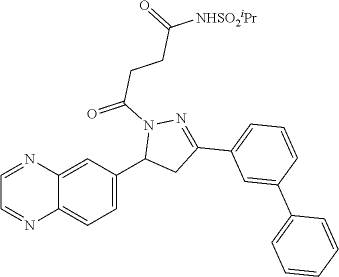 | | 81.8 ± 2.7 | 11.9 ± 2.7 |
| 120 | 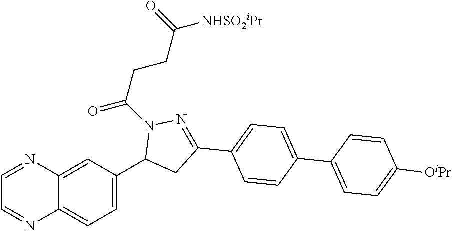 | | 70.4 ± 5.5 | 12.6 ± 1.4 |
| 121 | 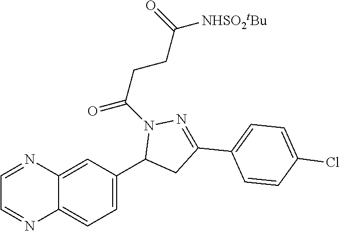 | | 9.8 ± 2.2 | |
| 122 | 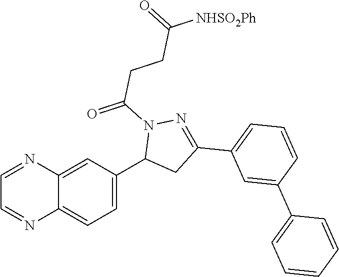 | | 78.9 ± 2.3 | 7.4 ± 4.7 |
| 123 | 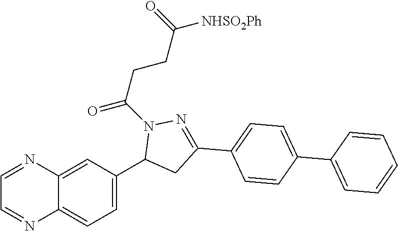 | | 77.0 ± 4.2 | 7.4 ± 0.7 |
| 124 | 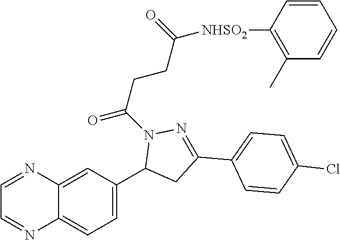 | | 12.7 ± 0.8 | |
| 125 | 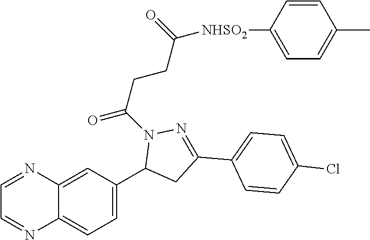 | | 21.8 ± 5.7 | |
| 126 | 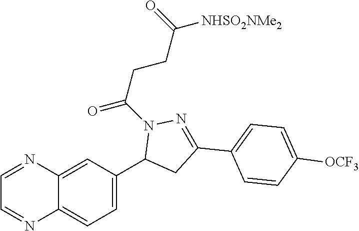 | | 25.5 ± 3.8 | |
| 127 | 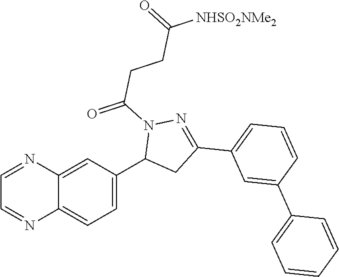 | | 84.1 ± 3.0 | 16.8 ± 0.7 |
| 128 | 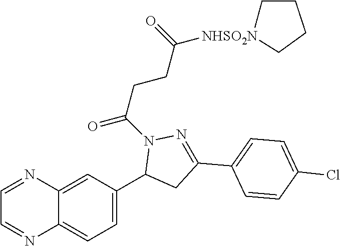 | | 53.0 ± 5.7 | |
| Compound of Formula II 129 | 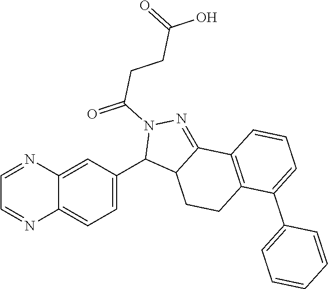 [SMILES: O=C(O)CCC(=O)N1N=C2c3cccc(-c4ccccc4)c3CCC2C1c1ccc2nccnc2c1] | | 42.5 ± 15.8 | |

### In Vivo Studies

The animal experiments described in this specification were all reviewed and approved by the Grasslands Animal Ethics Committee (Palmerston North, NZ) and all animals were cared for according the Code of Ethical Conduct (Animal Welfare Act, 1999) and its amendments.

### Toxicity Testing in Mice

The potential toxicology of Compound 19 was tested according to OECD 423. Mice testing used two groups with three mice (female Swiss mice, non-pregnant), all at approximately 8-9 weeks of age. On the day of testing, food was withdrawn 1-2 hours prior to dosing. Following fasting, the animals were weighed and the test substances administered. Access to food was resumed 1-2 hours after administration of the compounds. The compounds were administered in a single dose by oral gavage in corn oil (maximum of 0.3 ml per dose) at the selected dose and the time recorded. The mice were observed individually after dosing at least once during the first 30 minutes, periodically over the first 24 hours, with special attention given to the first 4 hours, and then daily thereafter for a total of 14 days. The mice were weighed every seven days (0, 7 and 14 days). All observations were systematically recorded for each individual animal. These observations included (if present): tremors, convulsions, salivation, diarrhoea, lethargy, sleep, coma, and changes in fur, skin, eyes, mucus membranes, respiration, motor activity and behaviour. No adverse effects were noted for Compound 19 at 300 mg/kg.

### In Vivo Trial Methods

### Example 1—In Vivo Study on Sheep Methane Emissions

The trial for testing the effects of Compound 19 on methane emissions from sheep over a 96 hour period in methane measurement chambers was approved by the Grasslands Animal Ethics Committee, and the use of the compounds in animals by the New Zealand Food Safety Authority, as required under ACVM legislation. Three sheep were used for each Compound 19 treatment group. The animals received a general-purpose diet (GP) at 1.5× maintenance energy requirements throughout the trials consisting of 500 g hay, 100 g soy bean meal, 290 g barley, 100 g molasses and 10 g mineral mix. During the time in the respiration chambers the animals had free access to water, with the feed offered twice a day at approximately 9:00 and 16:00 h in equal amounts. Feed refusals were weighed.

The animals were adapted to GP diet for 14 days prior to entering the methane measurement chambers. Rumen contents were sampled by stomach tubing before methane emissions were measured in the respiration chambers and the day after being released from the chambers to allow for the future examination of any effects on volatile fatty acids (VFAs). Rumen samples from stomach tubing were immediately subsampled for short chain fatty acid (SCFA) analysis (1.8 ml). The samples were centrifuged (20,000 g, 10 min, 4° C.). An aliquot of 0.9 ml of the supernatant was collected into 0.1 ml of internal standard (19.8 mM ethylbutyrate in 20% v/v phosphoric acid) for SCFA analysis and stored at −20° C. SCFA samples were thawed and centrifuged (20,000 g, 10 min, 4° C.) and 800 μl of the supernatant was collected into a crimp cap glass vial. SCFA were analysed by a HP 6890 gas chromatograph (Attwood et al., 1998).

After the 14-day acclimatisation period, methane emissions were measured in open circuit respiration chambers as described in Chapter 1 of the technical manual on respiration chamber design (http://www.globalresearchalliance.org/wp-content/uploads/2012/03/GRA-MAN-Facility-BestPract-2012-ch13.pdf). Dry matter intake (DMI, kg/d) was recorded during methane measurements from the weight difference of feed offered and refused. The first day in the chambers (24-hour period) served as a control for each animal. For the next three days the sheep received Compound 19 at 121.3 mg per day (72-hour period, Period 2).

### Results

Methane gas outputs for the 96-hour period (first 24 hours for background measurements (Period 1), followed by three days of dosing) in open circuit respiration chambers were determined and are summarised in Table 1.

TABLE 1
% methane inhibition resulting from once-daily drenching with
Compound 19 at a 121.3 mg/day dose - averaged over the 3
animals in each of the control group and the Compound 19 group.
CH₄ Inhibition [%]

| | Experimental Period | | |
|---|---|---|---|
| Treatment 1 | Day 1 | Day 2 | Day 3 |
| Control | −0.9 | 4.4 | 5.9 |
| Compound 19 | −6.5 | 8.9 | 11.4 |

It is anticipated that the RIV results for the compounds of Formula I will be somewhat predictive of in vivo activity in reducing methane production in rumens.

### Example 2—In Vivo Study on Sheep Methane Emissions

The trial for testing the effects of Compound 60, Compound 69, Compound 67 and Compound 64 on methane emissions from sheep over a 96-hour period in methane measurement chambers was approved by the Grasslands Animal Ethics Committee. Three sheep were used for each Compound 60 and Compound 69 treatment group and six sheep were used for each Compound 67 and Compound 64 treatment group. The animals received a general-purpose diet (GP) at 1.5× maintenance energy requirements throughout the trials consisting of 500 g hay, 100 g soybean meal, 290 g barley, 100 g molasses and 10 g mineral mix. During the time in the respiration chambers the animals had free access to water, with the feed offered twice a day at approximately 9:00 and 16:00 h in equal amounts. Feed refusals were weighed.

The animals were adapted to GP diet for 14 days prior to entering the methane measurement chambers. Rumen contents were sampled by stomach tubing before methane emissions were measured in the respiration chambers and the day after being released from the chambers to allow for the future examination of any effects on volatile fatty acids (VFAs). Rumen samples from stomach tubing were immediately subsampled for short chain fatty acid (SCFA) analysis (1.8 ml). The samples were centrifuged (20,000 g, 10 min, 4° C.). An aliquot of 0.9 ml of the supernatant was collected into 0.1 ml of internal standard (19.8 mM ethylbutyrate in 20% v/v phosphoric acid) for SCFA analysis and stored at −20° C. SCFA samples were thawed and centrifuged (20,000 g, 10 min, 4° C.) and 800 μl of the supernatant was collected into a crimp cap glass vial. SCFA were analysed by a HP 6890 gas chromatograph (Attwood et al., 1998).

After the 14-day acclimatisation period, methane emissions were measured in open circuit respiration chambers as described in Chapter 1 of the technical manual on respiration chamber design (http://www.globalresearchalliance.org/wp-content/uploads/2012/03/GRA-MAN-Facility-BestPract-2012-ch13.pdf). Dry matter intake (DMI, kg/d) was recorded during methane measurements from the weight difference of feed offered and refused. The first day in the chambers (24-hour period; Period 1) served as a control for each animal. For the next three days the sheep received Compound 60, Compound 69, Compound 67 and Compound 64 at 412, 311, 414 and 344 mg per day, respectively (72-hour period; Period 2).

### Results

Methane gas outputs for the 96-hour period (first 24 hours for background measurements (Period 1), followed by three days of dosing (Period 2)) in open circuit respiration chambers were determined and are summarised in Table 2.

TABLE 2
% Methane inhibition resulting from dosing Compound 60,
Compound 69, Compound 67 and Compound 64 in feed to sheep,
twice daily. Methane inhibition is an average from all
animals in each of the control and treated groups.

| | | CH₄ Inhibition [%] | | | |
|---|---|---|---|---|---|
| | Dose | | | | Average |
| | [mg/day] | Day 1 | Day 2 | Day 3 | Days 1-3 |
| Control | 0 | −2.5 | −0.1 | 3.1 | 0.2 |
| Compound | 412 | 26.3 | 20.9 | 31.2 | 26.2 |
| 60 | | | | | |
| Compound | 311 | 11.9 | 26.3 | 32.4 | 23.5 |
| 69 | | | | | |
| Compound | 414 | 24.2 | 36.7 | 48.6 | 36.5 |
| 67 | | | | | |
| Compound | 344 | 81.9 | 85.6 | 89.8 | 85.8 |
| 64 | | | | | |

### Example 3 In Vivo Cattle Trial Study on Methane Emissions

The trial for testing the effects of various doses of Compound 64 on methane emissions from cattle over four 48-hour periods in methane measurement chambers was approved by the Grasslands Animal Ethics Committee. Four cattle (Friesian dairy heifers, approximately 400 kg) were used for each of two Compound 64 treatment groups. The animals received a general-purpose diet (GP) at 1.5× maintenance energy requirements throughout the trials consisting of 500 g hay, 100 g soybean meal, 290 g barley, 100 g molasses and 10 g mineral mix. During the time in the respiration chambers the animals had free access to water, with the feed offered twice a day at approximately 9:00 and 16:00 h in equal amounts. Feed refusals were weighed.

The animals were adapted to GP diet for 14 days prior to entering the methane measurement chambers. Rumen contents were sampled by stomach tubing before methane emissions were measured in the respiration chambers and the day after being released from the chambers to allow for the future examination of any effects on volatile fatty acids (VFAs). Rumen samples from stomach tubing were immediately subsampled for short chain fatty acid (SCFA) analysis (1.8 ml). The samples were centrifuged (20,000 g, 10 min, 4° C.). An aliquot of 0.9 ml of the supernatant was collected into 0.1 ml of internal standard (19.8 mM ethylbutyrate in 20% v/v phosphoric acid) for SCFA analysis and stored at −20° C. SCFA samples were thawed and centrifuged (20,000 g, 10 min, 4° C.) and 800 μl of the supernatant was collected into a crimp cap glass vial. SCFA were analysed by a HP 6890 gas chromatograph (Attwood et al., 1998).

After the 14-day acclimatisation period, methane emissions were measured in open circuit respiration chambers as described in Chapter 1 of the technical manual on respiration chamber design (http://www.globalresearchalliance.org/wp-content/uploads/2012/03/GRA-MAN-Facility-BestPract-012-ch13.pdf). Dry matter intake (DMI, kg/d) was recorded during methane measurements from the weight difference of feed offered and refused. Chamber measurements were made once a week for five weeks. The first period in the chambers (48-hour period; Period 1) served as a control for each animal. For the next three-five days the two groups of cattle (low dose and high dose groups) received Compound 64 at 50 and 150 mg per day, respectively, before measurement in the chambers (48-hour period; Period 2). The cattle then received Compound 64 at 150 and 450 mg per day, respectively, with methane measurements in the chambers being made once a week (48-hour periods; Period 3, 4 and 5).

### Results

Methane gas outputs for the four 48-hour treatment periods (Periods 2-5) in open circuit respiration chambers were determined and are summarised in Table 1. There were little or no feed refusals.

TABLE 1
% Methane inhibition resulting from dosing Compound 64 in
feed to cattle, twice daily. Methane inhibition is an average
from all animals in each of the treated groups.

| Group of animals | Period | Dose [mg/day] | CH₄ Inhibition [%] |
|---|---|---|---|
| Low dose group | 2 | 50 | −4.7 ± 2.9 |
| | 3 | 300 | 20.2 ± 9.8 |
| | 4 | 300 | 2.5 ± 4.6 |
| | 5 | 300 | −1.8 ± 4.0 |
| High dose group | 2 | 150 | 1.9 ± 5.2 |
| | 3 | 450 | 15.6 ± 11.7 |
| | 4 | 450 | 0.5 ± 5.8 |
| | 5 | 450 | 5.6 ± 3.0 |

The present invention and its examples have been described in detail. However, the scope of the present invention is not intended to be limited to the particular examples of the invention described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as examples described herein may be utilized according to such related examples of the present invention. Thus, the following claims are intended to encompass within their scope modifications, substitutions, and variations to the examples of the invention disclosed herein.

### Numbered Embodiments

1 A compound of Formula II

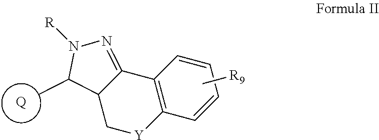

wherein R may be selected from:
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, —C(═O)—C₁-C₆—OH, or
—C(═O)—C₁-C₆—C(═O)—NH₂, —C(═O)—C₁-C₆—C(═O)—NH—C₁-C₆, —C(═O)—C₁-C₆—C(═O)—NH—C₃-C₈-cycloalkyl, —C(═O)—C₁-C₆—C(═O)—N(C₁-C₆)₂,

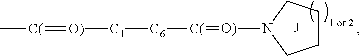

wherein J is a 5 or 6 membered heterocycle ring optionally including a second heteroatom selected from —NH—, —NC₁-C₆—, —S— or —O—; or
—C(═O)—C₁-C₆—C(═O)—NH—S(O)₂—C₁-C₆, —C(═O)—C₁-C₆—C(═O)—NH—S(O)₂—C₃-C₈-cycloalkyl, —C(═O)—C₁-C₆—C(═O)—NH—S(O)₂-aryl, C(═O)—C₁-C₆—C(═O)—NH—S(O)₂—C₁-C₆-aryl, —C(═O)—C₁-C₆—C(═O)—NH—S(O)₂-heteroaryl, —C(═O)—C₁-C₆—C(═O)—NH—S(O)₂—C₁-C₆-heteroaryl, or
—C(═O)—C₁-C₆—C(═O)—NH—S(O)₂—NH₂, —C(═O)—C₁-C₆—C(═O)—NH—S(O)₂—NH—C₁-C₆, —C(═O)—C₁-C₆—C(═O)—NH—S(O)₂—NH—C₃-C₈-cycloalkyl, —C(═O)—C₁-C₆—C(═O)—NH—S(O)₂—NH-aryl, —C(═O)—C₁-C₆—C(═O)—NH—S(O)₂—NH—C₁-C₆-aryl, —C(═O)—C₁-C₆—C(═O)—NH—S(O)₂—NH-heteroaryl, —C(═O)—C₁-C₆—C(═O)—NH—S(O)₂—NH—C₁-C₆-heteroaryl, —C(═O)—C₁-C₆—C(═O)—NH—S(O)₂—N(C₁-C₆)₂,

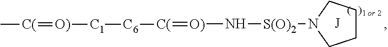

wherein J is a 5 or 6 membered heterocycle ring optionally including a second heteroatom selected from —NH—, NC₁-C₆—, —S— or —O—; or
—C(═O)—C₁-C₆—NH—C(═O)—NH—S(O)₂—C₁-C₆, —C(═O)—C₁-C₆—NH—C(═O)—NH—S(O)₂—C₃-C₈-cycloalkyl, —C(═O)—C₁-C₆—NH—C(═O)—NH—S(O)₂-aryl, —C(═O)—C₁-C₆—NH—C(═O)—NH—S(O)₂-heteroaryl, or
—C(═O)—C₁-C₆—NH—C(—O)—NH—S(O)₂—NH₂, —C(═O)—C₁-C₆—NH—C(═O)—NH—S(O)₂—NH—C₁-C₆, —C(═O)—C₁-C₆—NH—C(═O)—NH—S(O)₂—NH—C₃-C₈-cycloalkyl, —C(═O)—C₁-C₆—NH—C(═O)—NH—S(O)₂—NH-aryl, —C(═O)—C₁-C₆—C(═O)—NH—S(O)₂—NH—C₁-C₆-aryl, —C(═O)—C₁-C₆—NH—C(═O)—NH—S(O)₂—N(C₁-C₆)₂,

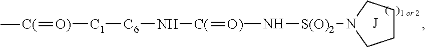

wherein J is a 5 or 6 membered heterocycle ring optionally including a second heteroatom selected from —NH—, NC₁-C₆—, —S— or —O—; or
—C₁-C₆—C(═O)—OH, —C₁-C₆—C(═O)—O—C₁-C₆, —C₁-C₆—C(═O)—O—C₁-C₆—O—(C═O)—C₁-C₆, —C₁-C₆—OH, or
—C₁-C₆—C(═O)—NH₂, —C₁-C₆—C(═O)—NH—C₁-C₆, —C₁-C₆—C(═O)—NH—C₃-C₈-cycloalkyl, —C₁-C₆—C(═O)—N(C₁-C₆)₂,

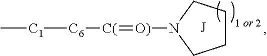

wherein J is a 5 or 6 membered heterocycle ring optionally including a second heteroatom selected from —NH—, —NC₁-C₆—, —S— or —O—; or
—C₁-C₆—C(═O)—NH—S(O)₂—C₁-C₆, —C₁-C₆—C(═O)—NH—S(O)₂—C₃-C₈-cycloalkyl, —C₁-C₆—C(═O)—NH—S(O)₂-aryl, —C₁-C₆—C(═O)—NH—S(O)₂—C₁-C₆-aryl, —C₁-C₆—C(═O)—NH—S(O)₂-heteroaryl, —C₁-C₆—C(═O)—NH—S(O)₂—C₁-C₆-heteroaryl, or
—C₁-C₆—C(═O)—NH—S(O)₂—NH₂, —C₁-C₆—C(═O)—NH—S(O)₂—NH—C₁-C₆, —C₁-C₆—C(═O)—NH—S(O)₂—NH—C₃-C₈-cycloalkyl, —C₁-C₆—C(═O)—NH—S(O)₂—NH-aryl, —C₁-C₆—C(═O)—NH—S(O)₂—NH—C₁-C₆-aryl, —C₁-C₆—C(═O)—NH—S(O)₂—NH-heteroaryl, —C₁-C₆—C(═O)—NH—S(O)₂—NH—C₁-C₆-heteroaryl, —C₁-C₆—C(═O)—NH—S(O)₂—N(C₁-C₆)₂,

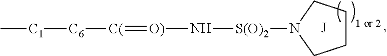

wherein J is a 5 or 6 membered heterocycle ring optionally including a second heteroatom selected from —NH—, NC₁-C₆—, —S— or —O—; or
—C₁-C₆—NH—C(═O)—NH—S(O)₂—C₁-C₆, —C₁-C₆—NH—C(═O)—NH—S(O)₂—C₃-C₈-cycloalkyl, —C₁-C₆—NH—C(═O)—NH—S(O)₂-aryl, —C₁-C₆—NH—C(═O)—NH—S(O)₂-heteroaryl, or
—C₁-C₆—NH—C(═O)—NH—S(O)₂—NH₂, —C₁-C₆—NH—C(═O)—NH—S(O)₂—NH—C₁-C₆, —C₁-C₆—NH—C(═O)—NH—S(O)₂—NH—C₃-C₈-cycloalkyl, —C₁-C₆—NH—C(═O)—NH—S(O)₂—NH-aryl, —C₁-C₆—C(═O)—NH—S(O)₂—NH—C₁-C₆-aryl, —C₁-C₆—NH—C(—O)—NH—S(O)₂—N(C₁-C₆)₂,

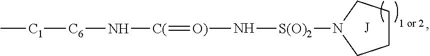

wherein J is a 5 or 6 membered heterocycle ring optionally including a second heteroatom selected from —NH—, NC₁-C₆—, —S— or —O—; or
or
—C(═O)-aryl-C(═O)—OH, —C(═O)-aryl-C(═O)—O—C₁-C₆, —C(═O)-aryl-C(═O)—O—C₁-C₆—O—(C═O)—C₁-C₆, —C(═O)-aryl-OH, or
—C(═O)-aryl-C(═O)—NH₂, —C(═O)-aryl-C(═O)—NH—C₁-C₆, —C(═O)-aryl-C(═O)—NH—C₃-C₈-cycloalkyl, —C(═O)-aryl-C(═O)—N(C₁-C₆)₂,

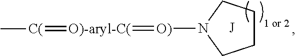

wherein J is a 5 or 6 membered heterocycle ring optionally including a second heteroatom selected from —NH—, —NC₁-C₆—, —S— or —O—; or
—C(═O)-aryl-C(═O)—NH—S(O)₂—C₁-C₆, —C(═O)-aryl-C(═O)—NH—S(O)₂—C₃-C₈-cycloalkyl, —C(═O)-aryl-C(═O)—NH—S(O)₂-aryl, —C(═O)-aryl-C(═O)—NH—S(O)₂—C₁-C₆-aryl, —C(═O)-aryl-C(═O)—NH—S(O)₂-heteroaryl, —C(═O)-aryl-C(═O)—NH—S(O)₂—C₁-C₆-heteroaryl, or
—C(═O)-aryl-C(═O)—NH—S(O)₂—NH₂, —C(═O)-aryl-C(═O)—NH—S(O)₂—NH—C₁-C₆, —C(═O)-aryl-C(═O)—NH—S(O)₂—NH—C₃-C₈-cycloalkyl, —C(═O)-aryl-C(═O)—NH—S(O)₂—NH-aryl, —C(═O)-aryl-C(═O)—NH—S(O)₂—NH—C₁-C₆-aryl, —C(═O)-aryl-C(═O)—NH—S(O)₂—NH-heteroaryl, —C(═O)-aryl-C(═O)—NH—S(O)₂—NH—C₁-C₆-heteroaryl, —C(═O)-aryl-C(═O)—NH—S(O)₂—N(C₁-C₆)₂,

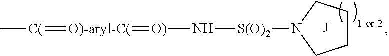

wherein J is a 5 or 6 membered heterocycle ring optionally including a second heteroatom selected from —NH—, NC₁-C₆—, —S— or —O—; or
—C(═O)-aryl-NH—C(═O)—NH—S(O)₂—C₁-C₆, —C(═O)-aryl-NH—C(═O)—NH—S(O)₂—C₃-C₈-cycloalkyl, —C(═O)-aryl-NH—C(═O)—NH—S(O)₂-aryl, —C(═O)-aryl-NH—C(═O)—NH—S(O)₂-heteroaryl, or
—C(═O)-aryl-NH—C(═O)—NH—S(O)₂—NH₂, —C(═O)-aryl-NH—C(═O)—NH—S(O)₂—NH—C₁-C₆, —C(═O)-aryl-NH—C(═O)—NH—S(O)₂—NH—C₃-C₈-cycloalkyl, —C(═O)-aryl-NH—C(═O)—NH—S(O)₂—NH-aryl, —C(═O)-aryl-C(═O)—NH—S(O)₂—NH—C₁-C₆-aryl, —C(═O)-aryl-NH—C(═O)—NH—S(O)₂—N(C₁-C₆)₂,

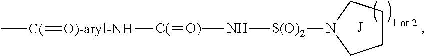

wherein J is a 5 or 6 membered heterocycle ring optionally including a second heteroatom selected from —NH—, NC₁-C₆—, —S— or —O—; or
or
-aryl-C(═O)—OH, -aryl-C(═O)—O—C₁-C₆, -aryl-C(═O)—O—C₁-C₆—O—(C═O)—C₁-C₆, -aryl-C₁-C₆—OH, or
-aryl-C(═O)—NH₂, -aryl-C(═O)—NH—C₁-C₆, -aryl-C(═O)—NH—C₃-C₈-cycloalkyl, -aryl-C(═O)—N(C₁-C₆)₂,

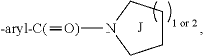

wherein J is a 5 or 6 membered heterocycle ring optionally including a second heteroatom selected from —NH—, —NC₁-C₆—, —S— or —O—; or
-aryl-C(═O)—NH—S(O)₂—C₁-C₆, -aryl-C(═O)—NH—S(O)₂—C₃-C₈-cycloalkyl, -aryl-C(═O)—NH—S(O)₂-aryl, -aryl-C(═O)—NH—S(O)₂—C₁-C₆-aryl, -aryl-C(═O)—NH—S(O)₂-heteroaryl, -aryl-C(═O)—NH—S(O)₂—C₁-C₆-heteroaryl, or
-aryl-C(═O)—NH—S(O)₂—NH₂, -aryl-C(═O)—NH—S(O)₂—NH—C₁-C₆, -aryl-C(═O)—NH—S(O)₂—NH—C₃-C₈-cycloalkyl, -aryl-C(═O)—NH—S(O)₂—NH-aryl, -aryl-C(═O)—NH—S(O)₂—NH—C₁-C₆-aryl, -aryl-C(═O)—NH—S(O)₂—NH-heteroaryl, -aryl-C(═O)—NH—S(O)₂—NH—C₁-C₆-heteroaryl, -aryl-C(═O)—NH—S(O)₂—N(C₁-C₆)₂,

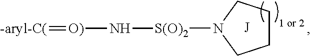

wherein J is a 5 or 6 membered heterocycle ring optionally including a second heteroatom selected from —NH—, NC₁-C₆—, —S— or —O—; or
-aryl-NH—C(═O)—NH—S(O)₂—C₁-C₆, -aryl-NH—C(═O)—NH—S(O)₂—C₃-C₈-cycloalkyl, -aryl-NH—C(═O)—NH—S(O)₂-aryl, -aryl-NH—C(═O)—NH—S(O)₂-heteroaryl, or
-aryl-NH—C(═O)—NH—S(O)₂—NH₂, -aryl-NH—C(═O)—NH—S(O)₂—NH—C₁-C₆, -aryl-NH—C(═O)—NH—S(O)₂—NH—C₃-C₈-cycloalkyl, -aryl-NH—C(═O)—NH—S(O)₂—NH-aryl, -aryl-C(═O)—NH—S(O)₂—NH—C₁-C₆-aryl, -aryl-NH—C(═O)—NH—S(O)₂—N(C₁-C₆)₂,

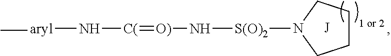

wherein J is a 5 or 6 membered heterocycle ring optionally including a second heteroatom selected from —NH—, NC₁-C₆—, —S— or —O—; or
-aryl-S(O)₂—OH, -aryl-S(O)₂—NH₂, -aryl-S(O)₂—NH—C₁-C₆, -aryl-S(O)₂—N(C₁-C₆)₂,

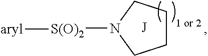

wherein J is a 5 or 6 membered heterocycle ring optionally including a second heteroatom selected from —NH—, —NC₁-C₆—, —S— or —O—; or
-aryl-S(O)₂—NH—C₃-C₈-cycloalkyl, or
—C(═O)-heteroaryl-C(═O)—OH, —C(═O)-heteroaryl-C(═O)—O—C₁-C₆, —C(═O)-heteroaryl-C(═O)—O—C₁-C₆—O—(C═O)—C₁-C₆, —C(═O)-aryl-OH, or
—C(═O)-heteroaryl-C(═O)—NH₂, —C(═O)-heteroaryl-C(═O)—NH—C₁-C₆, —C(═O)-heteroaryl-C(═O)—NH—C₃-C₈-cycloalkyl, —C(═O)-heteroaryl-C(═O)—N(C₁-C₆)₂,

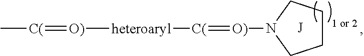

wherein J is a 5 or 6 membered heterocycle ring optionally including a second heteroatom selected from —NH—, —NC₁-C₆—, —S— or —O—; or
—C(═O)-heteroaryl-C(═O)—NH—S(O)₂—C₁-C₆, —C(═O)-heteroaryl-C(═O)—NH—S(O)₂—C₃-C₈-cycloalkyl, —C(═O)-heteroaryl-C(═O)—NH—S(O)₂-aryl, —C(═O)-heteroaryl-C(═O)—NH—S(O)₂—C₁-C₆-aryl, —C(═O)-heteroaryl-C(═O)—NH—S(O)₂-heteroaryl, —C(═O)-heteroaryl-C(═O)—NH—S(O)₂—C₁-C₆-heteroaryl, or
—C(═O)-heteroaryl-C(═O)—NH—S(O)₂—NH₂, —C(═O)-heteroaryl-C(═O)—NH—S(O)₂—NH—C₁-C₆, —C(═O)-heteroaryl-C(═O)—NH—S(O)₂—NH—C₃-C₈-cycloalkyl, —C(═O)-heteroaryl-C(═O)—NH—S(O)₂—NH-aryl, —C(═O)-heteroaryl-C(═O)—NH—S(O)₂—NH—C₁-C₆-aryl, —C(═O)-heteroaryl-C(═O)—NH—S(O)₂—NH-heteroaryl, —C(═O)-heteroaryl-C(═O)—NH—S(O)₂—NH—C₁-C₆-heteroaryl, —C(═O)-heteroaryl-C(═O)—NH—S(O)₂—N(C₁-C₆)₂,

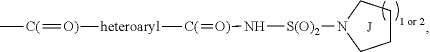

wherein J is a 5 or 6 membered heterocycle ring optionally including a second heteroatom selected from —NH—, NC₁-C₆—, —S— or —O—; or
—C(═O)-heteroaryl-NH—C(═O)—NH—S(O)₂—C₁-C₆, —C(═O)-heteroaryl-NH—C(═O)—NH—S(O)₂—C₃-C₈-cycloalkyl, —C(═O)-heteroaryl-NH—C(═O)—NH—S(O)₂-aryl, —C(═O)-heteroaryl-NH—C(═O)—NH—S(O)₂-heteroaryl, or
—C(═O)-heteroaryl-NH—C(═O)—NH—S(O)₂—NH₂, —C(═O)-heteroaryl-NH—C(═O)—NH—S(O)₂—NH—C₁-C₆, —C(═O)-heteroaryl-NH—C(═O)—NH—S(O)₂—NH—C₃-C₈-cycloalkyl, —C(═O)-heteroaryl-NH—C(═O)—NH—S(O)₂—NH-aryl, —C(═O)-heteroaryl-C(═O)—NH—S(O)₂—NH—C₁-C₆-aryl, —C(═O)-heteroaryl-NH—C(═O)—NH—S(O)₂—N(C₁-C₆)₂,

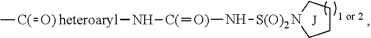

wherein J is a 5 or 6 membered heterocycle ring optionally including a second heteroatom selected from —NH—, NC₁-C₆—, —S— or —O—; or
or
-heteroaryl-C(═O)—OH, -heteroaryl-C(═O)—O—C₁-C₆, -heteroaryl-C(═O)—O—C₁-C₆—O—(C═O)—C₁-C₆, -heteroaryl-C₁-C₆—OH, or
heteroaryl-C(═O)—NH₂, -heteroaryl-C(═O)—NH—C₁-C₆, -heteroaryl-C(═O)—NH—C₃-C₈-cycloalkyl, -heteroaryl-C(═O)—N(C₁-C₆)₂,

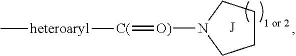

wherein J is a 5 or 6 membered heterocycle ring optionally including a second heteroatom selected from —NH—, —NC₁-C₆—, —S— or —O—; or
-heteroaryl-C(═O)—NH—S(O)₂—C₁-C₆, -heteroaryl-C(═O)—NH—S(O)₂—C₃-C₈-cycloalkyl, heteroaryl-C(═O)—NH—S(O)₂-aryl, -heteroaryl-C(═O)—NH—S(O)₂—C₁-C₆-aryl, -heteroaryl-C(═O)—NH—S(O)₂-heteroaryl, -heteroaryl-C(═O)—NH—S(O)₂—C₁-C₆-heteroaryl, or
-heteroaryl-C(═O)—NH—S(O)₂—NH₂, -heteroaryl-C(═O)—NH—S(O)₂—NH—C₁-C₆, -heteroaryl-C(═O)—NH—S(O)₂—NH—C₃-C₈-cycloalkyl, -heteroaryl-C(═O)—NH—S(O)₂—NH-aryl, -heteroaryl-C(═O)—NH—S(O)₂—NH—C₁-C₆-aryl, -heteroaryl-C(═O)—NH—S(O)₂—NH-heteroaryl, -heteroaryl-C(═O)—NH—S(O)₂—NH—C₁-C₆-heteroaryl, -heteroaryl-C(═O)—NH—S(O)₂—N(C₁-C₆)₂,

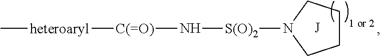

wherein J is a 5 or 6 membered heterocycle ring optionally including a second heteroatom selected from —NH—, NC₁-C₆—, —S— or —O—; or
-heteroaryl-NH—C(═O)—NH—S(O)₂—C₁-C₆, -heteroaryl-NH—C(═O)—NH—S(O)₂—C₃-C₈-cycloalkyl, -heteroaryl-NH—C(═O)—NH—S(O)₂-aryl, -heteroaryl-NH—C(═O)—NH—S(O)₂-heteroaryl, or
-heteroaryl-NH—C(═O)—NH—S(O)₂—NH₂, -heteroaryl-NH—C(═O)—NH—S(O)₂—NH—C₁-C₆, -heteroaryl-NH—C(═O)—NH—S(O)₂—NH—C₃-C₈-cycloalkyl, -heteroaryl-NH—C(═O)—NH—S(O)₂—NH-aryl, -heteroaryl-C(═O)—NH—S(O)₂—NH—C₁-C₆-aryl, -heteroaryl-NH—C(═O)—NH—S(O)₂—N(C₁-C₆)₂,

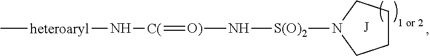

wherein J is a 5 or 6 membered heterocycle ring optionally including a second heteroatom selected from —NH—, NC₁-C₆—, —S— or —O—;
or
—C₁-C₆-aryl-C(═O)—OH, —C₁-C₆-aryl-C(═O)—O—C₁-C₆, —C₁-C₆-aryl-C(═O)—O—C₁-C₆—O—(C═O)—C₁-C₆, —C₁-C₆-aryl-C₁-C₆—OH, or
—C₁-C₆-aryl-C(═O)—NH₂, —C₁-C₆-aryl-C(═O)—NH—C₁-C₆, —C₁-C₆-aryl-C(═O)—NH—C₃-C₈-cycloalkyl, —C₁-C₆-aryl-C(═O)—N(C₁-C₆)₂,

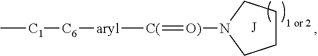

wherein J is a 5 or 6 membered heterocycle ring optionally including a second heteroatom selected from —NH—, —NC₁-C₆—, —S— or —O—; or
—C₁-C₆-aryl-C(═O)—NH—S(O)₂—C₁-C₆, —C₁-C₆-aryl-C(═O)—NH—S(O)₂—C₃-C₈-cycloalkyl, —C₁-C₆-aryl-C(═O)—NH—S(O)₂-aryl, —C₁-C₆-aryl-C(═O)—NH—S(O)₂—C₁-C₆-aryl, —C₁-C₆-aryl-C(═O)—NH—S(O)₂-heteroaryl, —C₁-C₆-aryl-C(═O)—NH—S(O)₂—C₁-C₆-heteroaryl, or
—C₁-C₆-aryl-C(═O)—NH—S(O)₂—NH₂, —C₁-C₆-aryl-C(═O)—NH—S(O)₂—NH—C₁-C₆, —C₁-C₆-aryl-C(═O)—NH—S(O)₂—NH—C₃-C₈-cycloalkyl, —C₁-C₆-aryl-C(═O)—NH—S(O)₂—NH-aryl, —C₁-C₆-aryl-C(═O)—NH—S(O)₂—NH—C₁-C₆-aryl, —C₁-C₆-aryl-C(═O)—NH—S(O)₂—NH-heteroaryl, —C₁-C₆-aryl-C(═O)—NH—S(O)₂—NH—C₁-C₆-heteroaryl, —C₁-C₆-aryl-C(═O)—NH—S(O)₂—N(C₁-C₆)₂,

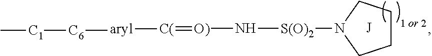

wherein J is a 5 or 6 membered heterocycle ring optionally including a second heteroatom selected from —NH—, NC₁-C₆—, —S— or —O—; or
—C₁-C₆-aryl-NH—C(═O)—NH—S(O)₂—C₁-C₆, —C₁-C₆-aryl-NH—C(═O)—NH—S(O)₂—C₃-C₈-cycloalkyl, —C₁-C₆-aryl-NH—C(═O)—NH—S(O)₂-aryl, —C₁-C₆-aryl-NH—C(═O)—NH—S(O)₂-heteroaryl, or
—C₁-C₆-aryl-NH—C(═O)—NH—S(O)₂—NH₂, —C₁-C₆-aryl-NH—C(═O)—NH—S(O)₂—NH—C₁-C₆, —C₁-C₆-aryl-NH—C(═O)—NH—S(O)₂—NH—C₃-C₈-cycloalkyl, —C₁-C₆-aryl-NH—C(═O)—NH—S(O)₂—NH-aryl, —C₁-C₆-aryl-C(═O)—NH—S(O)₂—NH—C₁-C₆-aryl, —C₁-C₆-aryl-NH—C(═O)—NH—S(O)₂—N(C₁-C₆)₂,

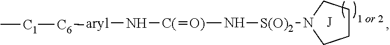

wherein J is a 5 or 6 membered heterocycle ring optionally including a second heteroatom selected from —NH—, NC₁-C₆—, —S— or —O—; or
—C₁-C₆-aryl-S(O)₂—OH, —C₁-C₆-aryl-S(O)₂—NH₂, —C₁-C₆-aryl-S(O)₂—NH—C₁-C₆, —C₁-C₆-aryl-S(O)₂—N(C₁-C₆)₂,

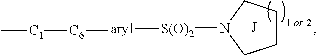

wherein J is a 5 or 6 membered heterocycle ring optionally including a second heteroatom selected from —NH—, —NC₁-C₆—, —S— or —O—; or
—C₁-C₆-aryl-S(O)₂—NH—C₃-C₈-cycloalkyl, or
—C₁-C₆-heteroaryl-C(═O)—OH, —C₁-C₆-heteroaryl-C(═O)—O—C₁-C₆, —C₁-C₆-heteroaryl-C(═O)—O—C₁-C₆—O—(C═O)—C₁-C₆, —C₁-C₆-heteroaryl-C₁-C₆—OH, or
—C₁-C₆-heteroaryl-C(═O)—NH₂, —C₁-C₆-heteroaryl-C(═O)—NH—C₁-C₆, —C₁-C₆-heteroaryl-C(═O)—NH—C₃-C₈-cycloalkyl, —C₁-C₆-heteroaryl-C(═O)—N(C₁-C₆)₂,

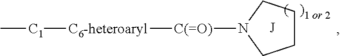

wherein J is a 5 or 6 membered heterocycle ring optionally including a second heteroatom selected from —NH—, —NC₁-C₆—, —S— or —O—; or
—C₁-C₆-heteroaryl-C(═O)—NH—S(O)₂—C₁-C₆, —C₁-C₆-heteroaryl-C(═O)—NH—S(O)₂—C₃-C₈-cycloalkyl, —C₁-C₆-heteroaryl-C(═O)—NH—S(O)₂-aryl, —C₁-C₆-heteroaryl-C(═O)—NH—S(O)₂—C₁-C₆-aryl, —C₁-C₆-heteroaryl-C(═O)—NH—S(O)₂-heteroaryl, —C₁-C₆-heteroaryl-C(═O)—NH—S(O)₂—C₁-C₆-heteroaryl, or
—C₁-C₆-heteroaryl-C(═O)—NH—S(O)₂—NH₂, —C₁-C₆-heteroaryl-C(═O)—NH—S(O)₂—NH—C₁-C₆, —C₁-C₆heteroaryl-C(═O)—NH—S(O)₂—NH—C₃-C₈-cycloalkyl, —C₁-C₆-heteroaryl-C(═O)—NH—S(O)₂—NH-aryl, —C₁-C₆-heteroaryl-C(═O)—NH—S(O)₂—NH—C₁-C₆-aryl, —C₁-C₆-heteroaryl-C(═O)—NH—S(O)₂—NH-heteroaryl, —C₁-C₆-heteroaryl-C(═O)—NH—S(O)₂—NH—C₁-C₆-heteroaryl, —C₁-C₆-heteroaryl-C(═O)—NH—S(O)₂—N(C₁-C₆)₂,

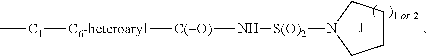

wherein J is a 5 or 6 membered heterocycle ring optionally including a second heteroatom selected from —NH—, NC₁-C₆—, —S— or —O—; or
—C₁-C₆-heteroaryl-NH—C(═O)—NH—S(O)₂—C₁-C₆, —C₁-C₆-heteroaryl-NH—C(═O)—NH—S(O)₂—C₃-C₈-cycloalkyl, —C₁-C₆-heteroaryl-NH—C(═O)—NH—S(O)₂-aryl, —C₁-C₆-heteroaryl-NH—C(═O)—NH—S(O)₂-heteroaryl, or
—C₁-C₆-heteroaryl-NH—C(═O)—NH—S(O)₂—NH₂, —C₁-C₆-heteroaryl-NH—C(═O)—NH—S(O)₂—NH—C₁-C₆, —C₁-C₆-heteroaryl-NH—C(═O)—NH—S(O)₂—NH—C₃-C₈-cycloalkyl, —C₁-C₆-heteroaryl-NH—C(═O)—NH—S(O)₂—NH-aryl, —C₁-C₆-heteroaryl-C(═O)—NH—S(O)₂—NH—C₁-C₆-aryl, —C₁-C₆-heteroaryl-NH—C(═O)—NH—S(O)₂—N(C₁-C₆)₂,

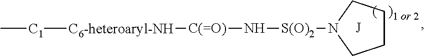

wherein J is a 5 or 6 membered heterocycle ring optionally including a second heteroatom selected from —NH—, NC₁-C₆—, —S— or —O—;
or
wherein Q may be selected from

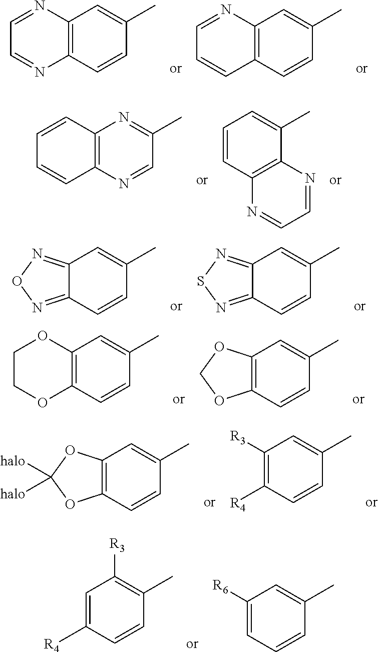

and
wherein R₃, R₄, or R₆ are independently selected from —H, halo, hydroxy, C₁-C₆ alkyl or C₁-C₆ alkoxy, alkoxy substituted with one or more halo, aryl optionally substituted with one or more halo, hydroxy, alkoxy, alkoxy substituted with one or more halo;
wherein R₉ is selected from —H, halo, hydroxy, C₁-C₆ alkyl, C₁-C₆ alkoxy, aryl, or —O-aryl, where each C₁-C₆ alkyl, C₁-C₆ alkoxy, aryl, or —O-aryl may be further substituted with one or more halo, hydroxy, C₁-C₆ alkoxy or C₁-C₆ alkoxy further substituted with one or more halo; and
wherein Y is —CH₂—, —CHR₅, —C(R₅)₂— wherein each R₅ is independently C₁-C₆ alkyl; —O— or —S—, —S(O), —S(O)₂, —NH—, —NR₁₀—, wherein R₁₀ is C₁-C₆ alkyl;
or a salt thereof.

2 The compound of embodiment 1, wherein R is selected from
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, or
—C(═O)—C₁-C₆—C(═O)—NH₂, —C(═O)—C₁-C₆—C(═O)—NH—C₁-C₆, —C(═O)—C₁-C₆—C(═O)—N(C₁-C₆)₂,
—C(═O)—C₁-C₆—C(═O)—NH—S(O)₂—C₁-C₆, —C(═O)—C₁-C₆—C(═O)—NH—S(O)₂-aryl, —C(═O)—C₁-C₆—C(═O)—NH—S(O)₂—N(C₁-C₆)₂,

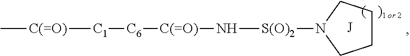

wherein J is a 5 or 6 membered heterocycle ring optionally including a second heteroatom selected from —NH—, NC₁-C₆—, —S— or —O—; or
—C₁-C₆—C(═O)—OH, or
-aryl-C(═O)—OH, or
-aryl-C(═O)—NH—S(O)₂—C₁-C₆.

3 The compound of embodiment 1 or 2, wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, —C₁-C₆—C(═O)—OH or -aryl-C(═O)—OH.

4 The compound of any of embodiment 1 to 3 wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂ or -aryl-C(═O)—OH.

5 The compound of any of embodiments 1 to 3, wherein R is —C(═O)—C₁-C₆—C(═O)—OH.

6 The compound of any one of embodiments 1 to 5 wherein R is —C(═O)—C₁-C₆—C(═O)—NH—S(O)₂—C₁-C₆.

7 The compound of any one of embodiments 1 to 6 wherein R is selected from —C(═O)—C₁-C₆—C(═O)—NH—S(O)₂—N(C₁-C₆)₂ or —C(═O)—C₁-C₆—C(═O)—NH—S(O)₂-aryl, such as —C(═O)—C₁-C₆—C(═O)—NH—S(O)₂-Ph.

8 The compound of any one of embodiments 1 to 4, wherein R is -aryl-C(═O)—OH.

9 The compound of any one of embodiments 1 to 4, wherein R is —C(═O)—CH₂—CH₂—C(═O)—OH.

10 The compound of embodiment 6, wherein R is selected from —C(═O)—CH₂—CH₂—C(═O)—NH—S(O)₂-Me or —C(═O)—CH₂—CH₂—C(═O)—NH—S(O)₂-iPr.

11 The compound of embodiment 7, wherein R is —C(═O)—CH₂—CH₂—C(═O)—NH—S(O)₂—NMe₂.

12 The compound of embodiment 8, wherein R is -phenyl-C(═O)—OH.

13 The compound of any one of embodiments 1 to 12 wherein Q is selected from:

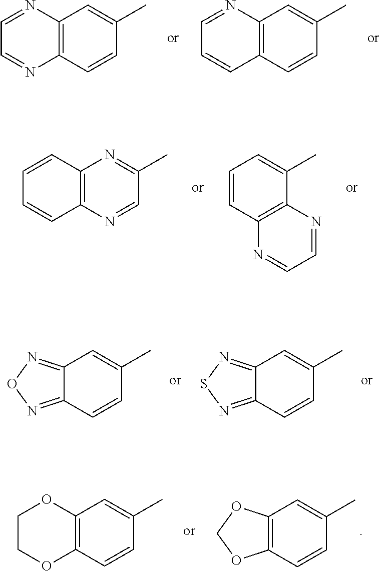

14 The compound of embodiment 13, wherein Q is selected from:

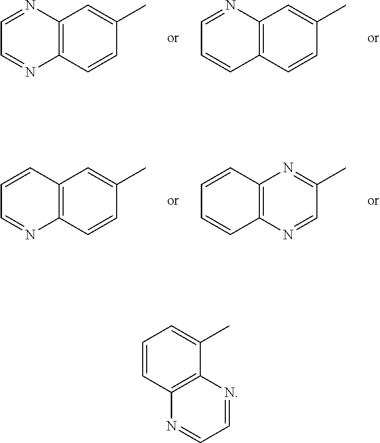

15 The compound of embodiment 13 or 14 wherein Q is

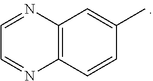

16 The compound of any one of embodiments 1 to 15, wherein R₉ is aryl, or aryl substituted with one or more —OC₁-C₆alkyl, or halo.

17 The compound of any one of embodiments 1 to 16, wherein Y is —CH₂—.

18 The compound of any one of embodiments 1 to 17, wherein the compound of Formula II is selected from

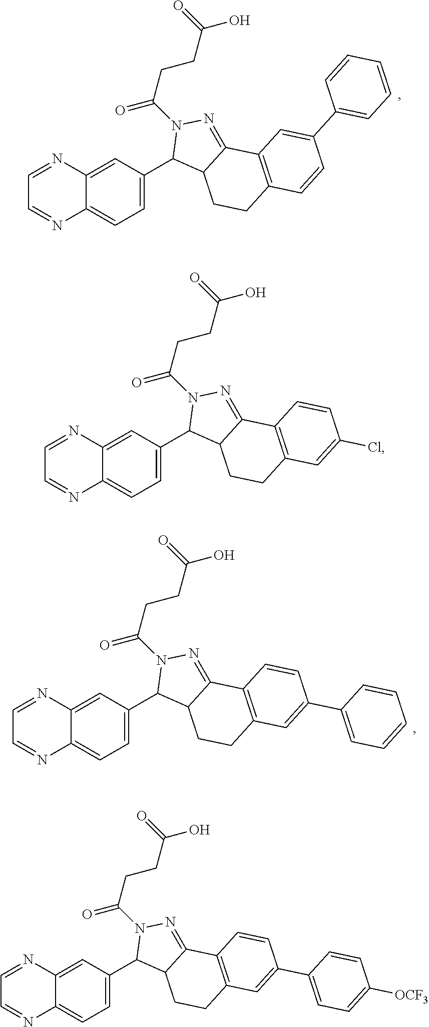 [SMILES: O=C(O)CCC(=O)N1N=C2c3cc(-c4ccccc4)ccc3CCC2C1c1ccc2nccnc2c1 ; O=C(O)CCC(=O)N1N=C2c3ccc(Cl)cc3CCC2C1c1ccc2nccnc2c1 ; O=C(O)CCC(=O)N1N=C2c3ccc(-c4ccccc4)cc3CCC2C1c1ccc2nccnc2c1 ; O=C(O)CCC(=O)N1N=C2c3ccc(-c4ccc(OC(F)(F)F)cc4)cc3CCC2C1c1ccc2nccnc2c1]

or a salt thereof.

19 A compound of Formula III

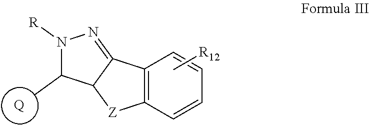

wherein R may be selected from:
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, —C(═O)—C₁-C₆—OH, or —C(═O)—C₁-C₆—C(═O)—NH₂, —C(═O)—C₁-C₆—C(═O)—NH—C₁-C₆, —C(═O)—C₁-C₆—C(═O)—NH—C₃-C₈-cycloalkyl, —C(═O)—C₁-C₆—C(═O)—N(C₁-C₆)₂,

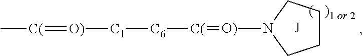

wherein J is a 5 or 6 membered heterocycle ring optionally including a second heteroatom selected from —NH—, —NC₁-C₆—, —S— or —O—; or
—C(═O)—C₁-C₆—C(═O)—NH—S(O)₂—C₁-C₆, —C(═O)—C₁-C₆—C(═O)—NH—S(O)₂—C₃-C₈-cycloalkyl, —C(═O)—C₁-C₆—C(═O)—NH—S(O)₂-aryl, C(═O)—C₁-C₆—C(═O)—NH—S(O)₂—C₁-C₆-aryl, —C(═O)—C₁-C₆—C(═O)—NH—S(O)₂-heteroaryl, —C(═O)—C₁-C₆—C(═O)—NH—S(O)₂—C₁-C₆-heteroaryl, or
—C(═O)—C₁-C₆—C(═O)—NH—S(O)₂—NH₂, —C(═O)—C₁-C₆—C(═O)—NH—S(O)₂—NH—C₁-C₆, —C(═O)—C₁-C₆—C(═O)—NH—S(O)₂—NH—C₃-C₈-cycloalkyl, —C(═O)—C₁-C₆—C(═O)—NH—S(O)₂—NH-aryl, —C(═O)—C₁-C₆—C(═O)—NH—S(O)₂—NH—C₁-C₆-aryl, —C(═O)—C₁-C₆—C(═O)—NH—S(O)₂—NH-heteroaryl, —C(═O)—C₁-C₆—C(═O)—NH—S(O)₂—NH—C₁-C₆-heteroaryl, —C(═O)—C₁-C₆—C(═O)—NH—S(O)₂—N(C₁-C₆)₂,

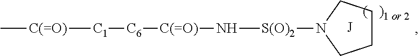

wherein J is a 5 or 6 membered heterocycle ring optionally including a second heteroatom selected from —NH—, NC₁-C₆—, —S— or —O—; or
—C(═O)—C₁-C₆—NH—C(═O)—NH—S(O)₂—C₁-C₆, —C(═O)—C₁-C₆—NH—C(═O)—NH—S(O)₂—C₃-C₈-cycloalkyl, —C(═O)—C₁-C₆—NH—C(═O)—NH—S(O)₂-aryl, —C(═O)—C₁-C₆—NH—C(═O)—NH—S(O)₂-heteroaryl, or
—C(═O)—C₁-C₆—NH—C(═O)—NH—S(O)₂—NH₂, —C(═O)—C₁-C₆—NH—C(═O)—NH—S(O)₂—NH—C₁-C₆, —C(═O)—C₁-C₆—NH—C(═O)—NH—S(O)₂—NH—C₃-C₈-cycloalkyl, —C(═O)—C₁-C₆—NH—C(═O)—NH—S(O)₂—NH-aryl, —C(═O)—C₁-C₆—C(═O)—NH—S(O)₂—NH—C₁-C₆-aryl, —C(═O)—C₁-C₆—NH—C(═O)—NH—S(O)₂—N(C₁-C₆)₂,

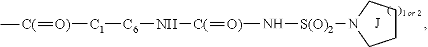

wherein J is a 5 or 6 membered heterocycle ring optionally including a second heteroatom selected from —NH—, NC₁-C₆—, —S— or —O—; or
—C₁-C₆—C(═O)—OH, —C₁-C₆—C(═O)—O—C₁-C₆, —C₁-C₆—C(═O)—O—C₁-C₆—O—(C═O)—C₁-C₆, —C₁-C₆—OH, or
—C₁-C₆—C(═O)—NH₂, —C₁-C₆—C(═O)—NH—C₁-C₆, —C₁-C₆—C(═O)—NH—C₃-C₈-cycloalkyl, —C₁-C₆—C(═O)—N(C₁-C₆)₂,

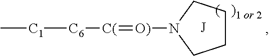

wherein J is a 5 or 6 membered heterocycle ring optionally including a second heteroatom selected from —NH—, —NC₁-C₆—, —S— or —O—; or
—C₁-C₆—C(═O)—NH—S(O)₂—C₁-C₆, —C₁-C₆—C(═O)—NH—S(O)₂—C₃-C₈-cycloalkyl, —C₁-C₆—C(═O)—NH—S(O)₂-aryl, —C₁-C₆—C(═O)—NH—S(O)₂—C₁-C₆-aryl, —C₁-C₆—C(═O)—NH—S(O)₂-heteroaryl, —C₁-C₆—C(═O)—NH—S(O)₂—C₁-C₆-heteroaryl, or
—C₁-C₆—C(═O)—NH—S(O)₂—NH₂, —C₁-C₆—C(═O)—NH—S(O)₂—NH—C₁-C₆, —C₁-C₆—C(═O)—NH—S(O)₂—NH—C₃-C₈-cycloalkyl, —C₁-C₆—C(═O)—NH—S(O)₂—NH-aryl, —C₁-C₆—C(═O)—NH—S(O)₂—NH—C₁-C₆-aryl, —C₁-C₆—C(═O)—NH—S(O)₂—NH-heteroaryl, —C₁-C₆—C(═O)—NH—S(O)₂—NH—C₁-C₆-heteroaryl, —C₁-C₆—C(═O)—NH—S(O)₂—N(C₁-C₆)₂,

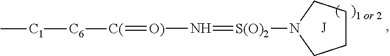

wherein J is a 5 or 6 membered heterocycle ring optionally including a second heteroatom selected from —NH—, NC₁-C₆—, —S— or —O—; or
—C₁-C₆—NH—C(═O)—NH—S(O)₂—C₁-C₆, —C₁-C₆—NH—C(═O)—NH—S(O)₂—C₃-C₈-cycloalkyl, —C₁-C₆—NH—C(═O)—NH—S(O)₂-aryl, —C₁-C₆—NH—C(═O)—NH—S(O)₂-heteroaryl, or
—C₁-C₆—NH—C(═O)—NH—S(O)₂—NH₂, —C₁-C₆—NH—C(═O)—NH—S(O)₂—NH—C₁-C₆, —C₁-C₆—NH—C(═O)—NH—S(O)₂—NH—C₃-C₈-cycloalkyl, —C₁-C₆—NH—C(═O)—NH—S(O)₂—NH-aryl, —C₁-C₆—C(═O)—NH—S(O)₂—NH—C₁-C₆-aryl, —C₁-C₆—NH—C(—O)—NH—S(O)₂—N(C₁-C₆)₂,

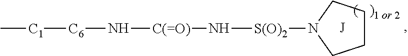

wherein J is a 5 or 6 membered heterocycle ring optionally including a second heteroatom selected from —NH—, NC₁-C₆—, —S— or —O—; or
or
—C(═O)-aryl-C(═O)—OH, —C(═O)-aryl-C(═O)—O—C₁-C₆, —C(═O)-aryl-C(═O)—O—C₁-C₆—O—(C═O)—C₁-C₆, —C(═O)-aryl-OH, or
—C(═O)-aryl-C(═O)—NH₂, —C(═O)-aryl-C(═O)—NH—C₁-C₆, —C(═O)-aryl-C(═O)—NH—C₃-C₈-cycloalkyl, —C(═O)-aryl-C(═O)—N(C₁-C₆)₂,

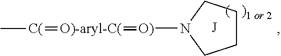

wherein J is a 5 or 6 membered heterocycle ring optionally including a second heteroatom selected from —NH—, —NC₁-C₆—, —S— or —O—; or
—C(═O)-aryl-C(═O)—NH—S(O)₂—C₁-C₆, —C(═O)-aryl-C(═O)—NH—S(O)₂—C₃-C₈-cycloalkyl, —C(═O)-aryl-C(═O)—NH—S(O)₂-aryl, —C(═O)-aryl-C(═O)—NH—S(O)₂—C₁-C₆-aryl, —C(═O)-aryl-C(═O)—NH—S(O)₂-heteroaryl, —C(═O)-aryl-C(═O)—NH—S(O)₂—C₁-C₆-heteroaryl, or
—C(═O)-aryl-C(═O)—NH—S(O)₂—NH₂, —C(═O)-aryl-C(═O)—NH—S(O)₂—NH—C₁-C₆, —C(═O)-aryl-C(═O)—NH—S(O)₂—NH—C₃-C₈-cycloalkyl, —C(═O)-aryl-C(═O)—NH—S(O)₂—NH-aryl, —C(═O)-aryl-C(═O)—NH—S(O)₂—NH—C₁-C₆-aryl, —C(═O)-aryl-C(═O)—NH—S(O)₂—NH-heteroaryl, —C(═O)-aryl-C(═O)—NH—S(O)₂—NH—C₁-C₆-heteroaryl, —C(═O)-aryl-C(═O)—NH—S(O)₂—N(C₁-C₆)₂,

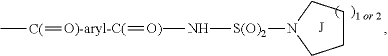

wherein J is a 5 or 6 membered heterocycle ring optionally including a second heteroatom selected from —NH—, NC₁-C₆—, —S— or —O—; or
—C(═O)-aryl-NH—C(═O)—NH—S(O)₂—C₁-C₆, —C(═O)-aryl-NH—C(═O)—NH—S(O)₂—C₃-C₈-cycloalkyl, —C(═O)-aryl-NH—C(═O)—NH—S(O)₂-aryl, —C(═O)-aryl-NH—C(═O)—NH—S(O)₂-heteroaryl, or
—C(═O)-aryl-NH—C(═O)—NH—S(O)₂—NH₂, —C(═O)-aryl-NH—C(═O)—NH—S(O)₂—NH—C₁-C₆, —C(═O)-aryl-NH—C(═O)—NH—S(O)₂—NH—C₃-C₈-cycloalkyl, —C(═O)-aryl-NH—C(═O)—NH—S(O)₂—NH-aryl, —C(═O)-aryl-C(═O)—NH—S(O)₂—NH—C₁-C₆-aryl, —C(═O)-aryl-NH—C(═O)—NH—S(O)₂—N(C₁-C₆)₂,

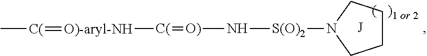

wherein J is a 5 or 6 membered heterocycle ring optionally including a second heteroatom selected from —NH—, NC₁-C₆—, —S— or —O—; or
or
-aryl-C(═O)—OH, -aryl-C(═O)—O—C₁-C₆, -aryl-C(═O)—O—C₁-C₆—O—(C═O)—C₁-C₆, -aryl-C₁-C₆—OH, or
-aryl-C(═O)—NH₂, -aryl-C(═O)—NH—C₁-C₆, -aryl-C(═O)—NH—C₃-C₈-cycloalkyl, -aryl-C(═O)—N(C₁-C₆)₂,

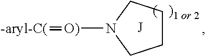

wherein J is a 5 or 6 membered heterocycle ring optionally including a second heteroatom selected from —NH—, —NC₁-C₆—, —S— or —O—; or
-aryl-C(═O)—NH—S(O)₂—C₁-C₆, -aryl-C(═O)—NH—S(O)₂—C₃-C₈-cycloalkyl, -aryl-C(═O)—NH—S(O)₂-aryl, -aryl-C(═O)—NH—S(O)₂—C₁-C₆-aryl, -aryl-C(═O)—NH—S(O)₂-heteroaryl, -aryl-C(═O)—NH—S(O)₂—C₁-C₆-heteroaryl, or
-aryl-C(═O)—NH—S(O)₂—NH₂, -aryl-C(═O)—NH—S(O)₂—NH—C₁-C₆, -aryl-C(═O)—NH—S(O)₂—NH—C₃-C₈-cycloalkyl, -aryl-C(═O)—NH—S(O)₂—NH-aryl, -aryl-C(═O)—NH—S(O)₂—NH—C₁-C₆-aryl, -aryl-C(═O)—NH—S(O)₂—NH-heteroaryl, -aryl-C(═O)—NH—S(O)₂—NH—C₁-C₆-heteroaryl, -aryl-C(═O)—NH—S(O)₂—N(C₁-C₆)₂,

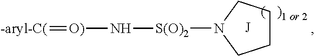

wherein J is a 5 or 6 membered heterocycle ring optionally including a second heteroatom selected from —NH—, NC₁-C₆—, —S— or —O—; or
-aryl-NH—C(═O)—NH—S(O)₂—C₁-C₆, -aryl-NH—C(═O)—NH—S(O)₂—C₃-C₈-cycloalkyl, -aryl-NH—C(═O)—NH—S(O)₂-aryl, -aryl-NH—C(═O)—NH—S(O)₂-heteroaryl, or
-aryl-NH—C(═O)—NH—S(O)₂—NH₂, -aryl-NH—C(═O)—NH—S(O)₂—NH—C₁-C₆, -aryl-NH—C(═O)—NH—S(O)₂—NH—C₃-C₈-cycloalkyl, -aryl-NH—C(═O)—NH—S(O)₂—NH-aryl, -aryl-C(═O)—NH—S(O)₂—NH—C₁-C₆-aryl, -aryl-NH—C(═O)—NH—S(O)₂—N(C₁-C₆)₂,

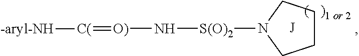

wherein J is a 5 or 6 membered heterocycle ring optionally including a second heteroatom selected from —NH—, NC₁-C₆—, —S— or —O—; or
-aryl-S(O)₂—OH, -aryl-S(O)₂—NH₂, -aryl-S(O)₂—NH—C₁-C₆, -aryl-S(O)₂—N(C₁-C₆)₂,

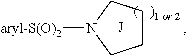

wherein J is a 5 or 6 membered heterocycle ring optionally including a second heteroatom selected from —NH—, —NC₁-C₆—, —S— or —O—; or
-aryl-S(O)₂—NH—C₃-C₈-cycloalkyl, or
—C(═O)-heteroaryl-C(═O)—OH, —C(═O)-heteroaryl-C(═O)—O—C₁-C₆, —C(═O)-heteroaryl-C(═O)—O—C₁-C₆—O—(C═O)—C₁-C₆, —C(═O)-aryl-OH, or
—C(═O)-heteroaryl-C(═O)—NH₂, —C(═O)-heteroaryl-C(═O)—NH—C₁-C₆, —C(═O)-heteroaryl-C(═O)—NH—C₃-C₈-cycloalkyl, —C(═O)-heteroaryl-C(═O)—N(C₁-C₆)₂,

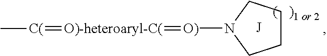

wherein J is a 5 or 6 membered heterocycle ring optionally including a second heteroatom selected from —NH—, —NC₁-C₆—, —S— or —O—; or
—C(═O)-heteroaryl-C(═O)—NH—S(O)₂—C₁-C₆, —C(═O)-heteroaryl-C(═O)—NH—S(O)₂—C₃-C₈-cycloalkyl, —C(═O)-heteroaryl-C(═O)—NH—S(O)₂-aryl, —C(═O)-heteroaryl-C(═O)—NH—S(O)₂—C₁-C₆-aryl, —C(═O)-heteroaryl-C(═O)—NH—S(O)₂-heteroaryl, —C(═O)-heteroaryl-C(═O)—NH—S(O)₂—C₁-C₆-heteroaryl, or
—C(═O)-heteroaryl-C(═O)—NH—S(O)₂—NH₂, —C(═O)-heteroaryl-C(═O)—NH—S(O)₂—NH—C₁-C₆, —C(═O)-heteroaryl-C(═O)—NH—S(O)₂—NH—C₃-C₈-cycloalkyl, —C(═O)-heteroaryl-C(═O)—NH—S(O)₂—NH-aryl, —C(═O)-heteroaryl-C(═O)—NH—S(O)₂—NH—C₁-C₆-aryl, —C(═O)-heteroaryl-C(═O)—NH—S(O)₂—NH-heteroaryl, —C(═O)-heteroaryl-C(═O)—NH—S(O)₂—NH—C₁-C₆-heteroaryl, —C(═O)-heteroaryl-C(═O)—NH—S(O)₂—N(C₁-C₆)₂,

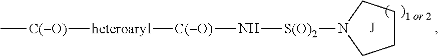

wherein J is a 5 or 6 membered heterocycle ring optionally including a second heteroatom selected from —NH—, NC₁-C₆—, —S— or —O—; or
—C(═O)-heteroaryl-NH—C(═O)—NH—S(O)₂—C₁-C₆, —C(═O)-heteroaryl-NH—C(═O)—NH—S(O)₂—C₃-C₈-cycloalkyl, —C(═O)-heteroaryl-NH—C(═O)—NH—S(O)₂-aryl, —C(═O)-heteroaryl-NH—C(═O)—NH—S(O)₂-heteroaryl, or
—C(═O)-heteroaryl-NH—C(═O)—NH—S(O)₂—NH₂, —C(═O)-heteroaryl-NH—C(═O)—NH—S(O)₂—NH—C₁-C₆, —C(═O)-heteroaryl-NH—C(═O)—NH—S(O)₂—NH—C₃-C₈-cycloalkyl, —C(═O)-heteroaryl-NH—C(═O)—NH—S(O)₂—NH-aryl, —C(═O)-heteroaryl-C(═O)—NH—S(O)₂—NH—C₁-C₆-aryl, —C(═O)-heteroaryl-NH—C(═O)—NH—S(O)₂—N(C₁-C₆)₂,

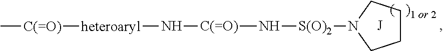

wherein J is a 5 or 6 membered heterocycle ring optionally including a second heteroatom selected from —NH—, NC₁-C₆—, —S— or —O—; or
or
-heteroaryl-C(═O)—OH, -heteroaryl-C(═O)—O—C₁-C₆, -heteroaryl-C(═O)—O—C₁-C₆—O—(C═O)—C₁-C₆, -heteroaryl-C₁-C₆—OH, or
-heteroaryl-C(═O)—NH₂, -heteroaryl-C(═O)—NH—C₁-C₆, -heteroaryl-C(═O)—NH—C₃-C₈-cycloalkyl, -heteroaryl-C(═O)—N(C₁-C₆)₂,

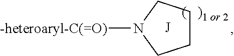

wherein J is a 5 or 6 membered heterocycle ring optionally including a second heteroatom selected from —NH—, —NC₁-C₆—, —S— or —O—; or
-heteroaryl-C(═O)—NH—S(O)₂—C₁-C₆, -heteroaryl-C(═O)—NH—S(O)₂—C₃-C₈-cycloalkyl, heteroaryl-C(═O)—NH—S(O)₂-aryl, -heteroaryl-C(═O)—NH—S(O)₂—C₁-C₆-aryl, -heteroaryl-C(═O)—NH—S(O)₂-heteroaryl, -heteroaryl-C(═O)—NH—S(O)₂—C₁-C₆-heteroaryl, or
-heteroaryl-C(═O)—NH—S(O)₂—NH₂, -heteroaryl-C(═O)—NH—S(O)₂—NH—C₁-C₆, -heteroaryl-C(═O)—NH—S(O)₂—NH—C₃-C₈-cycloalkyl, -heteroaryl-C(═O)—NH—S(O)₂—NH-aryl, -heteroaryl-C(═O)—NH—S(O)₂—NH—C₁-C₆-aryl, -heteroaryl-C(═O)—NH—S(O)₂—NH-heteroaryl, -heteroaryl-C(═O)—NH—S(O)₂—NH—C₁-C₆-heteroaryl, -heteroaryl-C(═O)—NH—S(O)₂—N(C₁-C₆)₂,

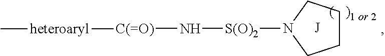

wherein J is a 5 or 6 membered heterocycle ring optionally including a second heteroatom selected from —NH—, NC₁-C₆—, —S— or —O—; or
-heteroaryl-NH—C(═O)—NH—S(O)₂—C₁-C₆, -heteroaryl-NH—C(═O)—NH—S(O)₂—C₃-C₈-cycloalkyl, -heteroaryl-NH—C(═O)—NH—S(O)₂-aryl, -heteroaryl-NH—C(═O)—NH—S(O)₂-heteroaryl, or
-heteroaryl-NH—C(═O)—NH—S(O)₂—NH₂, -heteroaryl-NH—C(═O)—NH—S(O)₂—NH—C₁-C₆, -heteroaryl-NH—C(═O)—NH—S(O)₂—NH—C₃-C₈-cycloalkyl, -heteroaryl-NH—C(═O)—NH—S(O)₂—NH-aryl, -heteroaryl-C(═O)—NH—S(O)₂—NH—C₁-C₆-aryl, -heteroaryl-NH—C(═O)—NH—S(O)₂—N(C₁-C₆)₂,

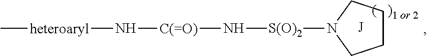

wherein J is a 5 or 6 membered heterocycle ring optionally including a second heteroatom selected from —NH—, NC₁-C₆—, —S— or —O—;
or
—C₁-C₆-aryl-C(═O)—OH, —C₁-C₆-aryl-C(═O)—O—C₁-C₆, —C₁-C₆-aryl-C(═O)—O—C₁-C₆—O—(C═O)—C₁-C₆, —C₁-C₆-aryl-C₁-C₆—OH, or
—C₁-C₆-aryl-C(═O)—NH₂, —C₁-C₆-aryl-C(═O)—NH—C₁-C₆, —C₁-C₆-aryl-C(═O)—NH—C₃-C₈-cycloalkyl, —C₁-C₆-aryl-C(═O)—N(C₁-C₆)₂,

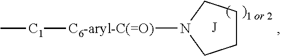

wherein J is a 5 or 6 membered heterocycle ring optionally including a second heteroatom selected from —NH—, —NC₁-C₆—, —S— or —O—; or
—C₁-C₆-aryl-C(═O)—NH—S(O)₂—C₁-C₆, —C₁-C₆-aryl-C(═O)—NH—S(O)₂—C₃-C₈-cycloalkyl, —C₁-C₆-aryl-C(═O)—NH—S(O)₂-aryl, —C₁-C₆-aryl-C(═O)—NH—S(O)₂—C₁-C₆-aryl, —C₁-C₆-aryl-C(═O)—NH—S(O)₂-heteroaryl, —C₁-C₆-aryl-C(═O)—NH—S(O)₂—C₁-C₆-heteroaryl, or
—C₁-C₆-aryl-C(═O)—NH—S(O)₂—NH₂, —C₁-C₆-aryl-C(═O)—NH—S(O)₂—NH—C₁-C₆, —C₁-C₆-aryl-C(═O)—NH—S(O)₂—NH—C₃-C₈-cycloalkyl, —C₁-C₆-aryl-C(═O)—NH—S(O)₂—NH-aryl, —C₁-C₆-aryl-C(═O)—NH—S(O)₂—NH—C₁-C₆-aryl, —C₁-C₆-aryl-C(═O)—NH—S(O)₂—NH-heteroaryl, —C₁-C₆-aryl-C(═O)—NH—S(O)₂—NH—C₁-C₆-heteroaryl, —C₁-C₆-aryl-C(═O)—NH—S(O)₂—N(C₁-C₆)₂,

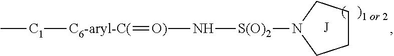

wherein J is a 5 or 6 membered heterocycle ring optionally including a second heteroatom selected from —NH—, NC₁-C₆—, —S— or —O—; or
—C₁-C₆-aryl-NH—C(═O)—NH—S(O)₂—C₁-C₆, —C₁-C₆-aryl-NH—C(═O)—NH—S(O)₂—C₃-C₈-cycloalkyl, —C₁-C₆-aryl-NH—C(═O)—NH—S(O)₂-aryl, —C₁-C₆-aryl-NH—C(═O)—NH—S(O)₂-heteroaryl, or
—C₁-C₆-aryl-NH—C(═O)—NH—S(O)₂—NH₂, —C₁-C₆-aryl-NH—C(═O)—NH—S(O)₂—NH—C₁-C₆, —C₁-C₆-aryl-NH—C(═O)—NH—S(O)₂—NH—C₃-C₈-cycloalkyl, —C₁-C₆-aryl-NH—C(═O)—NH—S(O)₂—NH-aryl, —C₁-C₆-aryl-C(═O)—NH—S(O)₂—NH—C₁-C₆-aryl, —C₁-C₆-aryl-NH—C(═O)—NH—S(O)₂—N(C₁-C₆)₂,

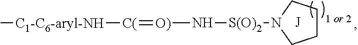

wherein J is a 5 or 6 membered heterocycle ring optionally including a second heteroatom selected from —NH—, NC₁-C₆—, —S— or —O—; or
—C₁-C₆-aryl-S(O)₂—OH, —C₁-C₆-aryl-S(O)₂—NH₂, —C₁-C₆-aryl-S(O)₂—NH—C₁-C₆, —C₁-C₆-aryl-S(O)₂—N(C₁-C₆)₂,

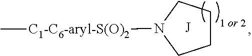

wherein J is a 5 or 6 membered heterocycle ring optionally including a second heteroatom selected from —NH—, —NC₁-C₆—, —S— or —O—; or
—C₁-C₆-aryl-S(O)₂—NH—C₃-C₈-cycloalkyl,
—C₁-C₆-heteroaryl-C(═O)—OH, —C₁-C₆-heteroaryl-C(═O)—O—C₁-C₆, —C₁-C₆-heteroaryl-C(═O)—O—C₁-C₆—O—(C═O)—C₁-C₆, —C₁-C₆-heteroaryl-C₁-C₆—OH, or
—C₁-C₆-heteroaryl-C(═O)—NH₂, —C₁-C₆-heteroaryl-C(═O)—NH—C₁-C₆, —C₁-C₆-heteroaryl-C(═O)—NH—C₃-C₈-cycloalkyl, —C₁-C₆-heteroaryl-C(═O)—N(C₁-C₆)₂,

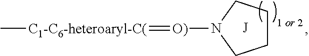

wherein J is a 5 or 6 membered heterocycle ring optionally including a second heteroatom selected from —NH—, —NC₁-C₆—, —S— or —O—; or
—C₁-C₆-heteroaryl-C(═O)—NH—S(O)₂—C₁-C₆, —C₁-C₆-heteroaryl-C(═O)—NH—S(O)₂—C₃-C₈-cycloalkyl, —C₁-C₆-heteroaryl-C(═O)—NH—S(O)₂-aryl, —C₁-C₆-heteroaryl-C(═O)—NH—S(O)₂—C₁-C₆-aryl, —C₁-C₆-heteroaryl-C(═O)—NH—S(O)₂-heteroaryl, —C₁-C₆-heteroaryl-C(═O)—NH—S(O)₂—C₁-C₆-heteroaryl, or
—C₁-C₆-heteroaryl-C(═O)—NH—S(O)₂—NH₂, —C₁-C₆-heteroaryl-C(═O)—NH—S(O)₂—NH—C₁-C₆, —C₁-C₆heteroaryl-C(═O)—NH—S(O)₂—NH—C₃-C₈-cycloalkyl, —C₁-C₆-heteroaryl-C(═O)—NH—S(O)₂—NH-aryl, —C₁-C₆-heteroaryl-C(═O)—NH—S(O)₂—NH—C₁-C₆-aryl, —C₁-C₆-heteroaryl-C(═O)—NH—S(O)₂—NH-heteroaryl, —C₁-C₆-heteroaryl-C(═O)—NH—S(O)₂—NH—C₁-C₆-heteroaryl, —C₁-C₆-heteroaryl-C(═O)—NH—S(O)₂—N(C₁-C₆)₂,

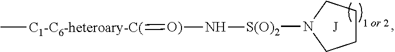

wherein J is a 5 or 6 membered heterocycle ring optionally including a second heteroatom selected from —NH—, NC₁-C₆—, —S— or —O—; or
—C₁-C₆-heteroaryl-NH—C(═O)—NH—S(O)₂—C₁-C₆, —C₁-C₆-heteroaryl-NH—C(═O)—NH—S(O)₂—C₃-C₈-cycloalkyl, —C₁-C₆-heteroaryl-NH—C(═O)—NH—S(O)₂-aryl, —C₁-C₆-heteroaryl-NH—C(═O)—NH—S(O)₂-heteroaryl, or
—C₁-C₆-heteroaryl-NH—C(═O)—NH—S(O)₂—NH₂, —C₁-C₆-heteroaryl-NH—C(═O)—NH—S(O)₂—NH—C₁-C₆, —C₁-C₆-heteroaryl-NH—C(═O)—NH—S(O)₂—NH—C₃-C₈-cycloalkyl, —C₁-C₆-heteroaryl-NH—C(═O)—NH—S(O)₂—NH-aryl, —C₁-C₆-heteroaryl-C(═O)—NH—S(O)₂—NH—C₁-C₆-aryl, —C₁-C₆-heteroaryl-NH—C(═O)—NH—S(O)₂—N(C₁-C₆)₂,

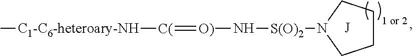

wherein J is a 5 or 6 membered heterocycle ring optionally including a second heteroatom selected from —NH—, NC₁-C₆—, —S— or —O—;
or
wherein Q may be selected from

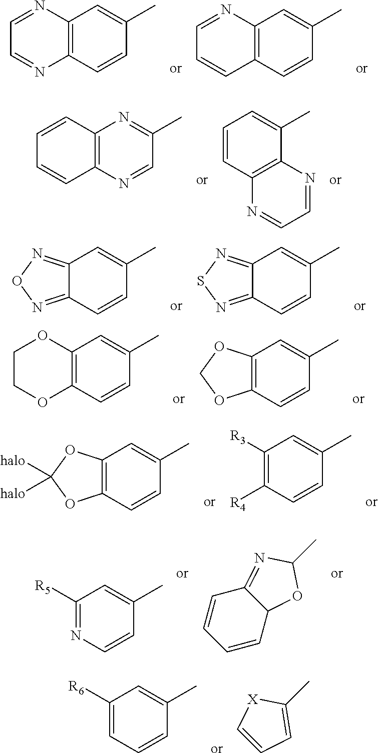

and
wherein R₃, R₄, R₅ or R₆ are independently selected from halo, —C₁-C₆-alkyl, —C₁-C₆-alkyl substituted with one or more halo, —CN, hydroxy, —O—C₁-C₆-alkyl, —O—C₁-C₆-alkyl substituted with one or more halo; and
wherein X is —O— or —S— or —NH— or —N(R₁₁)—, wherein Ri is selected from —C₁-C₆ alkyl or —C(═O)—C₁-C₆;
wherein R₁₂ is selected from halo, hydroxy, C₁-C₆ alkyl, C₁-C₆ alkoxy, aryl, or —O-aryl, where each C₁-C₆ alkyl, C₁-C₆ alkoxy, aryl, or —O-aryl may be further substituted with one or more halo, hydroxy, C₁-C₆ alkoxy or C₁-C₆ alkoxy further substituted with one or more halo; and
wherein Z is —C—, —O— or —S—, —S(O), —S(O)₂, —NH—, —NR₁₂—, wherein R₁₂ is C₁-C₆ alkyl;
or a salt thereof.

20 The compound of embodiment 19, wherein R is selected from
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, or
—C(═O)—C₁-C₆—C(═O)—NH₂, —C(═O)—C₁-C₆—C(═O)—NH—C₁-C₆, —C(═O)—C₁-C₆—C(═O)—N(C₁-C₆)₂,
—C(═O)—C₁-C₆—C(═O)—NH—S(O)₂—C₁-C₆, —C(═O)—C₁-C₆—C(═O)—NH—S(O)₂-aryl, —C(═O)—C₁-C₆—C(═O)—NH—S(O)₂—N(C₁-C₆)₂,

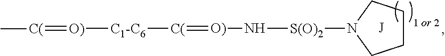

wherein J is a 5 or 6 membered heterocycle ring optionally including a second heteroatom selected from —NH—, NC₁-C₆—, —S— or —O—; or
—C₁-C₆—C(═O)—OH, or
-aryl-C(═O)—OH, or
-aryl-C(═O)—NH—S(O)₂—C₁-C₆.

21 The compound of embodiment 19 or embodiment 20 wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, —C₁-C₆—C(═O)—OH, -aryl-C(═O)—OH.

22 The compound of any one of embodiments 19 to 21, wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, -aryl-C(═O)—OH.

23 The compound of any one of embodiments 19 to 22 wherein R is selected from —C(═O)—C₁-C₆—C(═O)—OH.

24 The compound of any one of embodiments 19 to 22, wherein R is selected from —C(═O)—C₁-C₆—C(═O)—NH—S(O)₂—C₁-C₆.

25 The compound of any one of embodiments 19 to 22, wherein R is selected from —C(═O)—C₁-C₆—C(═O)—NH—S(O)₂—N(C₁-C₆)₂ or —C(═O)—C₁-C₆—C(═O)—NH—S(O)₂-aryl, such as —C(═O)—CH₂—CH₂—C(═O)—NH—S(O)₂-Ph.

26 The compound of any one of embodiments 19 to 22, wherein R is selected from -aryl-C(═O)—OH.

27 The compound of embodiment 23, wherein R is selected from —C(═O)—CH₂—CH₂—C(═O)—OH.

28 The compound of embodiment 24, wherein R is selected from —C(═O)—CH₂—CH₂—C(═O)—NH—S(O)₂-Me or —C(═O)—CH₂—CH₂—C(═O)—NH—S(O)₂-iPr.

29 The compound of embodiment 25, wherein R is selected from —C(═O)—CH₂—CH₂—C(═O)—NH—S(O)₂—NMe₂.

30 The compound of embodiment 26, wherein R is selected from -phenyl-C(═O)—OH.

31 The compound of any one of embodiments 19 to 30, wherein Q is selected from:

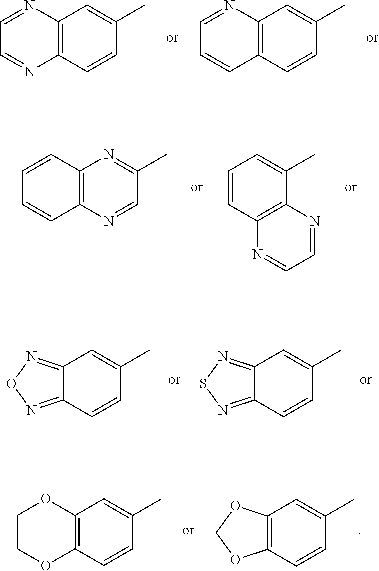

32 The compound of embodiment 31 wherein Q is selected from:

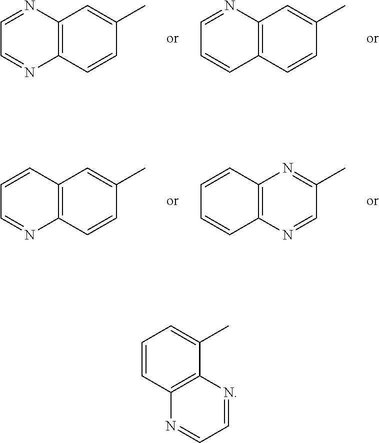

33 The compound of embodiment 31 or 32, wherein Q is

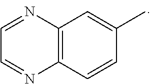

34 The compound of any one of embodiments 19 to 33 wherein R₁₂ is phenyl, or halo.

35 The compound of any one of embodiments 19 to 34 wherein Z is —O—.

36 The compound of any one of embodiment 19, wherein the compound of Formula III is

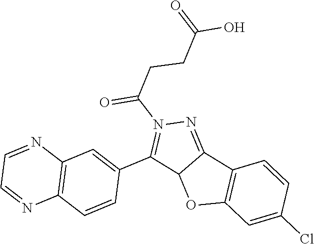

or a salt thereof.

37 A compound of Formula IV

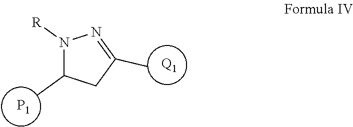

wherein R may be selected from:
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, —C(═O)—C₁-C₆—OH, or
—C(═O)—C₁-C₆—C(═O)—NH₂, —C(═O)—C₁-C₆—C(═O)—NH—C₁-C₆, —C(═O)—C₁-C₆—C(═O)—NH—C₃-C₈-cycloalkyl, —C(═O)—C₁-C₆—C(═O)—N(C₁-C₆)₂,

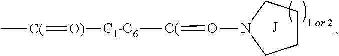

wherein J is a 5 or 6 membered heterocycle ring optionally including a second heteroatom selected from —NH—, —NC₁-C₆—, —S— or —O—; or
—C(═O)—C₁-C₆—C(═O)—NH—S(O)₂—C₁-C₆, —C(═O)—C₁-C₆—C(═O)—NH—S(O)₂—C₃-C₈-cycloalkyl, —C(═O)—C₁-C₆—C(═O)—NH—S(O)₂-aryl, C(═O)—C₁-C₆—C(═O)—NH—S(O)₂—C₁-C₆-aryl, —C(═O)—C₁-C₆—C(═O)—NH—S(O)₂-heteroaryl, —C(═O)—C₁-C₆—C(═O)—NH—S(O)₂—C₁-C₆-heteroaryl, or
—C(═O)—C₁-C₆—C(═O)—NH—S(O)₂—NH₂, —C(═O)—C₁-C₆—C(═O)—NH—S(O)₂—NH—C₁-C₆, —C(═O)—C₁-C₆—C(═O)—NH—S(O)₂—NH—C₃-C₈-cycloalkyl, —C(═O)—C₁-C₆—C(═O)—NH—S(O)₂—NH-aryl, —C(═O)—C₁-C₆—C(═O)—NH—S(O)₂—NH—C₁-C₆-aryl, —C(═O)—C₁-C₆—C(═O)—NH—S(O)₂—NH-heteroaryl, —C(═O)—C₁-C₆—C(═O)—NH—S(O)₂—NH—C₁-C₆-heteroaryl, —C(═O)—C₁-C₆—C(═O)—NH—S(O)₂—N(C₁-C₆)₂,

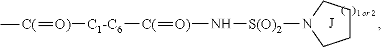

wherein J is a 5 or 6 membered heterocycle ring optionally including a second heteroatom selected from —NH—, NC₁-C₆—, —S— or —O—; or
—C(═O)—C₁-C₆—NH—C(═O)—NH—S(O)₂—C₁-C₆, —C(═O)—C₁-C₆—NH—C(═O)—NH—S(O)₂—C₃-C₈-cycloalkyl, —C(═O)—C₁-C₆—NH—C(═O)—NH—S(O)₂-aryl, —C(═O)—C₁-C₆—NH—C(═O)—NH—S(O)₂-heteroaryl, or
—C(═O)—C₁-C₆—NH—C(═O)—NH—S(O)₂—NH₂, —C(═O)—C₁-C₆—NH—C(═O)—NH—S(O)₂—NH—C₁-C₆, —C(═O)—C₁-C₆—NH—C(═O)—NH—S(O)₂—NH—C₃-C₈-cycloalkyl, —C(═O)—C₁-C₆—NH—C(═O)—NH—S(O)₂—NH-aryl, —C(═O)—C₁-C₆—C(═O)—NH—S(O)₂—NH—C₁-C₆-aryl, —C(═O)—C₁-C₆—NH—C(═O)—NH—S(O)₂—N(C₁-C₆)₂,

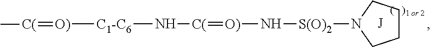

wherein J is a 5 or 6 membered heterocycle ring optionally including a second heteroatom selected from —NH—, NC₁-C₆—, —S— or —O—; or
—C₁-C₆—C(═O)—OH, —C₁-C₆—C(═O)—O—C₁-C₆, C₁-C₆—C(═O)—O—C₁-C₆—O—(C═O)—C₁-C₆, —C₁- C₆—OH, or
—C₁-C₆—C(═O)—NH₂, —C₁-C₆—C(═O)—NH—C₁-C₆, —C₁-C₆—C(═O)—NH—C₃-C₈-cycloalkyl, —C₁-C₆—C(═O)—N(C₁-C₆)₂,

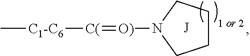

wherein J is a 5 or 6 membered heterocycle ring optionally including a second heteroatom selected from —NH—, —NC₁-C₆—, —S— or —O—; or
—C₁-C₆—C(═O)—NH—S(O)₂—C₁-C₆, —C₁-C₆—C(═O)—NH—S(O)₂—C₃-C₈-cycloalkyl, —C₁-C₆—C(═O)—NH—S(O)₂-aryl, —C₁-C₆—C(═O)—NH—S(O)₂—C₁-C₆-aryl, —C₁-C₆—C(═O)—NH—S(O)₂-heteroaryl, —C₁-C₆—C(═O)—NH—S(O)₂—C₁-C₆-heteroaryl, or
—C₁-C₆—C(═O)—NH—S(O)₂—NH₂, —C₁-C₆—C(═O)—NH—S(O)₂—NH—C₁-C₆, —C₁-C₆—C(═O)—NH—S(O)₂—NH—C₃-C₈-cycloalkyl, —C₁-C₆—C(═O)—NH—S(O)₂—NH-aryl, —C₁-C₆—C(═O)—NH—S(O)₂—NH—C₁-C₆-aryl, —C₁-C₆—C(═O)—NH—S(O)₂—NH-heteroaryl, —C₁-C₆—C(═O)—NH—S(O)₂—NH—C₁-C₆-heteroaryl, —C₁-C₆—C(═O)—NH—S(O)₂—N(C₁-C₆)₂,

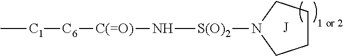

wherein J is a 5 or 6 membered heterocycle ring optionally including a second heteroatom selected from —NH—, NC₁-C₆—, —S— or —O—; or
—C₁-C₆—NH—C(═O)—NH—S(O)₂—C₁-C₆, —C₁-C₆—NH—C(═O)—NH—S(O)₂—C₃-C₈-cycloalkyl, —C₁-C₆—NH—C(═O)—NH—S(O)₂-aryl, —C₁-C₆—NH—C(═O)—NH—S(O)₂-heteroaryl, or
—C₁-C₆—NH—C(═O)—NH—S(O)₂—NH₂, —C₁-C₆—NH—C(═O)—NH—S(O)₂—NH—C₁-C₆, —C₁-C₆—NH—C(═O)—NH—S(O)₂—NH—C₃-C₈-cycloalkyl, —C₁-C₆—NH—C(═O)—NH—S(O)₂—NH-aryl, —C₁-C₆—C(═O)—NH—S(O)₂—NH—C₁-C₆-aryl, —C₁-C₆—NH—C(═O)—NH—S(O)₂—N(C₁-C₆)₂,

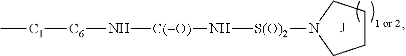

wherein J is a 5 or 6 membered heterocycle ring optionally including a second heteroatom selected from —NH—, NC₁-C₆—, —S— or —O—; or
or
—C(═O)-aryl-C(═O)—OH, —C(═O)-aryl-C(═O)—O—C₁-C₆, —C(═O)-aryl-C(═O)—O—C₁-C₆—O—(C═O)—C₁-C₆, —C(═O)-aryl-OH, or
—C(═O)-aryl-C(═O)—NH₂, —C(═O)-aryl-C(═O)—NH—C₁-C₆, —C(═O)-aryl-C(═O)—NH—C₃-C₈-cycloalkyl, —C(═O)-aryl-C(═O)—N(C₁-C₆)₂,

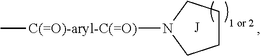

wherein J is a 5 or 6 membered heterocycle ring optionally including a second heteroatom selected from —NH—, —NC₁-C₆—, —S— or —O—; or
—C(═O)-aryl-C(═O)—NH—S(O)₂—C₁-C₆, —C(═O)-aryl-C(═O)—NH—S(O)₂—C₃-C₈-cycloalkyl, —C(═O)-aryl-C(═O)—NH—S(O)₂-aryl, —C(═O)-aryl-C(═O)—NH—S(O)₂—C₁-C₆-aryl, —C(═O)-aryl-C(═O)—NH—S(O)₂-heteroaryl, —C(═O)-aryl-C(═O)—NH—S(O)₂—C₁-C₆-heteroaryl, or
—C(═O)-aryl-C(═O)—NH—S(O)₂—NH₂, —C(═O)-aryl-C(═O)—NH—S(O)₂—NH—C₁-C₆, —C(═O)-aryl-C(═O)—NH—S(O)₂—NH—C₃-C₈-cycloalkyl, —C(═O)-aryl-C(═O)—NH—S(O)₂—NH-aryl, —C(═O)-aryl-C(═O)—NH—S(O)₂—NH—C₁-C₆-aryl, —C(═O)-aryl-C(═O)—NH—S(O)₂—NH-heteroaryl, C(═O)-aryl-C(═O)—NH—S(O)₂—NH—C₁-C₆-heteroaryl, —C(═O)-aryl-C(═O)—NH—S(O)₂—N(C₁—C₆)₂,

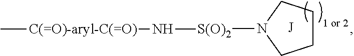

wherein J is a 5 or 6 membered heterocycle ring optionally including a second heteroatom selected from —NH—, NC₁-C₆—, —S— or —O—; or
—C(═O)-aryl-NH—C(═O)—NH—S(O)₂—C₁-C₆, —C(═O)-aryl-NH—C(═O)—NH—S(O)₂—C₃-C₈-cycloalkyl, —C(═O)-aryl-NH—C(═O)—NH—S(O)₂-aryl, —C(═O)-aryl-NH—C(═O)—NH—S(O)₂-heteroaryl, or
—C(═O)-aryl-NH—C(═O)—NH—S(O)₂—NH₂, —C(═O)-aryl-NH—C(═O)—NH—S(O)₂—NH—C₁-C₆, —C(═O)-aryl-NH—C(═O)—NH—S(O)₂—NH—C₃-C₈-cycloalkyl, —C(═O)-aryl-NH—C(═O)—NH—S(O)₂—NH-aryl, —C(═O)-aryl-C(═O)—NH—S(O)₂—NH—C₁-C₆-aryl, —C(═O)-aryl-NH—C(═O)—NH—S(O)₂—N(C₁-C₆)₂,

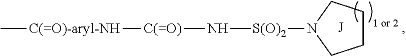

wherein J is a 5 or 6 membered heterocycle ring optionally including a second heteroatom selected from —NH—, NC₁-C₆—, —S— or —O—; or
or
-aryl-C(═O)—OH, -aryl-C(═O)—O—C₁-C₆, -aryl-C(═O)—O—C₁-C₆—O—(C═O)—C₁-C₆, -aryl-C₁-C₆—OH, or
-aryl-C(═O)—NH₂, -aryl-C(═O)—NH—C₁-C₆, -aryl-C(═O)—NH—C₃-C₈-cycloalkyl, -aryl-C(═O)—N(C₁-C₆)₂,

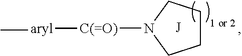

wherein J is a 5 or 6 membered heterocycle ring optionally including a second heteroatom selected from —NH—, —NC₁-C₆—, —S— or —O—; or
-aryl-C(═O)—NH—S(O)₂—C₁-C₆, -aryl-C(═O)—NH—S(O)₂—C₃-C₈-cycloalkyl, -aryl-C(═O)—NH—S(O)₂-aryl, -aryl-C(═O)—NH—S(O)₂—C₁-C₆-aryl, -aryl-C(═O)—NH—S(O)₂-heteroaryl, -aryl-C(═O)—NH—S(O)₂—C₁-C₆-heteroaryl, or
-aryl-C(═O)—NH—S(O)₂—NH₂, -aryl-C(═O)—NH—S(O)₂—NH—C₁-C₆, -aryl-C(═O)—NH—S(O)₂—NH—C₃-C₈-cycloalkyl, -aryl-C(═O)—NH—S(O)₂—NH-aryl, -aryl-C(═O)—NH—S(O)₂—NH—C₁-C₆-aryl, -aryl-C(═O)—NH—S(O)₂—NH-heteroaryl, -aryl-C(═O)—NH—S(O)₂—NH—C₁-C₆-heteroaryl, -aryl-C(═O)—NH—S(O)₂—N(C₁-C₆)₂,

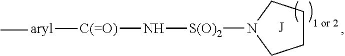

wherein J is a 5 or 6 membered heterocycle ring optionally including a second heteroatom selected from —NH—, NC₁-C₆—, —S— or —O—; or
-aryl-NH—C(═O)—NH—S(O)₂—C₁-C₆, -aryl-NH—C(═O)—NH—S(O)₂—C₃-C₈-cycloalkyl, -aryl-NH—C(═O)—NH—S(O)₂-aryl, -aryl-NH—C(═O)—NH—S(O)₂-heteroaryl, or
-aryl-NH—C(═O)—NH—S(O)₂—NH₂, -aryl-NH—C(═O)—NH—S(O)₂—NH—C₁-C₆, -aryl-NH—C(═O)—NH—S(O)₂—NH—C₃-C₈-cycloalkyl, -aryl-NH—C(═O)—NH—S(O)₂—NH-aryl, -aryl-C(═O)—NH—S(O)₂—NH—C₁-C₆-aryl, -aryl-NH—C(═O)—NH—S(O)₂—N(C₁-C₆)₂,

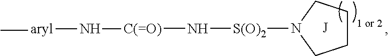

wherein J is a 5 or 6 membered heterocycle ring optionally including a second heteroatom selected from —NH—, NC₁-C₆—, —S— or —O—; or
-aryl-S(O)₂—OH, -aryl-S(O)₂—NH₂, -aryl-S(O)₂—NH—C₁-C₆, -aryl-S(O)₂—N(C₁-C₆)₂,

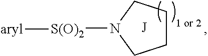

wherein J is a 5 or 6 membered heterocycle ring optionally including a second heteroatom selected from —NH—, —NC₁-C₆—, —S— or —O—; or
-aryl-S(O)₂—NH—C₃-C₈-cycloalkyl, or
—C(═O)-heteroaryl-C(═O)—OH, —C(═O)-heteroaryl-C(═O)—O—C₁-C₆, —C(═O)-heteroaryl-C(═O)—O—C₁-C₆—O—(C═O)—C₁-C₆, —C(═O)-aryl-OH, or
—C(═O)-heteroaryl-C(═O)—NH₂, —C(═O)-heteroaryl-C(═O)—NH—C₁-C₆, —C(═O)-heteroaryl-C(═O)—NH—C₃-C₈-cycloalkyl, —C(═O)-heteroaryl-C(═O)—N(C₁-C₆)₂,

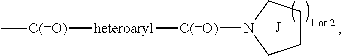

wherein J is a 5 or 6 membered heterocycle ring optionally including a second heteroatom selected from —NH—, —NC₁-C₆—, —S— or —O—; or
—C(═O)-heteroaryl-C(═O)—NH—S(O)₂—C₁-C₆, —C(═O)-heteroaryl-C(═O)—NH—S(O)₂—C₃-C₈-cycloalkyl, —C(═O)-heteroaryl-C(═O)—NH—S(O)₂-aryl, —C(═O)-heteroaryl-C(═O)—NH—S(O)₂—C₁-C₆-aryl, —C(═O)-heteroaryl-C(═O)—NH—S(O)₂-heteroaryl, —C(═O)-heteroaryl-C(═O)—NH—S(O)₂—C₁-C₆-heteroaryl, or
—C(═O)-heteroaryl-C(═O)—NH—S(O)₂—NH₂, —C(═O)-heteroaryl-C(═O)—NH—S(O)₂—NH—C₁-C₆, —C(═O)-heteroaryl-C(═O)—NH—S(O)₂—NH—C₃-C₈-cycloalkyl, —C(═O)-heteroaryl-C(═O)—NH—S(O)₂—NH-aryl, —C(═O)-heteroaryl-C(═O)—NH—S(O)₂—NH—C₁-C₆-aryl, —C(═O)-heteroaryl-C(═O)—NH—S(O)₂—NH-heteroaryl, —C(═O)-heteroaryl-C(═O)—NH—S(O)₂—NH—C₁-C₆-heteroaryl, —C(═O)-heteroaryl-C(═O)—NH—S(O)₂—N(C₁-C₆)₂,

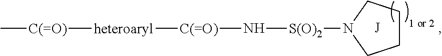

wherein J is a 5 or 6 membered heterocycle ring optionally including a second heteroatom selected from —NH—, NC₁-C₆—, —S— or —O—; or
—C(═O)-heteroaryl-NH—C(═O)—NH—S(O)₂—C₁-C₆, —C(═O)-heteroaryl-NH—C(═O)—NH—S(O)₂—C₃-C₈-cycloalkyl, —C(═O)-heteroaryl-NH—C(═O)—NH—S(O)₂-aryl, —C(═O)-heteroaryl-NH—C(═O)—NH—S(O)₂-heteroaryl, or
—C(═O)-heteroaryl-NH—C(═O)—NH—S(O)₂—NH₂, —C(═O)-heteroaryl-NH—C(═O)—NH—S(O)₂—NH—C₁-C₆, —C(═O)-heteroaryl-NH—C(═O)—NH—S(O)₂—NH—C₃-C₈-cycloalkyl, —C(═O)-heteroaryl-NH—C(═O)—NH—S(O)₂—NH-aryl, —C(═O)-heteroaryl-C(═O)—NH—S(O)₂—NH—C₁-C₆-aryl, —C(═O)-heteroaryl-NH—C(═O)—NH—S(O)₂—N(C₁-C₆)₂,

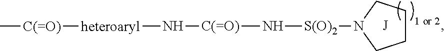

wherein J is a 5 or 6 membered heterocycle ring optionally including a second heteroatom selected from —NH—, NC₁-C₆—, —S— or —O—; or
or
-heteroaryl-C(═O)—OH, -heteroaryl-C(═O)—O—C₁-C₆, -heteroaryl-C(═O)—O—C₁-C₆—O—(C═O)—C₁-C₆, -heteroaryl-C₁-C₆—OH, or
-heteroaryl-C(═O)—NH₂, -heteroaryl-C(═O)—NH—C₁-C₆, -heteroaryl-C(═O)—NH—C₃-C₈-cycloalkyl, -heteroaryl-C(═O)—N(C₁-C₆)₂,

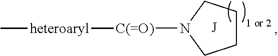

wherein J is a 5 or 6 membered heterocycle ring optionally including a second heteroatom selected from —NH—, —NC₁-C₆—, —S— or —O—; or
-heteroaryl-C(═O)—NH—S(O)₂—C₁-C₆, -heteroaryl-C(═O)—NH—S(O)₂—C₃-C₈-cycloalkyl, heteroaryl-C(═O)—NH—S(O)₂-aryl, -heteroaryl-C(═O)—NH—S(O)₂—C₁-C₆-aryl, -heteroaryl-C(═O)—NH—S(O)₂-heteroaryl, -heteroaryl-C(═O)—NH—S(O)₂—C₁-C₆-heteroaryl, or
-heteroaryl-C(═O)—NH—S(O)₂—NH₂, -heteroaryl-C(═O)—NH—S(O)₂—NH—C₁-C₆, -heteroaryl-C(═O)—NH—S(O)₂—NH—C₃-C₈-cycloalkyl, -heteroaryl-C(═O)—NH—S(O)₂—NH-aryl, -heteroaryl-C(═O)—NH—S(O)₂—NH—C₁-C₆-aryl, -heteroaryl-C(═O)—NH—S(O)₂—NH-heteroaryl, -heteroaryl-C(═O)—NH—S(O)₂—NH—C₁-C₆-heteroaryl, -heteroaryl-C(═O)—NH—S(O)₂—N(C₁-C₆)₂,

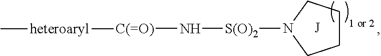

wherein J is a 5 or 6 membered heterocycle ring optionally including a second heteroatom selected from —NH—, NC₁-C₆—, —S— or —O—; or
-heteroaryl-NH—C(═O)—NH—S(O)₂—C₁-C₆, -heteroaryl-NH—C(═O)—NH—S(O)₂—C₃-C₈-cycloalkyl, -heteroaryl-NH—C(═O)—NH—S(O)₂-aryl, -heteroaryl-NH—C(═O)—NH—S(O)₂-heteroaryl, or
-heteroaryl-NH—C(═O)—NH—S(O)₂—NH₂, -heteroaryl-NH—C(═O)—NH—S(O)₂—NH—C₁-C₆, -heteroaryl-NH—C(═O)—NH—S(O)₂—NH—C₃-C₈-cycloalkyl, -heteroaryl-NH—C(═O)—NH—S(O)₂—NH-aryl, -heteroaryl-C(═O)—NH—S(O)₂—NH—C₁-C₆-aryl, -heteroaryl-NH—C(═O)—NH—S(O)₂—N(C₁-C₆)₂,

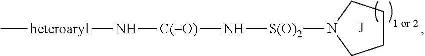

wherein J is a 5 or 6 membered heterocycle ring optionally including a second heteroatom selected from —NH—, NC₁-C₆—, —S— or —O—;

or
—C₁-C₆-aryl-C(═O)—OH, —C₁-C₆-aryl-C(═O)—O—C₁-C₆, —C₁-C₆-aryl-C(═O)—O—C₁-C₆—O—(C═O)—C₁-C₆, —C₁-C₆-aryl-C₁-C₆—OH, or
—C₁-C₆-aryl-C(═O)—NH₂, —C₁-C₆-aryl-C(═O)—NH—C₁-C₆, —C₁-C₆-aryl-C(═O)—NH—C₃-C₈-cycloalkyl, —C₁-C₆-aryl-C(═O)—N(C₁-C₆)₂,

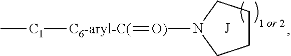

wherein J is a 5 or 6 membered heterocycle ring optionally including a second heteroatom selected from —NH—, —NC₁-C₆—, —S— or —O—; or
—C₁-C₆-aryl-C(═O)—NH—S(O)₂—C₁-C₆, —C₁-C₆-aryl-C(═O)—NH—S(O)₂—C₃-C₈-cycloalkyl, —C₁-C₆-aryl-C(═O)—NH—S(O)₂-aryl, —C₁-C₆-aryl-C(═O)—NH—S(O)₂—C₁-C₆-aryl, —C₁-C₆-aryl-C(═O)—NH—S(O)₂-heteroaryl, —C₁-C₆-aryl-C(═O)—NH—S(O)₂—C₁-C₆-heteroaryl, or
—C₁-C₆-aryl-C(═O)—NH—S(O)₂—NH₂, —C₁-C₆-aryl-C(═O)—NH—S(O)₂—NH—C₁-C₆, —C₁-C₆-aryl-C(═O)—NH—S(O)₂—NH—C₃-C₈-cycloalkyl, —C₁-C₆-aryl-C(═O)—NH—S(O)₂—NH-aryl, —C₁-C₆-aryl-C(═O)—NH—S(O)₂—NH—C₁-C₆-aryl, —C₁-C₆-aryl-C(═O)—NH—S(O)₂—NH-heteroaryl, —C₁-C₆-aryl-C(═O)—NH—S(O)₂—NH—C₁-C₆-heteroaryl, —C₁-C₆-aryl-C(═O)—NH—S(O)₂—N(C₁-C₆)₂,

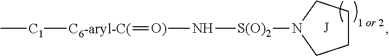

wherein J is a 5 or 6 membered heterocycle ring optionally including a second heteroatom selected from —NH—, NC₁-C₆—, —S— or —O—; or
—C₁-C₆-aryl-NH—C(═O)—NH—S(O)₂—C₁-C₆, —C₁-C₆-aryl-NH—C(═O)—NH—S(O)₂—C₃-C₈-cycloalkyl, —C₁-C₆-aryl-NH—C(═O)—NH—S(O)₂-aryl, —C₁-C₆-aryl-NH—C(═O)—NH—S(O)₂-heteroaryl, or
—C₁-C₆-aryl-NH—C(═O)—NH—S(O)₂—NH₂, —C₁-C₆-aryl-NH—C(═O)—NH—S(O)₂—NH—C₁-C₆, —C₁-C₆-aryl-NH—C(═O)—NH—S(O)₂—NH—C₃-C₈-cycloalkyl, —C₁-C₆-aryl-NH—C(═O)—NH—S(O)₂—NH-aryl, —C₁-C₆-aryl-C(═O)—NH—S(O)₂—NH—C₁-C₆-aryl, —C₁-C₆-aryl-NH—C(═O)—NH—S(O)₂—N(C₁—C₆)₂,

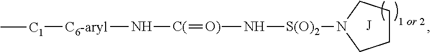

wherein J is a 5 or 6 membered heterocycle ring optionally including a second heteroatom selected from —NH—, NC₁-C₆—, —S— or —O—; or
—C₁-C₆-aryl-S(O)₂—OH, —C₁-C₆-aryl-S(O)₂—NH₂, —C₁-C₆-aryl-S(O)₂—NH—C₁-C₆, —C₁-C₆-aryl-S(O)₂—N(C₁-C₆)₂,

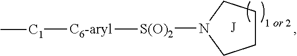

wherein J is a 5 or 6 membered heterocycle ring optionally including a second heteroatom selected from —NH—, —NC₁-C₆—, —S— or —O—; or
—C₁-C₆-aryl-S(O)₂—NH—C₃-C₈-cycloalkyl,
—C₁-C₆-heteroaryl-C(═O)—OH, —C₁-C₆-heteroaryl-C(═O)—O—C₁-C₆, —C₁-C₆-heteroaryl-C(═O)—O—C₁-C₆—O—(C═O)—C₁-C₆, —C₁-C₆-heteroaryl-C₁-C₆—OH, or
—C₁-C₆-heteroaryl-C(═O)—NH₂, —C₁-C₆-heteroaryl-C(═O)—NH—C₁-C₆, —C₁-C₆-heteroaryl-C(═O)—NH—C₃-C₈-cycloalkyl, —C₁-C₆-heteroaryl-C(═O)—N(C₁-C₆)₂,

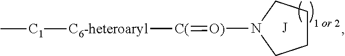

wherein J is a 5 or 6 membered heterocycle ring optionally including a second heteroatom selected from —NH—, —NC₁-C₆—, —S— or —O—; or
C₁-C₆-heteroaryl-C(═O)—NH—S(O)₂—C₁-C₆, —C₁-C₆-heteroaryl-C(═O)—NH—S(O)₂—C₃-C₈-cycloalkyl, —C₁-C₆-heteroaryl-C(═O)—NH—S(O)₂-aryl, —C₁-C₆-heteroaryl-C(═O)—NH—S(O)₂—C₁-C₆-aryl, —C₁-C₆-heteroaryl-C(═O)—NH—S(O)₂-heteroaryl, —C₁-C₆-heteroaryl-C(═O)—NH—S(O)₂—C₁-C₆-heteroaryl, or
—C₁-C₆-heteroaryl-C(═O)—NH—S(O)₂—NH₂, —C₁-C₆-heteroaryl-C(═O)—NH—S(O)₂—NH—C₁-C₆, —C₁-C₆heteroaryl-C(═O)—NH—S(O)₂—NH—C₃-C₈-cycloalkyl, —C₁-C₆-heteroaryl-C(═O)—NH—S(O)₂—NH-aryl, —C₁-C₆-heteroaryl-C(═O)—NH—S(O)₂—NH—C₁-C₆-aryl, —C₁-C₆-heteroaryl-C(═O)—NH—S(O)₂—NH-heteroaryl, —C₁-C₆-heteroaryl-C(═O)—NH—S(O)₂—NH—C₁-C₆-heteroaryl, —C₁-C₆-heteroaryl-C(═O)—NH—S(O)₂—N(C₁-C₆)₂,

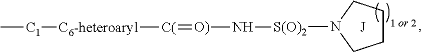

wherein J is a 5 or 6 membered heterocycle ring optionally including a second heteroatom selected from —NH—, NC₁-C₆—, —S— or —O—; or
—C₁-C₆-heteroaryl-NH—C(═O)—NH—S(O)₂—C₁-C₆, —C₁-C₆-heteroaryl-NH—C(═O)—NH—S(O)₂—C₃-C₈-cycloalkyl, —C₁-C₆-heteroaryl-NH—C(═O)—NH—S(O)₂-aryl, —C₁-C₆-heteroaryl-NH—C(═O)—NH—S(O)₂-heteroaryl, or
—C₁-C₆-heteroaryl-NH—C(═O)—NH—S(O)₂—NH₂, —C₁-C₆-heteroaryl-NH—C(═O)—NH—S(O)₂—NH—C₁-C₆, —C₁-C₆-heteroaryl-NH—C(═O)—NH—S(O)₂—NH—C₃-C₈-cycloalkyl, —C₁-C₆-heteroaryl-NH—C(═O)—NH—S(O)₂—NH-aryl, —C₁-C₆-heteroaryl-C(═O)—NH—S(O)₂—NH—C₁-C₆-aryl, —C₁-C₆-heteroaryl-NH—C(═O)—NH—S(O)₂—N(C₁-C₆)₂,

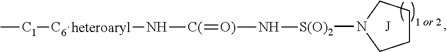

wherein J is a 5 or 6 membered heterocycle ring optionally including a second heteroatom selected from —NH—, NC₁-C₆—, —S— or —O—;
or
wherein P₁ may be a 5 to 10 membered aryl ring or rings optionally containing one or more heteroatoms, and wherein P may be further substituted with one or more halo, —C₁-C₆-alkyl, —C₁-C₆-alkyl substituted with one or more halo or hydroxy; —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

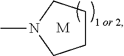

wherein M is a 5 or 6 membered heterocycle ring optionally including a second heteroatom selected from —NH—, —NC₁-C₆—, —S— or —O—; or aryl, aryl substituted with one or more, halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

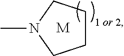

wherein M is a 5 or 6 membered heterocycle ring optionally including a second heteroatom selected from —NH—, —NC₁-C₆—, —S— or —O—; or
C₁-C₆-aryl; C₁-C₆-aryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

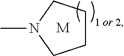

wherein M is a 5 or 6 membered heterocycle ring optionally including a second heteroatom selected from —NH—, —NC₁-C₆—, —S— or —O—; or
heteroaryl; heteroaryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

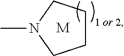

wherein M is a 5 or 6 membered heterocycle ring optionally including a second heteroatom selected from —NH—, —NC₁-C₆—, —S— or —O—; or
C₁-C₆-heteroaryl; C₁-C₆-heteroaryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

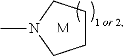

wherein M is a 5 or 6 membered heterocycle ring optionally including a second heteroatom selected from —NH—, —NC₁-C₆—, —S— or —O—; or
O-aryl; —O-aryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

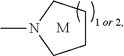

wherein M is a 5 or 6 membered heterocycle ring optionally including a second heteroatom selected from —NH—, —NC₁-C₆—, —S— or —O—; or
—O—C₁-C₆-aryl; —O—C₁-C₆-aryl substituted with one or more halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂,

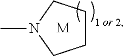

wherein M is a 5 or 6 membered heterocycle ring optionally including a second heteroatom selected from —NH—, —NC₁-C₆—, —S— or —O—; or
wherein Q₁ may be selected from

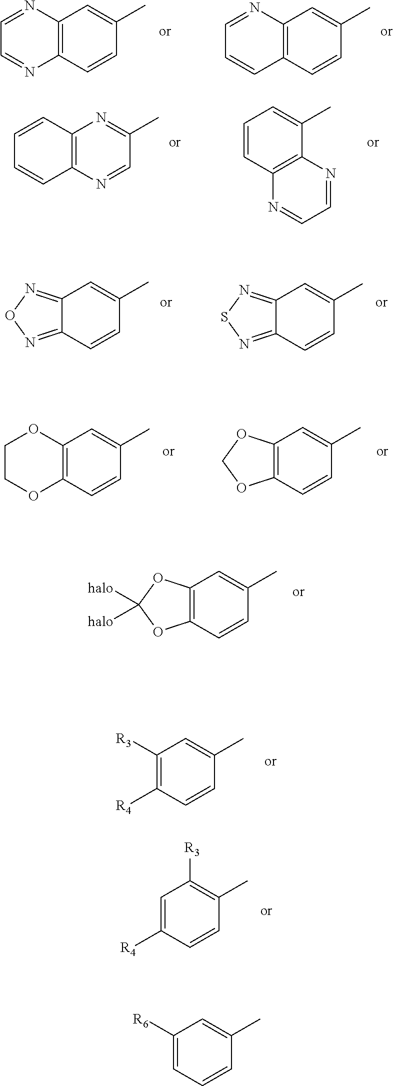

and
wherein R₃, R₄, or R₆ are independently selected from —H, halo, hydroxy, C₁-C₆ alkyl or C₁-C₆ alkoxy, alkoxy substituted with one or more halo, aryl optionally substituted with one or more halo, hydroxy, alkoxy, alkoxy substituted with one or more halo;
or a salt thereof.

38 The compound of embodiment 37 wherein R may be selected from:
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, or
—C(═O)—C₁-C₆—C(═O)—NH₂, —C(═O)—C₁-C₆—C(═O)—NH—C₁-C₆, —C(═O)—C₁-C₆—C(═O)—N(C₁-C₆)₂,
—C(═O)—C₁-C₆—C(═O)—NH—S(O)₂—C₁-C₆, —C(═O)—C₁-C₆—C(═O)—NH—S(O)₂-aryl, —C(═O)—C₁-C₆—C(═O)—NH—S(O)₂—N(C₁-C₆)₂,

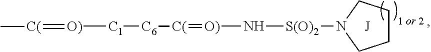

wherein J is a 5 or 6 membered heterocycle ring optionally including a second heteroatom selected from —NH—, NC₁-C₆—, —S— or —O—; or
—C₁-C₆—C(═O)—OH, or
-aryl-C(═O)—OH, or
-aryl-C(═O)—NH—S(O)₂—C₁-C₆.

38a The compound of embodiment 37 wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, —C₁-C₆—C(═O)—OH, -aryl-C(═O)—OH.

39 The compound of embodiment 37 or 38, wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, -aryl-C(═O)—OH.

40 The compound of any one of embodiments 37 to 39, wherein R is selected from —C(═O)—C₁-C₆—C(═O)—OH.

41 The compound of any one of embodiments 37 to 39 wherein R is —C(═O)—C₁-C₆—C(═O)—NH—S(O)₂—C₁-C₆.

42 The compound of any one of embodiments 37 to 39, wherein R is selected from —C(═O)—C₁-C₆—C(═O)—NH—S(O)₂—N(C₁-C₆)₂, such as —C(═O)—C₁-C₆—C(═O)—NH—S(O)₂—N(C₁-C₆ alkyl)₂; or —C(═O)—C₁-C₆—C(═O)—NH—S(O)₂-aryl, such as —C(═O)—CH₂—CH₂—C(═O)—NH—S(O)₂-Ph.

43 The compound of embodiment 37, wherein R is -aryl-C(═O)—OH.

44 The compound of embodiment 37, wherein R is —C(═O)—CH₂—CH₂—C(═O)—OH.

45 The compound of embodiment 37 or embodiment 42, wherein R is selected from —C(═O)—CH₂—CH₂—C(═O)—NH—S(O)₂-Me, or —C(═O)—CH₂—CH₂—C(═O)—NH—S(O)₂-iPr.

46 The compound of embodiment 37 or embodiment 42, wherein R is selected from —C(═O)—CH₂—CH₂—C(═O)—NH—S(O)₂—NMe₂.

47 The compound of embodiments 43 wherein R is -phenyl-C(═O)—OH.

48 The compound of any one of embodiments 37 to 47, wherein P₁ is aryl or an aryl substituted with one or more halo, —C₁-C₆-alkyl, or —O—C₁-C₆-alkyl.

49 The compound of any one of embodiments 37 to 47 wherein P₁ is aryl substituted with aryl or an aryl substituted with halo, —C₁-C₆-alkyl, —O—C₁-C₆-alkyl or OC₁-C₆-alkyl substituted with one or more halo.

50 The compound of embodiment 49 wherein P₁ is phenyl or a phenyl substituted with one or more halo, —C₁-C₆-alkyl, —O—C₁-C₆-alkyl or OC₁-C₆-alkyl substituted with one or more halo.

51 The compound of embodiment 49, wherein P₁ is phenyl substituted with phenyl or a phenyl substituted with halo, —C₁-C₆-alkyl, —O—C₁-C₆-alkyl or OC₁-C₆-alkyl substituted with one or more halo.

52 The compound of embodiment 51 wherein P₁ is phenyl substituted with a phenyl substituted with halo.

53 The compound of embodiment 52 wherein P₁ is phenyl substituted with a phenyl substituted with —C₁.

54 The compound of embodiment 51, wherein P₁ is phenyl substituted with a phenyl substituted with —O—C₁-C₆-alkyl, such as phenyl substituted with a phenyl substituted with —OⁱPr.

55 The compound of embodiment 54, wherein P₁ is phenyl substituted with a phenyl substituted with —O—C₃-alkyl.

56 The compound of embodiment 55, wherein, P₁ is phenyl substituted with a phenyl substituted with —OCF₃ or P is phenyl substituted with a phenyl substituted with —OCF₃ and —F.

57 The compound of any one of embodiments 37 to 56 wherein Q₁ is selected from:

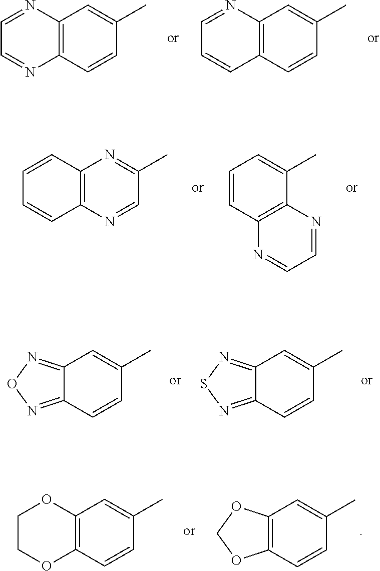

58 The compound of embodiment 57, wherein Q₁ is selected from:

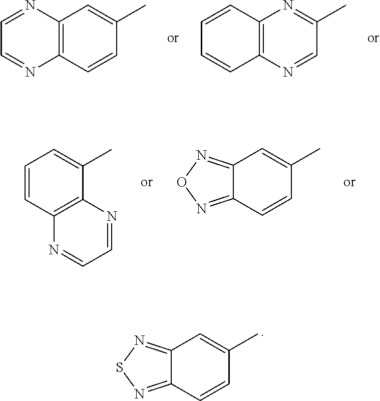

59 The compound of embodiment 57 or 58 wherein Q₁ is

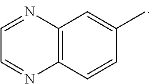

60 The compound of embodiment 37 being

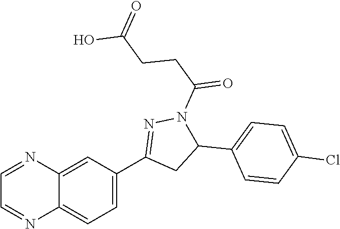 [SMILES: O=C(O)CCC(=O)N1N=C(c2ccc3nccnc3c2)CC1c1ccc(Cl)cc1]

or a salt thereof.

61 The compound of Formula II, III or IV as defined in any one of embodiments 1 to 60 for reducing the formation of methane from the digestive actions of ruminants and/or for improving ruminant performance.

62 The compound of Formula II, III or IV as defined in any one of embodiments 1 to 60 is for reducing the formation of methane from the digestive actions of ruminants.

63 The compound of Formula II, III or IV as defined in any one of embodiments 1 to 60 for reducing the formation of methane from the digestive actions of ruminants by at least 10%.

64 The compound of Formula II, III or IV as defined in any one of embodiments 1 to 60 is for reducing the formation of methane from the digestive actions of ruminants by at least 15%.

65 The compound of Formula II, III or IV as defined in any one of embodiments 1 to 60 is for reducing the formation of methane from the digestive actions of ruminants by at least 20%.

66 A method for reducing the production of methane emanating from a ruminant and/or for improving ruminant animal performance, comprising administering orally to the ruminant an effective amount of at least one compound of Formula II, III or IV or a salt thereof as defined in any one of embodiments 1 to 60 to the ruminant.

67 The method as claimed in claim 87, wherein the effective amount of at least one compound of Formula II, III or IV or a salt thereof as defined in any one of embodiments 1 to 60 is administered at least once-daily to the ruminant.

68 The method as claimed in claim 87 or claim 88, wherein the effective amount of at least one compound of Formula II, III or IV or a salt thereof as defined in any one of embodiments 1 to 60 reduces the production of methane emanating from the ruminant by at least 10% per day.

69 The method as claimed in any one of claims 87 to 89, wherein the effective amount of at least one compound of Formula II, III or IV or a salt thereof as defined in any one of embodiments 1 to 60 reduces the production of methane emanating from the ruminant by at least 15% per day.

70 The method as claimed in any one of claims 87 to 90 wherein the effective amount of at least one compound of Formula II, III or IV or a salt thereof as defined in any one of embodiments 1 to 60 reduces the production of methane emanating from the ruminant by at least 20% per day.

71 A composition for oral administration comprising at least one compound of Formula II, III or IV or a salt thereof as defined in any one of embodiments 1 to 60 for reducing the production of methane emanating from a ruminant, and the composition further including at least one agriculturally and orally acceptable excipient.

72 The composition of embodiment 71, being adapted for use as a feed additive.

73 The composition of embodiment 71, being adapted for use as a water additive.

74 The composition of embodiment 71, being adapted for use as a ruminant lick.

75 The composition of embodiment 71, being adapted for use as an oral drench.

76 The composition of embodiment 71, being adapted for use as a rumen bolus or capsule.

77 The composition of any one of embodiments 71 to 76, being adapted to reduce the production of methane emanating from the ruminant by at least 10% per day.

78 The composition of any one of embodiments 71 to 77, being adapted to reduce the production of methane emanating from the ruminant by at least 15% per day.

79 The composition of any one of embodiments 71 to 78, being to reduce the production of methane emanating from the ruminant by at least 20% per day.

80 The composition of any one of embodiments 71 to 79, wherein the excipient may include one or more minerals and/or one or more vitamins.

81 The composition of any one of embodiments 71 to 80, wherein the excipient may include one or more vitamins selected from vitamin A, vitamin D3, vitamin E, and vitamin K, e.g. vitamin K3, vitamin B12, biotin and choline, vitamin B1, vitamin B2, vitamin B6, niacin, folic acid or the like.

82 The composition of any one of embodiments 71 to 81, wherein the excipient may include one or more minerals selected from calcium, phosphorus, sodium, manganese, zinc, iron, copper, chlorine, sulphur, magnesium, iodine, selenium, and cobalt or the like.

83 The composition of any one of embodiments 71 to 82 further including sunflower oil, electrolytes such as ammonium chloride, calcium carbonates, starch, proteins or the like.

1. A compound of Formula I 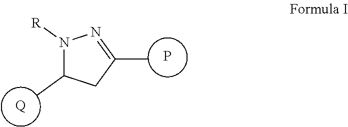
wherein R may be selected from:
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C═O)—C₁-C₆, or
—C(═O)—C₁-C₆—C(═O)—NH₂, —C(═O)—C₁-C₆—C(═O)—NH—C₁-C₆, —C(═O)—C₁-C₆—C(═O)—N(C₁-C₆)₂,
—C(═O)—C₁-C₆—C(═O)—NH—S(O)₂—C₁-C₆, —C(═O)—C₁-C₆—C(═O)—NH—S(O)₂-aryl, —C(═O)—C₁-C₆—C(═O)—NH—S(O)₂—N(C₁-C₆)₂, 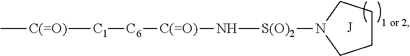
wherein J is a 5 or 6 membered heterocycle ring optionally including a second heteroatom selected from —NH—, NC₁-C₆—, —S— or —O—; or
—C₁-C₆—C(═O)—OH, or
-aryl-C(═O)—OH, or
-aryl-C(═O)—NH—S(O)₂—C₁-C₆;
wherein P may be a 5 to 10 membered aryl ring or rings optionally containing one or more heteroatoms, and wherein P may be further substituted with one or more halo, —C₁-C₆-alkyl, —C₁-C₆-alkyl substituted with one or more halo, hydroxy, —C₃-C₈-cycloalkyl, —CN, hydroxy, —O—C₁-C₆-alkyl, —O—C₁-C₆-alkyl substituted with one or more halo, —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂, 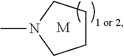
wherein M is a 5 or 6 membered heterocycle ring optionally including a second heteroatom selected from —NH—, —NC₁-C₆—, —S— or —O—; or C₂-C₈ cycloheteroalkyl including one or more heteroatoms selected from —NH—, —NC₁-C₆—, —S— or —O—; or
aryl, aryl substituted with one or more, halo, —C₁-C₆-alkyl, —C₁-C₆-alkyl substituted with one or more halo or hydroxy, —C₃-C₈-cycloalkyl, —CN, hydroxy, —O—C₁-C₆-alkyl, —O—C₁-C₆-alkyl substituted with one or more halo, —C₁-C₆-alkyl substituted with one or more —O—C₁-C₆-alkyl; —O—C₁-C₆-alkyl substituted with one or more —O—C₁-C₆-alkyl; —O—C₃-C₈-cycloalkyl, —SH, —S—C₁-C₆-alkyl, —S—C₁-C₆-alkyl substituted with one or more halo, —SF₆, —NO₂, —NH₂, —NH—C₁-C₆-alkyl, —N(C₁-C₆-alkyl)₂, 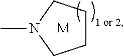
wherein M is a 5 or 6 membered heterocycle ring optionally including a second heteroatom selected from —NH—, —NC₁-C₆—, —S— or —O—; or
heteroaryl; heteroaryl substituted with one or more —O—C₁-C₆-alkyl, or
aryl fused to a 5-6 membered heterocyclic ring having one or more —O— or —N— groups, the heterocyclic ring being optionally substituted with one or more halo or —C₁-C₆-alkyl or O—C₁-C₆-alkyl, or
—O-aryl;
wherein Q may be selected from 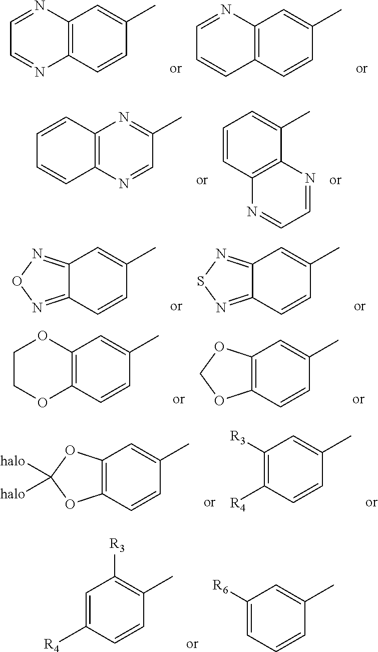
wherein R₃, R₄, or R₆ are independently selected from —H, halo, —C₁-C₆-alkyl, —C₁-C₆-alkyl substituted with one or more halo, —CN, hydroxy, —O—C₁-C₆-alkyl, —O—C₁-C₆-alkyl substituted with one or more halo; and
or a salt thereof;
with the proviso that the following compounds 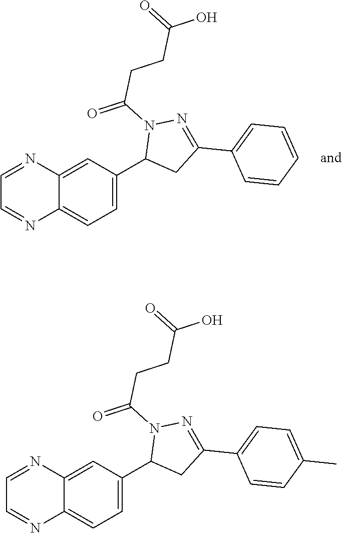 [SMILES: O=C(O)CCC(=O)N1N=C(c2ccccc2)CC1c1ccc2nccnc2c1 ; Cc1ccc(C2=NN(C(=O)CCC(=O)O)C(c3ccc4nccnc4c3)C2)cc1]
are excluded from the compounds of Formula I.

2. The compound as claimed in claim 1, wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, —C₁-C₆—C(═O)—OH, -aryl-C(═O)—OH.

3. The compound as claimed in claim 1 or claim 2, wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, -aryl-C(═O)—OH.

4. The compound as claimed in any one of claims 1 to 3, wherein R is selected from —C(═O)—C₁-C₆—C(═O)—OH.

5. The compound as claimed in claim 1, wherein R is —C(═O)—C₁-C₆—C(═O)—NH—S(O)₂—C₁-C₆.

6. The compound as claimed in claim 1, wherein R is selected from —C(═O)—C₁-C₆—C(═O)—NH—S(O)₂—N(C₁-C₆)₂, such as —C(═O)—C₁-C₆—C(═O)—NH—S(O)₂—N(C₁-C₆ alkyl)₂:
or —C(═O)—C₁-C₆—C(═O)—NH—S(O)₂-aryl, such as —C(═O)—CH₂—CH₂—C(═O)—NH—S(O)₂-Ph.

7. The compound as claimed in claim 1, wherein R is -aryl-C(═O)—OH.

8. The compound as claimed in claim 1, wherein R is —C(═O)—CH₂—CH₂—C(═O)—OH.

9. The compound as claimed in claim 1 or claim 5, wherein R is selected from —C(═O)—CH₂—CH₂—C(═O)—NH—S(O)₂-Me, or —C(═O)—CH₂—CH₂—C(═O)—NH—S(O)₂-iPr.

10. The compound as claimed in claim 1, wherein R is selected from —C(═O)—CH₂—CH₂—C(═O)—NH—S(O)₂—NMe₂.

11. The compound as claimed in claim 1, wherein R is -phenyl-C(═O)—OH.

12. The compound as claimed in any one of claims 1 to 11, wherein P is aryl or an aryl substituted with one or more halo, —C₁-C₆-alkyl, or —O—C₁-C₆-alkyl.

13. The compound as claimed in claim 12, wherein P is aryl substituted with aryl or an aryl substituted with halo, —C₁-C₆-alkyl, —O—C₁-C₆-alkyl or —OC₁-C₆-alkyl substituted with one or more halo.

14. The compound as claimed in claim 12 or claim 13, wherein P is phenyl or a phenyl substituted with one or more halo, —C₁-C₆-alkyl, —O—C₁-C₆-alkyl or —OC₁-C₆-alkyl substituted with one or more halo.

15. The compound as claimed in claim 13, wherein P is phenyl substituted with phenyl or a phenyl substituted with halo, —C₁-C₆-alkyl, —C₁-C₆-alkyl substituted with one or more halo, —O—C₁-C₆-alkyl or —OC₁-C₆-alkyl substituted with one or more halo.

16. The compound as claimed in claim 15, wherein P is phenyl substituted with a phenyl substituted with halo.

17. The compound as claimed in claim 16, wherein P is phenyl substituted with a phenyl substituted with —Cl.

18. The compound as claimed in claim 15, wherein P is phenyl substituted with a phenyl substituted with —O—C₁-C₆-alkyl, such as phenyl substituted with a phenyl substituted with —OⁱPr.

19. The compound as claimed in claim 18, wherein P is phenyl substituted with a phenyl substituted with —O—C₃-alkyl.

20. The compound as claimed in claim 18, wherein, P is phenyl substituted with a phenyl substituted with —OCF₃ or P is phenyl substituted with a phenyl substituted with —OCF₃ and —F.

21. The compound as claimed in any one of claims 1 to 20, wherein Q is selected from:

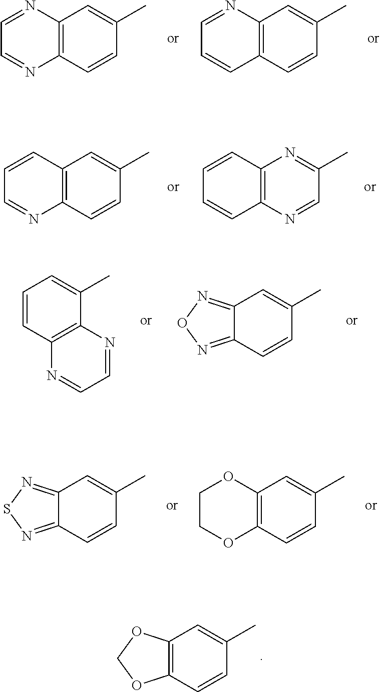

22. The compound as claimed in claim 21, wherein Q is selected from:

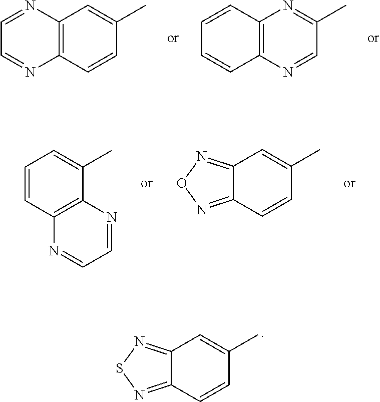

23. The compound as claimed in claim 21 or claim 22 wherein Q is

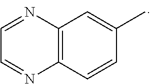

24. The compound as claimed in any one of claims 1 to 23, wherein the compound of Formula I is selected from one or more of the following:

| Compound No | Structure |
|---|---|
| 1 | 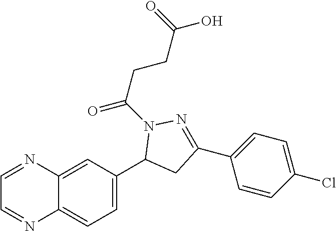 [SMILES: O=C(O)CCC(=O)N1N=C(c2ccc(Cl)cc2)CC1c1ccc2nccnc2c1] |
| 2 | 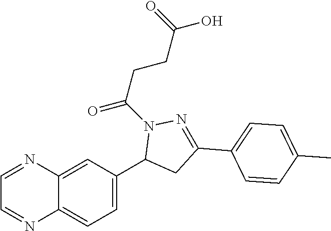 [SMILES: Cc1ccc(C2=NN(C(=O)CCC(=O)O)C(c3ccc4nccnc4c3)C2)cc1] |
| 3 | 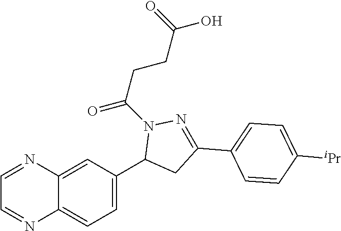 [SMILES: CC(C)c1ccc(C2=NN(C(=O)CCC(=O)O)C(c3ccc4nccnc4c3)C2)cc1] |
| 4 | 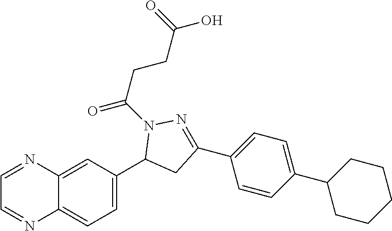 [SMILES: O=C(O)CCC(=O)N1N=C(c2ccc(C3CCCCC3)cc2)CC1c1ccc2nccnc2c1] |
| 5 | 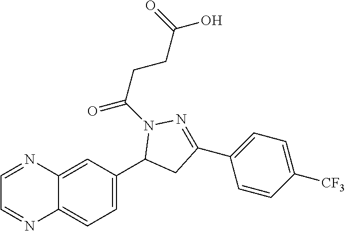 [SMILES: O=C(O)CCC(=O)N1N=C(c2ccc(C(F)(F)F)cc2)CC1c1ccc2nccnc2c1] |
| 6 | 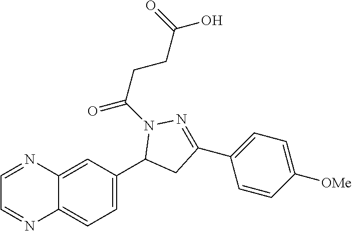 [SMILES: COc1ccc(C2=NN(C(=O)CCC(=O)O)C(c3ccc4nccnc4c3)C2)cc1] |
| 7 | 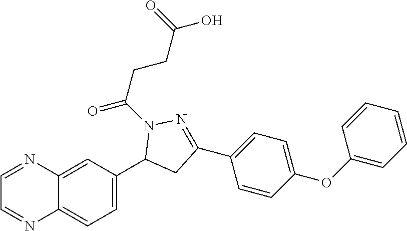 [SMILES: O=C(O)CCC(=O)N1N=C(c2ccc(Oc3ccccc3)cc2)CC1c1ccc2nccnc2c1] |
| 8 | 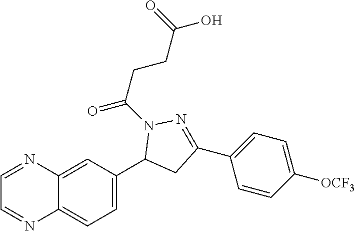 [SMILES: O=C(O)CCC(=O)N1N=C(c2ccc(OC(F)(F)F)cc2)CC1c1ccc2nccnc2c1] |
| 9 | 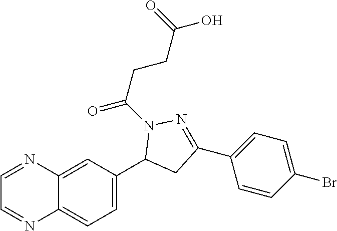 [SMILES: O=C(O)CCC(=O)N1N=C(c2ccc(Br)cc2)CC1c1ccc2nccnc2c1] |
| 10 | 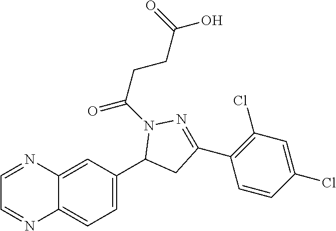 [SMILES: O=C(O)CCC(=O)N1N=C(c2ccc(Cl)cc2Cl)CC1c1ccc2nccnc2c1] |
| 11 | 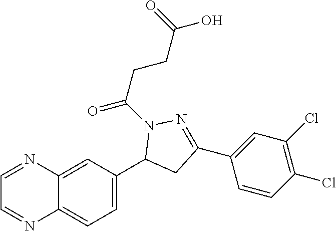 [SMILES: O=C(O)CCC(=O)N1N=C(c2ccc(Cl)c(Cl)c2)CC1c1ccc2nccnc2c1] |
| 12 | 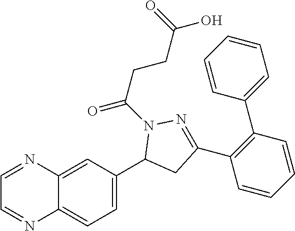 [SMILES: O=C(O)CCC(=O)N1N=C(c2ccccc2-c2ccccc2)CC1c1ccc2nccnc2c1] |
| 13 | 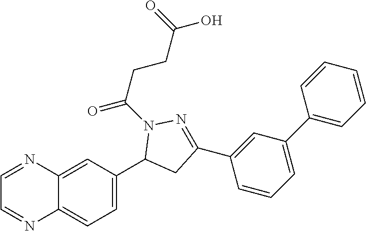 [SMILES: O=C(O)CCC(=O)N1N=C(c2cccc(-c3ccccc3)c2)CC1c1ccc2nccnc2c1] |
| 14 | 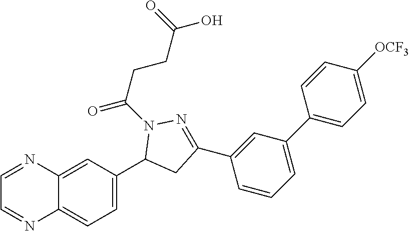 [SMILES: O=C(O)CCC(=O)N1N=C(c2cccc(-c3ccc(OC(F)(F)F)cc3)c2)CC1c1ccc2nccnc2c1] |
| 15 | 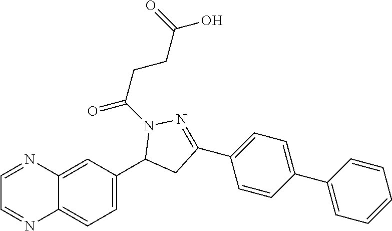 [SMILES: O=C(O)CCC(=O)N1N=C(c2ccc(-c3ccccc3)cc2)CC1c1ccc2nccnc2c1] |
| 16 | 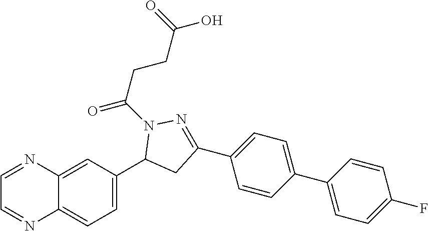 [SMILES: O=C(O)CCC(=O)N1N=C(c2ccc(-c3ccc(F)cc3)cc2)CC1c1ccc2nccnc2c1] |
| 17 | 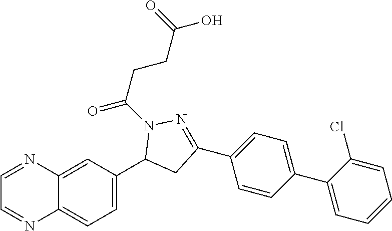 [SMILES: O=C(O)CCC(=O)N1N=C(c2ccc(-c3ccccc3Cl)cc2)CC1c1ccc2nccnc2c1] |
| 18 | 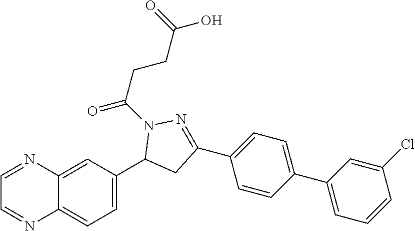 [SMILES: O=C(O)CCC(=O)N1N=C(c2ccc(-c3cccc(Cl)c3)cc2)CC1c1ccc2nccnc2c1] |
| 19 | 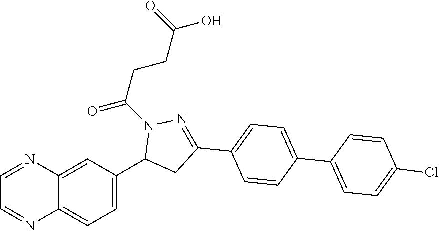 [SMILES: O=C(O)CCC(=O)N1N=C(c2ccc(-c3ccc(Cl)cc3)cc2)CC1c1ccc2nccnc2c1] |
| 20 | 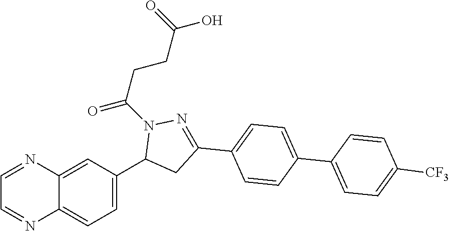 [SMILES: O=C(O)CCC(=O)N1N=C(c2ccc(-c3ccc(C(F)(F)F)cc3)cc2)CC1c1ccc2nccnc2c1] |
| 21 | 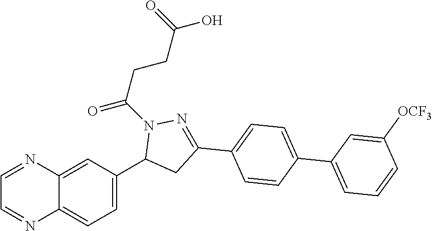 [SMILES: O=C(O)CCC(=O)N1N=C(c2ccc(-c3cccc(OC(F)(F)F)c3)cc2)CC1c1ccc2nccnc2c1] |
| 22 | 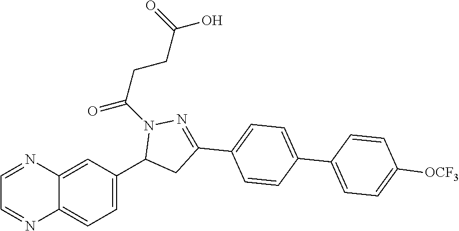 [SMILES: O=C(O)CCC(=O)N1N=C(c2ccc(-c3ccc(OC(F)(F)F)cc3)cc2)CC1c1ccc2nccnc2c1] |
| 23 | 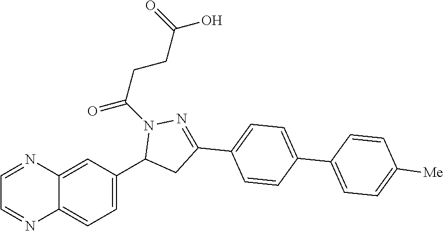 [SMILES: Cc1ccc(-c2ccc(C3=NN(C(=O)CCC(=O)O)C(c4ccc5nccnc5c4)C3)cc2)cc1] |
| 24 | 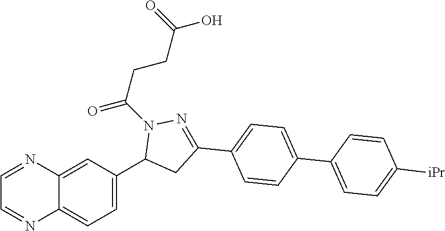 [SMILES: CC(C)c1ccc(-c2ccc(C3=NN(C(=O)CCC(=O)O)C(c4ccc5nccnc5c4)C3)cc2)cc1] |
| 25 | 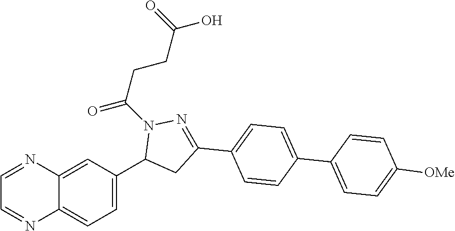 [SMILES: COc1ccc(-c2ccc(C3=NN(C(=O)CCC(=O)O)C(c4ccc5nccnc5c4)C3)cc2)cc1] |
| 26 | 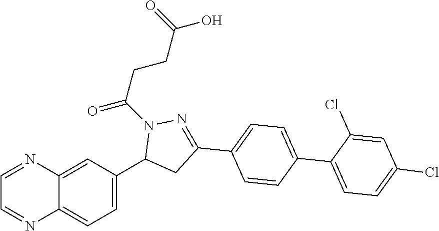 [SMILES: O=C(O)CCC(=O)N1N=C(c2ccc(-c3ccc(Cl)cc3Cl)cc2)CC1c1ccc2nccnc2c1] |
| 27 | 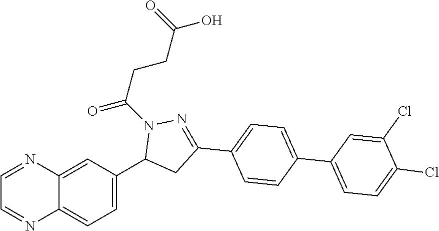 [SMILES: O=C(O)CCC(=O)N1N=C(c2ccc(-c3ccc(Cl)c(Cl)c3)cc2)CC1c1ccc2nccnc2c1] |
| 28 | 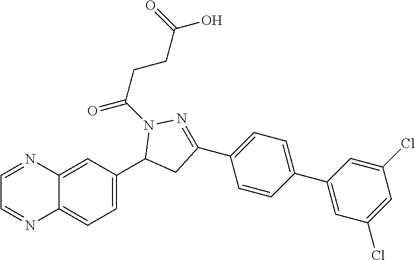 [SMILES: O=C(O)CCC(=O)N1N=C(c2ccc(-c3cc(Cl)cc(Cl)c3)cc2)CC1c1ccc2nccnc2c1] |
| 29 | 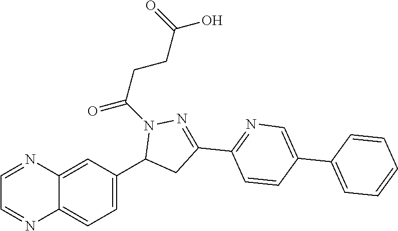 [SMILES: O=C(O)CCC(=O)N1N=C(c2ccc(-c3ccccc3)cn2)CC1c1ccc2nccnc2c1] |
| 30 | 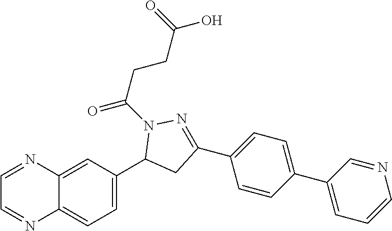 [SMILES: O=C(O)CCC(=O)N1N=C(c2ccc(-c3cccnc3)cc2)CC1c1ccc2nccnc2c1] |
| 31 | 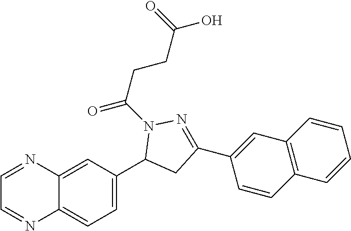 [SMILES: O=C(O)CCC(=O)N1N=C(c2ccc3ccccc3c2)CC1c1ccc2nccnc2c1] |
| 32 | 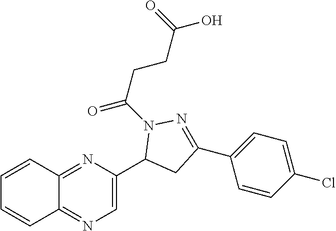 [SMILES: O=C(O)CCC(=O)N1N=C(c2ccc(Cl)cc2)CC1c1cnc2ccccc2n1] |
| 33 | 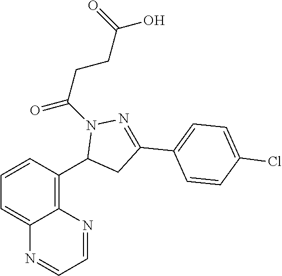 [SMILES: O=C(O)CCC(=O)N1N=C(c2ccc(Cl)cc2)CC1c1cccc2nccnc12] |
| 34 | 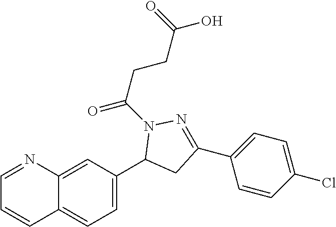 [SMILES: O=C(O)CCC(=O)N1N=C(c2ccc(Cl)cc2)CC1c1ccc2cccnc2c1] |
| 35 | 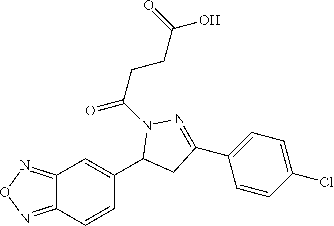 [SMILES: O=C(O)CCC(=O)N1N=C(c2ccc(Cl)cc2)CC1c1ccc2nonc2c1] |
| 36 | 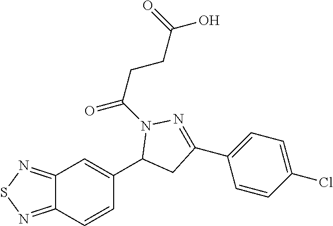 [SMILES: O=C(O)CCC(=O)N1N=C(c2ccc(Cl)cc2)CC1c1ccc2nsnc2c1] |
| 37 | 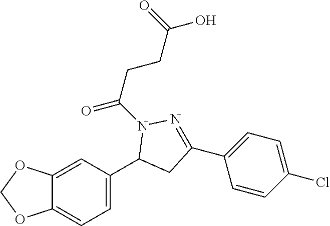 [SMILES: O=C(O)CCC(=O)N1N=C(c2ccc(Cl)cc2)CC1c1ccc2c(c1)OCO2] |
| 38 | 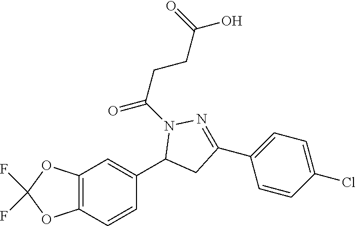 [SMILES: O=C(O)CCC(=O)N1N=C(c2ccc(Cl)cc2)CC1c1ccc2c(c1)OC(F)(F)O2] |
| 39 | 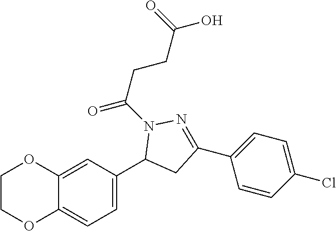 [SMILES: O=C(O)CCC(=O)N1N=C(c2ccc(Cl)cc2)CC1c1ccc2c(c1)OCCO2] |
| 40 | 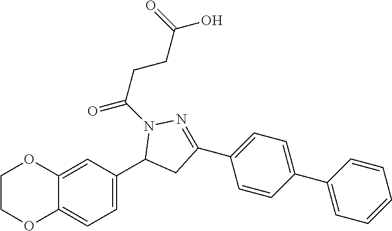 [SMILES: O=C(O)CCC(=O)N1N=C(c2ccc(-c3ccccc3)cc2)CC1c1ccc2c(c1)OCCO2] |
| 41 | 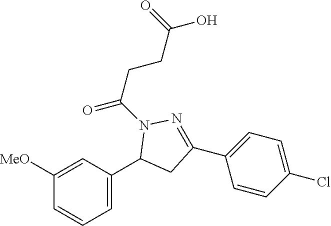 [SMILES: COc1cccc(C2CC(c3ccc(Cl)cc3)=NN2C(=O)CCC(=O)O)c1] |
| 42 | 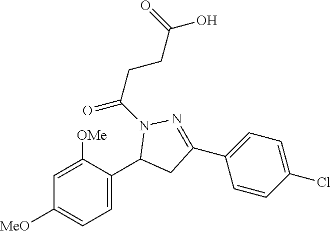 [SMILES: COc1ccc(C2CC(c3ccc(Cl)cc3)=NN2C(=O)CCC(=O)O)c(OC)c1] |
| 43 | 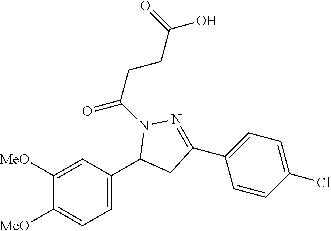 [SMILES: COc1ccc(C2CC(c3ccc(Cl)cc3)=NN2C(=O)CCC(=O)O)cc1OC] |
| 44 | 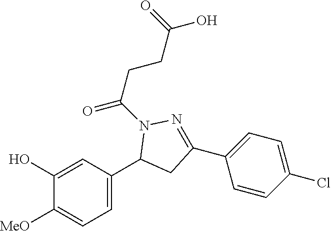 [SMILES: COc1ccc(C2CC(c3ccc(Cl)cc3)=NN2C(=O)CCC(=O)O)cc1O] |
| 45 | 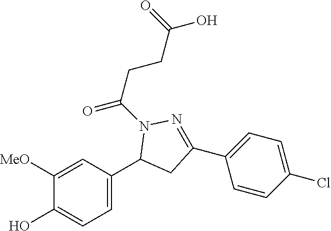 [SMILES: COc1cc(C2CC(c3ccc(Cl)cc3)=NN2C(=O)CCC(=O)O)ccc1O] |
| 46 | 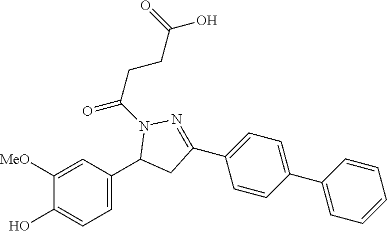 [SMILES: COc1cc(C2CC(c3ccc(-c4ccccc4)cc3)=NN2C(=O)CCC(=O)O)ccc1O] |
| 47 | 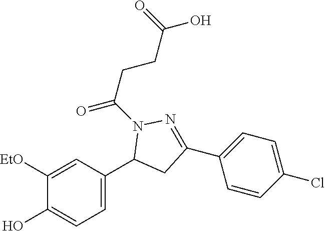 [SMILES: CCOc1cc(C2CC(c3ccc(Cl)cc3)=NN2C(=O)CCC(=O)O)ccc1O] |
| 48 | 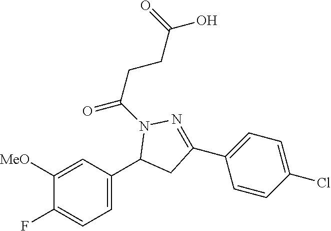 [SMILES: COc1cc(C2CC(c3ccc(Cl)cc3)=NN2C(=O)CCC(=O)O)ccc1F] |
| 49 | 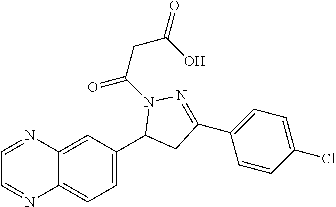 [SMILES: O=C(O)CC(=O)N1N=C(c2ccc(Cl)cc2)CC1c1ccc2nccnc2c1] |
| 50 | 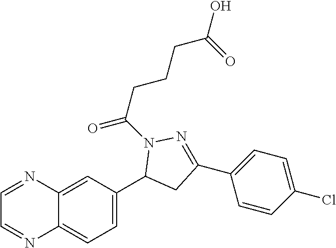 [SMILES: O=C(O)CCCC(=O)N1N=C(c2ccc(Cl)cc2)CC1c1ccc2nccnc2c1] |
| 51 | 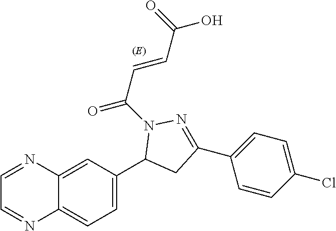 [SMILES: O=C(O)/C=C/C(=O)N1N=C(c2ccc(Cl)cc2)CC1c1ccc2nccnc2c1] |
| 52 | 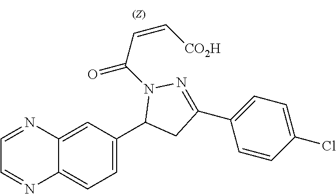 [SMILES: O=C(O)/C=C\C(=O)N1N=C(c2ccc(Cl)cc2)CC1c1ccc2nccnc2c1] |
| 53 | 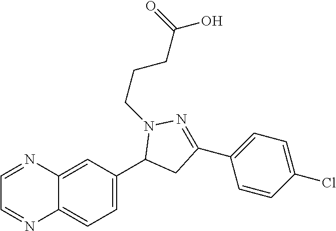 [SMILES: O=C(O)CCCN1N=C(c2ccc(Cl)cc2)CC1c1ccc2nccnc2c1] |
| 54 | 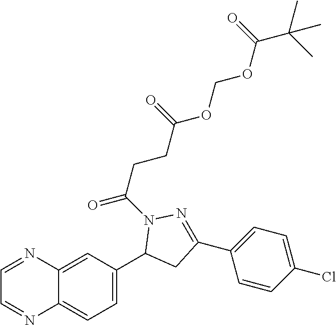 [SMILES: CC(C)(C)C(=O)OCOC(=O)CCC(=O)N1N=C(c2ccc(Cl)cc2)CC1c1ccc2nccnc2c1] |
| 55 | 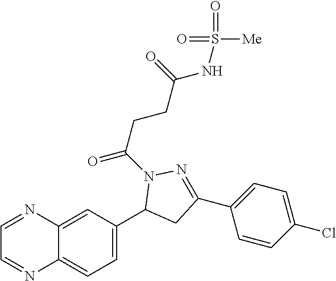 [SMILES: CS(=O)(=O)NC(=O)CCC(=O)N1N=C(c2ccc(Cl)cc2)CC1c1ccc2nccnc2c1] |
| 56 | 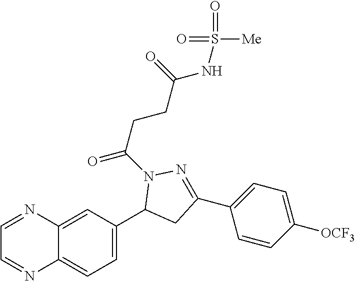 [SMILES: CS(=O)(=O)NC(=O)CCC(=O)N1N=C(c2ccc(OC(F)(F)F)cc2)CC1c1ccc2nccnc2c1] |
| 57 | 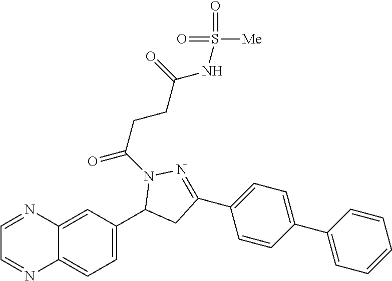 [SMILES: CS(=O)(=O)NC(=O)CCC(=O)N1N=C(c2ccc(-c3ccccc3)cc2)CC1c1ccc2nccnc2c1] |
| 58 | 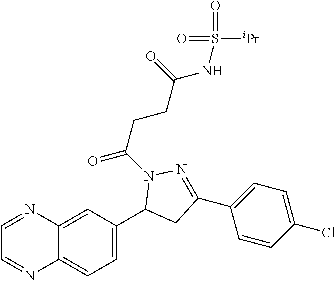 [SMILES: CC(C)S(=O)(=O)NC(=O)CCC(=O)N1N=C(c2ccc(Cl)cc2)CC1c1ccc2nccnc2c1] |
| 59 | 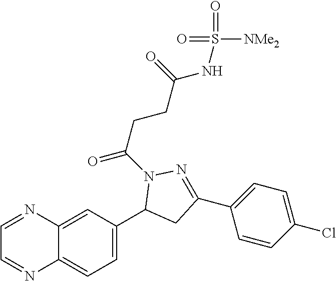 [SMILES: CN(C)S(=O)(=O)NC(=O)CCC(=O)N1N=C(c2ccc(Cl)cc2)CC1c1ccc2nccnc2c1] |
| 60 | 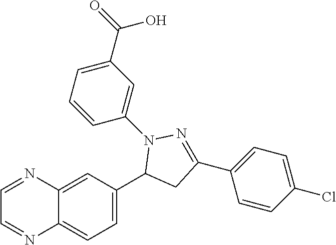 [SMILES: O=C(O)c1cccc(N2N=C(c3ccc(Cl)cc3)CC2c2ccc3nccnc3c2)c1] |
| 61 | 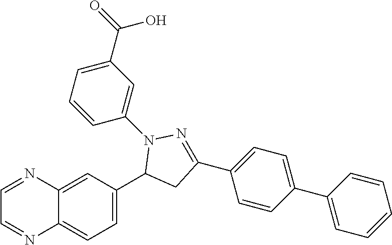 [SMILES: O=C(O)c1cccc(N2N=C(c3ccc(-c4ccccc4)cc3)CC2c2ccc3nccnc3c2)c1] |
| 62 | 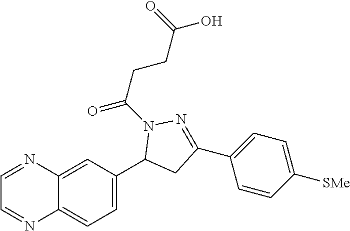 [SMILES: CSc1ccc(C2=NN(C(=O)CCC(=O)O)C(c3ccc4nccnc4c3)C2)cc1] |
| 63 | 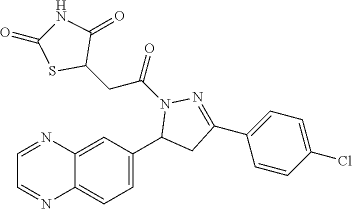 [SMILES: O=C1NC(=O)C(CC(=O)N2N=C(c3ccc(Cl)cc3)CC2c2ccc3nccnc3c2)S1] |
| 64 | 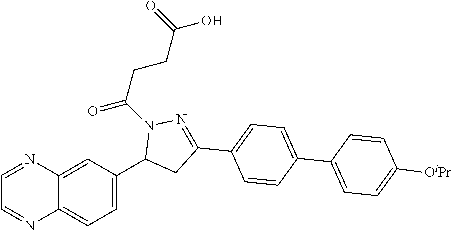 [SMILES: CC(C)Oc1ccc(-c2ccc(C3=NN(C(=O)CCC(=O)O)C(c4ccc5nccnc5c4)C3)cc2)cc1] |
| 65 | 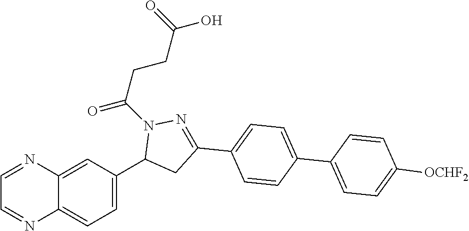 [SMILES: O=C(O)CCC(=O)N1N=C(c2ccc(-c3ccc(OC(F)F)cc3)cc2)CC1c1ccc2nccnc2c1] |
| 66 | 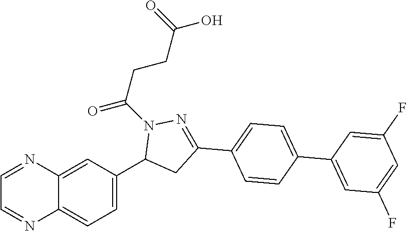 [SMILES: O=C(O)CCC(=O)N1N=C(c2ccc(-c3cc(F)cc(F)c3)cc2)CC1c1ccc2nccnc2c1] |
| 67 | 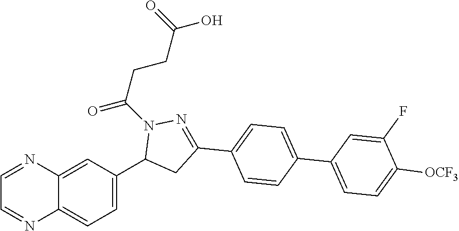 [SMILES: O=C(O)CCC(=O)N1N=C(c2ccc(-c3ccc(OC(F)(F)F)c(F)c3)cc2)CC1c1ccc2nccnc2c1] |
| 68 | 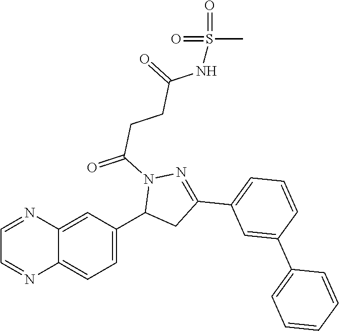 [SMILES: CS(=O)(=O)NC(=O)CCC(=O)N1N=C(c2cccc(-c3ccccc3)c2)CC1c1ccc2nccnc2c1] |
| 69 | 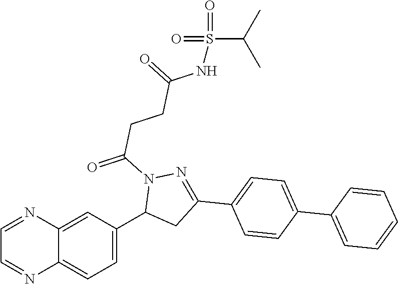 [SMILES: CC(C)S(=O)(=O)NC(=O)CCC(=O)N1N=C(c2ccc(-c3ccccc3)cc2)CC1c1ccc2nccnc2c1] |
| 70 | 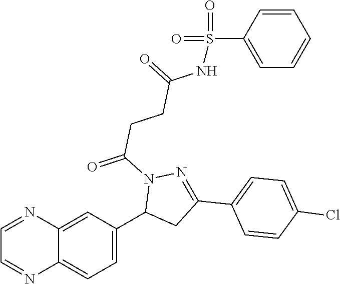 [SMILES: O=C(CCC(=O)N1N=C(c2ccc(Cl)cc2)CC1c1ccc2nccnc2c1)NS(=O)(=O)c1ccccc1] |
| 71 | 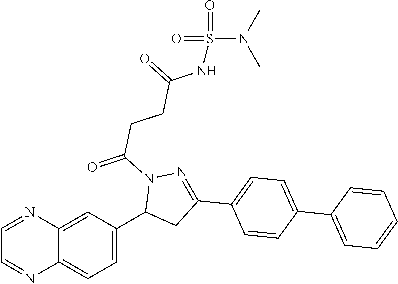 [SMILES: CN(C)S(=O)(=O)NC(=O)CCC(=O)N1N=C(c2ccc(-c3ccccc3)cc2)CC1c1ccc2nccnc2c1] |
| 72 | 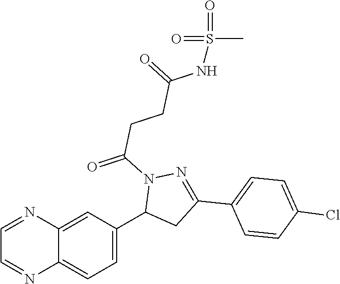 [SMILES: CS(=O)(=O)NC(=O)CCC(=O)N1N=C(c2ccc(Cl)cc2)CC1c1ccc2nccnc2c1] |
| 78 | 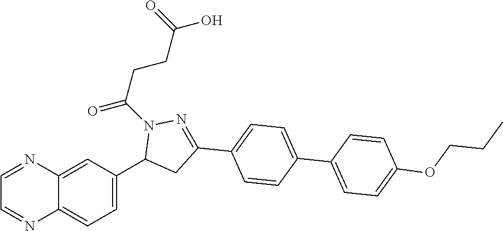 [SMILES: CCCOc1ccc(-c2ccc(C3=NN(C(=O)CCC(=O)O)C(c4ccc5nccnc5c4)C3)cc2)cc1] |
| 79 | 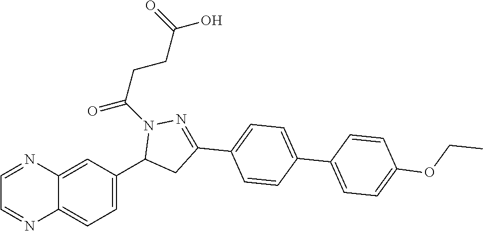 [SMILES: CCOc1ccc(-c2ccc(C3=NN(C(=O)CCC(=O)O)C(c4ccc5nccnc5c4)C3)cc2)cc1] |
| 80 | 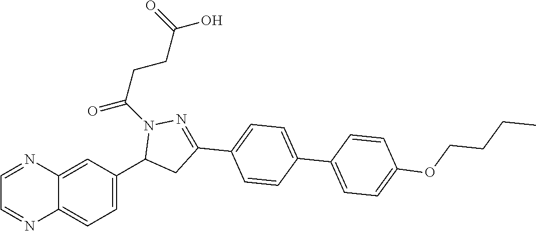 [SMILES: CCCCOc1ccc(-c2ccc(C3=NN(C(=O)CCC(=O)O)C(c4ccc5nccnc5c4)C3)cc2)cc1] |
| 81 | 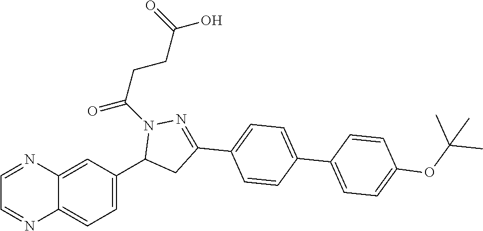 [SMILES: CC(C)(C)Oc1ccc(-c2ccc(C3=NN(C(=O)CCC(=O)O)C(c4ccc5nccnc5c4)C3)cc2)cc1] |
| 82 | 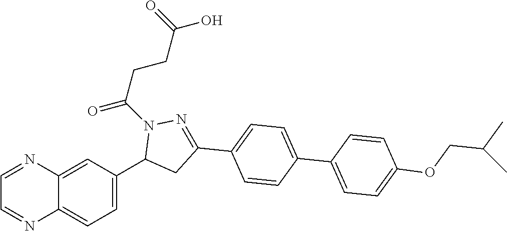 [SMILES: CC(C)COc1ccc(-c2ccc(C3=NN(C(=O)CCC(=O)O)C(c4ccc5nccnc5c4)C3)cc2)cc1] |
| 83 | 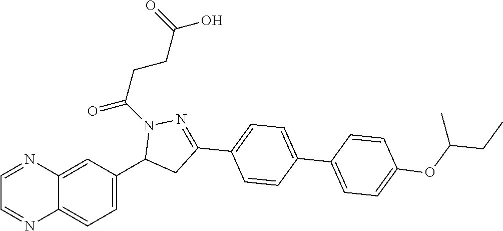 [SMILES: CCC(C)Oc1ccc(-c2ccc(C3=NN(C(=O)CCC(=O)O)C(c4ccc5nccnc5c4)C3)cc2)cc1] |
| 84 | 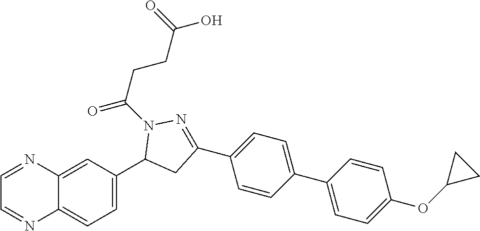 [SMILES: O=C(O)CCC(=O)N1N=C(c2ccc(-c3ccc(OC4CC4)cc3)cc2)CC1c1ccc2nccnc2c1] |
| 85 | 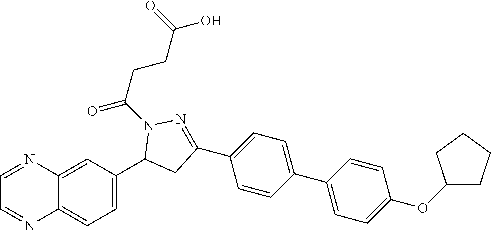 [SMILES: O=C(O)CCC(=O)N1N=C(c2ccc(-c3ccc(OC4CCCC4)cc3)cc2)CC1c1ccc2nccnc2c1] |
| 86 | 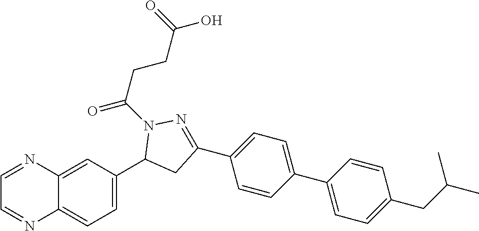 [SMILES: CC(C)Cc1ccc(-c2ccc(C3=NN(C(=O)CCC(=O)O)C(c4ccc5nccnc5c4)C3)cc2)cc1] |
| 87 | 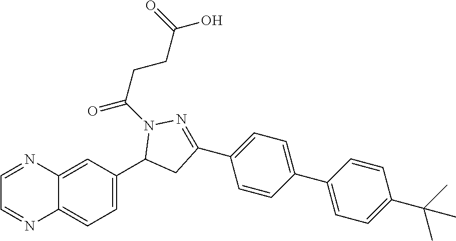 [SMILES: CC(C)(C)c1ccc(-c2ccc(C3=NN(C(=O)CCC(=O)O)C(c4ccc5nccnc5c4)C3)cc2)cc1] |
| 88 | 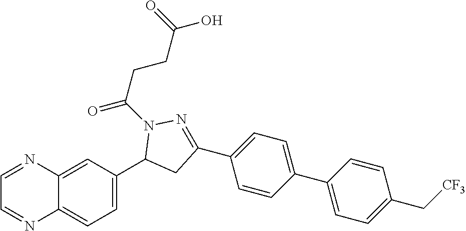 [SMILES: O=C(O)CCC(=O)N1N=C(c2ccc(-c3ccc(CC(F)(F)F)cc3)cc2)CC1c1ccc2nccnc2c1] |
| 89 | 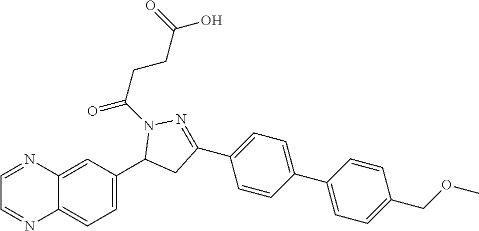 [SMILES: COCc1ccc(-c2ccc(C3=NN(C(=O)CCC(=O)O)C(c4ccc5nccnc5c4)C3)cc2)cc1] |
| 90 | 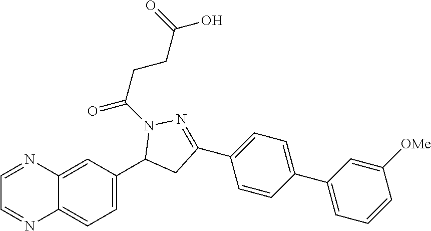 [SMILES: COc1cccc(-c2ccc(C3=NN(C(=O)CCC(=O)O)C(c4ccc5nccnc5c4)C3)cc2)c1] |
| 91 | 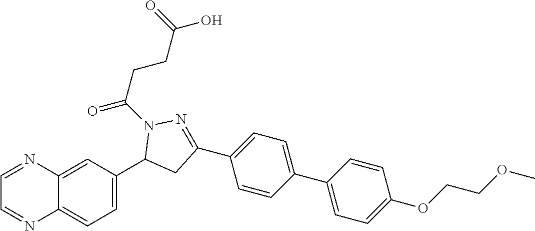 [SMILES: COCCOc1ccc(-c2ccc(C3=NN(C(=O)CCC(=O)O)C(c4ccc5nccnc5c4)C3)cc2)cc1] |
| 92 | 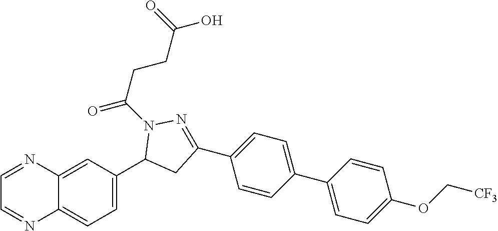 [SMILES: O=C(O)CCC(=O)N1N=C(c2ccc(-c3ccc(OCC(F)(F)F)cc3)cc2)CC1c1ccc2nccnc2c1] |
| 93 | 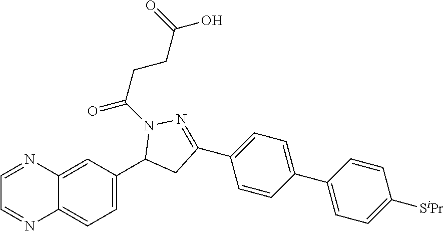 |
| 94 | 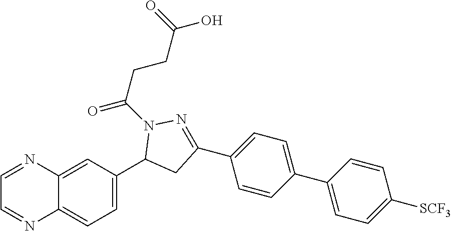 |
| 95 | 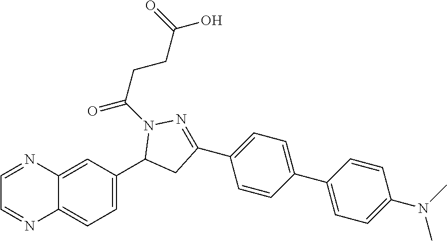 [SMILES: CN(C)c1ccc(-c2ccc(C3=NN(C(=O)CCC(=O)O)C(c4ccc5nccnc5c4)C3)cc2)cc1] |
| 96 | 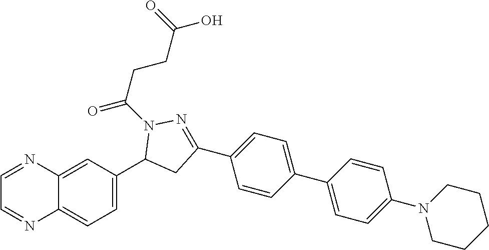 [SMILES: O=C(O)CCC(=O)N1N=C(c2ccc(-c3ccc(N4CCCCC4)cc3)cc2)CC1c1ccc2nccnc2c1] |
| 97 | 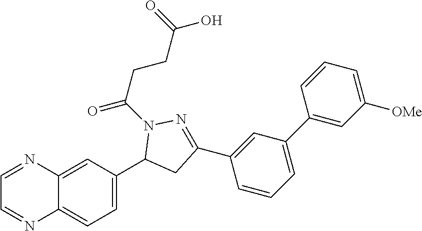 [SMILES: COc1cccc(-c2cccc(C3=NN(C(=O)CCC(=O)O)C(c4ccc5nccnc5c4)C3)c2)c1] |
| 98 | 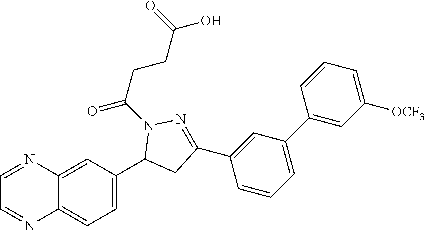 [SMILES: O=C(O)CCC(=O)N1N=C(c2cccc(-c3cccc(OC(F)(F)F)c3)c2)CC1c1ccc2nccnc2c1] |
| 99 | 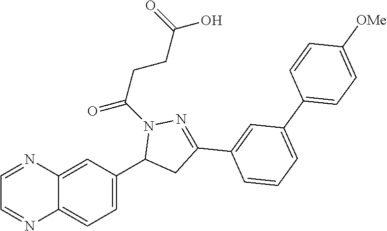 [SMILES: COc1ccc(-c2cccc(C3=NN(C(=O)CCC(=O)O)C(c4ccc5nccnc5c4)C3)c2)cc1] |
| 100 | 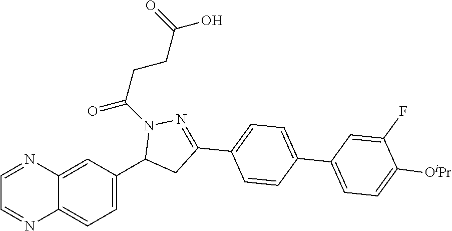 [SMILES: CC(C)Oc1ccc(-c2ccc(C3=NN(C(=O)CCC(=O)O)C(c4ccc5nccnc5c4)C3)cc2)cc1F] |
| 101 | 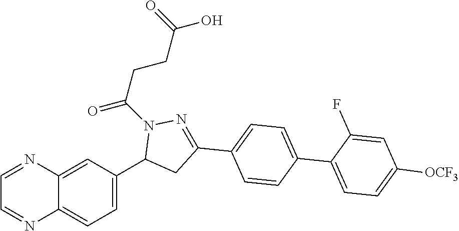 [SMILES: O=C(O)CCC(=O)N1N=C(c2ccc(-c3ccc(OC(F)(F)F)cc3F)cc2)CC1c1ccc2nccnc2c1] |
| 102 | 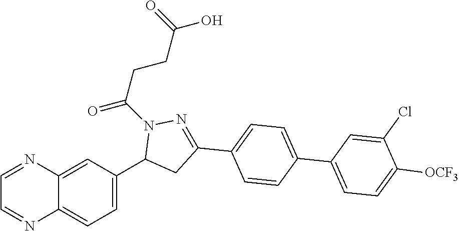 [SMILES: O=C(O)CCC(=O)N1N=C(c2ccc(-c3ccc(OC(F)(F)F)c(Cl)c3)cc2)CC1c1ccc2nccnc2c1] |
| 103 | 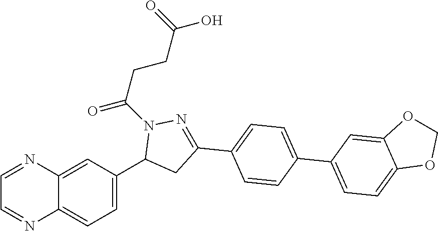 [SMILES: O=C(O)CCC(=O)N1N=C(c2ccc(-c3ccc4c(c3)OCO4)cc2)CC1c1ccc2nccnc2c1] |
| 104 | 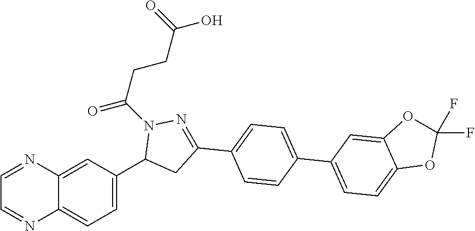 [SMILES: O=C(O)CCC(=O)N1N=C(c2ccc(-c3ccc4c(c3)OC(F)(F)O4)cc2)CC1c1ccc2nccnc2c1] |
| 105 | 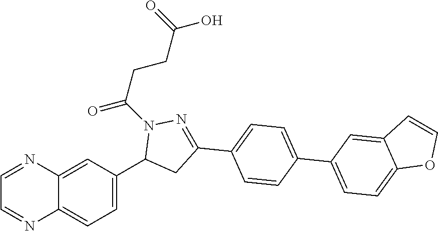 [SMILES: O=C(O)CCC(=O)N1N=C(c2ccc(-c3ccc4occc4c3)cc2)CC1c1ccc2nccnc2c1] |
| 106 | 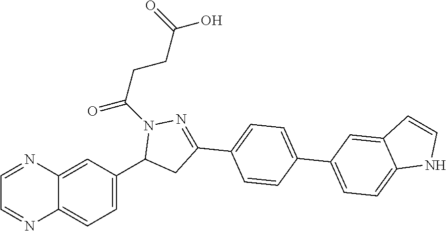 [SMILES: O=C(O)CCC(=O)N1N=C(c2ccc(-c3ccc4[nH]ccc4c3)cc2)CC1c1ccc2nccnc2c1] |
| 107 | 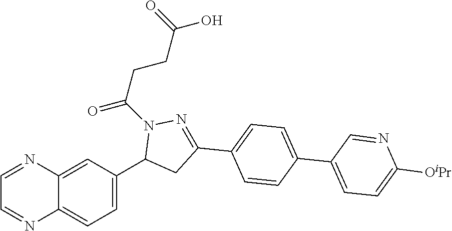 [SMILES: CC(C)Oc1ccc(-c2ccc(C3=NN(C(=O)CCC(=O)O)C(c4ccc5nccnc5c4)C3)cc2)cn1] |
| 108 | 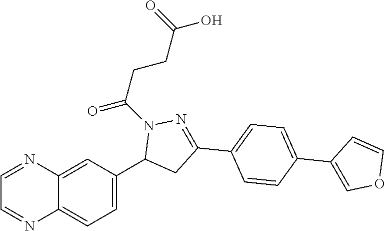 [SMILES: O=C(O)CCC(=O)N1N=C(c2ccc(-c3ccoc3)cc2)CC1c1ccc2nccnc2c1] |
| 109 | 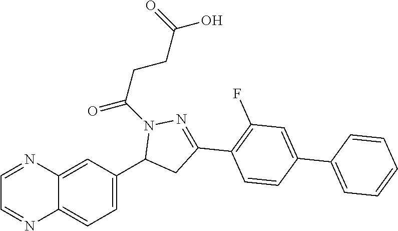 [SMILES: O=C(O)CCC(=O)N1N=C(c2ccc(-c3ccccc3)cc2F)CC1c1ccc2nccnc2c1] |
| 110 | 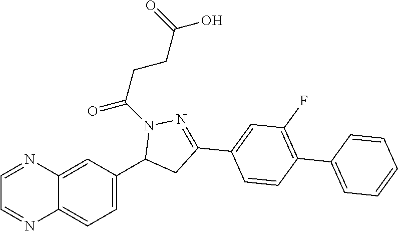 [SMILES: O=C(O)CCC(=O)N1N=C(c2ccc(-c3ccccc3)c(F)c2)CC1c1ccc2nccnc2c1] |
| 111 | 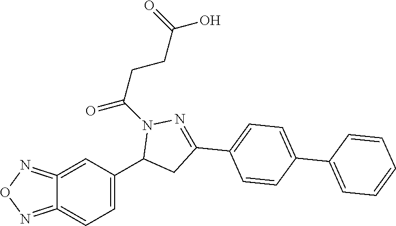 |
| 112 | 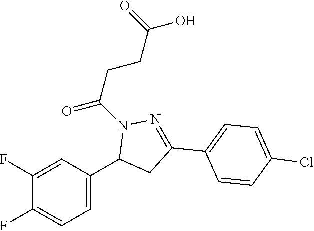 [SMILES: O=C(O)CCC(=O)N1N=C(c2ccc(Cl)cc2)CC1c1ccc(F)c(F)c1] |
| 113 | 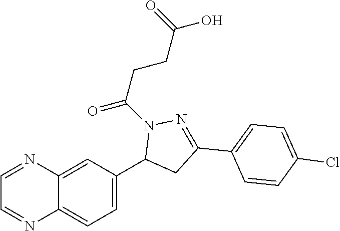 [SMILES: O=C(O)CCC(=O)N1N=C(c2ccc(Cl)cc2)CC1c1ccc2nccnc2c1] |
| 114 | 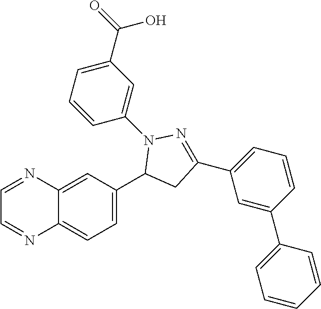 [SMILES: O=C(O)c1cccc(N2N=C(c3cccc(-c4ccccc4)c3)CC2c2ccc3nccnc3c2)c1] |
| 115 | 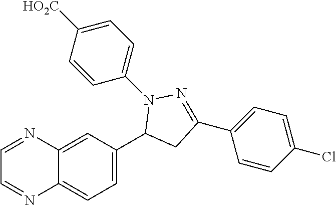 [SMILES: O=C(O)c1ccc(N2N=C(c3ccc(Cl)cc3)CC2c2ccc3nccnc3c2)cc1] |
| 116 | 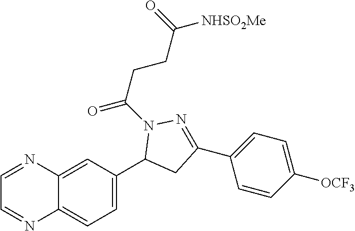 |
| 117 | 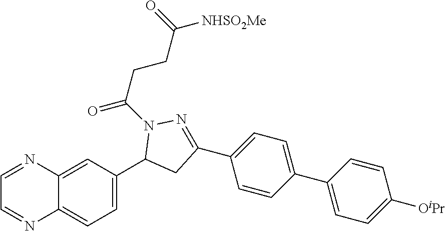 |
| 118 | 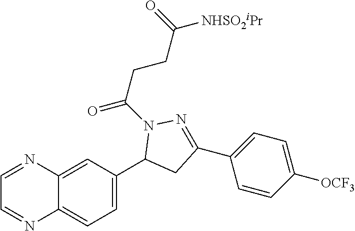 |
| 119 | 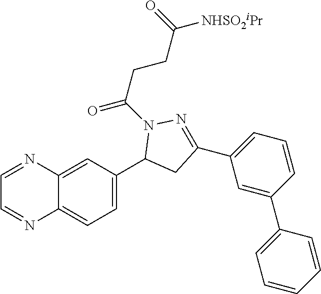 |
| 120 | 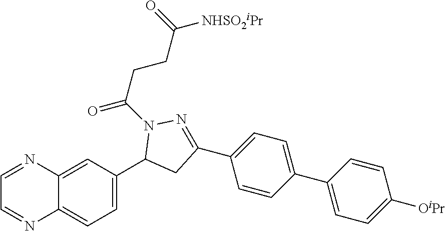 |
| 121 | 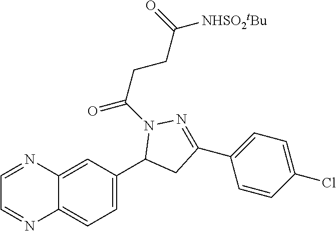 |
| 122 | 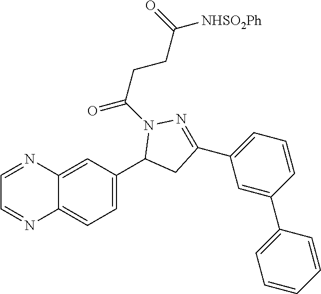 |
| 123 | 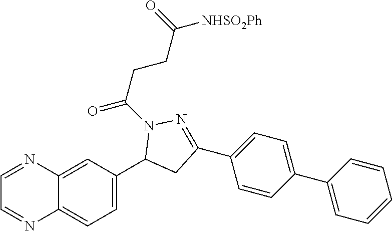 |
| 124 | 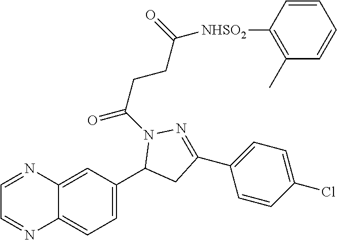 |
| 125 | 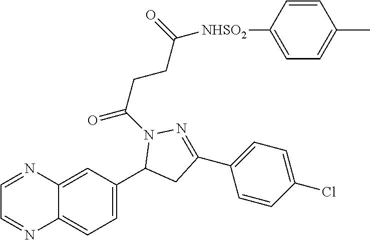 |
| 126 | 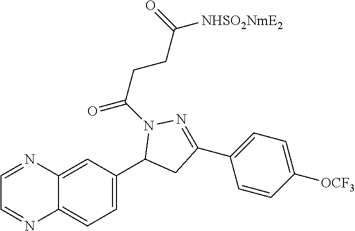 |
| 127 | 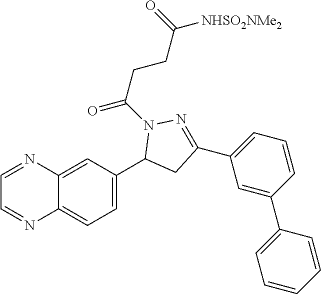 |
| 128 | 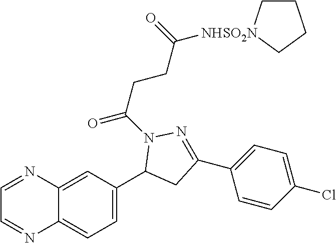 |

or a salt thereof.

25. A compound of Formula I being 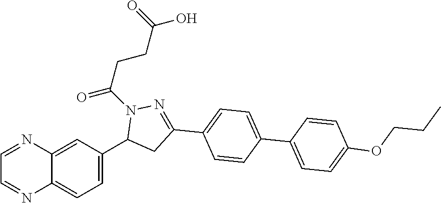 [SMILES: CCCOc1ccc(-c2ccc(C3=NN(C(=O)CCC(=O)O)C(c4ccc5nccnc5c4)C3)cc2)cc1]
or a salt thereof.

26. A compound of Formula I being 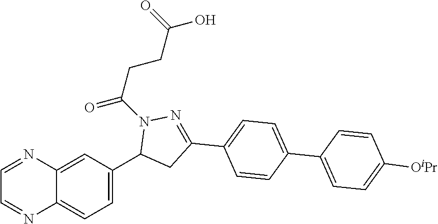 [SMILES: CC(C)Oc1ccc(-c2ccc(C3=NN(C(=O)CCC(=O)O)C(c4ccc5nccnc5c4)C3)cc2)cc1]
or a salt thereof.

27. A compound of Formula I being 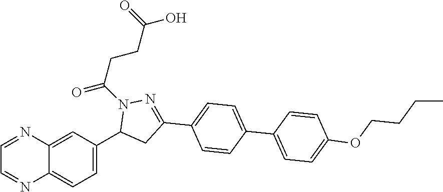 [SMILES: CCCCOc1ccc(-c2ccc(C3=NN(C(=O)CCC(=O)O)C(c4ccc5nccnc5c4)C3)cc2)cc1]
or a salt thereof.

28. A compound of Formula I being 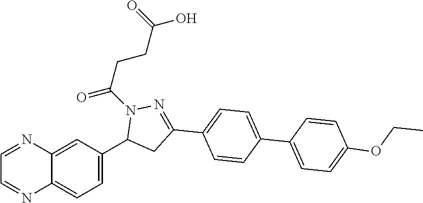 [SMILES: CCOc1ccc(-c2ccc(C3=NN(C(=O)CCC(=O)O)C(c4ccc5nccnc5c4)C3)cc2)cc1]
or a salt thereof.

29. A compound of Formula I being 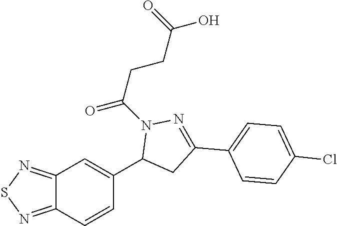 [SMILES: O=C(O)CCC(=O)N1N=C(c2ccc(Cl)cc2)CC1c1ccc2nsnc2c1]
or a salt thereof.

30. A compound of Formula I being 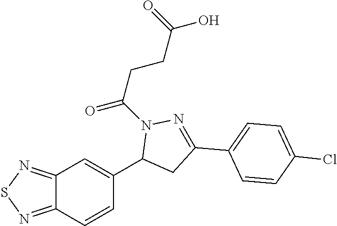 [SMILES: O=C(O)CCC(=O)N1N=C(c2ccc(Cl)cc2)CC1c1ccc2nsnc2c1]
or a salt thereof.

31. A compound of Formula I being 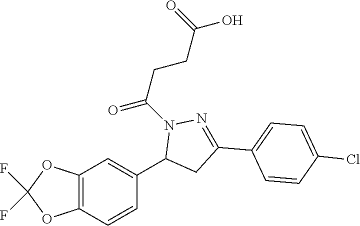 [SMILES: O=C(O)CCC(=O)N1N=C(c2ccc(Cl)cc2)CC1c1ccc2c(c1)OC(F)(F)O2]
or a salt thereof.

32. A compound of Formula I being 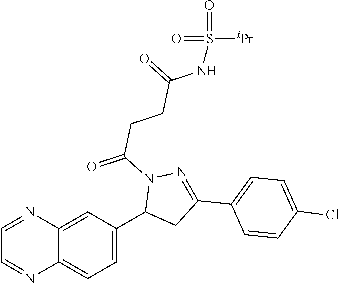 [SMILES: CC(C)S(=O)(=O)NC(=O)CCC(=O)N1N=C(c2ccc(Cl)cc2)CC1c1ccc2nccnc2c1]
or a salt thereof.

33. A compound of Formula I being 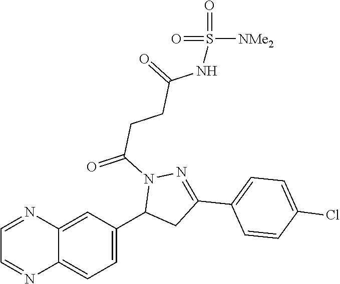 [SMILES: CN(C)S(=O)(=O)NC(=O)CCC(=O)N1N=C(c2ccc(Cl)cc2)CC1c1ccc2nccnc2c1]
or a salt thereof.

34. A compound of Formula I being 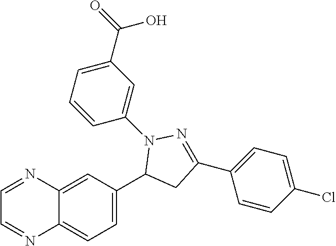 [SMILES: O=C(O)c1cccc(N2N=C(c3ccc(Cl)cc3)CC2c2ccc3nccnc3c2)c1]
or a salt thereof.

35. A compound of Formula I being 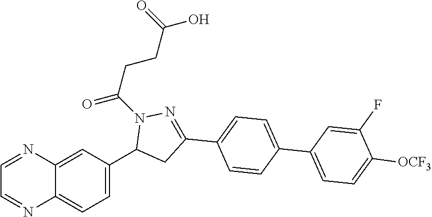 [SMILES: O=C(O)CCC(=O)N1N=C(c2ccc(-c3ccc(OC(F)(F)F)c(F)c3)cc2)CC1c1ccc2nccnc2c1]
or a salt thereof.

36. A compound of Formula I being 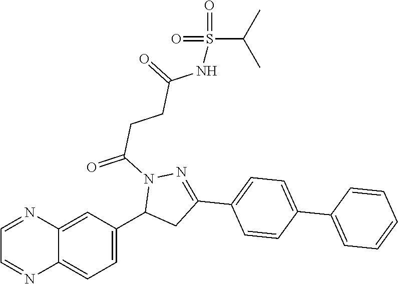 [SMILES: CC(C)S(=O)(=O)NC(=O)CCC(=O)N1N=C(c2ccc(-c3ccccc3)cc2)CC1c1ccc2nccnc2c1]
or a salt thereof.

37. A compound of Formula Ia 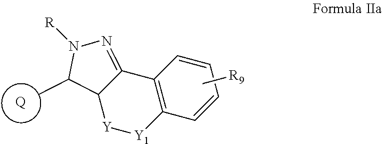
wherein R may be selected from:
—C(═O)—C₁-C₆—C(═O)—OH, —C(═O)—C₁-C₆—C(═O)—O—C₁-C₆, —C(═O)—C₁-C₆—C(═O)—O—C₁-C₆—O—(C—O)—C₁-C₆, or
—C(═O)—C₁-C₆—C(═O)—NH₂, —C(═O)—C₁-C₆—C(═O)—NH—C₁-C₆, —C(═O)—C₁-C₆—C(═O)—N(C₁-C₆)₂,
—C(═O)—C₁-C₆—C(═O)—NH—S(O)₂—C₁-C₆, —C(═O)—C₁-C₆—C(═O)—NH—S(O)₂-aryl, —C(═O)—C₁-C₆—C(═O)—NH—S(O)₂—N(C₁-C₆)₂, 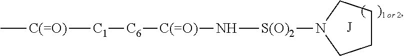
wherein J is a 5 or 6 membered heterocycle ring optionally including a second heteroatom selected from —NH—, NC₁-C₆—, —S— or —O—; or
—C₁-C₆—C(═O)—OH, or
-aryl-C(═O)—OH, or
-aryl-C(═O)—NH—S(O)₂—C₁-C₆;
wherein Q may be selected from 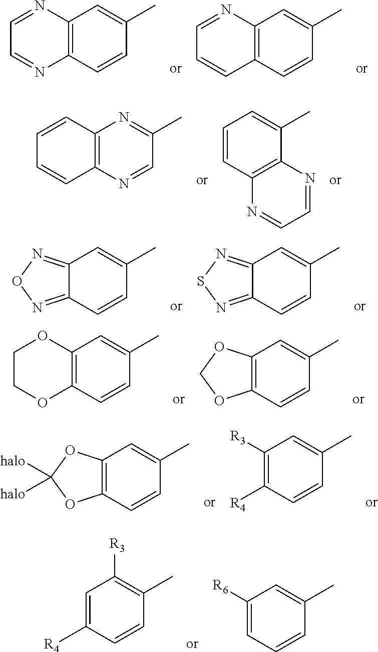
and
wherein R₃, R₄, or R₆ are independently selected from —H, halo, hydroxy, C₁-C₆ alkyl or C₁-C₆ alkoxy, alkoxy substituted with one or more halo, aryl optionally substituted with one or more halo, hydroxy, alkoxy, alkoxy substituted with one or more halo;
wherein R₉ is selected from halo, hydroxy, C₁-C₆ alkyl, C₁-C₆ alkoxy, aryl, or —O-aryl, where each C₁-C₆ alkyl, C₁-C₆ alkoxy, aryl, or —O-aryl may be further substituted with one or more halo, hydroxy, C₁-C₆ alkoxy or C₁-C₆ alkoxy further substituted with one or more halo; and
wherein Y is —CH₂—, —CHR₅, —C(R₅)₂— wherein each R₅ is independently C₁-C₆ alkyl; —O— or —S—, —S(O), —S(O)₂, —NH—, —NR₁₀—, wherein R₁₀ is C₁-C₆ alkyl;
Wherein Y₁ is optionally present and is —CH₂—, —CHR₅, —C(R₅)₂— wherein each R₅ is independently C₁-C₆ alkyl; —O— or —S—, —S(O)₂, —S(O)₂, —NH—, —NR₁₁—, and wherein R₁₁ is C₁-C₆ alkyl;
or a salt thereof.

38. The compound as claimed in claim 37, wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂, —C₁-C₆—C(═O)—OH or -aryl-C(═O)—OH.

39. The compound as claimed in claim 37 or claim 38, wherein R is selected from —C(═O)—C₁-C₆—C(═O)—OH, —C(═O)—C₁-C₆—C(═O)—NH—S(O)₂—C₁-C₆, —C(═O)—C₁-C₆—C(═O)—NH—S(O)₂—N(C₁-C₆)₂ or -aryl-C(═O)—OH.

40. The compound as claimed in any one of claims 37 to 39, wherein R is —C(═O)—C₁-C₆—C(═O)—OH.

41. The compound as claimed in any one of claims 37 to 40 wherein R is —C(═O)—C₁-C₆—C(═O)—NH—S(O)₂—C₁-C₆.

42. The compound as claimed in any one of claims 37 to 41, wherein R is selected from —C(═O)—C₁-C₆—C(═O)—NH—S(O)₂—N(C₁-C₆)₂ or —C(═O)—C₁-C₆—C(═O)—NH—S(O)₂-aryl, such as —C(═O)—C₁-C₆—C(═O)—NH—S(O)₂-Ph.

43. The compound as claimed in any one of claims 37 to 39, wherein R is -aryl-C(═O)—OH.

44. The compound as claimed in any one of claims 37 to 39, wherein R is —C(═O)—CH₂—CH₂—C(═O)—OH.

45. The compound as claimed in claim 41, wherein R is selected from —C(═O)—CH₂—CH₂—C(═O)—NH—S(O)₂-Me or —C(═O)—CH₂—CH₂—C(═O)—NH—S(O)₂-iPr.

46. The compound as claimed in claim 42, wherein R is —C(═O)—CH₂—CH₂—C(═O)—NH—S(O)₂—NMe₂.

47. The compound as claimed in claim 43, wherein R is -phenyl-C(═O)—OH.

48. The compound as claimed in any one of claims 37 to 47 wherein Q is selected from:

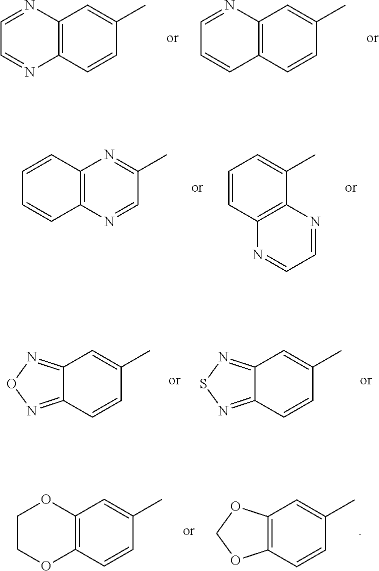

49. The compound as claimed in claim 48, wherein Q is selected from:

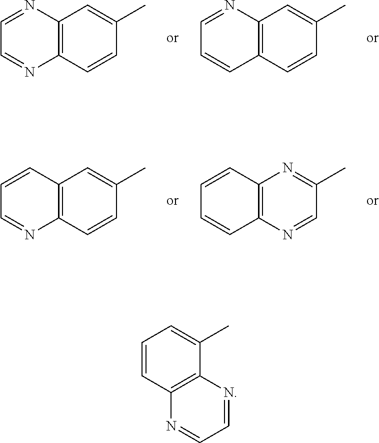

50. The compound as claimed in claim 48 or claim 49, wherein Q is

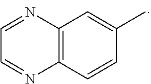

51. The compound as claimed in any one of claims 37 to 50, wherein R₉ is aryl or aryl substituted with C₁-C₆ alkoxy or C₁-C₆ alkoxy further substituted with one or more halo.

52. The compound as claimed in claim 51, wherein aryl is phenyl or phenyl substituted with C₁-C₆ alkoxy. further substituted with one or more halo.

53. The compound as claimed in any one of claims 37 to 52, wherein Y and Y₁ are each —CH₂—.

54. The compound as claimed in any one of claims 37 to 53, wherein the compound of Formula II is selected from 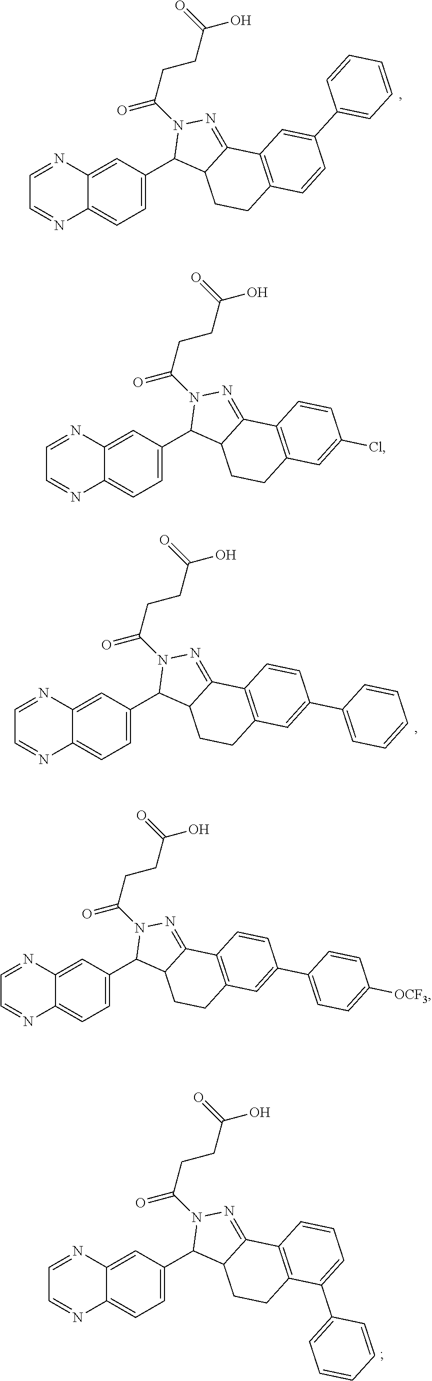 [SMILES: O=C(O)CCC(=O)N1N=C2c3cc(-c4ccccc4)ccc3CCC2C1c1ccc2nccnc2c1 ; O=C(O)CCC(=O)N1N=C2c3ccc(Cl)cc3CCC2C1c1ccc2nccnc2c1 ; O=C(O)CCC(=O)N1N=C2c3ccc(-c4ccccc4)cc3CCC2C1c1ccc2nccnc2c1 ; O=C(O)CCC(=O)N1N=C2c3ccc(-c4ccc(OC(F)(F)F)cc4)cc3CCC2C1c1ccc2nccnc2c1 ; O=C(O)CCC(=O)N1N=C2c3cccc(-c4ccccc4)c3CCC2C1c1ccc2nccnc2c1]
or a salt thereof.

55. The compound of Formula I or Ila, as defined in any one of claims 1 to 54 for reducing the formation of methane from the digestive actions of ruminants and/or for improving ruminant performance.

56. The compound of Formula I or Ila, as defined in any one of claims 1 to 54 for reducing the formation of methane from the digestive actions of ruminants.

57. The compound of Formula I or Ila, as defined in any one of claims 1 to 54 for reducing the formation of methane from the digestive actions of ruminants by at least 10%.

58. The compound of Formula I or Ila, as defined in any one of claims 1 to 54 is for reducing the formation of methane from the digestive actions of ruminants by at least 15%.

59. The compound of Formula I or IIa, as defined in any one of claims 1 to 54 is for reducing the formation of methane from the digestive actions of ruminants by at least 20%.

60. A method for reducing the production of methane emanating from a ruminant and/or for improving ruminant animal performance, comprising administering orally to the ruminant an effective amount of at least one compound of Formula I or Ila, or a salt thereof as defined in any one of claims 1 to 54 to the ruminant.

61. The method as claimed in claim 60, wherein the effective amount of at least one compound of Formula I or Ila, or a salt thereof as defined in any one of claims 1 to 54 is administered at least once-daily to the ruminant.

62. The method as claimed in claim 60 or claim 61, wherein the effective amount of at least one compound of Formula I or Ila, or a salt thereof as defined in any one of claims 1 to 54 reduces the production of methane emanating from the ruminant by at least 10% per day.

63. The method as claimed in any one of claims 48 to 50, wherein the effective amount of at least one compound of Formula I or Ila, or a salt thereof as defined in any one of claims 1 to 54 reduces the production of methane emanating from the ruminant by at least 15% per day.

64. The method as claimed in any one of claims 48 to 51 wherein the effective amount of at least one compound of Formula I, or IIa, or a salt thereof as defined in any one of claims 1 to 54 reduces the production of methane emanating from the ruminant by at least 20% per day.

65. A composition for oral administration comprising at least one compound of Formula I or IIa, or a salt thereof as defined in any one of claims 1 to 54 for reducing the production of methane emanating from a ruminant, and the composition further including at least one agriculturally and orally acceptable excipient.

66. The composition as claimed in claim 65, being adapted for use as a feed additive.

67. The composition as claimed in claim 65, being adapted for use as a water additive.

68. The composition as claimed in claim 65, being adapted for use as a ruminant lick.

69. The composition as claimed in claim 65, being adapted for use as an oral drench.

70. The composition as claimed in claim 65, being adapted for use as a rumen bolus or capsule.

71. The composition as claimed in any one of claims 65 to 70, being adapted to reduce the production of methane emanating from the ruminant by at least 10% per day.

72. The composition as claimed in any one of claims 65 to 71, being adapted to reduce the production of methane emanating from the ruminant by at least 15% per day.

73. The composition as claimed in any one of claims 65 to 72, being to reduce the production of methane emanating from the ruminant by at least 20% per day.

74. The composition as claimed in any one of claims 65 to 73, wherein the excipient may include one or more minerals and/or one or more vitamins.

75. The composition as claimed in any one of claims 65 to 74, wherein the excipient may include one or more vitamins selected from vitamin A, vitamin D3, vitamin E, and vitamin K, e.g. vitamin K3, vitamin B12, biotin and choline, vitamin B1, vitamin B2, vitamin B6, niacin, folic acid or the like.

76. The composition as claimed in any one of claims 65 to 75, wherein the excipient may include one or more minerals selected from calcium, phosphorus, sodium, manganese, zinc, iron, copper, chlorine, sulphur, magnesium, iodine, selenium, and cobalt or the like.

77. The composition as claimed in any one of claims 65 to 76 further including sunflower oil, electrolytes such as ammonium chloride, calcium carbonates, starch, proteins or the like.
